SEQUENCE LISTING (271 sequences)

SEQ ID NO: 1 - protein, 221 aa, Unknown
  Exemplary wild-type canine IgG-A Fc Protein A - C1q - CD16 -
PVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 2 - protein, 220 aa, Unknown
  Exemplary wild-type canine IgG-B Fc Protein A + C1q + CD16 +
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 3 - protein, 237 aa, Unknown
  Exemplary wild-type canine IgG-B Fc with hinge  Protein A + C1q + CD16 +
PKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 4 - protein, 220 aa, Unknown
  Exemplary wild-type canine IgG-C Fc Protein A -  C1q + CD16 +
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 5 - protein, 235 aa, Unknown
  Exemplary wild-type canine IgG-C Fc with hinge   Protein A -  C1q + CD16 +
AKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 6 - protein, 221 aa, Unknown
  Exemplary wild-type canine IgG-D Fc Protein A - C1q - CD16 -
PVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 7 - protein, 221 aa, Artificial Sequence
  Synthetic Exemplary variant canine IgG-A Fc C1q - Protein A + I(21)T R(23)L T(25)A E(80)G T(205)A Q(207)H
PVPEPLGGPSVLIFPPKPKDTLLIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 8 - protein, 220 aa, Artificial Sequence
  Synthetic Exemplary variant canine IgG-C Fc C1q + Protein A + I(21)T V(23)L T(24)I
PGCGLLGGPSVFIFPPKPKDTLLIARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 9 - protein, 221 aa, Artificial Sequence
  Synthetic Exemplary variant canine IgG-D Fc C1q - Protein A + I(21)T R(23)L T(25)A E(80)G Q(207)H
PVPESLGGPSVFIFPPKPKDTLLIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 10 - protein, 221 aa, Artificial Sequence
  Synthetic Exemplary variant canine IgG-A Fc C1q - Protein A + I(21)T Q(207)H
PVPEPLGGPSVLIFPPKPKDTLRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLHNHYTDLSLSHSPGK

SEQ ID NO: 11 - protein, 220 aa, Artificial Sequence
  Synthetic Exemplary variant canine IgG-C Fc C1q + Protein A + I(21)T
PGCGLLGGPSVFIFPPKPKDTLVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 12 - protein, 221 aa, Artificial Sequence
  Synthetic Exemplary variant canine IgG-D Fc C1q - Protein A + I(21)T Q(207)H
PVPESLGGPSVFIFPPKPKDTLRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 13 - protein, 220 aa, Artificial Sequence
  Synthetic Exemplary variant canine IgG-B Fc Protein A + C1q - K(93)R
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 14 - protein, 220 aa, Artificial Sequence
  Synthetic Exemplary variant canine IgG-C Fc Protein A - C1q - CD16 + K(93)R
PGCGLLGGPSVFIFPPKPKDILVTARTPTVTCVVVDLDPENPEVQISWFVDSKQVQTANTQPREEQSNGTYRVVSVLPIGHQDWLSGKQFKCRVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 15 - protein, 220 aa, Artificial Sequence
  Synthetic Exemplary variant canine IgG-B Fc Protein A + C1q + CD16 -  M(5)P
PAPEPLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 16 - protein, 220 aa, Artificial Sequence
  Synthetic Exemplary variant canine IgG-B Fc Protein A + C1q + CD16 -  P(39)R
PAPEMLGGPSVFIFPPKPKDTLLIARTPEVTCVVVDLDREDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 17 - protein, 220 aa, Artificial Sequence
  Synthetic Exemplary variant canine IgG-B Fc Protein A + C1q + CD16 -  D(38)G
PAPEMLGGPSVFIFPPKPKDTLLIARTPEVTCVVVDLGPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 18 - protein, 220 aa, Artificial Sequence
  Synthetic Exemplary variant canine IgG-B Fc Protein A + C1q + CD16 -  D(38)G P(39)R
PAPEMLGGPSVFIFPPKPKDTLLIARTPEVTCVVVDLGREDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 19 - protein, 220 aa, Artificial Sequence
  Synthetic Exemplary variant canine IgG-B Fc Protein A + C1q + CD16 -  K(97)I
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNIALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 20 - protein, 220 aa, Artificial Sequence
  Synthetic Exemplary variant canine IgG-B Fc Protein A + C1q + CD16 -  A(98)G
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 21 - protein, 220 aa, Artificial Sequence
  Synthetic Exemplary variant canine IgG-B Fc Protein A + C1q + CD16 - D(38)G  K(97)I A(98)G
PAPEMLGGPSVFIFPPKPKDTLLIARTPEVTCVVVDLGPEDPEVQISWFVDGKQMQTAKTQPREEQFNGTYRVVSVLPIGHQDWLKGKQFTCKVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 22 - protein, 220 aa, Artificial Sequence
  Synthetic Exemplary variant canine IgG-B Fc Protein A + C1q + CD16 -  M(5)P P(39)R
PAPEPLGGPSVFIFPPKPKDTLLIARTPEVTCVVVDLDREDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 23 - protein, 220 aa, Artificial Sequence
  Synthetic Exemplary variant canine IgG-C Fc Protein A - C1q + CD16 -  L(5)P
PGCGPLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 24 - protein, 220 aa, Artificial Sequence
  Synthetic Exemplary variant canine IgG-C Fc Protein A - C1q + CD16 -  P(39)R
PGCGLLGGPSVFIFPPKPKDILVTARTPTVTCVVVDLDRENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 25 - protein, 220 aa, Artificial Sequence
  Synthetic Exemplary variant canine IgG-C Fc Protein A - C1q + CD16 -  D(38)G
PGCGLLGGPSVFIFPPKPKDILVTARTPTVTCVVVDLGPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 26 - protein, 220 aa, Artificial Sequence
  Synthetic Exemplary variant canine IgG-C Fc Protein A - C1q + CD16 -  K(97)I
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNIALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 27 - protein, 220 aa, Artificial Sequence
  Synthetic Exemplary variant canine IgG-C Fc Protein A - C1q + CD16 -  A(98)G
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKGLPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 28 - protein, 220 aa, Artificial Sequence
  Synthetic Exemplary variant canine IgG-C Fc Protein A - C1q + CD16 -  L(5)P P(39)R
PGCGPLGGPSVFIFPPKPKDILVTARTPTVTCVVVDLDRENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 29 - protein, 220 aa, Artificial Sequence
  Synthetic Exemplary variant canine IgG-C Fc Protein A - C1q + CD16 - D(38)G  K(97)I A(98)G
PGCGLLGGPSVFIFPPKPKDILVTARTPTVTCVVVDLGPENPEVQISWFVDSKQVQTANTQPREEQSNGTYRVVSVLPIGHQDWLSGKQFKCKVNNIGLPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 30 - protein, 220 aa, Artificial Sequence
  Synthetic Exemplary variant canine IgG-C Fc C1q - K(93)R Protein A + I(21)T V(23)L T(24)I
PGCGLLGGPSVFIFPPKPKDTLLIARTPTVTCVVVDLDPENPEVQISWFVDSKQVQTANTQPREEQSNGTYRVVSVLPIGHQDWLSGKQFKCRVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 31 - protein, 220 aa, Artificial Sequence
  Synthetic Exemplary variant canine IgG-B Fc Protein A +  C1q - CD16 - D(38)G  K(93)R K(97)I A(98)G
PAPEMLGGPSVFIFPPKPKDTLLIARTPEVTCVVVDLGPEDPEVQISWFVDGKQMQTAKTQPREEQFNGTYRVVSVLPIGHQDWLKGKQFTCRVNNIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 32 - protein, 220 aa, Artificial Sequence
  Synthetic Exemplary variant canine IgG-B Fc Protein A + C1q - CD16 - M(5)P P(39)R K(93)R
PAPEPLGGPSVFIFPPKPKDTLLIARTPEVTCVVVDLDREDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 33 - protein, 220 aa, Artificial Sequence
  Synthetic Exemplary variant canine IgG-C Fc Protein A - C1q - CD16 - D(38)G  K(93)R K(97)I A(98)G
PGCGLLGGPSVFIFPPKPKDILVTARTPTVTCVVVDLGPENPEVQISWFVDSKQVQTANTQPREEQSNGTYRVVSVLPIGHQDWLSGKQFKCRVNNIGLPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 34 - protein, 220 aa, Artificial Sequence
  Synthetic Exemplary variant canine IgG-C Fc Protein A - C1q - CD16 - M(5)P P(39)R K(93)R
PGCGPLGGPSVFIFPPKPKDILVTARTPTVTCVVVDLDRENPEVQISWFVDSKQVQTANTQPREEQSNGTYRVVSVLPIGHQDWLSGKQFKCRVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 35 - protein, 221 aa, Artificial Sequence
  Synthetic Exemplary variant canine IgG-A Fc Protein A - C1q + CD16 - R(93)K
PVPEPLGGPSVLIFPPKPKDILRITRTPEVTCVVLDLGREDPEVQISWFVDGKEVHTAKTQSREQQFNGTYRVVSVLPIEHQDWLTGKEFKCKVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 36 - protein, 221 aa, Artificial Sequence
  Synthetic Exemplary variant canine IgG-A Fc Protein A - C1q + CD16 + V(2)A P(5)M L(35)V G(38)D R(39)P Q(65) E H(96)N I(97)K D(98)A
PAPEMLGGPSVLIFPPKPKDILRITRTPEVTCVVVDLDPEDPEVQISWFVDGKEVHTAKTQSREEQFNGTYRVVSVLPIEHQDWLTGKEFKCRVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 37 - protein, 221 aa, Artificial Sequence
  Synthetic Exemplary variant canine IgG-A Fc Protein A - C1q - CD16 + V(2)A P(5)L L(35)V G(38)D R(39)P Q(65) E H(96)N I(97)K D(98)A
PAPELLGGPSVLIFPPKPKDILRITRTPEVTCVVVDLDPEDPEVQISWFVDGKEVHTAKTQSREEQFNGTYRVVSVLPIEHQDWLTGKEFKCRVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 38 - protein, 221 aa, Artificial Sequence
  Synthetic Exemplary variant canine IgG-A Fc Protein A - C1q + CD16 + V(2)A P(5)M L(35)V G(38)D R(39)P Q(65)E R (93)K H(96)N I(97)K D(98)A
PAPEMLGGPSVLIFPPKPKDILRITRTPEVTCVVVDLDPEDPEVQISWFVDGKEVHTAKTQSREEQFNGTYRVVSVLPIEHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 39 - protein, 221 aa, Artificial Sequence
  Synthetic Exemplary variant canine IgG-A Fc Protein A - C1q + CD16 + V(2)A P(5)L L(35)V G(38)D R(39)P Q(65)E R (93)K H(96)N I(97)K D(98)A
PAPELLGGPSVLIFPPKPKDILRITRTPEVTCVVVDLDPEDPEVQISWFVDGKEVHTAKTQSREEQFNGTYRVVSVLPIEHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 40 - protein, 221 aa, Artificial Sequence
  Synthetic Exemplary variant canine IgG-A Fc Protein A + C1q + CD16 + V(2)A P(5)M I(21)T R(23)L T(25)A L(35)V G (38)D R(39)P Q(65)E E(80)G R(93)K H(96)N I(97)K D(98)A T(205) A Q(207)H
PAPEMLGGPSVLIFPPKPKDTLLIARTPEVTCVVVDLDPEDPEVQISWFVDGKEVHTAKTQSREEQFNGTYRVVSVLPIGHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 41 - protein, 221 aa, Artificial Sequence
  Synthetic Exemplary variant canine IgG-A Fc Protein A + C1q + CD16 + V(2)A P(5)L I(21)T R(23)L T(25)A L(35)V G (38)D R(39)P Q(65)E E(80)G R(93)K H(96)N I(97)K D(98)A T(205) A Q(207)H
PAPELLGGPSVLIFPPKPKDTLLIARTPEVTCVVVDLDPEDPEVQISWFVDGKEVHTAKTQSREEQFNGTYRVVSVLPIGHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 42 - protein, 221 aa, Artificial Sequence
  Synthetic Exemplary variant canine IgG-D Fc Protein A - C1q + CD16 - R(93)K
PVPESLGGPSVFIFPPKPKDILRITRTPEITCVVLDLGREDPEVQISWFVDGKEVHTAKTQPREQQFNSTYRVVSVLPIEHQDWLTGKEFKCKVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 43 - protein, 221 aa, Artificial Sequence
  Synthetic Exemplary variant canine IgG-D Fc Protein A - C1q - CD16 + V(2)A S(5)M L(35)V G(38)D R(39)P Q(65) E H(96)N I(97)K G(98)A
PAPEMLGGPSVFIFPPKPKDILRITRTPEITCVVVDLDPEDPEVQISWFVDGKEVHTAKTQPREEQFNSTYRVVSVLPIEHQDWLTGKEFKCRVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 44 - protein, 221 aa, Artificial Sequence
  Synthetic Exemplary variant canine IgG-D Fc Protein A - C1q - CD16 + V(2)A S(5)L L(35)V G(38)D R(39)P Q(65) E H(96)N I(97)K G(98)A
PAPELLGGPSVFIFPPKPKDILRITRTPEITCVVVDLDPEDPEVQISWFVDGKEVHTAKTQPREEQFNSTYRVVSVLPIEHQDWLTGKEFKCRVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 45 - protein, 221 aa, Artificial Sequence
  Synthetic Exemplary variant canine IgG-D Fc Protein A - C1q + CD16 + V(2)A S(5)M L(35)V G(38)D R(39)P Q(65) E R(93)K H(96)N I(97)K G(98)A
PAPEMLGGPSVFIFPPKPKDILRITRTPEITCVVVDLDPEDPEVQISWFVDGKEVHTAKTQPREEQFNSTYRVVSVLPIEHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 46 - protein, 221 aa, Artificial Sequence
  Synthetic Exemplary variant canine IgG-D Fc Protein A - C1q + CD16 + V(2)A S(5)L L(35)V G(38)D R(39)P Q(65) E R(93)K H(96)N I(97)K G(98)A
PAPELLGGPSVFIFPPKPKDILRITRTPEITCVVVDLDPEDPEVQISWFVDGKEVHTAKTQPREEQFNSTYRVVSVLPIEHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 47 - protein, 221 aa, Artificial Sequence
  Synthetic Exemplary variant canine IgG-D Fc Protein A + C1q + CD16 + V(2)A S(5)M I(21)T R(23)L T(25)A L(35)V G (38)D R(39)P Q(65)E E(80)G R(93)K H(96)N I(97)K G(98)A Q(207)H
PAPEMLGGPSVFIFPPKPKDTLLIARTPEITCVVVDLDPEDPEVQISWFVDGKEVHTAKTQPREEQFNSTYRVVSVLPIGHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 48 - protein, 221 aa, Artificial Sequence
  Synthetic Exemplary variant canine IgG-D Fc Protein A + C1q + CD16 + V(2)A S(5)L I(21)T R(23)L T(25)A L(35)V G (38)D R(39)P Q(65)E E(80)G R(93)K H(96)N I(97)K G(98)A Q(207)H
PAPELLGGPSVFIFPPKPKDTLLIARTPEITCVVVDLDPEDPEVQISWFVDGKEVHTAKTQPREEQFNSTYRVVSVLPIGHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 49 - protein, 214 aa, Unknown
  Exemplary wild-type equine IgG1 Fc Protein A + C1q +
GGPSVFLFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDESKKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 50 - protein, 214 aa, Unknown
  Exemplary wild-type equine IgG2 Fc Protein A - C1q -
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 51 - protein, 244 aa, Unknown
  Exemplary wild-type equine IgG2 Fc with hinge Protein A - C1q -
PPCVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 52 - protein, 214 aa, Unknown
  Exemplary wild-type equine IgG3 Fc Protein A + C1q+
GGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFYPPDITVEWQSNEHPEPEGKYRTTEAQKDSDGSYFLYSKLTVEKDRWQQGTTFTCVVMHEALHNHVMQKNISKNPGK

SEQ ID NO: 53 - protein, 214 aa, Unknown
  Exemplary wild-type equine IgG4 Fc Protein A + C1q +
VGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISAPTGQPREPQVYVLAPHRDELSKNKVSVTCLVKDFYPPDIDIEWKSNGQPEPETKYSTTPAQLDSDGSYFLYSKLTVETNRWQQGTTFTCAVMHEALHNHYTEKSVSKSPGK

SEQ ID NO: 54 - protein, 214 aa, Unknown
  Exemplary wild-type equine IgG5 Fc Protein A - C1q -
GGPSVFIFPPKPKDVLMISRKPEVTCVVVDLGHDDPDVQFTWFVDGVETHTATTEPKEEQFNSTYRVVSVLPIQHQDWLSGKEFKCSVTSKALPAPVERTISKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 55 - protein, 214 aa, Unknown
  Exemplary wild-type equine IgG6 Fc Protein A - C1q -
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPQVYILAPHPDEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 56 - protein, 214 aa, Unknown
  Exemplary wild-type equine IgG7 Fc Protein A + C1q +
VGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHPDELSKNKVSVTCLVKDFYPPDIDIEWKSNGQPEPETKYSTTPAQLDGDGSYFLYSKLTVETNRWQQGTTFTCAVMHEALHNHYTEKSVSKSPGK

SEQ ID NO: 57 - protein, 214 aa, Artificial Sequence
  Synthetic Exemplary variant equine IgG2 Fc C1q - Protein A + F(203)Y
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQGESFTCAVMHEALHNHYTKTDISESLGK

SEQ ID NO: 58 - protein, 214 aa, Artificial Sequence
  Synthetic Exemplary variant equine IgG2 Fc C1q - Protein A + A(15)T F(203)Y
GGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 59 - protein, 214 aa, Artificial Sequence
  Synthetic Exemplary variant equine IgG5 Fc C1q - Protein A + V(199)L E(200)H
GGPSVFIFPPKPKDVLMISRKPEVTCVVVDLGHDDPDVQFTWFVDGVETHTATTEPKEEQFNSTYRVVSVLPIQHQDWLSGKEFKCSVTSKALPAPVERTISKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEALHNHYTQKNVSHSPGK

SEQ ID NO: 60 - protein, 214 aa, Artificial Sequence
  Synthetic Exemplary variant equine IgG6 Fc C1q - Protein A + I(199)L  R(200)H H(201)N T(202)H
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPQVYILAPHPDEVTKNTVSVTCLVKDFYPPDINVEWQSNEEPEPEVKYSTTPAQLDGDGSYFLYSKLTVETDRWEQGESFTCVVMHEALHNHYRQKSITNFPGK

SEQ ID NO: 61 - protein, 214 aa, Artificial Sequence
  Synthetic Exemplary variant equine IgG1 Fc Protein A + C1q - K(87)S
GGPSVFLFPPNPKDTLMITRTPEVTCVVVDVSQENPDVKFNWYMDGVEVRTATTRPKEEQFNSTYRVVSVLRIQHQDWLSGKEFKCSVNNQALPQPIERTITKTKGRSQEPQVYVLAPHPDESKKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 62 - protein, 214 aa, Artificial Sequence
  Synthetic Exemplary variant equine IgG3 Fc Protein A + C1q - K(87)S
GGPSVFIFPPKPKDVLMITRMPEVTCLVVDVSHDSSDVLFTWYVDGTEVKTAKTMPNEEQNNSTYRVVSVLRIQHQDWLNGKKFKCSVNNQALPAPVERTISKATGQTRVPQVYVLAPHPDELSKNKVSVTCLVKDFYPPDITVEWQSNEHPEPEGKYRTTEAQKDSDGSYFLYSKLTVEKDRWQQGTTFTCVVMHEALHNHVMQKNISKNPGK

SEQ ID NO: 63 - protein, 214 aa, Artificial Sequence
  Synthetic Exemplary variant equine IgG4 Fc Protein A + C1q - K(87)S
VGPSVFIFPPKPKDVLMISRTPTVTCVVVDVGHDFPDVQFNWYVDGVETHTATTEPKQEQFNSTYRVVSVLPIQHKDWLSGKEFKCSVNNKALPAPVERTISAPTGQPREPQVYVLAPHRDELSKNKVSVTCLVKDFYPPDIDIEWKSNGQPEPETKYSTTPAQLDSDGSYFLYSKLTVETNRWQQGTTFTCAVMHEALHNHYTEKSVSKSPGK

SEQ ID NO: 64 - protein, 214 aa, Artificial Sequence
  Synthetic Exemplary variant equine IgG7 Fc Protein A + C1q - K(87)S
VGPSVFIFPPKPKDVLMISRTPTVTCVVVDVGHDFPDVQFNWYVDGVETHTATTEPKQEQNNSTYRVVSILAIQHKDWLSGKEFKCSVNNQALPAPVQKTISKPTGQPREPQVYVLAPHPDELSKNKVSVTCLVKDFYPPDIDIEWKSNGQPEPETKYSTTPAQLDGDGSYFLYSKLTVETNRWQQGTTFTCAVMHEALHNHYTEKSVSKSPGK

SEQ ID NO: 65 - protein, 237 aa, Unknown
  Exemplary wild-type feline IgG1a Fc Protein A + C1q +
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 66 - protein, 237 aa, Unknown
  Exemplary wild-type feline IgG1a Fc Protein A + C1q +
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 67 - protein, 237 aa, Unknown
  Exemplary wild-type feline IgG1b Fc Protein A + C1q +
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 68 - protein, 237 aa, Unknown
  Exemplary wild-type feline IgG1b Fc Protein A + C1q +
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 69 - protein, 237 aa, Unknown
  Exemplary wild-type feline IgG2 Fc Protein A + C1q -
PKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 70 - protein, 237 aa, Artificial Sequence; identical to SEQ ID NO: 66
  Synthetic Exemplary variant feline IgG1a Fc Protein A + C1q - P(198)A

SEQ ID NO: 71 - protein, 237 aa, Artificial Sequence; identical to SEQ ID NO: 65
  Synthetic Exemplary variant feline IgG1a Fc Protein A + C1q - P(198)A

SEQ ID NO: 72 - protein, 237 aa, Artificial Sequence; identical to SEQ ID NO: 68
  Synthetic Exemplary variant feline IgG1b Fc Protein A + C1q - P(198)A

SEQ ID NO: 73 - protein, 237 aa, Artificial Sequence; identical to SEQ ID NO: 67
  Synthetic Exemplary variant feline IgG1b Fc Protein A + C1q - P(198)A

SEQ ID NO: 74 - protein, 221 aa, Artificial Sequence
  Synthetic Exemplary variant canine IgG-A Fc Heterodimer chain 1  T(138)Y
PVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSIYCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 75 - protein, 220 aa, Artificial Sequence
  Synthetic Exemplary variant canine IgG-B Fc Heterodimer chain 1  T(137)Y
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLYCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 76 - protein, 220 aa, Artificial Sequence
  Synthetic Exemplary variant canine IgG-C Fc Heterodimer chain 1  T(137)Y
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLYCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 77 - protein, 221 aa, Artificial Sequence
  Synthetic Exemplary variant canine IgG-D Fc Heterodimer chain 1  T(138)Y
PVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLYCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 78 - protein, 221 aa, Artificial Sequence
  Synthetic Exemplary variant canine IgG-A Fc Heterodimer chain 1 T(138)W
PVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSIWCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 79 - protein, 220 aa, Artificial Sequence
  Synthetic Exemplary variant canine IgG-B Fc Heterodimer chain 1 T(137)W
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLWCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 80 - protein, 220 aa, Artificial Sequence
  Synthetic Exemplary variant canine IgG-C Fc Heterodimer chain 1 T(137)W
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLWCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 81 - protein, 221 aa, Artificial Sequence
  Synthetic Exemplary variant canine IgG-D Fc Heterodimer chain 1 T(138)W
PVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLWCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 82 - protein, 221 aa, Artificial Sequence
  Synthetic Exemplary variant canine IgG-A Fc Heterodimer chain 2 Y(181)T
PVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLTSKLSVDKSRWQQGDPFTCAVMHETLQNHYTDLSLSHSPGK

SEQ ID NO: 83 - protein, 220 aa, Artificial Sequence
  Synthetic Exemplary variant canine IgG-B Fc Heterodimer chain 2 Y(180)T
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLTSKLSVDKSRWQRGDTFICAVMHEALHNHYTQESLSHSPGK

SEQ ID NO: 84 - protein, 220 aa, Artificial Sequence
  Synthetic Exemplary variant canine IgG-C Fc Heterodimer chain 2 Y(180)T
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLTSKLSVDKSRWQRGDTFICAVMHEALHNHYTQISLSHSPGK

SEQ ID NO: 85 - protein, 221 aa, Artificial Sequence
  Synthetic Exemplary variant canine IgG-D Fc Heterodimer chain 2 Y(181)T
PVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLTSKLSVDKSRWQQGDTFTCAVMHEALQNHYTDLSLSHSPGK

SEQ ID NO: 86 - protein, 221 aa, Artificial Sequence
  Synthetic Exemplary variant canine IgG-A Fc Heterodimer chain 2 T(138)S L(140)A Y(181)T
PVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSISCAIKDFYPPDIDVEWQSNGQQEPERKHRMTPPQLDEDGSYFLTSKLSVDKSRWQQGDPFTCAVMHETLQNHYTDLSLSHSPGK

SEQ ID NO: 87 - protein, 220 aa, Artificial Sequence
  Synthetic Exemplary variant canine IgG-B Fc Heterodimer chain 2 T(137)S L(139)A Y(180)T
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLSCAIKDFFPPDIDVEWQSNGQQEPESKYRTTPPQLDEDGSYFLTSKLSVDKSRWQRGDTFICAVMHEALHNHYTQESLSHSPGK

SEQ ID NO: 88 - protein, 220 aa, Artificial Sequence
  Synthetic Exemplary variant canine IgG-C Fc Heterodimer chain 2 T(137)S L(139)A Y(180)T
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLSCAVKDFFPPEIDVEWQSNGQQEPESKYRMTPPQLDEDGSYFLTSKLSVDKSRWQRGDTFICAVMHEALHNHYTQISLSHSPGK

SEQ ID NO: 89 - protein, 221 aa, Artificial Sequence
  Synthetic Exemplary variant canine IgG-D Fc Heterodimer chain 2 T(138)S L(140)A Y(181)T
PVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLSCAIKDFFPPEIDVEWQSNGQPEPESKYHTTAPQLDEDGSYFLTSKLSVDKSRWQQGDTFTCAVMHEALQNHYTDLSLSHSPGK

SEQ ID NO: 90 - protein, 221 aa, Artificial Sequence
  Synthetic Exemplary variant canine IgG-A Fc Heterodimer chain 2 T(138)S L(140)A
PVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSISCAIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 91 - protein, 220 aa, Artificial Sequence
  Synthetic Exemplary variant canine IgG-B Fc Heterodimer chain 2 T(137)S L(139)A
PAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLSCAIKDFFPPDIDVEWQSNGQQEPESKYRTTPPQLDEDGSYFLYSKLSVDKSRWQRGDTFICAVMHEALHNHYTQESLSHSPGK

SEQ ID NO: 92 - protein, 220 aa, Artificial Sequence
  Synthetic Exemplary variant canine IgG-C Fc Heterodimer chain 2 T(137)S L(139)A
PGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLSCAVKDFFPPEIDVEWQSNGQQEPESKYRMTPPQLDEDGSYFLYSKLSVDKSRWQRGDTFICAVMHEALHNHYTQISLSHSPGK

SEQ ID NO: 93 - protein, 221 aa, Artificial Sequence
  Synthetic Exemplary variant canine IgG-D Fc Heterodimer chain 2 T(138)S L(140)A
PVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLSCAIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 94 - protein, 237 aa, Artificial Sequence
  Synthetic Exemplary variant feline IgG1a Fc Heterodimer chain 1 T(154)Y
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVYCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 95 - protein, 237 aa, Artificial Sequence
  Synthetic Exemplary variant feline IgG1a Fc Heterodimer chain 1 T(154)Y
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVYCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 96 - protein, 237 aa, Artificial Sequence
  Synthetic Exemplary variant feline IgG1b Fc Heterodimer chain 1 T(154)Y
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVYCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 97 - protein, 237 aa, Artificial Sequence
  Synthetic Exemplary variant feline IgG1b Fc Heterodimer chain 1 T(154)Y
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVYCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 98 - protein, 237 aa, Artificial Sequence
  Synthetic Exemplary variant feline IgG2 Fc Heterodimer chain 1 T(154)W
PKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVWCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 99 - protein, 237 aa, Artificial Sequence
  Synthetic Exemplary variant feline IgG1a Fc Heterodimer chain 1 T(154)W
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVWCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 100 - protein, 237 aa, Artificial Sequence
  Synthetic Exemplary variant feline IgG1a Fc Heterodimer chain 1 T(154)W
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVWCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 101 - protein, 237 aa, Artificial Sequence
  Synthetic Exemplary variant feline IgG1b Fc Heterodimer chain 1 T(154)W
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVWCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 102 - protein, 237 aa, Artificial Sequence
  Synthetic Exemplary variant feline IgG1b Fc Heterodimer chain 1 T(154)W
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVWCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 103 - protein, 237 aa, Artificial Sequence; identical to SEQ ID NO: 98
  Synthetic Exemplary variant feline IgG2 Fc Heterodimer chain 1 T(154)W

SEQ ID NO: 104 - protein, 237 aa, Artificial Sequence
  Synthetic Exemplary variant feline IgG1a Fc Heterodimer chain 2 T(154)S L(156)A
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVSCAIKSFHPPDIAVEWEITGQPEPENNYRTTPPQLDSDGTYFVYSKLSVDRSHWQRGNTYTCSVSHEALHSHHTQKSLTQSPGK

SEQ ID NO: 105 - protein, 237 aa, Artificial Sequence
  Synthetic Exemplary variant feline IgG1a Fc Heterodimer chain 2 T(154)S L(156)A Y(197)T
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVSCAIKSFHPPDIAVEWEITGQPEPENNYRTTPPQLDSDGTYFVTSKLSVDRSHWQRGNTYTCSVSHEALHSHHTQKSLTQSPGK

SEQ ID NO: 106 - protein, 237 aa, Artificial Sequence
  Synthetic Exemplary variant feline IgG1a Fc Heterodimer chain 2 T(154)S L(156)A
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVSCAIKSFHPPDIAVEWEITGQPEPENNYRTTPPQLDSDGTYFVYSKLSVDRSHWQRGNTYTCSVSHEALHSHHTQKSLTQSPGK

SEQ ID NO: 107 - protein, 237 aa, Artificial Sequence
  Synthetic Exemplary variant feline IgG1a Fc Heterodimer chain 2 T(154)S L(156)A Y(197)T
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVSCAIKSFHPPDIAVEWEITGQPEPENNYRTTPPQLDSDGTYFVTSKLSVDRSHWQRGNTYTCSVSHEALHSHHTQKSLTQSPGK

SEQ ID NO: 108 - protein, 237 aa, Artificial Sequence
  Synthetic Exemplary variant feline IgG1b Fc Heterodimer chain 2 T(154)S L(156)A
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVSCAIEGFYPSDIAVEWEITGQPEPENNYRTTPPQLDSDGTYFLYSRLSVDRSRWQRGNTYTCSVSHEALHSHHTQKSLTQSPGK

SEQ ID NO: 109 - protein, 237 aa, Artificial Sequence
  Synthetic Exemplary variant feline IgG1b Fc Heterodimer chain 2 T(154)S L(156)A Y(197)T
RKTDHPPGPKTGEGPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVSCAIEGFYPSDIAVEWEITGQPEPENNYRTTPPQLDSDGTYFLTSRLSVDRSRWQRGNTYTCSVSHEALHSHHTQKSLTQSPGK

SEQ ID NO: 110 - protein, 237 aa, Artificial Sequence
  Synthetic Exemplary variant feline IgG1b Fc Heterodimer chain 2 T(154)S L(156)A
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVSCAIEGFYPSDIAVEWEITGQPEPENNYRTTPPQLDSDGTYFLYSRLSVDRSRWQRGNTYTCSVSHEALHSHHTQKSLTQSPGK

SEQ ID NO: 111 - protein, 237 aa, Artificial Sequence
  Synthetic Exemplary variant feline IgG1b Fc Heterodimer chain 2 T(154)S L(156)A Y(197)T
RKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVSCAIEGFYPSDIAVEWEITGQPEPENNYRTTPPQLDSDGTYFLTSRLSVDRSRWQRGNTYTCSVSHEALHSHHTQKSLTQSPGK

SEQ ID NO: 112 - protein, 237 aa, Artificial Sequence
  Synthetic Exemplary variant feline IgG2 Fc Heterodimer chain 2 T(154)S L(156)A
PKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVSCAIKGFHPPDIAVEWEITGQPEPENNYQTTPPQLDSDGTYFLYSRLSVDRSHWQRGNTYTCSVSHEALHSHHTQKSLTQSPGK

SEQ ID NO: 113 - protein, 237 aa, Artificial Sequence
  Synthetic Exemplary variant feline IgG2 Fc Heterodimer chain 2 T(154)S L(156)A Y(197)T
PKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVSCAIKGFHPPDIAVEWEITGQPEPENNYQTTPPQLDSDGTYFLTSRLSVDRSHWQRGNTYTCSVSHEALHSHHTQKSLTQSPGK

SEQ ID NO: 114 - protein, 214 aa, Artificial Sequence
  Synthetic Exemplary variant equine IgG1 Fc Heterodimer chain 1 T(131)Y
GGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVYCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 115 - protein, 214 aa, Artificial Sequence
  Synthetic Exemplary variant equine IgG2 Fc Heterodimer chain 1 T(131)Y
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVYCLVKDFYPPDISVEWQSNRWPELEGKYSTTPAQLDGDGSYFLYSKLSLETSRWQQVESFTCAVMHEALHNHFTKTDISESLGK

SEQ ID NO: 116 - protein, 214 aa, Artificial Sequence
  Synthetic Exemplary variant equine IgG3 Fc Heterodimer chain 1 T(131)Y
GGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVYCLVKDFYPPDITVEWQSNEHPEPEGKYRTTEAQKDSDGSYFLYSKLTVEKDRWQQGTTFTCVVMHEALHNHVMQKNISKNPGK

SEQ ID NO: 117 - protein, 214 aa, Artificial Sequence
  Synthetic Exemplary variant equine IgG4 Fc Heterodimer chain 1 T(131)Y
VGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISAPTGQPREPQVYVLAPHRDELSKNKVSVYCLVKDFYPPDIDIEWKSNGQPEPETKYSTTPAQLDSDGSYFLYSKLTVETNRWQQGTTFTCAVMHEALHNHYTEKSVSKSPGK

SEQ ID NO: 118 - protein, 214 aa, Artificial Sequence
  Synthetic Exemplary variant equine IgG5 Fc Heterodimer chain 1 T(131)Y
GGPSVFIFPPKPKDVLMISRKPEVTCVVVDLGHDDPDVQFTWFVDGVETHTATTEPKEEQFNSTYRVVSVLPIQHQDWLSGKEFKCSVTSKALPAPVERTISKAKGQLRVPQVYVLAPHPDELAKNTVSVYCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 119 - protein, 214 aa, Artificial Sequence
  Synthetic Exemplary variant equine IgG6 Fc Heterodimer chain 1 T(131)Y
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVYCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 120 - protein, 214 aa, Artificial Sequence
  Synthetic Exemplary variant equine IgG7 Fc Heterodimer chain 1 T(131)Y
VGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHPDELSKNKVSVYCLVKDFYPPDIDIEWKSNGQPEPETKYSTTPAQLDGDGSYFLYSKLTVETNRWQQGTTFTCAVMHEALHNHYTEKSVSKSPGK

SEQ ID NO: 121 - protein, 214 aa, Artificial Sequence
  Synthetic Exemplary variant equine IgG1 Fc Heterodimer chain 1 T(131)W
GGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVWCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 122 - protein, 214 aa, Artificial Sequence
  Synthetic Exemplary variant equine IgG2 Fc Heterodimer chain 1 T(131)W
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVWCLVKDFYPPDISVEWQSNRWPELEGKYSTTPAQLDGDGSYFLYSKLSLETSRWQQVESFTCAVMHEALHNHFTKTDISESLGK

SEQ ID NO: 123 - protein, 214 aa, Artificial Sequence
  Synthetic Exemplary variant equine IgG3 Fc Heterodimer chain 1 T(131)W
GGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVWCLVKDFYPPDITVEWQSNEHPEPEGKYRTTEAQKDSDGSYFLYSKLTVEKDRWQQGTTFTCVVMHEALHNHVMQKNISKNPGK

SEQ ID NO: 124 - protein, 214 aa, Artificial Sequence
  Synthetic Exemplary variant equine IgG4 Fc Heterodimer chain 1 T(131)W
VGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISAPTGQPREPQVYVLAPHRDELSKNKVSVWCLVKDFYPPDIDIEWKSNGQPEPETKYSTTPAQLDSDGSYFLYSKLTVETNRWQQGTTFTCAVMHEALHNHYTEKSVSKSPGK

SEQ ID NO: 125 - protein, 214 aa, Artificial Sequence
  Synthetic Exemplary variant equine IgG5 Fc Heterodimer chain 1 T(131)W
GGPSVFIFPPKPKDVLMISRKPEVTCVVVDLGHDDPDVQFTWFVDGVETHTATTEPKEEQFNSTYRVVSVLPIQHQDWLSGKEFKCSVTSKALPAPVERTISKAKGQLRVPQVYVLAPHPDELAKNTVSVWCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 126 - protein, 214 aa, Artificial Sequence
  Synthetic Exemplary variant equine IgG6 Fc Heterodimer chain 1 T(131)W
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVWCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 127 - protein, 214 aa, Artificial Sequence
  Synthetic Exemplary variant equine IgG7 Fc Heterodimer chain 1 T(131)W
VGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHPDELSKNKVSVWCLVKDFYPPDIDIEWKSNGQPEPETKYSTTPAQLDGDGSYFLYSKLTVETNRWQQGTTFTCAVMHEALHNHYTEKSVSKSPGK

SEQ ID NO: 128 - protein, 214 aa, Artificial Sequence
  Synthetic Exemplary variant equine IgG1 Fc Heterodimer chain 2 T(131)S L(133)A
GGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVSCAVKDFYPPEINIEWQSNGQPELETKYSTTQAQQDSDGSYFLYSKLSVDRNRWQQGTTFTCGVMHEALHNHYTQKNVSKNPGK

SEQ ID NO: 129 - protein, 214 aa, Artificial Sequence
  Synthetic Exemplary variant equine IgG2 Fc Heterodimer chain 2 T(131)S L(133)A
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVSCAVKDFYPPDISVEWQSNRWPELEGKYSTTPAQLDGDGSYFLYSKLSLETSRWQQVESFTCAVMHEALHNHFTKTDISESLGK

SEQ ID NO: 130 - protein, 214 aa, Artificial Sequence
  Synthetic Exemplary variant equine IgG3 Fc Heterodimer chain 2 T(131)S L(133)A
GGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVSCAVKDFYPPDITVEWQSNEHPEPEGKYRTTEAQKDSDGSYFLYSKLTVEKDRWQQGTTFTCVVMHEALHNHVMQKNISKNPGK

SEQ ID NO: 131 - protein, 214 aa, Artificial Sequence
  Synthetic Exemplary variant equine IgG4 Fc Heterodimer chain 2 T(131)S L(133)A
VGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISAPTGQPREPQVYVLAPHRDELSKNKVSVSCAVKDFYPPDIDIEWKSNGQPEPETKYSTTPAQLDSDGSYFLYSKLTVETNRWQQGTTFTCAVMHEALHNHYTEKSVSKSPGK

SEQ ID NO: 132 - protein, 214 aa, Artificial Sequence
  Synthetic Exemplary variant equine IgG5 Fc Heterodimer chain 2 T(131)S L(133)A
GGPSVFIFPPKPKDVLMISRKPEVTCVVVDLGHDDPDVQFTWFVDGVETHTATTEPKEEQFNSTYRVVSVLPIQHQDWLSGKEFKCSVTSKALPAPVERTISKAKGQLRVPQVYVLAPHPDELAKNTVSVSCAVKDFYPPEIDVEWQSNEHPEPEGKYSTTPAQLNSDGSYFLYSKLSVETSRWKQGESFTCGVMHEAVENHYTQKNVSHSPGK

SEQ ID NO: 133 - protein, 214 aa, Artificial Sequence
  Synthetic Exemplary variant equine IgG6 Fc Heterodimer chain 2 T(131)S L(133)A
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVSCAVKDFYPPDINVEWQSNEEPEPEVKYSTTPAQLDGDGSYFLYSKLTVETDRWEQGESFTCVVMHEAIRHTYRQKSITNFPGK

SEQ ID NO: 134 - protein, 214 aa, Artificial Sequence
  Synthetic Exemplary variant equine IgG7 Fc Heterodimer chain 2 T(131)S L(133)A
VGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHPDELSKNKVSVSCAVKDFYPPDIDIEWKSNGQPEPETKYSTTPAQLDGDGSYFLYSKLTVETNRWQQGTTFTCAVMHEALHNHYTEKSVSKSPGK

SEQ ID NO: 135 - protein, 214 aa, Artificial Sequence
  Synthetic Exemplary variant equine IgG1 Fc Heterodimer chain 2 T(131)S L(133)A Y(174)T
GGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVSCAVKDFYPPEINIEWQSNGQPELETKYSTTQAQQDSDGSYFLTSKLSVDRNRWQQGTTFTCGVMHEALHNHYTQKNVSKNPGK

SEQ ID NO: 136 - protein, 214 aa, Artificial Sequence
  Synthetic Exemplary variant equine IgG2 Fc Heterodimer chain 2 T(131)S L(133)A Y(174)T
GGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVSCAVKDFYPPDISVEWQSNRWPELEGKYSTTPAQLDGDGSYFLTSKLSLETSRWQQVESFTCAVMHEALHNHFTKTDISESLGK

SEQ ID NO: 137 - protein, 214 aa, Artificial Sequence
  Synthetic Exemplary variant equine IgG3 Fc Heterodimer chain 2 T(131)S L(133)A Y(174)T
GGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVSCAVKDFYPPDITVEWQSNEHPEPEGKYRTTEAQKDSDGSYFLTSKLTVEKDRWQQGTTFTCVVMHEALHNHVMQKNISKNPGK

SEQ ID NO: 138 - protein, 214 aa, Artificial Sequence
  Synthetic Exemplary variant equine IgG4 Fc Heterodimer chain 2 T(131)S L(133)A Y(174)T
VGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISAPTGQPREPQVYVLAPHRDELSKNKVSVSCAVKDFYPPDIDIEWKSNGQPEPETKYSTTPAQLDSDGSYFLTSKLTVETNRWQQGTTFTCAVMHEALHNHYTEKSVSKSPGK

SEQ ID NO: 139 - protein, 214 aa, Artificial Sequence; identical to SEQ ID NO: 132
  Synthetic Exemplary variant equine IgG5 Fc Heterodimer chain 2 T(131)S L(133)A Y(174)T

SEQ ID NO: 140 - protein, 214 aa, Artificial Sequence
  Synthetic Exemplary variant equine IgG6 Fc Heterodimer chain 2 T(131)S L(133)A Y(174)T
GRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVSCAVKDFYPPDINVEWQSNEEPEPEVKYSTTPAQLDGDGSYFLTSKLTVETDRWEQGESFTCVVMHEAIRHTYRQKSITNFPGK

SEQ ID NO: 141 - protein, 214 aa, Artificial Sequence; identical to SEQ ID NO: 134
  Synthetic Exemplary variant equine IgG7 Fc Heterodimer chain 2 T(131)S L(133)A Y(174)T

SEQ ID NO: 142 - protein, 98 aa, Unknown
  Wild-type canine IgG-A CH1
ASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPV

SEQ ID NO: 143 - protein, 98 aa, Unknown
  Wild-type canine IgG-B CH1
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPV

SEQ ID NO: 144 - protein, 98 aa, Unknown
  Wild-type canine IgG-C CH1
ASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPV

SEQ ID NO: 145 - protein, 98 aa, Unknown
  Wild-type canine IgG-D CH1
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPV

SEQ ID NO: 146 - protein, 98 aa, Artificial Sequence
  Synthetic Variant canine IgG-A CH1 A(24)L S(30)D
ASTTAPSVFPLAPSCGSTSGSTVLLACLVDGYFPEPVTVSWNSGSLTSGVHTFPSVLQSSGLHSLSSMVTVPSSRWPSETFTCNVVHPASNTKVDKPV

SEQ ID NO: 147 - protein, 98 aa, Artificial Sequence
  Synthetic Variant canine IgG-B CH1 A(24)L S(30)D
ASTTAPSVFPLAPSCGSTSGSTVLLACLVDGYFPEPVTVSWNSGSLTSGVHTFPSVLQSSGLYSLSSMVTVPSSRWPSETFTCNVAHPASKTKVDKPV

SEQ ID NO: 148 - protein, 98 aa, Artificial Sequence
  Synthetic Variant canine IgG-C CH1 A(24)L S(30)D
ASTTAPSVFPLAPSCGSQSGSTVLLACLVDGYIPEPVTVSWNSVSLTSGVHTFPSVLQSSGLYSLSSMVTVPSSRWPSETFTCNVAHPATNTKVDKPV

SEQ ID NO: 149 - protein, 98 aa, Artificial Sequence
  Synthetic Variant canine IgG-D CH1 A(24)L S(30)D
ASTTAPSVFPLAPSCGSTSGSTVLLACLVDGYFPEPVTVSWNSGSLTSGVHTFPSVLQSSGLYSLSSTVTVPSSRWPSETFTCNVVHPASNTKVDKPV

SEQ ID NO: 150 - protein, 110 aa, Unknown
  Wild-type canine kappa constant region
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 151 - protein, 110 aa, Artificial Sequence
  Synthetic Variant canine kappa constant region F(11)A S(22)R
RNDAQPAVYLAQPSPDQLHTGRASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 152 - protein, 98 aa, Unknown
  Wild-type feline IgG1 CH1
ASTTAPSVFPLAPSCGTTSGATVALACLVSGYFPEPVTVSWNSGALTSGVHTFPAVLQASGLYSLSSMVTVPSSRWLSDTFTCNVAHPPSNTKVDKTV

SEQ ID NO: 153 - protein, 98 aa, Unknown
  Wild-type feline IgG2 CH1
ASTTASSVFPLAPSCGTTSGATVALACLSLGYFPEPVTVSWNSGALTSGVHTFPSVLQASGLYSLSSMVTVPSSRWLSDTFTCNVAHRPSSTKVDKTV

SEQ ID NO: 154 - protein, 98 aa, Artificial Sequence
  Synthetic Variant feline IgG1 CH1 A(24)L S(30)D
ASTTAPSVFPLAPSCGTTSGATVLLACLVDGYFPEPVTVSWNSGALTSGVHTFPAVLQASGLYSLSSMVTVPSSRWLSDTFTCNVAHPPSNTKVDKTV

SEQ ID NO: 155 - protein, 98 aa, Artificial Sequence
  Synthetic Variant feline IgG2 CH1 A(24)L S(29)D
ASTTASSVFPLAPSCGTTSGATVLLACLDLGYFPEPVTVSWNSGALTSGVHTFPSVLQASGLYSLSSMVTVPSSRWLSDTFTCNVAHRPSSTKVDKTV

SEQ ID NO: 156 - protein, 110 aa, Unknown
  Wild-type feline kappa constant region
RSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 157 - protein, 110 aa, Artificial Sequence
  Synthetic Variant feline kappa constant region F(11)A S(22)R
RSDAQPSVFLAQPSLDELHTGRASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 158 - protein, 1 aa, Artificial Sequence
  Synthetic 1G extension
G

SEQ ID NO: 159 - protein, 2 aa, Artificial Sequence
  Synthetic 2G extension
GG

SEQ ID NO: 160 - protein, 3 aa, Artificial Sequence
  Synthetic 3G extension
GGG

SEQ ID NO: 161 - protein, 4 aa, Artificial Sequence
  Synthetic 4G extension
GGGG

SEQ ID NO: 162 - protein, 5 aa, Artificial Sequence
  Synthetic 5G extension
GGGGG

SEQ ID NO: 163 - protein, 6 aa, Artificial Sequence
  Synthetic 6G extension
GGGGGG

SEQ ID NO: 164 - protein, 7 aa, Artificial Sequence
  Synthetic 7G extension
GGGGGGG

SEQ ID NO: 165 - protein, 8 aa, Artificial Sequence
  Synthetic 8G extension
GGGGGGGG

SEQ ID NO: 166 - protein, 237 aa, Artificial Sequence
  Synthetic Exemplary variant feline IgG2 Fc Hinge Cys G(14)C
PKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 167 - protein, 237 aa, Artificial Sequence
  Synthetic Exemplary variant feline IgG1a Fc with modified hinge K(16)P
RKTDHPPGPKTGEGPPCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 168 - protein, 237 aa, Artificial Sequence
  Synthetic Exemplary variant feline IgG1a Fc with modified hinge K(16)P
RKTDHPPGPKPCDCPPCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 169 - protein, 237 aa, Artificial Sequence
  Synthetic Exemplary variant feline IgG1b Fc with modified hinge K(16)P
RKTDHPPGPKTGEGPPCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 170 - protein, 237 aa, Artificial Sequence
  Synthetic Exemplary variant feline IgG1b Fc with modified hinge K(16)P
RKTDHPPGPKPCDCPPCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 171 - protein, 237 aa, Artificial Sequence
  Synthetic Exemplary variant feline IgG2 Fc with modified hinge K(16)P
PKTASTIESKTGEGPPCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 172 - protein, 244 aa, Artificial Sequence
  Synthetic Exemplary variant equine IgG2 Fc with modified hinge Protein A - C1q - Q(20)P
PPCVLSAEGVIPIPSVPKPPCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 173 - protein, 244 aa, Artificial Sequence
  Synthetic Exemplary variant equine IgG2 Fc with modified hinge Protein A - C1q - C(3)S
PPSVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 174 - protein, 244 aa, Artificial Sequence
  Synthetic Exemplary variant equine IgG2 Fc with modified hinge Protein A - C1q - C(3)S Q(20)P
PPSVLSAEGVIPIPSVPKPPCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 175 - protein, 244 aa, Artificial Sequence
  Synthetic Exemplary variant equine IgG2 Fc with hinge  Protein A + C1q - A(45)T F(233)Y
PPCVLSAEGVIPIPSVPKPQCPPYTHSKFLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 176 - protein, 244 aa, Artificial Sequence
  Synthetic Exemplary variant equine IgG2 Fc with modified hinge Protein A + C1q - Q(20)P A(45)T F(233)Y
PPCVLSAEGVIPIPSVPKPPCPPYTHSKFLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 177 - protein, 244 aa, Artificial Sequence
  Synthetic Exemplary variant equine IgG2 Fc with modified hinge Protein A + C1q - C(3)S Q(20)P A(45)T F(233)Y
PPSVLSAEGVIPIPSVPKPPCPPYTHSKFLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 178 - protein, 237 aa, Artificial Sequence
  Synthetic Exemplary variant feline IgG2 Fc with feline IgG1 hinge
RKTDHPPGPKPCDCPKCPPPEMLGGPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 179 - protein, 228 aa, Artificial Sequence
  Synthetic Exemplary variant equine Fc IgG2 (with equine IgG1 hinge)  Protein A -  C1q -
DMSKCPKCPAPELLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 180 - protein, 228 aa, Artificial Sequence
  Synthetic Exemplary variant equine IgG2 Fc (with equine IgG1 hinge)  C1q - Protein A + A(29)T F(217)Y
DMSKCPKCPAPELLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 181 - protein, 29 aa, Artificial Sequence
  Synthetic Exemplary variant GLP1 (7-35)  X8 may be G or S
  misc_feature (2)..(2): Xaa can be any naturally occurring amino acid
HXEGTFTSDVSSYLEGQAAKEFIAWLVKG

SEQ ID NO: 182 - protein, 29 aa, Artificial Sequence
  Synthetic Glucagon (Gluc)
HSQGTFTSDYSKYLDSRRAQDFVQWLMNT

SEQ ID NO: 183 - protein, 39 aa, Artificial Sequence
  Synthetic Extendin-4
HGEGTFTSDLSKQMEEEAVRLFIEWLKNGGPSSGAPPPS

SEQ ID NO: 184 - protein, 304 aa, Artificial Sequence
  Synthetic ssGLP1-G8_I_VARfeIgG2
MAVLGLLFCLVTFPSCVLSHGEGTFTSDVSSYLEGQAAKEFIAWLVKGGGGSGGGGSGGGGSGGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 185 - protein, 344 aa, Artificial Sequence
  Synthetic ssGLP1-G8/GLP1-2G_III_ VARfeIgG2
MAVLGLLFCLVTFPSCVLSHGEGTFTSDVSSYLEGQAAKEFIAWLVKGGGGSGGGGSGGGGSGGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGKGGGGSGGGGHAEGTFTSDVSSYLEGQAAKEFIAWLVKGGG

SEQ ID NO: 186 - protein, 325 aa, Artificial Sequence
  Synthetic GLP1-G8/GLP1-2G_III_ VARfeIgG2
HGEGTFTSDVSSYLEGQAAKEFIAWLVKGGGGSGGGGSGGGGSGGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGKGGGGSGGGGHAEGTFTSDVSSYLEGQAAKEFIAWLVKGGG

SEQ ID NO: 187 - protein, 285 aa, Artificial Sequence
  Synthetic GLP1-G8_I_VARfeIgG2
HGEGTFTSDVSSYLEGQAAKEFIAWLVKGGGGSGGGGSGGGGSGGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 188 - protein, 325 aa, Artificial Sequence
  Synthetic GLP1-G8/GLP-2G_III_ WTfeIgG2
HGEGTFTSDVSSYLEGQAAKEFIAWLVKGGGGSGGGGSGGGGSGGGGSPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGKGGGGSGGGGHAEGTFTSDVSSYLEGQAAKEFIAWLVKGGG

SEQ ID NO: 189 - protein, 285 aa, Artificial Sequence
  Synthetic GLP1-G8_I_WTfeIgG2
HGEGTFTSDVSSYLEGQAAKEFIAWLVKGGGGSGGGGSGGGGSGGGGSPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 190 - protein, 315 aa, Artificial Sequence
  Synthetic Exemplary canine IL13R ECD
TETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPT

SEQ ID NO: 191 - protein, 305 aa, Artificial Sequence
  Synthetic Exemplary canine IL13R ECD
QPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSI

SEQ ID NO: 192 - protein, 315 aa, Artificial Sequence
  Synthetic Exemplary feline IL13R ECD
SQTQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSIGENTDPT

SEQ ID NO: 193 - protein, 305 aa, Artificial Sequence
  Synthetic Exemplary feline IL13R ECD
QPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSI

SEQ ID NO: 194 - protein, 315 aa, Artificial Sequence
  Synthetic Exemplary equine IL13R ECD
TESQPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSIGKKADPT

SEQ ID NO: 195 - protein, 305 aa, Artificial Sequence
  Synthetic Exemplary equine IL13R ECD
QPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSI

SEQ ID NO: 196 - protein, 205 aa, Artificial Sequence
  Synthetic Exemplary canine IL4R ECD
SGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEP

SEQ ID NO: 197 - protein, 184 aa, Artificial Sequence
  Synthetic Exemplary canine IL4R ECD
KVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNS

SEQ ID NO: 198 - protein, 270 aa, Artificial Sequence
  Synthetic Exemplary feline IL4R ECD
SGSVKVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEP

SEQ ID NO: 199 - protein, 266 aa, Artificial Sequence
  Synthetic Exemplary feline IL4R ECD
KVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEP

SEQ ID NO: 200 - protein, 205 aa, Artificial Sequence
  Synthetic Exemplary equine IL4R ECD
SGSVKVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQP

SEQ ID NO: 201 - protein, 201 aa, Artificial Sequence
  Synthetic Exemplary equine IL4R ECD
KVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQP

SEQ ID NO: 202 - protein, 780 aa, Artificial Sequence
  Synthetic IL13R ECD -  IL4R ECD - variant canine IgG-B Fc
MAVLGLLFCLVTFPSCVLSTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTGGGSGSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 203 - protein, 279 aa, Artificial Sequence
  Synthetic Exemplary canine IL17Ra ECD
MGRLGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCPEIPDAPVSIADYIPL

SEQ ID NO: 204 - protein, 261 aa, Artificial Sequence
  Synthetic Exemplary canine IL17Ra ECD
LGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCP

SEQ ID NO: 205 - protein, 273 aa, Artificial Sequence
  Synthetic Exemplary human IL17Ra ECD
SLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPDITVETLEAHQLRVSFTLWNESTHYQILLTSFPHMENHSCFEHMHHIPAPRPEEFHQRSDVTLTLRNLKGCCRHQVQIQPFFSSCLNDCLRHSATVSCP

SEQ ID NO: 206 - protein, 286 aa, Artificial Sequence
  Synthetic Exemplary feline IL17Ra ECD
SPRLLDYPAPVCSQQGLNCVVKNSTCLDDSWIHLRNLTPSSPKDVQVHLDFVQTQHGDLLPVAGIRWTLQTDASILYLEGAELSVLQLNTNERLCVKFEFLTRLKHHHKRWHFTFSHFVVEPGQEYEVTVHHLPKPIPDGDPNHQSRNFPVPGCEDPRMKMITPCVGSGSLWDPNITVETLEARQLWVSFTLWNESTHYQILLTSFPHTENHSCFQHTLMVPEPAYQDSRQRSNVTLTLSDSNWCCRHRVQIQPFFSSCLNDCLRHSITVPCPEIPDPPVSIADYI

SEQ ID NO: 207 - protein, 286 aa, Artificial Sequence
  Synthetic Exemplary equine IL17Ra ECD
SPRLLEHPAPVCSQQGLNCTVKNSTCLDDSWLHPPHLTPSSPKDVQIQLHFAHTQQGDLLPVIHIEWTLQTDASILYLEGAELSVLQLSTNERLCVTFEFLSRLKHHHKRWRFTFAHFVVEPGQEYEVTVHHLPKPFPHGDPNHQSRNFLVPDCMDPRMRITTPCVSSGSLWDPNITVETLEAHRLRVDFTLWNESARYQILLSSFPHMENQSCFDDVQNILKHTPEASHQRANITLTLSDFNWCCRHHVQIQPFFSSCLNDCLRHTVTVPCPEIPDTPDSTADYM

SEQ ID NO: 208 - protein, 460 aa, Artificial Sequence
  Synthetic Exemplary human IL17RC ECD
LERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSL

SEQ ID NO: 209 - protein, 339 aa, Artificial Sequence
  Synthetic Exemplary human IL17RC ECD
VLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSL

SEQ ID NO: 210 - protein, 389 aa, Artificial Sequence
  Synthetic Exemplary canine IL17RC ECD
LEKLMGPQDTARCSPGLSCHLWDGDVLCLPGSIVSAPGPVLVPTRLQTELVLRCYQETDCDLCVRVAIHLAVHGHWEEPKDEDKFGRAADPELEEPRNAFLQAQVVLSFQAYPTARCVLLEVQVPAALVQPGQSVGSVVFDCFEAALGAEVRIWSYTQPRYQKELNFTQQLPDCKGLEVRDSIQSCWALPWLNVSADGDDVYLVLDVSEEQRFGLSLYWNQIQGPTKPWWHRNLTGPQTITLNHTDLFPCLCIQVWPLEPDSVRTSVCPFREDPRAHRNLWRAARLQLLPPRGWRLDAPCSLLAEATLCWQAPGGGPCQSLVPPLYQANVTVNKTLELPLLNAHPNLCVQVSSWEKLQLQECLWADSLRALKDDLLLVETRGLQDNRSL

SEQ ID NO: 211 - protein, 238 aa, Artificial Sequence
  Synthetic Exemplary canine IL17RC ECD
RIWSYTQPRYQKELNFTQQLPDCKGLEVRDSIQSCWALPWLNVSADGDDVYLVLDVSEEQRFGLSLYWNQIQGPTKPWWHRNLTGPQTITLNHTDLFPCLCIQVWPLEPDSVRTSVCPFREDPRAHRNLWRAARLQLLPPRGWRLDAPCSLLAEATLCWQAPGGGPCQSLVPPLYQANVTVNKTLELPLLNAHPNLCVQVSSWEKLQLQECLWADSLRALKDDLLLVETRGLQDNRSL

SEQ ID NO: 212 - protein, 389 aa, Artificial Sequence
  Synthetic Exemplary feline IL17RC ECD
LERLVGPQDTARCSPGLSCHLWDGDVLCLPGSIVSAPGPVLVPTRLQTELVLRCYQETDCDLCVRVAIHLAVHGHWEEPKGEEKFGGAADPELEESRNAFLQAQVVLSFQAYPTARCVLLEVQVPAALVQPGQSVGSVVFDCFEAALGAEVRIWSYTQPRYQKELNLTQHLPDCKGLEVRDSIQSCWALPWLNVSADGDDVHLVLDVSEDQRFGLSLYWNQVQGPTKPWWHRNLTGPQTITLNHTDLFPCLCIQVWPLEPDSVRTSICPFREDPRAHRNLWRAARLQLLPPRGWRLDAPCSLPAEATLCWQAPGGGPCQSLVPPLPPANVTVNKALELPLLNVHPNLCVQVSSWEKLQLQECLWVDSLGPLKDDMLLVETRDPHNNRSL

SEQ ID NO: 213 - protein, 238 aa, Artificial Sequence
  Synthetic Exemplary feline IL17RC ECD
RIWSYTQPRYQKELNLTQHLPDCKGLEVRDSIQSCWALPWLNVSADGDDVHLVLDVSEDQRFGLSLYWNQVQGPTKPWWHRNLTGPQTITLNHTDLFPCLCIQVWPLEPDSVRTSICPFREDPRAHRNLWRAARLQLLPPRGWRLDAPCSLPAEATLCWQAPGGGPCQSLVPPLPPANVTVNKALELPLLNVHPNLCVQVSSWEKLQLQECLWVDSLGPLKDDMLLVETRDPHNNRSL

SEQ ID NO: 214 - protein, 391 aa, Artificial Sequence
  Synthetic Exemplary equine IL17RC ECD
LERLEGLQDAARCSPGLSCHLWDGDVVCLPGSIVSAPGPVLVPTSLQTELVRRCYQETDCDLCVRVAVHLAVHGHWEKPEDEEKLGRAADPEPEEPRNASLQAQVVLSFQAYPTARCVLLEVQVPAALVQPGQSVGSVVFDCFEAALGTEVQIWSYTQPRYQKELNLTRQLPDCRGLEVQDSIQSCRALPWLSVTADGDNVHLVLDVSEEQSFGLSLYWNQVQGPVKPWWHRNLTGPQTIPLNQTDIVPCLCIQAWPLEPDSVRTSICPFTEDPRAHRNLWRAARLQLLPPRGWRLDAPCSLHAQATLCWQAPSRGPCQPLVPPLPRENVTVNMALEFPLLKGHPNLCVQVSSWEKMQLQLQECLWADSLGPLKDDMLLVEAGGPQDNRSF

SEQ ID NO: 215 - protein, 240 aa, Artificial Sequence
  Synthetic Exemplary equine IL17RC ECD
QIWSYTQPRYQKELNLTRQLPDCRGLEVQDSIQSCRALPWLSVTADGDNVHLVLDVSEEQSFGLSLYWNQVQGPVKPWWHRNLTGPQTIPLNQTDIVPCLCIQAWPLEPDSVRTSICPFTEDPRAHRNLWRAARLQLLPPRGWRLDAPCSLHAQATLCWQAPSRGPCQPLVPPLPRENVTVNMALEFPLLKGHPNLCVQVSSWEKMQLQLQECLWADSLGPLKDDMLLVEAGGPQDNRSF

SEQ ID NO: 216 - protein, 907 aa, Unknown
  Exemplary canine IL17Ra ECD - canine IL17RC ECD - wildtype IgG-B-Fc
MGRLGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCPEIPDAPVSIADYIGSLEKLMGPQDTARCSPGLSCHLWDGDVLCLPGSIVSAPGPVLVPTRLQTELVLRCYQETDCDLCVRVAIHLAVHGHWEEPKDEDKFGRAADPELEEPRNAFLQAQVVLSFQAYPTARCVLLEVQVPAALVQPGQSVGSVVFDCFEAALGAEVRIWSYTQPRYQKELNFTQQLPDCKGLEVRDSIQSCWALPWLNVSADGDDVYLVLDVSEEQRFGLSLYWNQIQGPTKPWWHRNLTGPQTITLNHTDLFPCLCIQVWPLEPDSVRTSVCPFREDPRAHRNLWRAARLQLLPPRGWRLDAPCSLLAEATLCWQAPGGGPCQSLVPPLYQANVTVNKTLELPLLNAHPNLCVQVSSWEKLQLQECLWADSLRALKDDLLLVETRGLQDNRSLGSPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 217 - protein, 756 aa, Unknown
  Exemplary canine IL17Ra ECD - canine IL17RC ECD - wildtype IgG-B-Fc
MGRLGEGLNCTVKNSTCLDDSWIHPRNLTPSSPKDVQVHLDFAQTQHGDLLPIIGIRWTLQTDASILFLEGAELSVLQLNTNERVCVKFEFLSKLKHHHKRWHFTFSHFVVEPGQEYEVTVHHLPKPIPDGDPNHQSKNFLVPGCEDPRMRMTTPCVSSGSLWDPNITAEALEAHQLQVHFTLWNESAQYQILLTSFPHTENRSCFHRVLMVPEPTLKEHHQRANIMLTGSSSNWCCRHQVQIQPFFSSCLNDCLRHSVTVPCPEIPDAPVSIADYIGSRIWSYTQPRYQKELNFTQQLPDCKGLEVRDSIQSCWALPWLNVSADGDDVYLVLDVSEEQRFGLSLYWNQIQGPTKPWWHRNLTGPQTITLNHTDLFPCLCIQVWPLEPDSVRTSVCPFREDPRAHRNLWRAARLQLLPPRGWRLDAPCSLLAEATLCWQAPGGGPCQSLVPPLYQANVTVNKTLELPLLNAHPNLCVQVSSWEKLQLQECLWADSLRALKDDLLLVETRGLQDNRSLGSPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 218 - protein, 125 aa, Artificial Sequence
  Synthetic Exemplary canine TrkA ECD
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGD

SEQ ID NO: 219 - protein, 101 aa, Artificial Sequence
  Synthetic Exemplary canine TrkA ECD
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNP

SEQ ID NO: 220 - protein, 99 aa, Artificial Sequence
  Synthetic Exemplary canine TrkA ECD
FPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNP

SEQ ID NO: 221 - protein, 125 aa, Artificial Sequence
  Synthetic Exemplary feline TrkA ECD
VSFPASVQLHAAVELHHWCIPFSVDGQPAPSLRWLFNGSVLNETSFIFTEFLEPAANETVRHGCLRLNQPTHVNNGNYTLLAANPSGRAAASVLAAFMDNPFEFNPEDPIPVSFSPVDSNSTSGD

SEQ ID NO: 222 - protein, 101 aa, Artificial Sequence
  Synthetic Exemplary feline TrkA ECD
VSFPASVQLHAAVELHHWCIPFSVDGQPAPSLRWLFNGSVLNETSFIFTEFLEPAANETVRHGCLRLNQPTHVNNGNYTLLAANPSGRAAASVLAAFMDNP

SEQ ID NO: 223 - protein, 99 aa, Artificial Sequence
  Synthetic Exemplary feline TrkA ECD
FPASVQLHAAVELHHWCIPFSVDGQPAPSLRWLFNGSVLNETSFIFTEFLEPAANETVRHGCLRLNQPTHVNNGNYTLLAANPSGRAAASVLAAFMDNP

SEQ ID NO: 224 - protein, 125 aa, Artificial Sequence
  Synthetic Exemplary equine TrkA ECD
VSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPFEFNPEDPIPVSFSPVDTNSTSRD

SEQ ID NO: 225 - protein, 101 aa, Artificial Sequence
  Synthetic Exemplary equine TrkA ECD
VSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNP

SEQ ID NO: 226 - protein, 99 aa, Artificial Sequence
  Synthetic Exemplary equine TrkA ECD
FPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNP

SEQ ID NO: 227 - protein, 125 aa, Artificial Sequence
  Synthetic Exemplary human TrkA ECD
VSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGD

SEQ ID NO: 228 - protein, 101 aa, Artificial Sequence [sequence omitted]

SEQ ID NO: 229 - protein, 99 aa, Artificial Sequence
  Synthetic Exemplary human TrkA ECD
FPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNP

SEQ ID NO: 230 - protein, 360 aa, Unknown
  Exemplary canine TrkA ECD - wildtype canine Fc-IgG-B
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 231 - protein, 384 aa, Unknown
  Exemplary canine TrkA ECD - wildtype canine IgG-B Fc
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 232 - protein, 360 aa, Artificial Sequence
  Synthetic Exemplary canine TrkA ECD - variant canine IgGB Fc Variant canine IgG-B Fc Protein A+ C1q - CD16 -
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSRPPDCPKCPAPEPLGGPSVFIFPPKPKDTLLIARTPEVTCVVVDLDREDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 233 - protein, 384 aa, Artificial Sequence
  Synthetic Exemplary canine TrkA ECD - variant canine IgGB Fc Variant canine IgG-B Fc Protein A+ C1q - CD16 -
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSRPPDCPKCPAPEPLGGPSVFIFPPKPKDTLLIARTPEVTCVVVDLDREDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 234 - protein, 364 aa, Unknown
  Exemplary canine TrkA ECD - wildtype canine IgGA Fc
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 235 - protein, 388 aa, Unknown
  Exemplary canine TrkA ECD - wildtype canine IgGA Fc
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 236 - protein, 364 aa, Artificial Sequence
  Synthetic Exemplary canine TrkA ECD - variant canine IgGA Fc Variant canine IgG-A Fc Protein A+ C1q - CD16 -
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSFNECRCTDTPCPVPEPLGGPSVLIFPPKPKDTLLIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 237 - protein, 388 aa, Artificial Sequence
  Synthetic Exemplary canine TrkA ECD - variant canine IgGA Fc Variant canine IgG-A Fc Protein A+ C1q - CD16 -
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSFNECRCTDTPCPVPEPLGGPSVLIFPPKPKDTLLIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 238 - protein, 365 aa, Unknown
  Exemplary canine TrkA ECD - wildtype canine IgGD Fc
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 239 - protein, 389 aa, Unknown
  Exemplary canine TrkA ECD - wildtype canine IgGD Fc
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 240 - protein, 365 aa, Artificial Sequence
  Synthetic Exemplary canine TrkA ECD - variant canine IgGD Fc Variant canine IgG-A Fc Protein A+ C1q - CD16 -
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSPKESTCKCISPCPVPESLGGPSVFIFPPKPKDTLLIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 241 - protein, 389 aa, Artificial Sequence
  Synthetic Exemplary canine TrkA ECD - variant canine IgGD Fc Variant canine IgG-A Fc Protein A+ C1q - CD16 -
MDMRVPAQLLGLLLLWLRGARCVSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSPKESTCKCISPCPVPESLGGPSVFIFPPKPKDTLLIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 242 - protein, 369 aa, Artificial Sequence
  Synthetic Exemplary feline TrkA ECD - variant feline IgG2 Fc Variant feline IgG2 Fc Hinge Cys
MDMRVPAQLLGLLLLWLRGARCVSFPASVQLHAAVELHHWCIPFSVDGQPAPSLRWLFNGSVLNETSFIFTEFLEPAANETVRHGCLRLNQPTHVNNGNYTLLAANPSGRAAASVLAAFMDNPSGGGSGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 243 - protein, 393 aa, Artificial Sequence
  Synthetic Exemplary feline TrkA ECD - variant feline IgG2 Fc Variant feline IgG2 Fc Hinge Cys
MDMRVPAQLLGLLLLWLRGARCVSFPASVQLHAAVELHHWCIPFSVDGQPAPSLRWLFNGSVLNETSFIFTEFLEPAANETVRHGCLRLNQPTHVNNGNYTLLAANPSGRAAASVLAAFMDNPFEFNPEDPIPVSFSPVDSNSTSGDSGGGSGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 244 - protein, 365 aa, Artificial Sequence
  Synthetic Exemplary equine TrkA ECD - variant equine IgG2 Fc Variant equine IgG2 Fc Protein A+ C1q-
METDTLLLWVLLLWVPGSTGVSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPPPCVLSAEGVIPIPSVPKPQCPPYTHSKFLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEQQSNRWPELEGKYSTTPAQLDGDGSYFLYSKLSLETSRWQQVESFTCAVMHEALHNHYTKTDISESLGK

SEQ ID NO: 245 - protein, 389 aa, Artificial Sequence
  Synthetic Exemplary equine TrkA ECD - variant equine IgG2 Fc Variant equine IgG2 Fc Protein A+ C1q-
METDTLLLWVLLLWVPGSTGVSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPFEFNPEDPIPVSFSPVDTNSTSRDPPCVLSAEGVIPIPSVPKPQCPPYTHSKFLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 246 - protein, 349 aa, Artificial Sequence
  Synthetic Exemplary equine TrkA ECD - variant equine IgG2 Fc Variant equine IgG2 Fc with equine IgG1 hinge Protein A+ C1q-
METDTLLLWVLLLWVPGSTGVSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPDMSKCPKCPAPELLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 247 - protein, 373 aa, Artificial Sequence
  Synthetic Exemplary equine TrkA ECD - variant equine IgG2 Fc Variant equine IgG2 Fc with equine IgG1 hinge Protein A+ C1q-
METDTLLLWVLLLWVPGSTGVSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPFEFNPEDPIPVSFSPVDTNSTSRDDMSKCPKCPAPELLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 248 - protein, 350 aa, Artificial Sequence
  Synthetic Exemplary human TrkA ECD - variant human IgG4 Fc Variant human IgG4 S to P
METDTLLLWVLLLWVPGSTGVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 249 - protein, 374 aa, Artificial Sequence
  Synthetic Exemplary human TrkA ECD - variant human IgG4 Fc Variant human IgG4 S to P
METDTLLLWVLLLWVPGSTGVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 250 - protein, 338 aa, Unknown
  Exemplary canine TrkA ECD - wildtype canine Fc-IgG-B
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 251 - protein, 362 aa, Unknown
  Exemplary canine TrkA ECD - wildtype canine IgG-B Fc
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 252 - protein, 338 aa, Artificial Sequence
  Synthetic Exemplary canine TrkA ECD - variant canine IgGB Fc Variant canine IgG-B Fc Protein A+ C1q - CD16 -
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSRPPDCPKCPAPEPLGGPSVFIFPPKPKDTLLIARTPEVTCVVVDLDREDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 253 - protein, 362 aa, Artificial Sequence
  Synthetic Exemplary canine TrkA ECD - variant canine IgGB Fc Variant canine IgG-B Fc Protein A+ C1q - CD16 -
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSRPPDCPKCPAPEPLGGPSVFIFPPKPKDTLLIARTPEVTCVVVDLDREDPEVQISWFVDGKQMQTAKTQPREEQFNGTYRVVSVLPIGHQDWLKGKQFTCRVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 254 - protein, 342 aa, Unknown
  Exemplary canine TrkA ECD - wildtype canine IgGA Fc
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 255 - protein, 366 aa, Unknown
  Exemplary canine TrkA ECD - wildtype canine IgGA Fc
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSFNECRCTDTPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 256 - protein, 342 aa, Artificial Sequence
  Synthetic Exemplary canine TrkA ECD - variant canine IgGA Fc Variant canine IgG-A Fc Protein A+ C1q - CD16 -
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSFNECRCTDTPCPVPEPLGGPSVLIFPPKPKDTLLIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 257 - protein, 366 aa, Artificial Sequence
  Synthetic Exemplary canine TrkA ECD - variant canine IgGA Fc Variant canine IgG-A Fc Protein A+ C1q - CD16 -
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSFNECRCTDTPCPVPEPLGGPSVLIFPPKPKDTLLIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 258 - protein, 343 aa, Unknown
  Exemplary canine TrkA ECD - wildtype canine IgGD Fc
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 259 - protein, 367 aa, Unknown
  Exemplary canine TrkA ECD - wildtype canine IgGD Fc
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 260 - protein, 343 aa, Artificial Sequence
  Synthetic Exemplary canine TrkA ECD - variant canine IgGD Fc Variant canine IgG-A Fc Protein A+ C1q - CD16 -
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPSGGGSGGGSPKESTCKCISPCPVPESLGGPSVFIFPPKPKDTLLIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 261 - protein, 367 aa, Artificial Sequence
  Synthetic Exemplary canine TrkA ECD - variant canine IgGD Fc Variant canine IgG-A Fc Protein A+ C1q - CD16 -
VSFPASVQLHEAVELHHWCIPFSVDGQPAPSLRWLFNGSVLNETSFIFTEFLEPVANETVRHGCLRLNQPTHVNNGNYTLLAANPSGRAAAFVMAAFMDNPFEFNPEDPIPVSFSPVDTNSTSGDSGGGSGGGSPKESTCKCISPCPVPESLGGPSVFIFPPKPKDTLLIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 262 - protein, 347 aa, Artificial Sequence
  Synthetic Exemplary feline TrkA ECD - variant feline IgG2 Fc Variant feline IgG2 Fc Hinge Cys
VSFPASVQLHAAVELHHWCIPFSVDGQPAPSLRWLFNGSVLNETSFIFTEFLEPAANETVRHGCLRLNQPTHVNNGNYTLLAANPSGRAAASVLAAFMDNPSGGGSGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 263 - protein, 371 aa, Artificial Sequence
  Synthetic Exemplary feline TrkA ECD - variant feline IgG2 Fc Variant feline IgG2 Fc Hinge Cys
VSFPASVQLHAAVELHHWCIPFSVDGQPAPSLRWLFNGSVLNETSFIFTEFLEPAANETVRHGCLRLNQPTHVNNGNYTLLAANPSGRAAASVLAAFMDNPFEFNPEDPIPVSFSPVDSNSTSGDSGGGSGGGSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 264 - protein, 345 aa, Artificial Sequence
  Synthetic Exemplary equine TrkA ECD - variant equine IgG2 Fc Variant equine IgG2 Fc Protein A+ C1q-
VSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPPPCVLSAEGVIPIPSVPKPQCPPYTHSKFLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEQQSNRWPELEGKYSTTPAQLDGDGSYFLYSKLSLETSRWQQVESFTCAVMHEALHNHYTKTDISESLGK

SEQ ID NO: 265 - protein, 369 aa, Artificial Sequence
  Synthetic Exemplary equine TrkA ECD - variant equine IgG2 Fc Variant equine IgG2 Fc Protein A+ C1q-
VSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPFEFNPEDPIPVSFSPVDTNSTSRDPPCVLSAEGVIPIPSVPKPQCPPYTHSKFLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 266 - protein, 329 aa, Artificial Sequence
  Synthetic Exemplary equine TrkA ECD - variant equine IgG2 Fc Variant equine IgG2 Fc with equine IgG1 hinge Protein A+ C1q-
VSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPDMSKCPKCPAPELLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 267 - protein, 353 aa, Artificial Sequence
  Synthetic Exemplary equine TrkA ECD - variant equine IgG2 Fc Variant equine IgG2 Fc with equine IgG1 hinge Protein A+ C1q-
VSFPASVHLQTAVEQHHWCIPFSVDGQPAPTLRWLFNGSVLNETSFIFTEFLESAANETMRHGCLRLNQPTHVNNGNYTLLATNPYGQDSASVMVAFMDNPFEFNPEDPIPVSFSPVDTNSTSRDDMSKCPKCPAPELLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 268 - protein, 330 aa, Artificial Sequence
  Synthetic Exemplary human TrkA ECD - variant human IgG4 Fc Variant human IgG4 S to P
VSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 269 - protein, 354 aa, Artificial Sequence
  Synthetic Exemplary human TrkA ECD - variant human IgG4 Fc Variant human IgG4 S to P
VSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 270 - protein, 229 aa, Artificial Sequence
  Synthetic Exemplary variant human IgG4 S(10)P
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 271 - protein, 780 aa, Unknown
  IL13R ECD - IL4R ECD - wildtype canine IgG-B Fc
MAVLGLLFCLVTFPSCVLSTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTGGGSGSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

(19) United States
(12) Patent Application Publication
(10) Pub. No.: US 2022/0064263 A1
(43) Pub. Date: Mar. 3, 2022

(54) IGG FC Variants for Veterinary Use

(71) Applicant: Kindred Biosciences, Inc., Burlingame, CA (US)

(72) Inventors: Hangjun Zhan, Foster City, CA (US); Lam Nguyen, Union City, CA (US); Yongzhong Li, Rockville, MD (US); Fawn Qian, Burlingame, CA (US); Shyr Jiann Li, Millbrae, CA (US); Richard Chin, San Francisco, CA (US)

(73) Assignee: Kindred Biosciences, Inc., Burlingame, CA (US)

(21) Appl. No.: 17/414,637

(22) PCT Filed: Dec. 26, 2019
(86) PCT No.: PCT/US19/68629

§ 371 Date: Jun. 16, 2021

Related U.S. Application Data
(60) Provisional application No. 62/785,680, filed on Dec. 27, 2018.

(51) Int. Cl. C07K 14/715; C07K 14/605

(52) U.S. Cl. CPC C07K 14/7155; C07K 14/605; C07K 2317/24; C07K 2317/35; A61K 38/00; C07K 2317/71; C07K 2317/92; C07K 2317/94; C07K 2317/522

(57) ABSTRACT

Provided are various embodiments relating to variant IgG Fc polypeptides of companion animals having increased Protein A binding for ease of purification, decreased C1q binding for reduced complement-mediated immune responses, decreased CD 16 binding (e.g., for reduced antibody-dependent cellular cytotoxicity (ADCC) induction), increased stability, and/or the ability to form multimeric proteins. In addition, various embodiments relating to antibodies and fusion proteins comprising such variant IgG Fc polypeptides are provided. In various embodiments, such polypeptides may be used to treat companion animals, such as canines, felines, and equines.

### CROSS-REFERENCE TO RELATED APPLICATIONS

This application claims the benefit of priority of U.S. Provisional Application No. 62/785,680, filed Dec. 27, 2018, which is incorporated by reference herein in its entirety for any purpose.

### FIELD

The present disclosure relates to variant IgG Fc polypeptides of companion animals with enhanced features, including increased Protein A binding (e.g., for ease of purification), decreased C1q binding (e.g., for reduced complement-mediated immune responses), decreased CD16 binding (e.g., for reduced antibody-dependent cellular cytotoxicity (ADCC) induction), increased stability, and/or the ability to form heterodimeric proteins. The variant IgG Fc polypeptides of the present disclosure may have broad applicability in companion animal therapeutics. For example, variant IgG Fc polypeptides may be used in the design and production of long-acting GLP1 polypeptides for treating, for example, diabetes, obesity, or related indications, in companion animals, such as canines, felines, and equines. In addition, variant IgG Fc polypeptides may be used in the design and production of antibodies or fusion proteins for treating various disorders in companion animals.

### BACKGROUND

IgG Fc plays an important role in Fc-mediated functions though interactions with FcRn, Fc receptor, and C1q. In companion animals, various IgG subtypes possess differences in these functions, which are often considered when choosing a particular IgG antibody or IgG Fc fusion protein for therapeutic or diagnostic applications. For example, the ability of an IgG subtype to have weak or no measurable binding affinity to C1q or CD16 may be advantageous. In addition, IgG Fc's ability to bind Protein A may be useful for purification using a Protein A affinity purification platform.

However, most IgG Fc subtypes of canine, feline, and equine do not possess Protein A binding properties, weak or no measurable binding affinity to CD16, and weak or no measurable binding affinity to C1q. For example, of the four canine IgG Fc subtypes (IgG-A, IgG-B, IgG-C, and IgG-D), only canine IgG-B Fc has appreciable affinity to Protein A. Meanwhile only canine IgG-A Fc and IgG-D Fc have no or weak C1q binding or CD16 binding. Antibodies and Fc fusion proteins comprising variant IgG Fc polypeptides that have reduced binding to C1q and/or CD16, and/or that able to bind Protein A are desirable.

### SUMMARY OF THE INVENTION

Embodiment 1. A polypeptide comprising at least one therapeutic polypeptide and/or at least one antibody, and a variant IgG Fc polypeptide, wherein the variant IgG Fc polypeptide comprises:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide;
c) at least one amino acid modification relative to a wild-type IgG Fc polypeptide of a companion animal species, wherein the variant IgG Fc polypeptide has reduced binding affinity to CD16 relative to the wild-type IgG Fc polypeptide;
d) a hinge region comprising at least one amino acid modification to relative to a wild-type feline or equine IgG Fc polypeptide, wherein the variant IgG Fc polypeptide has increased recombinant production and/or increased hinge disulfide formation relative to the wild-type IgG Fc polypeptide, as determined by SDS-PAGE analysis under reducing and/or nonreducing conditions;
e) at least one amino acid substitution relative to a wild-type feline IgG Fc polypeptide, wherein the at least one amino acid substitution is a cysteine, and wherein the variant IgG Fc polypeptide is capable of forming at least one additional inter-chain disulfide linkage relative to the wild-type feline IgG Fc polypeptide;
f) at least one amino acid substitution relative to a wild-type canine IgG-A or IgG-D Fc polypeptide, wherein the variant IgG Fc polypeptide has increased binding affinity to C1q and/or CD16 relative to the wild-type canine IgG-A or IgG-D Fc polypeptide; and/or
g) a CH1 region comprising at least one amino acid modification relative to a wild-type canine or feline IgG CH1 region, wherein the variant IgG Fc polypeptide comprises:
   i) at least one amino acid substitution at a position corresponding to position 24 and/or position 30 of SEQ ID NO: 142, of SEQ ID NO: 143, of SEQ ID NO: 144, or of SEQ ID NO: 145, or
   ii) at least one amino acid substitution at a position corresponding to position 24 and/or position 29 of SEQ ID NO: 152 or of SEQ ID NO: 153.
Embodiment 2. A contiguous polypeptide comprising:
i) a first therapeutic polypeptide and/or antibody (TPA1);
ii) a first linker (L1);
iii) a variant Fc polypeptide (Fc) of a companion animal species;
iv) optionally, a second linker (L2); and
v) optionally, a second therapeutic polypeptide and/or antibody (TPA2),
   wherein the variant IgG Fc polypeptide comprises:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide;
c) at least one amino acid modification relative to a wild-type IgG Fc polypeptide of a companion animal species, wherein the variant IgG Fc polypeptide has reduced binding affinity to CD16 relative to the wild-type IgG Fc polypeptide;
d) a hinge region comprising at least one amino acid modification to relative to a wild-type feline or equine IgG Fc polypeptide, wherein the variant IgG Fc polypeptide has increased recombinant production and/or increased hinge disulfide formation relative to the wild-type IgG Fc polypeptide, as determined by SDS-PAGE analysis under reducing and/or nonreducing conditions; and/or
e) at least one amino acid substitution relative to a wild-type feline IgG Fc polypeptide, wherein the at least one amino acid substitution is a cysteine, and wherein the variant IgG Fc polypeptide is capable of forming at least one additional inter-chain disulfide linkage relative to the wild-type feline IgG Fc polypeptide;
f) at least one amino acid substitution relative to a wild-type canine IgG-A or IgG-D Fc polypeptide, wherein the variant IgG Fc polypeptide has increased binding affinity to C1q and/or CD16 relative to the wild-type canine IgG-A or IgG-D Fc polypeptide; and/or
g) a CH1 region comprising at least one amino acid modification relative to a wild-type canine or feline IgG CH1 region, wherein the variant IgG Fc polypeptide comprises:
   i) at least one amino acid substitution at a position corresponding to position 24 and/or position 30 of SEQ ID NO: 142, of SEQ ID NO: 143, of SEQ ID NO: 144, or of SEQ ID NO: 145, or
   ii) at least one amino acid substitution at a position corresponding to position 24 and/or position 29 of SEQ ID NO: 152 or of SEQ ID NO: 153.
Embodiment 3. The contiguous polypeptide of embodiment 2 comprising:

TPA1-L1-Fc formula (I):

Fc-L1-TPA1; formula (II):

TPA1-L1-Fc-L2-TPA2; formula (III):

TPA1-L1-TPA2-L2-Fc; or formula (IV):

Fc-L1-TPA1-L2-TPA2. formula (V):
Embodiment 4. A multimeric protein comprising:

i) a first therapeutic polypeptide and/or an antibody (TPA1), and a first variant IgG Fc polypeptide comprising at least one amino acid modification relative to a first wild-type IgG Fc polypeptide, and

ii) a second therapeutic polypeptide and/or an antibody (TPA2), and a second variant IgG Fc polypeptide comprising at least one amino acid modification relative to a second wild-type IgG Fc polypeptide, wherein
a) the first variant IgG Fc polypeptide comprises:
   i) an amino acid substitution at a position corresponding to position 138 of SEQ ID NO: 1, position 137 of SEQ ID NO: 2, position 137 of SEQ ID NO: 4, or position 138 of SEQ ID NO: 6;
   ii) an amino acid substitution at a position corresponding to position 154 of SEQ ID NO: 65, of SEQ ID NO: 66, of SEQ ID NO: 67, of SEQ ID NO: 68, or of SEQ ID NO: 69; or
   iii) an amino acid substitution at a position corresponding to position 131 of SEQ ID NO: 49, of SEQ ID NO: 50, of SEQ ID NO: 52, of SEQ ID NO: 53, of SEQ ID NO: 54, of SEQ ID NO: 55, or of SEQ ID NO: 56; and
b) the second variant IgG Fc polypeptide comprises:
   i) an amino acid substitution at a position corresponding to position 138, position 140, and/or position 181 of SEQ ID NO: 1, position 137, position 139, and/or position 180 of SEQ ID NO: 2, position 137, position 139, and/or position 180 of SEQ ID NO: 3, or position 138, position 140, and/or position 181 of SEQ ID NO: 4;
   ii) an amino acid substitution at a position corresponding to position 154, position 156, and/or position 197 of SEQ ID NO: 6, of SEQ ID NO: 80, of SEQ ID NO: 81, of SEQ ID NO: 117, or of SEQ ID NO: 118; or
   iii) an amino acid substitution at a position corresponding to position 131, position 133, and/or position 174 of SEQ ID NO: 49, of SEQ ID NO: 50, of SEQ ID NO: 52, of SEQ ID NO: 53, of SEQ ID NO: 54, of SEQ ID NO: 55, or of SEQ ID NO: 56.
Embodiment 5. The multimeric protein of embodiment 4, wherein the first variant IgG Fc polypeptide and/or the second variant IgG Fc polypeptide comprises:
   a) at least one amino acid modification relative to a wild-type IgG Fc polypeptide of a companion animal species, wherein the first and/or second variant IgG Fc polypeptide has increased binding affinity to Protein A relative to the wild-type IgG Fc polypeptide;
   b) at least one amino acid modification relative to a wild-type IgG Fc polypeptide of a companion animal species, wherein the first and/or second variant IgG Fc polypeptide has reduced binding affinity to C1q relative to the wild-type IgG Fc polypeptide;
   c) at least one amino acid modification relative to a wild-type IgG Fc polypeptide of a companion animal species, wherein the first and/or second variant IgG Fc polypeptide has reduced binding affinity to CD16 relative to the wild-type IgG Fc polypeptide;
   d) a hinge region comprising at least one amino acid modification to relative to a wild-type feline or equine IgG Fc polypeptide, wherein the first and/or second variant IgG Fc polypeptide has increased recombinant production and/or increased hinge disulfide formation relative to the wild-type IgG Fc polypeptide, as determined by SDS-PAGE analysis under reducing and/or nonreducing conditions;
   e) at least one amino acid substitution relative to a wild-type feline IgG Fc polypeptide, wherein the at least one amino acid substitution is a cysteine, and wherein the first and/or second variant IgG Fc polypeptide is capable of forming at least one additional inter-chain disulfide linkage relative to the wild-type feline IgG Fc polypeptide; and/or
   f) at least one amino acid substitution relative to a wild-type canine IgG-A or IgG-D Fc polypeptide, wherein the variant IgG Fc polypeptide has increased binding affinity to C1q and/or CD16 relative to the wild-type canine IgG-A or IgG-D Fc polypeptide; and/or
   g) a CH1 region comprising at least one amino acid modification relative to a wild-type canine or feline IgG CH1 region, wherein the variant IgG Fc polypeptide comprises:
      i) at least one amino acid substitution at a position corresponding to position 24 and/or position 30 of SEQ ID NO: 142, of SEQ ID NO: 143, of SEQ ID NO: 144, or of SEQ ID NO: 145, or
      ii) at least one amino acid substitution at a position corresponding to position 24 and/or position 29 of SEQ ID NO: 152 or of SEQ ID NO: 153.
Embodiment 6. The multimeric protein of embodiment 4 or embodiment 5, wherein the first wild-type IgG Fc polypeptide and the second wild-type IgG Fc polypeptide are from the same IgG subtype.
Embodiment 7. The multimeric protein of embodiment 4 or embodiment 5, wherein the first wild-type IgG Fc polypeptide and the second wild-type IgG Fc polypeptide are from a different IgG subtype.
Embodiment 8. The polypeptide, the contiguous polypeptide, or the multimeric protein of any one of embodiments 2 to 7, wherein TPA2, if present, comprises a different amino acid sequence compared to TPA1.
Embodiment 9. The polypeptide, the contiguous polypeptide, or the multimeric protein of any one of embodiments 2 to 8, wherein TPA1 and TPA2 are different therapeutic polypeptides or are antibodies that bind to different targets.
Embodiment 10. The polypeptide, the contiguous polypeptide, or the multimeric protein of any one of the preceding embodiments, wherein the variant IgG Fc polypeptide binds to C1q and/or CD16 with a dissociation constant (K_{d}) of greater than 5×10⁻⁶ M, greater than 1×10⁻⁵ M, greater than 5×10⁻⁵ M, greater than 1×10⁻⁴ M, greater than 5×10⁻⁴ M, or greater than 1×10⁻³ M, as measured by biolayer interferometry.
Embodiment 11. The polypeptide, the contiguous polypeptide, or the multimeric protein of any one of the preceding embodiments, wherein the variant IgG Fc polypeptide binds to Protein A with a dissociation constant (K_{d}) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹M, less than 1×10⁻⁹M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.
Embodiment 12. The polypeptide, the contiguous polypeptide, or the multimeric protein of any one of the preceding embodiments, wherein the variant canine IgG-A or variant canine IgG-D Fc polypeptide binds to C1q and/or CD16 with a dissociation constant (K_{d}) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹M, less than 1×10⁻¹¹M, less than 5×10⁻¹² M, or less than 1×10⁻¹² M, as measured by biolayer interferometry.
Embodiment 13. The polypeptide, the contiguous polypeptide, or the multimeric protein of any one of the preceding embodiments, wherein the companion animal species is canine, feline, or equine.
Embodiment 14. The polypeptide, the contiguous polypeptide, or the multimeric protein of any one of the preceding embodiments, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1a Fc, IgG1b Fc, or IgG2 Fc.
Embodiment 15. The polypeptide, the contiguous polypeptide, or the multimeric protein of any one of the preceding embodiments, wherein the variant IgG Fc polypeptide comprises a CH1 region comprising at least one amino acid modification relative to a wild-type canine or feline IgG CH1 region, wherein the variant IgG Fc polypeptide comprises:

a) at least one amino acid substitution at position 24 and/or position 30 of SEQ ID NO: 142, of SEQ ID NO: 143, of SEQ ID NO: 144; or of SEQ ID NO: 145, or

b) at least one amino acid substitution at position 24 and/or position 29 of SEQ ID NO: 152 or of SEQ ID NO: 153.
Embodiment 16. The polypeptide, the contiguous polypeptide, or the multimeric protein of any one of the preceding embodiments, wherein the variant IgG Fc polypeptide comprises a CH1 region comprising at least one amino acid modification relative to a wild-type canine or feline IgG CH1 region, wherein the variant IgG Fc polypeptide comprises:

a) a leucine at a position corresponding to position 24 and/or an asparagine at a position corresponding to position 30 of SEQ ID NO: 142, of SEQ ID NO: 143, of SEQ ID NO: 144, or of SEQ ID NO: 145; or

b) a leucine at a position corresponding to position 24 and/or an asparagine at a position corresponding to position 29 of SEQ ID NO: 152 or of SEQ ID NO: 153.
Embodiment 17. The polypeptide, the contiguous polypeptide, or the multimeric protein of any one of the preceding embodiments, wherein the variant IgG Fc polypeptide comprises a CH1 region comprising at least one amino acid modification relative to a wild-type canine or feline IgG CH1 region, wherein the variant IgG Fc polypeptide comprises:

a) a leucine at position 24 and/or an asparagine at position 30 of SEQ ID NO: 142, of SEQ ID NO: 143, of SEQ ID NO: 144, or of SEQ ID NO: 145; or

b) a leucine at position 24 and/or an asparagine at position 29 of SEQ ID NO: 152 or of SEQ ID NO: 153.
Embodiment 18. The polypeptide, the contiguous polypeptide, or the multimeric protein of any one of the preceding embodiments comprising a wild-type or a variant canine or feline light chain constant region.
Embodiment 19. The polypeptide, the contiguous polypeptide, or the multimeric protein of any one of the preceding embodiments comprising a wild-type or a variant canine or feline light chain κ constant region.
Embodiment 20. The polypeptide, the contiguous polypeptide, or the multimeric protein of any one of the preceding embodiments comprising a variant light chain constant region comprising at least one amino acid modification relative to a wild-type canine or feline light chain κ constant region comprising:

a) at least one amino acid substitution at a position corresponding to position 11 and/or position 22 of SEQ ID NO: 150; or

b) at least one amino acid substitution at a position corresponding to position 11 and/or position 22 of SEQ ID NO: 156.
Embodiment 21. The polypeptide, the contiguous polypeptide, or the multimeric protein of any one of the preceding embodiments comprising a variant light chain constant region comprising at least one amino acid modification relative to a wild-type canine or feline light chain κ constant region comprising:

a) at least one amino acid substitution at a position corresponding to position 11 and/or position 22 of SEQ ID NO: 150; or

b) at least one amino acid substitution at a position corresponding to position 11 and/or position 22 of SEQ ID NO: 156.
Embodiment 22. The polypeptide, the contiguous polypeptide, or the multimeric protein of any one of the preceding embodiments comprising a variant light chain constant region comprising at least one amino acid modification relative to a wild-type canine or feline light chain κ constant region comprising:

a) an alanine at a position corresponding to position 11 and/or an arginine at a position corresponding to position 22 of SEQ ID NO: 150; or

b) an alanine at a position corresponding to position 11 and/or an arginine at a position corresponding to position 22 of SEQ ID NO: 156.
Embodiment 23. The polypeptide, the contiguous polypeptide, or the multimeric protein of any one of the preceding embodiments comprising a variant light chain constant region comprising at least one amino acid modification relative to a wild-type canine or feline light chain κ constant region comprising:
a) an alanine at position 11 and/or an arginine at position 22 of SEQ ID NO: 150; or
b) an alanine at position 11 and/or an arginine at position 22 of SEQ ID NO: 156.
Embodiment 24. The polypeptide, the contiguous polypeptide, or the multimeric protein of any one the preceding embodiments, wherein the variant IgG Fc polypeptide comprises:

a) at least one amino acid substitution relative to a wild-type feline IgG Fc polypeptide, wherein the variant IgG Fc polypeptide comprises an amino acid substitution at a position corresponding to position 16 of SEQ ID NO: 65, SEQ ID NO: 66, SEQ ID NO: 67, SEQ ID NO: 68, or SEQ ID NO: 69;

b) at least one amino acid substitution relative to a wild-type equine IgG Fc polypeptide, wherein the variant IgG Fc polypeptide comprises an amino acid substitution at a position corresponding to position 3 of SEQ ID NO: 51; and/or

c) at least one amino acid substitution relative to a wild-type equine IgG Fc polypeptide, wherein the variant IgG Fc polypeptide comprises an amino acid substitution at a position corresponding to position 20 of SEQ ID NO: 51.
Embodiment 25. A polypeptide comprising a variant IgG Fc polypeptide, wherein the variant IgG Fc polypeptide comprises:

a) at least one amino acid substitution relative to a wild-type feline IgG Fc polypeptide, wherein the variant IgG Fc polypeptide comprises an amino acid substitution at a position corresponding to position 16 of SEQ ID NO: 65, SEQ ID NO: 66, SEQ ID NO: 67, SEQ ID NO: 68, or SEQ ID NO: 69;

b) at least one amino acid substitution relative to a wild-type equine IgG Fc polypeptide, wherein the variant IgG Fc polypeptide comprises an amino acid substitution at a position corresponding to position 3 of SEQ ID NO: 51; and/or

c) at least one amino acid substitution relative to a wild-type equine IgG Fc polypeptide, wherein the variant IgG Fc polypeptide comprises an amino acid substitution at a position corresponding to position 20 of SEQ ID NO: 51.
Embodiment 26. The polypeptide, the contiguous polypeptide, or the multimeric protein of any one the preceding embodiments, wherein the variant IgG Fc polypeptide comprises:

a) at least one amino acid substitution relative to a wild-type feline IgG Fc polypeptide, wherein the variant IgG Fc polypeptide comprises an amino acid substitution at position 16 of SEQ ID NO: 65, SEQ ID NO: 66, SEQ ID NO: 67, SEQ ID NO: 68, or SEQ ID NO: 69;

b) at least one amino acid substitution relative to a wild-type equine IgG Fc polypeptide, wherein the variant IgG Fc polypeptide comprises an amino acid substitution at position 3 of SEQ ID NO: 51; and/or

c) at least one amino acid substitution relative to a wild-type equine IgG Fc polypeptide, wherein the variant IgG Fc polypeptide comprises an amino acid substitution at position 20 of SEQ ID NO: 51.
Embodiment 27. The polypeptide, the contiguous polypeptide, or the multimeric protein, wherein the variant IgG Fc polypeptide comprises:

a) at least one amino acid substitution relative to a wild-type feline IgG Fc polypeptide, wherein the variant IgG Fc polypeptide comprises a proline at a position corresponding to position 16 or at position 16 of SEQ ID NO: 65, SEQ ID NO: 66, SEQ ID NO: 67, SEQ ID NO: 68, or SEQ ID NO: 69;

b) at least one amino acid substitution relative to a wild-type equine IgG Fc polypeptide, wherein the variant IgG Fc polypeptide comprises a serine at a position corresponding to position 3 or at position 3 of SEQ ID NO: 51; and/or

c) at least one amino acid substitution relative to a wild-type equine IgG Fc polypeptide, wherein the variant IgG Fc polypeptide comprises a proline at a position corresponding to position 20 or at position 20 of SEQ ID NO: 51.
Embodiment 28. The polypeptide, the contiguous polypeptide, or the multimeric protein of any one the preceding embodiments, wherein the variant IgG Fc polypeptide comprises a hinge region or a portion of a hinge region from an IgG Fc polypeptide of a different isotype.
Embodiment 29. The polypeptide, the contiguous polypeptide, or the multimeric protein of any one of the preceding embodiments, wherein the variant IgG Fc polypeptide comprises a hinge region or a portion of a hinge region from a wild-type feline IgG-1 Fc polypeptide or from a wild-type equine IgG1 Fc polypeptide.
Embodiment 30. The polypeptide, the contiguous polypeptide, or the multimeric protein of any one of the preceding embodiments, wherein the variant IgG Fc polypeptide comprises a cysteine at a position corresponding to position 8, position 9, position 10, position 11, position 12, position 13, position 14, position 15, or position 16 of SEQ ID NO: 69.
Embodiment 31. A polypeptide comprising a variant IgG Fc polypeptide, wherein the variant IgG Fc polypeptide comprises a cysteine at a position corresponding to position 8, position 9, position 10, position 11, position 12, position 13, position 14, position 15, or position 16 of SEQ ID NO: 69.
Embodiment 32. The polypeptide, the contiguous polypeptide, or the multimeric protein of any one of the preceding embodiments, wherein the variant IgG Fc polypeptide comprises a cysteine at a position corresponding to position 14 of SEQ ID NO: 69.
Embodiment 33. The polypeptide, the contiguous polypeptide, or the multimeric protein of any one of the preceding embodiments, wherein the variant IgG Fc polypeptide comprises a cysteine at position 14 of SEQ ID NO: 69.
Embodiment 34. The polypeptide, the contiguous polypeptide, or the multimeric protein of any one of the preceding embodiments, wherein the variant IgG Fc polypeptide comprises:
a) an amino acid substitution at a position corresponding to position 21 of SEQ ID NO: 1, an amino acid substitution at a position corresponding to position 23 of SEQ ID NO: 1, an amino acid substitution at a position corresponding to position 25 of SEQ ID NO: 1, an amino acid substitution at a position corresponding to position 80 of SEQ ID NO: 1, an amino acid substitution at a position corresponding to position 205 of SEQ ID NO: 1, and/or an amino acid substitution at a position corresponding to position 207 of SEQ ID NO: 1;
b) an amino acid substitution at a position corresponding to position 21 of SEQ ID NO: 4, an amino acid substitution at a position corresponding to position 23 of SEQ ID NO: 4, and/or an amino acid substitution at a position corresponding to position 24 of SEQ ID NO: 4;
c) an amino acid substitution at a position corresponding to position 21 of SEQ ID NO: 6, an amino acid substitution at a position corresponding to position 23 of SEQ ID NO: 6, an amino acid substitution at a position corresponding to position 25 of SEQ ID NO: 6, an amino acid substitution at a position corresponding to position 80 of SEQ ID NO: 6, and/or an amino acid substitution at a position corresponding to position 207 of SEQ ID NO: 6;
d) an amino acid substitution at a position corresponding to position 15 of SEQ ID NO: 50, and/or an amino acid substitution at a position corresponding to position 203 of SEQ ID NO: 50;
e) an amino acid substitution at a position corresponding to position 199 of SEQ ID NO: 54, and/or an amino acid substitution at a position corresponding to position 200 of SEQ ID NO: 54; and/or
f) an amino acid substitution at a position corresponding to position 199 of SEQ ID NO: 55, an amino acid substitution at a position corresponding to position 200 of SEQ ID NO: 55, an amino acid substitution at a position corresponding to position 201 of SEQ ID NO: 55, and/or an amino acid substitution at a position corresponding to position 202 of SEQ ID NO: 55.
Embodiment 35. A polypeptide comprising a variant IgG Fc polypeptide, wherein the variant IgG Fc polypeptide comprises:
a) an amino acid substitution at a position corresponding to position 21 of SEQ ID NO: 1, an amino acid substitution at a position corresponding to position 23 of SEQ ID NO: 1, an amino acid substitution at a position corresponding to position 25 of SEQ ID NO: 1, an amino acid substitution at a position corresponding to position 80 of SEQ ID NO: 1, an amino acid substitution at a position corresponding to position 205 of SEQ ID NO: 1, and/or an amino acid substitution at a position corresponding to position 207 of SEQ ID NO: 1;
b) an amino acid substitution at a position corresponding to position 21 of SEQ ID NO: 4, an amino acid substitution at a position corresponding to position 23 of SEQ ID NO: 4, and/or an amino acid substitution at a position corresponding to position 24 of SEQ ID NO: 4;
c) an amino acid substitution at a position corresponding to position 21 of SEQ ID NO: 6, an amino acid substitution at a position corresponding to position 23 of SEQ ID NO: 6, an amino acid substitution at a position corresponding to position 25 of SEQ ID NO: 6, an amino acid substitution at a position corresponding to position 80 of SEQ ID NO: 6, and/or an amino acid substitution at a position corresponding to position 207 of SEQ ID NO: 6;
d) an amino acid substitution at a position corresponding to position 15 of SEQ ID NO: 50, and/or an amino acid substitution at a position corresponding to position 203 of SEQ ID NO: 50;
e) an amino acid substitution at a position corresponding to position 199 of SEQ ID NO: 54, and/or an amino acid substitution at a position corresponding to position 200 of SEQ ID NO: 54; and/or
f) an amino acid substitution at a position corresponding to position 199 of SEQ ID NO: 55, an amino acid substitution at a position corresponding to position 200 of SEQ ID NO: 55, an amino acid substitution at a position corresponding to position 201 of SEQ ID NO: 55, and/or an amino acid substitution at a position corresponding to position 202 of SEQ ID NO: 55.
Embodiment 36. The polypeptide, the contiguous polypeptide, or the multimeric protein of any one of the preceding embodiments, wherein the variant IgG Fc polypeptide comprises:
a) an amino acid substitution at position 21 of SEQ ID NO: 1, an amino acid substitution at position 23 of SEQ ID NO: 1, an amino acid substitution at position 25 of SEQ ID NO: 1, an amino acid substitution at position 80 of SEQ ID NO: 1, an amino acid substitution at position 205 of SEQ ID NO: 1, and/or an amino acid substitution at position 207 of SEQ ID NO: 1;
b) an amino acid substitution at position 21 of SEQ ID NO: 4, an amino acid substitution at position 23 of SEQ ID NO: 4, and/or an amino acid substitution at position 24 of SEQ ID NO: 4;
c) an amino acid substitution at position 21 of SEQ ID NO: 4, an amino acid substitution at position 23 of SEQ ID NO: 6, an amino acid substitution at position 25 of SEQ ID NO: 6, an amino acid substitution at position 80 of SEQ ID NO: 6, and/or an amino acid substitution at position 207 of SEQ ID NO: 6;
d) an amino acid substitution at position 15 of SEQ ID NO: 50, and/or an amino acid substitution at position 203 of SEQ ID NO: 50;
e) an amino acid substitution at position 199 of SEQ ID NO: 54, and/or an amino acid substitution at position 200 of SEQ ID NO: 54; and/or
f) an amino acid substitution at position 199 of SEQ ID NO: 55, an amino acid substitution at position 200 of SEQ ID NO: 55, an amino acid substitution at position 201 of SEQ ID NO: 55, and/or an amino acid substitution at position 202 of SEQ ID NO: 55.
Embodiment 37. The polypeptide, the contiguous polypeptide, or the multimeric protein of any one of the preceding embodiments, wherein the variant IgG Fc polypeptide comprises:
a) a threonine at a position corresponding to position 21 of SEQ ID NO: 1, a leucine at a position corresponding to position 23 of SEQ ID NO: 1, an alanine at a position corresponding to position 25 of SEQ ID NO: 1, a glycine at a position corresponding to position 80 of SEQ ID NO: 1, an alanine at a position corresponding to position 205 of SEQ ID NO: 1, and/or a histidine at a position corresponding to position 207 of SEQ ID NO: 1;
b) a threonine at a position corresponding to position 21 of SEQ ID NO: 4, a leucine at a position corresponding to position 23 of SEQ ID NO: 4, and/or an isoleucine at a position corresponding to position 24 of SEQ ID NO: 4;
c) a threonine at a position corresponding to position 21 of SEQ ID NO: 6, a leucine at a position corresponding to position 23 of SEQ ID NO: 6, an alanine at a position corresponding to position 25 of SEQ ID NO: 6, a glycine at a position corresponding to position 80 of SEQ ID NO:6, and/or a histidine at a position corresponding to position 207 of SEQ ID NO: 6;
d) a threonine or a valine at a position corresponding to position 15 of SEQ ID NO: 50, and/or a tyrosine or a valine at a position corresponding to position 203 of SEQ ID NO: 50;
e) a leucine at a position corresponding to position 199 of SEQ ID NO: 54, and/or a histidine at a position corresponding to position 200 of SEQ ID NO: 54; and/or
f) a leucine at a position corresponding to position 199 of SEQ ID NO: 55, a histidine at a position corresponding to position 200 of SEQ ID NO: 55, an asparagine at a position corresponding to position 201 of SEQ ID NO: 55, and/or a histidine at a position corresponding to position 202 of SEQ ID NO: 55.
Embodiment 38. The polypeptide, the contiguous polypeptide, or the multimeric protein of any one of the preceding embodiments, wherein the variant IgG Fc polypeptide comprises:
a) a threonine at position 21 of SEQ ID NO: 1, a leucine at position 23 of SEQ ID NO: 1, an alanine at position 25 of SEQ ID NO: 1, a glycine at position 80 of SEQ ID NO: 1, an alanine at position 205 of SEQ ID NO: 1, and/or a histidine at position 207 of SEQ ID NO: 1;
b) a threonine at position 21 of SEQ ID NO: 3, a leucine at position 23 of SEQ ID NO: 4, and/or an isoleucine at position 24 of SEQ ID NO: 4;
c) a threonine at a position 21 of SEQ ID NO: 6, a leucine at position 23 of SEQ ID NO: 6, an alanine at position 25 of SEQ ID NO: 6, a glycine at position 80 of SEQ ID NO: 6, and/or a histidine at position 207 of SEQ ID NO: 6;
d) a threonine or a valine at position 15 of SEQ ID NO: 50, and/or a tyrosine or a valine at position 203 of SEQ ID NO: 50;
e) a leucine at position 199 of SEQ ID NO: 54, and/or a histidine at position 200 of SEQ ID NO: 54; and/or
f) a leucine at position 199 of SEQ ID NO: 55, a histidine at position 200 of SEQ ID NO: 55, an asparagine at position 201 of SEQ ID NO: 55, and/or a histidine at position 202 of SEQ ID NO: 55.
Embodiment 39. The polypeptide, the contiguous polypeptide, or the multimeric protein of any one of the preceding embodiments, wherein the variant IgG Fc polypeptide comprises:
a) an amino acid substitution at a position corresponding to position 93 of SEQ ID NO: 2, or an amino acid substitution at a position corresponding to position 93 of SEQ ID NO: 4;
b) an amino acid substitution at a position corresponding to position 87 of SEQ ID NO: 49, an amino acid substitution at a position corresponding to position 87 of SEQ ID NO: 52, an amino acid substitution at a position corresponding to position 87 of SEQ ID NO: 53, or an amino acid substitution at a position corresponding to position 87 of SEQ ID NO: 56; or
c) an amino acid substitution at a position corresponding to position 198 of SEQ ID NO: 65, an amino acid substitution at a position corresponding to position 198 of SEQ ID NO: 66, an amino acid substitution at a position corresponding to position 198 of SEQ ID NO: 67, or an amino acid substitution at a position corresponding to position 198 of SEQ ID NO: 68.
Embodiment 40. A polypeptide comprising a variant IgG Fc polypeptide, wherein the variant IgG Fc polypeptide comprises:
a) an amino acid substitution at a position corresponding to position 93 of SEQ ID NO: 2, or an amino acid substitution at a position corresponding to position 93 of SEQ ID NO: 4;
b) an amino acid substitution at a position corresponding to position 87 of SEQ ID NO: 49, an amino acid substitution at a position corresponding to position 87 of SEQ ID NO: 52, an amino acid substitution at a position corresponding to position 87 of SEQ ID NO: 53, or an amino acid substitution at a position corresponding to position 87 of SEQ ID NO: 56; or
c) an amino acid substitution at a position corresponding to position 198 of SEQ ID NO: 65, an amino acid substitution at a position corresponding to position 198 of SEQ ID NO: 66, an amino acid substitution at a position corresponding to position 198 of SEQ ID NO: 67, or an amino acid substitution at a position corresponding to position 198 of SEQ ID NO: 68.
Embodiment 41. The polypeptide, the contiguous polypeptide, or the multimeric protein of any one of the preceding embodiments, wherein the variant IgG Fc polypeptide comprises:
a) an amino acid substitution at position 93 of SEQ ID NO: 2, or an amino acid substitution at position 93 of SEQ ID NO: 4;
b) an amino acid substitution at position 87 of SEQ ID NO: 49, an amino acid substitution at position 87 of SEQ ID NO: 52, an amino acid substitution at position 87 of SEQ ID NO: 53, or an amino acid substitution at position 87 of SEQ ID NO: 56; or
c) an amino acid substitution at position 198 of SEQ ID NO: 65, an amino acid substitution at position 198 of SEQ ID NO: 66, an amino acid substitution at position 198 of SEQ ID NO: 67, or an amino acid substitution at position 198 of SEQ ID NO: 68.
Embodiment 42. The polypeptide, the contiguous polypeptide, or the multimeric protein of any one of the preceding embodiments, wherein the variant IgG Fc polypeptide comprises:
a) an arginine at a position corresponding to position 93 of SEQ ID NO: 2, or an arginine at a position corresponding to position 93 of SEQ ID NO: 4;
b) a serine at a position corresponding to position 87 of SEQ ID NO: 49, a serine substitution at a position corresponding to position 87 of SEQ ID NO: 52, a serine at a position corresponding to position 87 of SEQ ID NO: 53, or a serine at a position corresponding to position 87 of SEQ ID NO: 56; or
c) an alanine at a position corresponding to position 198 of SEQ ID NO: 65, an alanine at a position corresponding to position 198 of SEQ ID NO: 66, an alanine at a position corresponding to position 198 of SEQ ID NO: 67, or an alanine at a position corresponding to position 198 of SEQ ID NO: 68.
Embodiment 43. The polypeptide, the contiguous polypeptide, or the multimeric protein of any one of the preceding embodiments, wherein the variant IgG Fc polypeptide comprises:
a) an arginine at position 93 of SEQ ID NO: 2, or an arginine at position 93 of SEQ ID NO: 4;
b) a serine at position 87 of SEQ ID NO: 49, a serine at position 87 of SEQ ID NO: 52, a serine at position 87 of SEQ ID NO: 53, or a serine at position 87 of SEQ ID NO: 56; or
c) an alanine at position 198 of SEQ ID NO: 65, an alanine at position 198 of SEQ ID NO: 66, an alanine at position 198 of SEQ ID NO: 67, or alanine at position 198 of SEQ ID NO: 68.
Embodiment 44. The polypeptide, the contiguous polypeptide, or the multimeric protein of any one of the preceding embodiments, wherein the variant IgG Fc polypeptide comprises:
a) an amino acid substitution at a position corresponding to position 5 of SEQ ID NO: 2, an amino acid substitution at a position corresponding to position 38 of SEQ ID NO: 2, an amino acid substitution at a position corresponding to position 39 of SEQ ID NO: 2, an amino acid substitution at a position corresponding to position 97 of SEQ ID NO: 2, and/or an amino acid substitution at a position corresponding to position 98 of SEQ ID NO: 2; or
b) an amino acid substitution at a position corresponding to position 5 of SEQ ID NO: 4, an amino acid substitution at a position corresponding to position 38 of SEQ ID NO: 4, an amino acid substitution at a position corresponding to position 39 of SEQ ID NO: 4, an amino acid substitution at a position corresponding to position 97 of SEQ ID NO: 4, and/or an amino acid substitution at a position corresponding to position 98 of SEQ ID NO: 4.
Embodiment 45. A polypeptide comprising a variant IgG Fc polypeptide, wherein the variant IgG Fc polypeptide comprises:
a) an amino acid substitution at a position corresponding to position 5 of SEQ ID NO: 2, an amino acid substitution at a position corresponding to position 38 of SEQ ID NO: 2, an amino acid substitution at a position corresponding to position 39 of SEQ ID NO: 2, an amino acid substitution at a position corresponding to position 97 of SEQ ID NO: 2, and/or an amino acid substitution at a position corresponding to position 98 of SEQ ID NO: 2; or
b) an amino acid substitution at a position corresponding to position 5 of SEQ ID NO: 4, an amino acid substitution at a position corresponding to position 38 of SEQ ID NO: 4, an amino acid substitution at a position corresponding to position 39 of SEQ ID NO: 4, an amino acid substitution at a position corresponding to position 97 of SEQ ID NO: 4, and/or an amino acid substitution at a position corresponding to position 98 of SEQ ID NO: 4.
Embodiment 46. The polypeptide, the contiguous polypeptide, or the multimeric protein of any one of the preceding embodiments, wherein the variant IgG Fc polypeptide comprises:
a) an amino acid substitution at position 5 of SEQ ID NO: 2, an amino acid substitution at position 38 of SEQ ID NO: 2, an amino acid substitution at position 39 of SEQ ID NO: 2, an amino acid substitution at position 97 of SEQ ID NO: 2, and/or an amino acid substitution at position 98 of SEQ ID NO: 2; or
b) an amino acid substitution at position 5 of SEQ ID NO: 4, an amino acid substitution at position 38 of SEQ ID NO: 4, an amino acid substitution at position 39 of SEQ ID NO: 4, an amino acid substitution at position 97 of SEQ ID NO: 4, and/or an amino acid substitution at position 98 of SEQ ID NO: 4.
Embodiment 47. The polypeptide, the contiguous polypeptide, or the multimeric protein of any one of the preceding embodiments, wherein the variant IgG Fc polypeptide comprises:
a) a proline at a position corresponding to position 5 of SEQ ID NO: 2, a glycine at a position corresponding to position 38 of SEQ ID NO: 2, an arginine at a position corresponding to position 39 of SEQ ID NO: 2, an isoleucine at a position corresponding to position 97 of SEQ ID NO: 2, and/or a glycine at a position corresponding to position 98 of SEQ ID NO: 2; or
b) a proline at a position corresponding to position 5 of SEQ ID NO: 4, a glycine at a position corresponding to position 38 of SEQ ID NO: 4, an arginine at a position corresponding to position 39 of SEQ ID NO: 4, an isoleucine at a position corresponding to position 97 of SEQ ID NO: 4, and/or a glycine at a position corresponding to position 98 of SEQ ID NO: 4.
Embodiment 48. The polypeptide, the contiguous polypeptide, or the multimeric protein of any one of the preceding embodiments, wherein the variant IgG Fc polypeptide comprises:
a) a proline at position 5 of SEQ ID NO: 2, a glycine at position 38 of SEQ ID NO: 2, an arginine at position 39 of SEQ ID NO: 2, an isoleucine at position 97 of SEQ ID NO: 2, and/or a glycine at position 98 of SEQ ID NO: 2; or
b) a proline at position 5 of SEQ ID NO: 4, a glycine at position 38 of SEQ ID NO: 4, an arginine at position 39 of SEQ ID NO: 4, an isoleucine at position 97 of SEQ ID NO: 4, and/or a glycine at position 98 of SEQ ID NO: 4.
Embodiment 49. The polypeptide, the contiguous polypeptide, or the multimeric protein of any one of the preceding embodiments comprises:
a) a variant canine IgG-A Fc polypeptide comprising an alanine at a position corresponding to position 2 of SEQ ID NO: 1, a methionine or a lysine at a position corresponding to position 5 of SEQ ID NO: 1, a threonine at a position corresponding to position 21 of SEQ ID NO: 1, a leucine at a position corresponding to position 23 of SEQ ID NO: 1, an alanine at a position corresponding to position 25 of SEQ ID NO: 1, a valine at a position corresponding to position 35 of SEQ ID NO: 1, an asparagine at a position corresponding to position 38 of SEQ ID NO: 1, a proline at a position corresponding to position 39 of SEQ ID NO: 1, a glutamic acid at a position corresponding to position 65 of SEQ ID NO: 1, a glycine at a position corresponding to position 80 of SEQ ID NO: 1, a lysine at a position corresponding to position 93 of SEQ ID NO: 1, a asparagine at a position corresponding to position 96 of SEQ ID NO: 1, a lysine at a position corresponding to position 97 of SEQ ID NO: 1, an alanine at a position corresponding to position 98 of SEQ ID NO: 1, an alanine at a position corresponding to position 205 of SEQ ID NO: 1, and/or a histidine at a position corresponding to position 207 of SEQ ID NO: 1; or
b) a variant canine IgG-D Fc polypeptide comprising an alanine at a position corresponding to position 2 of SEQ ID NO: 6, a methionine or a lysine at a position corresponding to position 5 of SEQ ID NO: 6, a threonine at a position corresponding to position 21 of SEQ ID NO: 6, a leucine at a position corresponding to position 23 of SEQ ID NO: 6, an alanine at a position corresponding to position 25 of SEQ ID NO: 6, a valine at a position corresponding to position 35 of SEQ ID NO: 6, an asparagine at a position corresponding to position 38 of SEQ ID NO: 6, a proline at a position corresponding to position 39 of SEQ ID NO: 6, a glutamic acid at a position corresponding to position 65 of SEQ ID NO: 6, a glycine at a position corresponding to position 80 of SEQ ID NO: 6, a lysine at a position corresponding to position 93 of SEQ ID NO: 6, a asparagine at a position corresponding to position 96 of SEQ ID NO: 6, a lysine at a position corresponding to position 97 of SEQ ID NO: 6, an alanine at a position corresponding to position 98 of SEQ ID NO: 6, and/or a histidine at a position corresponding to position 207 of SEQ ID NO: 6.
Embodiment 50. A polypeptide comprising:
a) a variant canine IgG-A Fc polypeptide comprising an alanine at a position corresponding to position 2 of SEQ ID NO: 1, a methionine or a lysine at a position corresponding to position 5 of SEQ ID NO: 1, a threonine at a position corresponding to position 21 of SEQ ID NO: 1, a leucine at a position corresponding to position 23 of SEQ ID NO: 1, an alanine at a position corresponding to position 25 of SEQ ID NO: 1, a valine at a position corresponding to position 35 of SEQ ID NO: 1, an asparagine at a position corresponding to position 38 of SEQ ID NO: 1, a proline at a position corresponding to position 39 of SEQ ID NO: 1, a glutamic acid at a position corresponding to position 65 of SEQ ID NO: 1, a glycine at a position corresponding to position 80 of SEQ ID NO: 1, a lysine at a position corresponding to position 93 of SEQ ID NO: 1, a asparagine at a position corresponding to position 96 of SEQ ID NO: 1, a lysine at a position corresponding to position 97 of SEQ ID NO: 1, an alanine at a position corresponding to position 98 of SEQ ID NO: 1, an alanine at a position corresponding to position 205 of SEQ ID NO: 1, and/or a histidine at a position corresponding to position 207 of SEQ ID NO: 1; or
b) a variant canine IgG-D Fc polypeptide comprising an alanine at a position corresponding to position 2 of SEQ ID NO: 6, a methionine or a lysine at a position corresponding to position 5 of SEQ ID NO: 6, a threonine at a position corresponding to position 21 of SEQ ID NO: 6, a leucine at a position corresponding to position 23 of SEQ ID NO: 6, an alanine at a position corresponding to position 25 of SEQ ID NO: 6, a valine at a position corresponding to position 35 of SEQ ID NO: 6, an asparagine at a position corresponding to position 38 of SEQ ID NO: 6, a proline at a position corresponding to position 39 of SEQ ID NO: 6, a glutamic acid at a position corresponding to position 65 of SEQ ID NO: 6, a glycine at a position corresponding to position 80 of SEQ ID NO: 6, a lysine at a position corresponding to position 93 of SEQ ID NO: 6, a asparagine at a position corresponding to position 96 of SEQ ID NO: 6, a lysine at a position corresponding to position 97 of SEQ ID NO: 6, an alanine at a position corresponding to position 98 of SEQ ID NO: 6, and/or a histidine at a position corresponding to position 207 of SEQ ID NO: 6.
Embodiment 51. The polypeptide, the contiguous polypeptide, or the multimeric protein of any one of the preceding embodiments comprises:
a) a variant canine IgG-A Fc polypeptide comprising an alanine at position 2 of SEQ ID NO: 1, a methionine or a lysine at position 5 of SEQ ID NO: 1, a threonine at position 21 of SEQ ID NO: 1, a leucine at position 23 of SEQ ID NO: 1, an alanine at position 25 of SEQ ID NO: 1, a valine at position 35 of SEQ ID NO: 1, an asparagine at position 38 of SEQ ID NO: 1, a proline at position 39 of SEQ ID NO: 1, a glutamic acid at position 65 of SEQ ID NO: 1, a glycine at position 80 of SEQ ID NO: 1, a lysine at position 93 of SEQ ID NO: 1, a asparagine at position 96 of SEQ ID NO: 1, a lysine at position 97 of SEQ ID NO: 1, an alanine at position 98 of SEQ ID NO: 1, an alanine at position 205 of SEQ ID NO: 1, and/or a histidine at position 207 of SEQ ID NO: 1; or
b) a variant canine IgG-D Fc polypeptide comprising an alanine at position 2 of SEQ ID NO: 6, a methionine or a lysine at position 5 of SEQ ID NO: 6, a threonine at position 21 of SEQ ID NO: 6, a leucine at position 23 of SEQ ID NO: 6, an alanine at position 25 of SEQ ID NO: 6, a valine at position 35 of SEQ ID NO: 6, an asparagine at position 38 of SEQ ID NO: 6, a proline at position 39 of SEQ ID NO: 6, a glutamic acid at position 65 of SEQ ID NO: 6, a glycine at position 80 of SEQ ID NO: 6, a lysine at position 93 of SEQ ID NO: 6, a asparagine at position 96 of SEQ ID NO: 6, a lysine at position 97 of SEQ ID NO: 6, an alanine at position 98 of SEQ ID NO: 6, and/or a histidine at position 207 of SEQ ID NO: 6.
Embodiment 52. The polypeptide, the contiguous polypeptide, or the multimeric protein of any one of the preceding embodiments comprising a variant IgG Fc polypeptide comprising:

a) a tyrosine or a tryptophan at a position corresponding to position 138 of SEQ ID NO: 1, a tyrosine or a tryptophan at a position corresponding to position 137 of SEQ ID NO: 2, a tyrosine or a tryptophan at a position corresponding to position 137 of SEQ ID NO: 4, or a tyrosine or a tryptophan at a position corresponding to position 138 of SEQ ID NO: 6;

b) a tyrosine or a tryptophan at a position corresponding to position 154 of SEQ ID NO: 69, a tyrosine or a tryptophan at a position corresponding to position 154 of SEQ ID NO: 65 or SEQ ID NO: 66, or a tyrosine or a tryptophan at a position corresponding to position 154 of SEQ ID NO: 67 or SEQ ID NO: 68; or

c) a tyrosine or a tryptophan at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56.
Embodiment 53. A polypeptide comprising a variant IgG Fc polypeptide, wherein the variant IgG Fc polypeptide comprises:
a) a tyrosine or a tryptophan at a position corresponding to position 138 of SEQ ID NO: 1, a tyrosine or a tryptophan at a position corresponding to position 137 of SEQ ID NO: 2, a tyrosine or a tryptophan at a position corresponding to position 137 of SEQ ID NO: 4, or a tyrosine or a tryptophan at a position corresponding to position 138 of SEQ ID NO: 6;
b) a tyrosine or a tryptophan at a position corresponding to position 154 of SEQ ID NO: 69, a tyrosine or a tryptophan at a position corresponding to position 154 of SEQ ID NO: 65 or SEQ ID NO: 66, or a tyrosine or a tryptophan at a position corresponding to position 154 of SEQ ID NO: 67 or SEQ ID NO: 68; or
c) a tyrosine or a tryptophan at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56.
Embodiment 54. The polypeptide, the contiguous polypeptide, or the multimeric protein of any one of the preceding embodiments, wherein the variant IgG Fc polypeptide comprises:

a) a tyrosine or a tryptophan at position 138 of SEQ ID NO: 1, a tyrosine or a tryptophan at position 137 of SEQ ID NO: 2, a tyrosine or a tryptophan at position 137 of SEQ ID NO: 4, or a tyrosine or a tryptophan at position 138 of SEQ ID NO: 6; or

b) a tyrosine or a tryptophan at position 154 of SEQ ID NO: 69, a tyrosine or a tryptophan at position 154 of SEQ ID NO: 65 or SEQ ID NO: 66, or a tyrosine or a tryptophan at position 154 of SEQ ID NO: 67 or SEQ ID NO: 68; and/or

c) a tyrosine or a tryptophan at position 131 of SEQ ID NO: 49, SEQ ID NO: 50, SEQ ID NO: 52, SEQ ID NO: 53, SEQ ID NO: 54, SEQ ID NO: 55, or SEQ ID NO: 56.
Embodiment 55. The polypeptide, the contiguous polypeptide, or the multimeric protein of any one of the preceding embodiments comprising a variant IgG Fc polypeptide comprising:

a) a serine at a position corresponding to position 138 of SEQ ID NO: 1, a serine at a position corresponding to position 137 of SEQ ID NO: 2, a serine at a position corresponding to position 137 of SEQ ID NO: 4, a serine at a position corresponding to position 138 of SEQ ID NO: 6, a serine at a position corresponding to position 154 of SEQ ID NO: 69, a serine at a position corresponding to position 154 of SEQ ID NO: 65 or SEQ ID NO: 66, a serine at a position corresponding to position 154 of SEQ ID NO: 67 or SEQ ID NO: 68, or a serine at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56;

b) an alanine at a position corresponding to position 140 of SEQ ID NO: 1, an alanine at a position corresponding to position 139 of SEQ ID NO: 2, an alanine at a position corresponding to position 139 of SEQ ID NO: 4, an alanine at a position corresponding to position 140 of SEQ ID NO: 6, an alanine at a position corresponding to position 156 of SEQ ID NO: 69, an alanine at a position corresponding to position 156 of SEQ ID NO: 65 or SEQ ID NO: 66, an alanine at a position corresponding to position 156 of SEQ ID NO: 67 or SEQ ID NO: 68, or an alanine at a position corresponding to position 133 of SEQ ID NO: 49, SEQ ID NO: 50, SEQ ID NO: 52, SEQ ID NO: 53, SEQ ID NO: 54, SEQ ID NO: 55, or SEQ ID NO: 56; and/or

c) a threonine at a position corresponding to position 181 of SEQ ID NO: 1, a threonine at a position corresponding to position 180 of SEQ ID NO: 2, a threonine at a position corresponding to position 180 of SEQ ID NO: 4, a threonine at a position corresponding to position 181 of SEQ ID NO: 6, a threonine at a position corresponding to position 197 of SEQ ID NO: 69, a threonine at a position corresponding to position 197 of SEQ ID NO: 65 or SEQ ID NO: 66, a threonine at a position corresponding to position 197 of SEQ ID NO: 67 or SEQ ID NO: 68, or a threonine at a position corresponding to position 174 of SEQ ID NO: 49, SEQ ID NO: 50, SEQ ID NO: 52, SEQ ID NO: 53, SEQ ID NO: 54, SEQ ID NO: 55, or SEQ ID NO: 56.
Embodiment 56. A polypeptide comprising a variant IgG Fc polypeptide, wherein the variant IgG Fc polypeptide comprises:
a) a serine at a position corresponding to position 138 of SEQ ID NO: 1, a serine at a position corresponding to position 137 of SEQ ID NO: 2, a serine at a position corresponding to position 137 of SEQ ID NO: 4, a serine at a position corresponding to position 138 of SEQ ID NO: 6, a serine at a position corresponding to position 154 of SEQ ID NO: 69, a serine at a position corresponding to position 154 of SEQ ID NO: 65 or SEQ ID NO: 66, a serine at a position corresponding to position 154 of SEQ ID NO: 67 or SEQ ID NO: 68, or a serine at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56;
b) an alanine at a position corresponding to position 140 of SEQ ID NO: 1, an alanine at a position corresponding to position 139 of SEQ ID NO: 2, an alanine at a position corresponding to position 139 of SEQ ID NO: 4, an alanine at a position corresponding to position 140 of SEQ ID NO: 6, an alanine at a position corresponding to position 156 of SEQ ID NO: 69, an alanine at a position corresponding to position 156 of SEQ ID NO: 65 or SEQ ID NO: 66, an alanine at a position corresponding to position 156 of SEQ ID NO: 67 or SEQ ID NO: 68, or an alanine at a position corresponding to position 133 of SEQ ID NO: 49, SEQ ID NO: 50, SEQ ID NO: 52, SEQ ID NO: 53, SEQ ID NO: 54, SEQ ID NO: 55, or SEQ ID NO: 56; and/or
c) a threonine at a position corresponding to position 181 of SEQ ID NO: 1, a threonine at a position corresponding to position 180 of SEQ ID NO: 2, a threonine at a position corresponding to position 180 of SEQ ID NO: 4, a threonine at a position corresponding to position 181 of SEQ ID NO: 6, a threonine at a position corresponding to position 197 of SEQ ID NO: 69, a threonine at a position corresponding to position 197 of SEQ ID NO: 65 or SEQ ID NO: 66, a threonine at a position corresponding to position 197 of SEQ ID NO: 67 or SEQ ID NO: 68, or a threonine at a position corresponding to position 174 of SEQ ID NO: 49, SEQ ID NO: 50, SEQ ID NO: 52, SEQ ID NO: 53, SEQ ID NO: 54, SEQ ID NO: 55, or SEQ ID NO: 56.
Embodiment 57. The polypeptide, the contiguous polypeptide, or the multimeric protein of any one of the preceding embodiments, wherein the variant IgG Fc polypeptide comprises:

a) a serine at position 138 of SEQ ID NO: 1, a serine at position 137 of SEQ ID NO: 2, a serine at position 137 of SEQ ID NO: 4, a serine at position 138 of SEQ ID NO: 6, a serine at position 154 of SEQ ID NO: 69, a serine at position 154 of SEQ ID NO: 65 or SEQ ID NO: 66, a serine at position 154 of SEQ ID NO: 67 or SEQ ID NO: 68, or a serine at position 131 of SEQ ID NO: 49, SEQ ID NO: 50, SEQ ID NO: 52, SEQ ID NO: 53, SEQ ID NO: 54, SEQ ID NO: 55, or SEQ ID NO: 56;

b) an alanine at position 140 of SEQ ID NO: 1, an alanine at position 139 of SEQ ID NO: 2, an alanine at position 139 of SEQ ID NO: 4, an alanine at position 140 of SEQ ID NO: 6, an alanine at position 156 of SEQ ID NO: 69, an alanine at position 156 of SEQ ID NO: 65 or SEQ ID NO: 66, an alanine at position 156 of SEQ ID NO: 67 or SEQ ID NO: 68, or an alanine at position 133 of SEQ ID NO: 49, SEQ ID NO: 50, SEQ ID NO: 52, SEQ ID NO: 53, SEQ ID NO: 54, SEQ ID NO: 55, or SEQ ID NO: 56; and/or;

c) a threonine at position 181 of SEQ ID NO: 1, a threonine at position 181 of SEQ ID NO: 2, a threonine at position 181 of SEQ ID NO: 4, a threonine at position 181 of SEQ ID NO: 6, a threonine at position 197 of SEQ ID NO: 69, a threonine at position 197 of SEQ ID NO: 65 or SEQ ID NO: 66, a threonine at position 197 of SEQ ID NO: 67 or SEQ ID NO: 68, or a threonine at position 174 of SEQ ID NO: 49, SEQ ID NO: 50, SEQ ID NO: 52, SEQ ID NO: 53, SEQ ID NO: 54, SEQ ID NO: 55, or SEQ ID NO: 56.
Embodiment 58. The polypeptide, the contiguous polypeptide, or the multimeric protein of any one of the preceding embodiments, wherein the polypeptide or the variant Fc polypeptide is glycoslylated.
Embodiment 59. The polypeptide, the contiguous polypeptide, or the multimeric protein of any one of the preceding embodiments, wherein the polypeptide or the variant Fc polypeptide is aglycosylated.
Embodiment 60. The polypeptide, the contiguous polypeptide, or the multimeric protein of any one of the preceding embodiments, wherein L1 and L2, if present, each independently is a flexible linker.
Embodiment 61. The polypeptide, the contiguous polypeptide, or the multimeric protein of any one of the preceding embodiments, wherein the amino acid sequence of L1 and L2, if present, each independently comprises 100%, at least 95%, at least 90%, at least 85% serine and/or glycine amino acid residues.
Embodiment 62. The polypeptide, the contiguous polypeptide, or the multimeric protein of any one of the preceding embodiments, wherein the polypeptide, contiguous polypeptide, or multimeric polypeptide comprises an extension at a C-terminus.
Embodiment 63. The polypeptide, the contiguous polypeptide, or the multimeric protein of any one of the preceding embodiments, wherein the polypeptide, contiguous polypeptide, or multimeric polypeptide comprises a glycine residue, two glycine residues, three glycine residues, four glycine residues, five glycine residues, six glycine residues, seven glycine residues, eight glycine residues, or greater than eight glycine residues at a C-terminus.
Embodiment 64. The polypeptide, the contiguous polypeptide, or the multimeric protein of any one of preceding embodiments, wherein the contiguous polypeptide comprises an amino acid sequence of SEQ ID NO: 158, SEQ ID NO: 159, SEQ ID NO: 160, SEQ ID NO: 161, SEQ ID NO: 162, SEQ ID NO: 163, SEQ ID NO: 164, or SEQ ID NO: 165 at its C-terminus.
Embodiment 65. The polypeptide, the contiguous polypeptide, or the multimeric protein of any one of the preceding embodiments, wherein the therapeutic polypeptide, TPA1, and/or TPA2 is selected from an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ECD of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.
Embodiment 66. The polypeptide, the contiguous polypeptide, or the multimeric protein of any one of the preceding embodiments, wherein the therapeutic polypeptide, TPA1, and/or TPA2 is a canine polypeptide, a feline polypeptide, or an equine polypeptide.
Embodiment 67. The polypeptide, the contiguous polypeptide, or the multimeric protein of any one of the preceding embodiments, wherein the antibody, TPA1, and/or TPA2 is an antibody that binds a target polypeptide selected from an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.
Embodiment 68. The polypeptide, the contiguous polypeptide, or the multimeric protein of any one of the preceding embodiments, wherein the antibody binds a canine target polypeptide, a feline target polypeptide, or an equine target polypeptide.
Embodiment 69. The polypeptide, the contiguous polypeptide, or the multimeric protein of any one of the preceding embodiments, wherein the variant IgG Fc polypeptide comprises an amino acid sequence having at least 90% identity, at least 95% identity, at least 97% identity, or at least 99% identity to the amino acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 166, 167, 168, 169, 170, 171, 172, 173, 174, 175, 176, 177, 178, 179, 180, and/or 250.
Embodiment 70. The polypeptide, the contiguous polypeptide, or the multimeric protein of any one of the preceding embodiments comprising an amino acid sequence of SEQ ID NO: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 57, 58, 59, 60, 61, 62, 63, 64, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 146, 147, 148, 149, 150, 151, 154, 155, 157, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 271, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, and/or 250.
Embodiment 71. A polypeptide comprising an amino acid sequence of SEQ ID NO: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 57, 58, 59, 60, 61, 62, 63, 64, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 146, 147, 148, 149, 150, 151, 154, 155, 157, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 271, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, and/or 250.
Embodiment 72. The polypeptide, the multimeric protein, or the contiguous polypeptide of any one of the preceding embodiments, wherein the at least one amino acid modification or substitution comprises an amino acid substitution with an amino acid derivative.
Embodiment 73. An isolated nucleic acid encoding the polypeptide, the multimeric protein, or the contiguous polypeptide of any one of the preceding embodiments.
Embodiment 74. A host cell comprising the nucleic acid of embodiment 74.
Embodiment 75. A method of producing a polypeptide comprising culturing the host cell of embodiment 74 and isolating the polypeptide.
Embodiment 76. A pharmaceutical composition comprising the polypeptide, the multimeric protein, or the contiguous polypeptide of any one of embodiments 1 to 72, and a pharmaceutically acceptable carrier.
Embodiment 77. A method of exposing a cell to the polypeptide, the multimeric protein, the contiguous polypeptide, or the pharmaceutical composition of any one of embodiments 1 to 72 or 76.
Embodiment 78. The method of embodiment 77, wherein the cell is exposed to the polypeptide, heterodimeric protein, contiguous polypeptide, or the pharmaceutical composition ex vivo.
Embodiment 79. The method of embodiment 77, wherein the cell is exposed to the polypeptide, heterodimeric protein, contiguous polypeptide, or the pharmaceutical composition in vivo.
Embodiment 80. The method of any one of embodiments 77 to 79, wherein the cell is a human cell, a canine cell, a feline cell, or an equine cell.
Embodiment 81. A method of delivering a polypeptide to a subject comprising administering the polypeptide, the multimeric protein, the contiguous polypeptide, or the pharmaceutical composition of any one of embodiments 1 to 72 or 76 parenterally.
Embodiment 82. A method of delivering a polypeptide to a subject comprising administering the polypeptide, the multimeric protein, the contiguous polypeptide, or the pharmaceutical composition of any one of embodiments 1 to 72 or 76 by an intramuscular route, an intraperitoneal route, an intracerebrospinal route, a subcutaneous route, an intra-arterial route, an intrasynovial route, an intrathecal route, or an inhalation route.
Embodiment 83. A method of treating a subject having diabetes or obesity, the method comprising administering to the subject a therapeutically effective amount of the polypeptide, the multimeric protein, the contiguous polypeptide, or the pharmaceutical composition of any one of embodiments 1 to 72 or 76.
Embodiment 84. The method of any one of embodiments 81 to 83, wherein the subject is a human subject.
Embodiment 85. The method of any one of embodiments 81 to 83, wherein the subject is a companion animal species.
Embodiment 86. The method of embodiment 85, wherein the companion animal species is canine, equine, or feline.

### BRIEF DESCRIPTION OF THE DRAWINGS

FIG. 1 shows an alignment of canine IgG-A, B, C, and D Fc sequences. The boxes indicate the regions likely in contact with Protein A.

FIG. 2A shows an SDS-PAGE analysis of GLP1-G8/GLP-2G_III_WTfeIgG2 (SEQ ID NO: 23; “GLP1 A variant” in this figure) and GLP1-G8_I_WTfeIgG2 (SEQ ID NO: 24; “GLP1 B variant” in this figure) having wild-type feline IgG2 hinge with one disulfide bond in the absence and presence of reducing agent (DTT).

FIG. 2B shows an SDS-PAGE analysis of GLP1-G8/GLP-2G_III_VARfeIgG2 (SEQ ID NO: 25; “GLP1 MA variant” in this figure) of GLP1-G8_I_VARfeIgG2 (SEQ ID NO: 26; “GLP1 MB variant” in this figure) having variant feline IgG2 hinge with two disulfide bonds in the absence and presence of reducing agent (DTT).

### DESCRIPTION OF THE SEQUENCES

TABLE 1
Table 1 provides a listing of exemplary sequences referenced herein.
Description of the Sequences

| SEQ ID NO: | SEQUENCE | DESCRIPTION |
|---|---|---|
| 1 | PVPEPLGGPSVLIFPPKPKDILRITRTPEVTC | Exemplary wild-type canine |
| | VVLDLGREDPEVQISWFVDGKEVHTAKTQSRE | IgG-A Fc |
| | QQFNGTYRVVSVLPIEHQDWLTGKEFKCRVNH | Protein A − |
| | IDLPSPIERTISKARGRAHKPSVYVLPPSPKE | C1q − |
| | LSSSDTVSITCLIKDFYPPDIDVEWQSNGQQE | CD16 − |
| | PERKHRMTPPQLDEDGSYFLYSKLSVDKSRWQ | |
| | QGDPFTCAVMHETLQNHYTDLSLSHSPGK | |
| 2 | PAPEMLGGPSVFIFPPKPKDTLLIARTPEVTC | Exemplary wild-type canine |
| | VVVDLDPEDPEVQISWFVDGKQMQTAKTQPRE | IgG-B Fc |
| | EQFNGTYRVVSVLPIGHQDWLKGKQFTCKVNN | Protein A + |
| | KALPSPIERTISKARGQAHQPSVYVLPPSREE | C1q + |
| | LSKNTVSLTCLIKDFFPPDIDVEWQSNGQQEP | CD16 + |
| | ESKYRTTPPQLDEDGSYFLYSKLSVDKSRWQR | |
| | GDTFICAVMHEALHNHYTQESLSHSPGK | |
| 3 | PKRENGRVPRPPDCPKCPAPEMLGGPSVFIFP | Exemplary wild-type canine |
| | PKPKDTLLIARTPEVTCVVVDLDPEDPEVQIS | IgG-B Fc with hinge |
| | WFVDGKQMQTAKTQPREEQFNGTYRVVSVLPI | Protein A + |
| | GHQDWLKGKQFTCKVNNKALPSPIERTISKAR | C1q + |
| | GQAHQPSVYVLPPSREELSKNTVSLTCLIKDF | CD16 + |
| | FPPDIDVEWQSNGQQEPESKYRTTPPQLDEDG | |
| | SYFLYSKLSVDKSRWQRGDTFICAVMHEALHN | |
| | HYTQESLSHSPGK | |
| 4 | PGCGLLGGPSVFIFPPKPKDILVTARTPTVTC | Exemplary wild-type canine |
| | VVVDLDPENPEVQISWFVDSKQVQTANTQPRE | IgG-C Fc |
| | EQSNGTYRVVSVLPIGHQDWLSGKQFKCKVNN | Protein A − |
| | KALPSPIEEIISKTPGQAHQPNVYVLPPSRDE | C1q + |
| | MSKNTVTLTCLVKDFFPPEIDVEWQSNGQQEP | CD16 + |
| | ESKYRMTPPQLDEDGSYFLYSKLSVDKSRWQR | |
| | GDTFICAVMHEALHNHYTQISLSHSPGK | |
| 5 | AKECECKCNCNNCPCPGCGLLGGPSVFIFPPK | Exemplary wild-type canine |
| | PKDILVTARTPTVTCVVVDLDPENPEVQISWF | IgG-C Fc with hinge |
| | VDSKQVQTANTQPREEQSNGTYRVVSVLPIGH | Protein A − |
| | QDWLSGKQFKCKVNNKALPSPIEEIISKTPGQ | C1q + |
| | AHQPNVYVLPPSRDEMSKNTVTLTCLVKDFFP | CD16 + |
| | PEIDVEWQSNGQQEPESKYRMTPPQLDEDGSY | |
| | FLYSKLSVDKSRWQRGDTFICAVMHEALHNHY | |
| | TQISLSHSPGK | |
| 6 | PVPESLGGPSVFIFPPKPKDILRITRTPEITC | Exemplary wild-type canine |
| | VVLDLGREDPEVQISWFVDGKEVHTAKTQPRE | IgG-D Fc |
| | QQFNSTYRVVSVLPIEHQDWLTGKEFKCRVNH | Protein A − |
| | IGLPSPIERTISKARGQAHQPSVYVLPPSPKE | C1q − |
| | LSSSDTVTLTCLIKDFFPPEIDVEWQSNGQPE | CD16 − |
| | PESKYHTTAPQLDEDGSYFLYSKLSVDKSRWQ | |
| | QGDTFTCAVMHEALQNHYTDLSLSHSPGK | |
| 7 | PVPEPLGGPSVLIFPPKPKDTLLIARTPEVTC | Exemplary variant canine |
| | VVLDLGREDPEVQISWFVDGKEVHTAKTQSRE | IgG-A Fc |
| | QQFNGTYRVVSVLPIGHQDWLTGKEFKCRVNH | C1q − |
| | IDLPSPIERTISKARGRAHKPSVYVLPPSPKE | Protein A + |
| | LSSSDTVSITCLIKDFYPPDIDVEWQSNGQQE | I(21)T |
| | PERKHRMTPPQLDEDGSYFLYSKLSVDKSRWQ | R(23)L |
| | QGDPFTCAVMHEALHNHYTDLSLSHSPGK | T(25)A |
| | | E(80)G |
| | | T(205)A |
| | | Q(207)H |
| 8 | PGCGLLGGPSVFIFPPKPKDTLLIARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | C1q + |
| | KALPSPIEEIISKTPGQAHQPNVYVLPPSRDE | Protein A + |
| | MSKNTVTLTCLVKDFFPPEIDVEWQSNGQQEP | I(21)T |
| | ESKYRMTPPQLDEDGSYFLYSKLSVDKSRWQR | V(23)L |
| | GDTFICAVMHEALHNHYTQISLSHSPGK | T(24)I |
| 9 | PVPESLGGPSVFIFPPKPKDTLLIARTPEITC | Exemplary variant canine |
| | VVLDLGREDPEVQISWFVDGKEVHTAKTQPRE | IgG-D Fc |
| | QQFNSTYRVVSVLPIGHQDWLTGKEFKCRVNH | C1q − |
| | IGLPSPIERTISKARGQAHQPSVYVLPPSPKE | Protein A + |
| | LSSSDTVTLTCLIKDFFPPEIDVEWQSNGQPE | I(21)T |
| | PESKYHTTAPQLDEDGSYFLYSKLSVDKSRWQ | R(23)L |
| | QGDTFTCAVMHEALHNHYTDLSLSHSPGK | T(25)A |
| | | E(80)G |
| | | Q(207)H |
| 10 | PVPEPLGGPSVLIFPPKPKDTLRITRTPEVTC | Exemplary variant canine |
| | VVLDLGREDPEVQISWFVDGKEVHTAKTQSRE | IgG-A Fc |
| | QQFNGTYRVVSVLPIEHQDWLTGKEFKCRVNH | C1q − |
| | IDLPSPIERTISKARGRAHKPSVYVLPPSPKE | Protein A + |
| | LSSSDTVSITCLIKDFYPPDIDVEWQSNGQQE | I(21)T |
| | PERKHRMTPPQLDEDGSYFLYSKLSVDKSRWQ | Q(207)H |
| | QGDPFTCAVMHETLHNHYTDLSLSHSPGK | |
| 11 | PGCGLLGGPSVFIFPPKPKDTLVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | C1q + |
| | KALPSPIEEIISKTPGQAHQPNVYVLPPSRDE | Protein A + |
| | MSKNTVTLTCLVKDFFPPEIDVEWQSNGQQEP | I(21)T |
| | ESKYRMTPPQLDEDGSYFLYSKLSVDKSRWQR | |
| | GDTFICAVMHEALHNHYTQISLSHSPGK | |
| 12 | PVPESLGGPSVFIFPPKPKDTLRITRTPEITC | Exemplary variant canine |
| | VVLDLGREDPEVQISWFVDGKEVHTAKTQPRE | IgG-D Fc |
| | QQFNSTYRVVSVLPIEHQDWLTGKEFKCRVNH | C1q − |
| | IGLPSPIERTISKARGQAHQPSVYVLPPSPKE | Protein A + |
| | LSSSDTVTLTCLIKDFFPPEIDVEWQSNGQPE | I(21)T |
| | PESKYHTTAPQLDEDGSYFLYSKLSVDKSRWQ | Q(207)H |
| | QGDTFTCAVMHEALHNHYTDLSLSHSPGK | |
| 13 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCRVNN | Protein A + |
| | KALPSPIERTISKARGQAHQPSVYVLPPSREE | C1q − |
| | LSKNTVSLTCLIKDFFPPDIDVEWQSNGQQEP | K(93)R |
| | ESKYRTTPPQLDEDGSYFLYSKLSVDKSRWQR | |
| | GDTFICAVMHEALHNHYTQESLSHSPGK | |
| 14 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCRVNN | Protein A − |
| | KALPSPIEEIISKTPGQAHQPNVYVLPPSRDE | C1q − |
| | MSKNTVTLTCLVKDFFPPEIDVEWQSNGQQEP | CD16 + |
| | ESKYRMTPPQLDEDGSYFLYSKLSVDKSRWQR | K(93)R |
| | GDTFICAVMHEALHNHYTQISLSHSPGK | |
| 15 | PAPEPLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Protein A + |
| | KALPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | M(5)P |
| | GDTFICAVMHEALHNHYTQESLSHSPGK | |
| 16 | PAPEMLGGPSVFIFPPKPKDTLLIARTPEVTC | Exemplary variant canine |
| | VVVDLDREDPEVQISWFVDGKQMQTAKTQPRE | IgG-B Fc |
| | EQFNGTYRVVSVLPIGHQDWLKGKQFTCKVNN | Protein A + |
| | KALPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | P(39)R |
| | GDTFICAVMHEALHNHYTQESLSHSPGK | |
| 17 | PAPEMLGGPSVFIFPPKPKDTLLIARTPEVTC | Exemplary variant canine |
| | VVVDLGPEDPEVQISWFVDGKQMQTAKTQPRE | IgG-B Fc |
| | EQFNGTYRVVSVLPIGHQDWLKGKQFTCKVNN | Protein A + |
| | KALPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | D(38)G |
| | GDTFICAVMHEALHNHYTQESLSHSPGK | |
| 18 | PAPEMLGGPSVFIFPPKPKDTLLIARTPEVTC | Exemplary variant canine |
| | VVVDLGREDPEVQISWFVDGKQMQTAKTQPRE | IgG-B Fc |
| | EQFNGTYRVVSVLPIGHQDWLKGKQFTCKVNN | Protein A + |
| | KALPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | D(38)G |
| | GDTFICAVMHEALHNHYTQESLSHSPGK | P(39)R |
| 19 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Protein A + |
| | IALPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | K(97)I |
| | GDTFICAVMHEALHNHYTQESLSHSPGK | |
| 20 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Protein A + |
| | KGLPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | A(98)G |
| | GDTFICAVMHEALHNHYTQESLSHSPGK | |
| 21 | PAPEMLGGPSVFIFPPKPKDTLLIARTPEVTC | Exemplary variant canine |
| | VVVDLGPEDPEVQISWFVDGKQMQTAKTQPRE | IgG-B Fc |
| | EQFNGTYRVVSVLPIGHQDWLKGKQFTCKVNN | Protein A + |
| | IGLPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | D(38)G |
| | GDTFICAVMHEALHNHYTQESLSHSPGK | K(97)I |
| | | A(98)G |
| 22 | PAPEPLGGPSVFIFPPKPKDTLLIARTPEVTC | Exemplary variant canine |
| | VVVDLDREDPEVQISWFVDGKQMQTAKTQPRE | IgG-B Fc |
| | EQFNGTYRVVSVLPIGHQDWLKGKQFTCKVNN | Protein A + |
| | KALPSPIERTISKARGQAHQPSVYVLPPSREE | C1q + |
| | LSKNTVSLTCLIKDFFPPDIDVEWQSNGQQEP | CD16 − |
| | ESKYRTTPPQLDEDGSYFLYSKLSVDKSRWQR | M(5)P |
| | GDTFICAVMHEALHNHYTQESLSHSPGK | P(39)R |
| 23 | PGCGPLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Protein A − |
| | KALPSPIEEIISKTPGQAHQPNVYVLPPSRDE | C1q + |
| | MSKNTVTLTCLVKDFFPPEIDVEWQSNGQQEP | CD16 − |
| | ESKYRMTPPQLDEDGSYFLYSKLSVDKSRWQR | L(5)P |
| | GDTFICAVMHEALHNHYTQISLSHSPGK | |
| 24 | PGCGLLGGPSVFIFPPKPKDILVTARTPTVTC | Exemplary variant canine |
| | VVVDLDRENPEVQISWFVDSKQVQTANTQPRE | IgG-C Fc |
| | EQSNGTYRVVSVLPIGHQDWLSGKQFKCKVNN | Protein A − |
| | KALPSPIEEIISKTPGQAHQPNVYVLPPSRDE | C1q + |
| | MSKNTVTLTCLVKDFFPPEIDVEWQSNGQQEP | CD16 − |
| | ESKYRMTPPQLDEDGSYFLYSKLSVDKSRWQR | P(39)R |
| | GDTFICAVMHEALHNHYTQISLSHSPGK | |
| 25 | PGCGLLGGPSVFIFPPKPKDILVTARTPTVTC | Exemplary variant canine |
| | VVVDLGPENPEVQISWFVDSKQVQTANTQPRE | IgG-C Fc |
| | EQSNGTYRVVSVLPIGHQDWLSGKQFKCKVNN | Protein A − |
| | KALPSPIEEIISKTPGQAHQPNVYVLPPSRDE | C1q + |
| | MSKNTVTLTCLVKDFFPPEIDVEWQSNGQQEP | CD16 − |
| | ESKYRMTPPQLDEDGSYFLYSKLSVDKSRWQR | D(38)G |
| | GDTFICAVMHEALHNHYTQISLSHSPGK | |
| 26 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Protein A − |
| | IALPSPIEEIISKTPGQAHQPNVYVLPPSRDE | C1q + |
| | MSKNTVTLTCLVKDFFPPEIDVEWQSNGQQEP | CD16 − |
| | ESKYRMTPPQLDEDGSYFLYSKLSVDKSRWQR | K(97)I |
| | GDTFICAVMHEALHNHYTQISLSHSPGK | |
| 27 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Protein A − |
| | KGLPSPIEEIISKTPGQAHQPNVYVLPPSRDE | C1q + |
| | MSKNIVTLICLVKDFFPPEIDVEWQSNGQQEP | CD16 − |
| | ESKYRMTPPQLDEDGSYFLYSKLSVDKSRWQR | A(98)G |
| | GDTFICAVMHEALHNHYTQISLSHSPGK | |
| 28 | PGCGPLGGPSVFIFPPKPKDILVTARTPTVTC | Exemplary variant canine |
| | VVVDLDRENPEVQISWFVDSKQVQTANTQPRE | IgG-C Fc |
| | EQSNGTYRVVSVLPIGHQDWLSGKQFKCKVNN | Protein A − |
| | KALPSPIEEIISKTPGQAHQPNVYVLPPSRDE | C1q + |
| | MSKNTVTLTCLVKDFFPPEIDVEWQSNGQQEP | CD16 − |
| | ESKYRMTPPQLDEDGSYFLYSKLSVDKSRWQR | L(5)P |
| | GDTFICAVMHEALHNHYTQISLSHSPGK | P(39)R |
| 29 | PGCGLLGGPSVFIFPPKPKDILVTARTPTVTC | Exemplary variant canine |
| | VVVDLGPENPEVQISWFVDSKQVQTANTQPRE | IgG-C Fc |
| | EQSNGTYRVVSVLPIGHQDWLSGKQFKCKVNN | Protein A − |
| | IGLPSPIEEIISKTPGQAHQPNVYVLPPSRDE | C1q + |
| | MSKNTVTLTCLVKDFFPPEIDVEWQSNGQQEP | CD16 − |
| | ESKYRMTPPQLDEDGSYFLYSKLSVDKSRWQR | D(38)G |
| | GDTFICAVMHEALHNHYTQISLSHSPGK | K(97)I |
| | | A(98)G |
| 30 | PGCGLLGGPSVFIFPPKPKDTLLIARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCRVNN | C1q − |
| | KALPSPIEEIISKTPGQAHQPNVYVLPPSRDE | K(93)R |
| | MSKNTVTLTCLVKDFFPPEIDVEWQSNGQQEP | Protein A + |
| | ESKYRMTPPQLDEDGSYFLYSKLSVDKSRWQR | I(21)T |
| | GDTFICAVMHEALHNHYTQISLSHSPGK | V(23)L |
| | | T(24)I |
| 31 | PAPEMLGGPSVFIFPPKPKDTLLIARTPEVTC | Exemplary variant canine |
| | VVVDLGPEDPEVQISWFVDGKQMQTAKTQPRE | IgG-B Fc |
| | EQFNGTYRVVSVLPIGHQDWLKGKQFTCRVNN | Protein A + |
| | IGLPSPIERTISKARGQAHQPSVYVLPPSREE | C1q − |
| | LSKNTVSLTCLIKDFFPPDIDVEWQSNGQQEP | CD16 − |
| | ESKYRTTPPQLDEDGSYFLYSKLSVDKSRWQR | D(38)G |
| | GDTFICAVMHEALHNHYTQESLSHSPGK | K(93)R |
| | | K(97)I |
| | | A(98)G |
| 32 | PAPEPLGGPSVFIFPPKPKDTLLIARTPEVTC | Exemplary variant canine |
| | VVVDLDREDPEVQISWFVDGKQMQTAKTQPRE | IgG-B Fc |
| | EQFNGTYRVVSVLPIGHQDWLKGKQFTCRVNN | Protein A + |
| | KALPSPIERTISKARGQAHQPSVYVLPPSREE | C1q − |
| | LSKNTVSLTCLIKDFFPPDIDVEWQSNGQQEP | CD16 − |
| | ESKYRTTPPQLDEDGSYFLYSKLSVDKSRWQR | M(5)P |
| | GDTFICAVMHEALHNHYTQESLSHSPGK | P(39)R |
| | | K(93)R |
| 33 | PGCGLLGGPSVFIFPPKPKDILVTARTPTVTC | Exemplary variant canine |
| | VVVDLGPENPEVQISWFVDSKQVQTANTQPRE | IgG-C Fc |
| | EQSNGTYRVVSVLPIGHQDWLSGKQFKCRVNN | Protein A − |
| | IGLPSPIEEIISKTPGQAHQPNVYVLPPSRDE | C1q − |
| | MSKNTVTLTCLVKDFFPPEIDVEWQSNGQQEP | CD16 − |
| | ESKYRMTPPQLDEDGSYFLYSKLSVDKSRWQR | D(38)G |
| | GDTFICAVMHEALHNHYTQISLSHSPGK | K(93)R |
| | | K(97)I |
| | | A(98)G |
| 34 | PGCGPLGGPSVFIFPPKPKDILVTARTPTVTC | Exemplary variant canine |
| | VVVDLDRENPEVQISWFVDSKQVQTANTQPRE | IgG-C Fc |
| | EQSNGTYRVVSVLPIGHQDWLSGKQFKCRVNN | Protein A − |
| | KALPSPIEEIISKTPGQAHQPNVYVLPPSRDE | C1q − |
| | MSKNTVTLTCLVKDFFPPEIDVEWQSNGQQEP | CD16 − |
| | ESKYRMTPPQLDEDGSYFLYSKLSVDKSRWQR | M(5)P |
| | GDTFICAVMHEALHNHYTQISLSHSPGK | P(39)R |
| | | K(93)R |
| 35 | PVPEPLGGPSVLIFPPKPKDILRITRTPEVTC | Exemplary variant canine |
| | VVLDLGREDPEVQISWFVDGKEVHTAKTQSRE | IgG-A Fc |
| | QQFNGTYRVVSVLPIEHQDWLTGKEFKCKVNH | Protein A − |
| | IDLPSPIERTISKARGRAHKPSVYVLPPSPKE | C1q + |
| | LSSSDTVSITCLIKDFYPPDIDVEWQSNGQQE | CD16 − |
| | PERKHRMTPPQLDEDGSYFLYSKLSVDKSRWQ | R(93)K |
| | QGDPFTCAVMHETLQNHYTDLSLSHSPGK | |
| 36 | PAPEMLGGPSVLIFPPKPKDILRITRTPEVTC | Exemplary variant canine |
| | VVVDLDPEDPEVQISWFVDGKEVHTAKTQSRE | IgG-A Fc |
| | EQFNGTYRVVSVLPIEHQDWLTGKEFKCRVNN | Protein A − |
| | KALPSPIERTISKARGRAHKPSVYVLPPSPKE | C1q + |
| | LSSSDTVSITCLIKDFYPPDIDVEWQSNGQQE | CD16 + |
| | PERKHRMTPPQLDEDGSYFLYSKLSVDKSRWQ | V(2)A |
| | QGDPFTCAVMHETLQNHYTDLSLSHSPGK | P(5)M |
| | | L(35)V |
| | | G(38)D |
| | | R(39)P |
| | | Q(65)E |
| | | H(96)N |
| | | I(97)K |
| | | D(98)A |
| 37 | PAPELLGGPSVLIFPPKPKDILRITRTPEVTC | Exemplary variant canine |
| | VVVDLDPEDPEVQISWFVDGKEVHTAKTQSRE | IgG-A Fc |
| | EQFNGTYRVVSVLPIEHQDWLTGKEFKCRVNN | Protein A − |
| | KALPSPIERTISKARGRAHKPSVYVLPPSPKE | C1q − |
| | LSSSDTVSITCLIKDFYPPDIDVEWQSNGQQE | CD16 + |
| | PERKHRMTPPQLDEDGSYFLYSKLSVDKSRWQ | V(2)A |
| | QGDPFTCAVMHETLQNHYTDLSLSHSPGK | P(5)L |
| | | L(35)V |
| | | G(38)D |
| | | R(39)P |
| | | Q(65)E |
| | | H(96)N |
| | | I(97)K |
| | | D(98)A |
| 38 | PAPEMLGGPSVLIFPPKPKDILRITRTPEVTC | Exemplary variant canine |
| | VVVDLDPEDPEVQISWFVDGKEVHTAKTQSRE | IgG-A Fc |
| | EQFNGTYRVVSVLPIEHQDWLTGKEFKCKVNN | Protein A − |
| | KALPSPIERTISKARGRAHKPSVYVLPPSPKE | C1q + |
| | LSSSDTVSITCLIKDFYPPDIDVEWQSNGQQE | CD16 + |
| | PERKHRMTPPQLDEDGSYFLYSKLSVDKSRWQ | V(2)A |
| | QGDPFTCAVMHETLQNHYTDLSLSHSPGK | P(5)M |
| | | L(35)V |
| | | G(38)D |
| | | R(39)P |
| | | Q(65)E |
| | | R(93)K |
| | | H(96)N |
| | | I(97)K |
| | | D(98)A |
| 39 | PAPELLGGPSVLIFPPKPKDILRITRTPEVTC | Exemplary variant canine |
| | VVVDLDPEDPEVQISWFVDGKEVHTAKTQSRE | IgG-A Fc |
| | EQFNGTYRVVSVLPIEHQDWLTGKEFKCKVNN | Protein A − |
| | KALPSPIERTISKARGRAHKPSVYVLPPSPKE | C1q + |
| | LSSSDTVSITCLIKDFYPPDIDVEWQSNGQQE | CD16 + |
| | PERKHRMTPPQLDEDGSYFLYSKLSVDKSRWQ | V(2)A |
| | QGDPFTCAVMHETLQNHYTDLSLSHSPGK | P(5)L |
| | | L(35)V |
| | | G(38)D |
| | | R(39)P |
| | | Q(65)E |
| | | R(93)K |
| | | H(96)N |
| | | I(97)K |
| | | D(98)A |
| 40 | PAPEMLGGPSVLIFPPKPKDTLLIARTPEVTC | Exemplary variant canine |
| | VVVDLDPEDPEVQISWFVDGKEVHTAKTQSRE | IgG-A Fc |
| | EQFNGTYRVVSVLPIGHQDWLTGKEFKCKVNN | Protein A + |
| | KALPSPIERTISKARGRAHKPSVYVLPPSPKE | C1q + |
| | LSSSDTVSITCLIKDFYPPDIDVEWQSNGQQE | CD16 + |
| | PERKHRMTPPQLDEDGSYFLYSKLSVDKSRWQ | V(2)A |
| | QGDPFTCAVMHEALHNHYTDLSLSHSPGK | P(5)M |
| | | I(21)T |
| | | R(23)L |
| | | T(25)A |
| | | L(35)V |
| | | G(38)D |
| | | R(39)P |
| | | Q(65)E |
| | | E(80)G |
| | | R(93)K |
| | | H(96)N |
| | | I(97)K |
| | | D(98)A |
| | | T(205)A |
| | | Q(207)H |
| 41 | PAPELLGGPSVLIFPPKPKDTLLIARTPEVTC | Exemplary variant canine |
| | VVVDLDPEDPEVQISWFVDGKEVHTAKTQSRE | IgG-A Fc |
| | EQFNGTYRVVSVLPIGHQDWLTGKEFKCKVNN | Protein A + |
| | KALPSPIERTISKARGRAHKPSVYVLPPSPKE | C1q + |
| | LSSSDTVSITCLIKDFYPPDIDVEWQSNGQQE | CD16 + |
| | PERKHRMTPPQLDEDGSYFLYSKLSVDKSRWQ | V(2)A |
| | QGDPFTCAVMHEALHNHYTDLSLSHSPGK | P(5)L |
| | | I(21)T |
| | | R(23)L |
| | | T(25)A |
| | | L(35)V |
| | | G(38)D |
| | | R(39)P |
| | | Q(65)E |
| | | E(80)G |
| | | R(93)K |
| | | H(96)N |
| | | I(97)K |
| | | D(98)A |
| | | T(205)A |
| | | Q(207)H |
| 42 | PVPESLGGPSVFIFPPKPKDILRITRTPEITC | Exemplary variant canine |
| | VVLDLGREDPEVQISWFVDGKEVHTAKTQPRE | IgG-D Fc |
| | QQFNSTYRVVSVLPIEHQDWLTGKEFKCKVNH | Protein A − |
| | IGLPSPIERTISKARGQAHQPSVYVLPPSPKE | C1q + |
| | LSSSDTVTLTCLIKDFFPPEIDVEWQSNGQPE | CD16 − |
| | PESKYHTTAPQLDEDGSYFLYSKLSVDKSRWQ | R(93)K |
| | QGDTFTCAVMHEALQNHYTDLSLSHSPGK | |
| 43 | PAPEMLGGPSVFIFPPKPKDILRITRTPEITC | Exemplary variant canine |
| | VVVDLDPEDPEVQISWFVDGKEVHTAKTQPRE | IgG-D Fc |
| | EQFNSTYRVVSVLPIEHQDWLTGKEFKCRVNN | Protein A − |
| | KALPSPIERTISKARGQAHQPSVYVLPPSPKE | C1q − |
| | LSSSDTVTLTCLIKDFFPPEIDVEWQSNGQPE | CD16 |
| | PESKYHTTAPQLDEDGSYFLYSKLSVDKSRWQ | V(2)A |
| | QGDTFTCAVMHEALQNHYTDLSLSHSPGK | S(5)M |
| | | L(35)V |
| | | G(38)D |
| | | R(39)P |
| | | Q(65)E |
| | | H(96)N |
| | | I(97)K |
| | | G(98)A |
| 44 | PAPELLGGPSVFIFPPKPKDILRITRTPEITC | Exemplary variant canine |
| | VVVDLDPEDPEVQISWFVDGKEVHTAKTQPRE | IgG-D Fc |
| | EQFNSTYRVVSVLPIEHQDWLTGKEFKCRVNN | Protein A − |
| | KALPSPIERTISKARGQAHQPSVYVLPPSPKE | C1q − |
| | LSSSDTVTLTCLIKDFFPPEIDVEWQSNGQPE | CD16 + |
| | PESKYHTTAPQLDEDGSYFLYSKLSVDKSRWQ | V(2)A |
| | QGDTFTCAVMHEALQNHYTDLSLSHSPGK | S(5)L |
| | | L(35)V |
| | | G(38)D |
| | | R(39)P |
| | | Q(65)E |
| | | H(96)N |
| | | I(97)K |
| | | G(98)A |
| 45 | PAPEMLGGPSVFIFPPKPKDILRITRTPEITC | Exemplary variant canine |
| | VVVDLDPEDPEVQISWFVDGKEVHTAKTQPRE | IgG-D Fc |
| | EQFNSTYRVVSVLPIEHQDWLTGKEFKCKVNN | Protein A − |
| | KALPSPIERTISKARGQAHQPSVYVLPPSPKE | C1q + |
| | LSSSDTVTLTCLIKDFFPPEIDVEWQSNGQPE | CD16 + |
| | PESKYHTTAPQLDEDGSYFLYSKLSVDKSRWQ | V(2)A |
| | QGDTFTCAVMHEALQNHYTDLSLSHSPGK | S(5)M |
| | | L(35)V |
| | | G(38)D |
| | | R(39)P |
| | | Q(65)E |
| | | R(93)K |
| | | H(96)N |
| | | I(97)K |
| | | G(98)A |
| 46 | PAPELLGGPSVFIFPPKPKDILRITRTPEITC | Exemplary variant canine |
| | VVVDLDPEDPEVQISWFVDGKEVHTAKTQPRE | IgG-D Fc |
| | EQFNSTYRVVSVLPIEHQDWLTGKEFKCKVNN | Protein A − |
| | KALPSPIERTISKARGQAHQPSVYVLPPSPKE | C1q + |
| | LSSSDTVTLTCLIKDFFPPEIDVEWQSNGQPE | CD16 + |
| | PESKYHTTAPQLDEDGSYFLYSKLSVDKSRWQ | V(2)A |
| | QGDTFTCAVMHEALQNHYTDLSLSHSPGK | S(5)L |
| | | L(35)V |
| | | G(38)D |
| | | R(39)P |
| | | Q(65)E |
| | | R(93)K |
| | | H(96)N |
| | | I(97)K |
| | | G(98)A |
| 47 | PAPEMLGGPSVFIFPPKPKDTLLIARTPEITC | Exemplary variant canine |
| | VVVDLDPEDPEVQISWFVDGKEVHTAKTQPRE | IgG-D Fc |
| | EQFNSTYRVVSVLPIGHQDWLTGKEFKCKVNN | Protein A + |
| | KALPSPIERTISKARGQAHQPSVYVLPPSPKE | C1q + |
| | LSSSDTVTLTCLIKDFFPPEIDVEWQSNGQPE | CD16 + |
| | PESKYHTTAPQLDEDGSYFLYSKLSVDKSRWQ | V(2)A |
| | QGDTFTCAVMHEALHNHYTDLSLSHSPGK | S(5)M |
| | | I(21)T |
| | | R(23)L |
| | | T(25)A |
| | | L(35)V |
| | | G(38)D |
| | | R(39)P |
| | | Q(65)E |
| | | E(80)G |
| | | R(93)K |
| | | H(96)N |
| | | I(97)K |
| | | G(98)A |
| | | Q(207)H |
| 48 | PAPELLGGPSVFIFPPKPKDTLLIARTPEITC | Exemplary variant canine |
| | VVVDLDPEDPEVQISWFVDGKEVHTAKTQPRE | IgG-D Fc |
| | EQFNSTYRVVSVLPIGHQDWLTGKEFKCKVNN | Protein A + |
| | KALPSPIERTISKARGQAHQPSVYVLPPSPKE | C1q + |
| | LSSSDTVTLTCLIKDFFPPEIDVEWQSNGQPE | CD16 + |
| | PESKYHTTAPQLDEDGSYFLYSKLSVDKSRWQ | V(2)A |
| | QGDTFTCAVMHEALHNHYTDLSLSHSPGK | S(5)L |
| | | I(21)T |
| | | R(23)L |
| | | T(25)A |
| | | L(35)V |
| | | G(38)D |
| | | R(39)P |
| | | Q(65)E |
| | | E(80)G |
| | | R(93)K |
| | | H(96)N |
| | | I(97)K |
| | | G(98)A |
| | | Q(207)H |
| 49 | GGPSVFLFPPNPKDTLMITRTPEVTCVVVDVS | Exemplary wild-type equine |
| | QENPDVKFNWYMDGVEVRTATTRPKEEQFNST | IgG1 Fc |
| | YRVVSVLRIQHQDWLSGKEFKCKVNNQALPQP | Protein A + |
| | IERTITKTKGRSQEPQVYVLAPHPDESKKSKV | C1q + |
| | SVTCLVKDFYPPEINIEWQSNGQPELETKYST | |
| | TQAQQDSDGSYFLYSKLSVDRNRWQQGTTFTC | |
| | GVMHEALHNHYTQKNVSKNPGK | |
| 50 | GGPSVFIFPPNPKDALMISRTPVVTCVVVNLS | Exemplary wild-type equine |
| | DQYPDVQFSWYVDNTEVHSAITKQREAQFNST | IgG2 Fc |
| | YRVVSVLPIQHQDWLSGKEFKCSVTNVGVPQP | Protein A − |
| | ISRAISRGKGPSRVPQVYVLPPHPDELAKSKV | C1q − |
| | SVTCLVKDFYPPDISVEWQSNRWPELEGKYST | |
| | TPAQLDGDGSYFLYSKLSLETSRWQQVESFTC | |
| | AVMHEALHNHFTKTDISESLGK | |
| 51 | PPCVLSAEGVIPIPSVPKPQCPPYTHSKFLGG | Exemplary wild-type equine |
| | PSVFIFPPNPKDALMISRTPVVTCVVVNLSDQ | IgG2 Fc with hinge |
| | YPDVQFSWYVDNTEVHSAITKQREAQFNSTYR | Protein A − |
| | VVSVLPIQHQDWLSGKEFKCSVTNVGVPQPIS | C1q − |
| | RAISRGKGPSRVPQVYVLPPHPDELAKSKVSV | |
| | TCLVKDFYPPDISVEWQSNRWPELEGKYSTTP | |
| | AQLDGDGSYFLYSKLSLETSRWQQVESFTCAV | |
| | MHEALHNHFTKTDISESLGK | |
| 52 | GGPSVFIFPPKPKDVLMITRMPEVTCLVVDVS | Exemplary wild-type equine |
| | HDSSDVLFTWYVDGTEVKTAKTMPNEEQNNST | IgG3 Fc |
| | YRVVSVLRIQHQDWLNGKKFKCKVNNQALPAP | Protein A + |
| | VERTISKATGQTRVPQVYVLAPHPDELSKNKV | C1q + |
| | SVTCLVKDFYPPDITVEWQSNEHPEPEGKYRT | |
| | TEAQKDSDGSYFLYSKLTVEKDRWQQGTTFTC | |
| | VVMHEALHNHVMQKNISKNPGK | |
| 53 | VGPSVFIFPPKPKDVLMISRTPTVTCVVVDVG | Exemplary wild-type equine |
| | HDFPDVQFNWYVDGVETHTATTEPKQEQFNST | IgG4 Fc |
| | YRVVSVLPIQHKDWLSGKEFKCKVNNKALPAP | Protein A + |
| | VERTISAPTGQPREPQVYVLAPHRDELSKNKV | C1q + |
| | SVTCLVKDFYPPDIDIEWKSNGQPEPETKYST | |
| | TPAQLDSDGSYFLYSKLTVETNRWQQGTTFTC | |
| | AVMHEALHNHYTEKSVSKSPGK | |
| 54 | GGPSVFIFPPKPKDVLMISRKPEVTCVVVDLG | Exemplary wild-type equine |
| | HDDPDVQFTWFVDGVETHTATTEPKEEQFNST | IgG5 Fc |
| | YRVVSVLPIQHQDWLSGKEFKCSVTSKALPAP | Protein A − |
| | VERTISKAKGQLRVPQVYVLAPHPDELAKNTV | C1q − |
| | SVTCLVKDFYPPEIDVEWQSNEHPEPEGKYST | |
| | TPAQLNSDGSYFLYSKLSVETSRWKQGESFTC | |
| | GVMHEAVENHYTQKNVSHSPGK | |
| 55 | GRPSVFIFPPNPKDTLMISRTPEVTCVVVDVS | Exemplary wild-type equine |
| | QENPDVKFNWYVDGVEAHTATTKAKEKQDNST | IgG6 Fc |
| | YRVVSVLPIQHQDWRRGKEFKCKVNNRALPAP | Protein A − |
| | VERTITKAKGELQDPQVYILAPHPDEVTKNTV | C1q − |
| | SVTCLVKDFYPPDINVEWQSNEEPEPEVKYST | |
| | TPAQLDGDGSYFLYSKLTVETDRWEQGESFTC | |
| | VVMHEAIRHTYRQKSITNFPGK | |
| 56 | VGPSVFIFPPKPKDVLMISRTPTVTCVVVDVG | Exemplary wild-type equine |
| | HDFPDVQFNWYVDGVETHTATTEPKQEQNNST | IgG7 Fc |
| | YRVVSILAIQHKDWLSGKEFKCKVNNQALPAP | Protein A + |
| | VQKTISKPTGQPREPQVYVLAPHPDELSKNKV | C1q + |
| | SVTCLVKDFYPPDIDIEWKSNGQPEPETKYST | |
| | TPAQLDGDGSYFLYSKLTVETNRWQQGTTFTC | |
| | AVMHEALHNHYTEKSVSKSPGK | |
| 57 | GGPSVFIFPPNPKDALMISRTPVVTCVVVNLS | Exemplary variant equine |
| | DQYPDVQFSWYVDNTEVHSAITKQREAQFNST | IgG2 Fc |
| | YRVVSVLPIQHQDWLSGKEFKCSVTNVGVPQP | C1q − |
| | ISRAISRGKGPSRVPQVYVLPPHPDELAKSKV | Protein A + |
| | SVTCLVKDFYPPDISVEWQSNRWPELEGKYST | F(203)Y |
| | TPAQLDGDGSYFLYSKLSLETSRWQQGESFTC | |
| | AVMHEALHNHYTKTDISESLGK | |
| 58 | GGPSVFIFPPNPKDTLMISRTPVVTCVVVNLS | Exemplary variant equine |
| | DQYPDVQFSWYVDNTEVHSAITKQREAQFNST | IgG2 Fc |
| | YRVVSVLPIQHQDWLSGKEFKCSVTNVGVPQP | C1q − |
| | ISRAISRGKGPSRVPQVYVLPPHPDELAKSKV | Protein A + |
| | SVTCLVKDFYPPDISVEWQSNRWPELEGKYST | A(15)T |
| | TPAQLDGDGSYFLYSKLSLETSRWQQVESFTC | F(203)Y |
| | AVMHEALHNHYTKTDISESLGK | |
| 59 | GGPSVFIFPPKPKDVLMISRKPEVTCVVVDLG | Exemplary variant equine |
| | HDDPDVQFTWFVDGVETHTATTEPKEEQFNST | IgG5 Fc |
| | YRVVSVLPIQHQDWLSGKEFKCSVTSKALPAP | C1q − |
| | VERTISKAKGQLRVPQVYVLAPHPDELAKNTV | Protein A + |
| | SVTCLVKDFYPPEIDVEWQSNEHPEPEGKYST | V(199)L |
| | TPAQLNSDGSYFLYSKLSVETSRWKQGESFTC | E(200)H |
| | GVMHEALHNHYTQKNVSHSPGK | |
| 60 | GRPSVFIFPPNPKDTLMISRTPEVTCVVVDVS | Exemplary variant equine |
| | QENPDVKFNWYVDGVEAHTATTKAKEKQDNST | IgG6 Fc |
| | YRVVSVLPIQHQDWRRGKEFKCKVNNRALPAP | C1q − |
| | VERTITKAKGELQDPQVYILAPHPDEVTKNTV | Protein A + |
| | SVTCLVKDFYPPDINVEWQSNEEPEPEVKYST | I(199)L |
| | TPAQLDGDGSYFLYSKLTVETDRWEQGESFTC | R(200)H |
| | VVMHEALHNHYRQKSITNFPGK | H(201)N |
| | | T(202)H |
| 61 | GGPSVFLFPPNPKDTLMITRTPEVTCVVVDVS | Exemplary variant equine |
| | QENPDVKFNWYMDGVEVRTATTRPKEEQFNST | IgG1 Fc |
| | YRVVSVLRIQHQDWLSGKEFKCSVNNQALPQP | Protein A + |
| | IERTITKTKGRSQEPQVYVLAPHPDESKKSKV | C1q − |
| | SVTCLVKDFYPPEINIEWQSNGQPELETKYST | K(87)S |
| | TQAQQDSDGSYFLYSKLSVDRNRWQQGTTFTC | |
| | GVMHEALHNHYTQKNVSKNPGK | |
| 62 | GGPSVFIFPPKPKDVLMITRMPEVTCLVVDVS | Exemplary variant equine |
| | HDSSDVLFTWYVDGTEVKTAKTMPNEEQNNST | IgG3 Fc |
| | YRVVSVLRIQHQDWLNGKKFKCSVNNQALPAP | Protein A + |
| | VERTISKATGQTRVPQVYVLAPHPDELSKNKV | C1q − |
| | SVTCLVKDFYPPDITVEWQSNEHPEPEGKYRT | K(87)S |
| | TEAQKDSDGSYFLYSKLTVEKDRWQQGTTFTC | |
| | VVMHEALHNHVMQKNISKNPGK | |
| 63 | VGPSVFIFPPKPKDVLMISRTPTVTCVVVDVG | Exemplary variant equine |
| | HDFPDVQFNWYVDGVETHTATTEPKQEQFNST | IgG4 Fc |
| | YRVVSVLPIQHKDWLSGKEFKCSVNNKALPAP | Protein A + |
| | VERTISAPTGQPREPQVYVLAPHRDELSKNKV | C1q − |
| | SVTCLVKDFYPPDIDIEWKSNGQPEPETKYST | K(87)S |
| | TPAQLDSDGSYFLYSKLTVETNRWQQGTTFTC | |
| | AVMHEALHNHYTEKSVSKSPGK | |
| 64 | VGPSVFIFPPKPKDVLMISRTPTVTCVVVDVG | Exemplary variant equine |
| | HDFPDVQFNWYVDGVETHTATTEPKQEQNNST | IgG7 Fc |
| | YRVVSILAIQHKDWLSGKEFKCSVNNQALPAP | Protein A + |
| | VQKTISKPTGQPREPQVYVLAPHPDELSKNKV | C1q − |
| | SVTCLVKDFYPPDIDIEWKSNGQPEPETKYST | K(87)S |
| | TPAQLDGDGSYFLYSKLTVETNRWQQGTTFTC | |
| | AVMHEALHNHYTEKSVSKSPGK | |
| 65 | RKTDHPPGPKTGEGPKCPPPEMLGGPSIFIFP | Exemplary wild-type feline |
| | PKPKDTLSISRTPEVTCLVVDLGPDDSDVQIT | IgG1a Fc |
| | WFVDNTQVYTAKTSPREEQFNSTYRVVSVLPI | Protein A + |
| | LHQDWLKGKEFKCKVNSKSLPSPIERTISKAK | C1q + |
| | GQPHEPQVYVLPPAQEELSENKVSVTCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 66 | RKTDHPPGPKPCDCPKCPPPEMLGGPSIFIFP | Exemplary wild-type feline |
| | PKPKDTLSISRTPEVTCLVVDLGPDDSDVQIT | IgG1a Fc |
| | WFVDNTQVYTAKTSPREEQFNSTYRVVSVLPI | Protein A + |
| | LHQDWLKGKEFKCKVNSKSLPSPIERTISKAK | C1q + |
| | GQPHEPQVYVLPPAQEELSENKVSVTCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 67 | RKTDHPPGPKTGEGPKCPPPEMLGGPSIFIFP | Exemplary wild-type feline |
| | PKPKDTLSISRTPEVTCLVVDLGPDDSDVQIT | IgG1b Fc |
| | WFVDNTQVYTAKTSPREEQFNSTYRVVSVLPI | Protein A + |
| | LHQDWLKGKEFKCKVNSKSLPSPIERTISKDK | C1q + |
| | GQPHEPQVYVLPPAQEELSENKVSVTCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 68 | RKTDHPPGPKPCDCPKCPPPEMLGGPSIFIFP | Exemplary wild-type feline |
| | PKPKDTLSISRTPEVTCLVVDLGPDDSDVQIT | IgG1b Fc |
| | WFVDNTQVYTAKTSPREEQFNSTYRVVSVLPI | Protein A + |
| | LHQDWLKGKEFKCKVNSKSLPSPIERTISKDK | C1q + |
| | GQPHEPQVYVLPPAQEELSENKVSVTCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 69 | PKTASTIESKTGEGPKCPVPEIPGAPSVFIFP | Exemplary wild-type feline |
| | PKPKDTLSISRTPEVTCLVVDLGPDDSNVQIT | IgG2 Fc |
| | WFVDNTEMHTAKTRPREEQFNSTYRVVSVLPI | Protein A + |
| | LHQDWLKGKEFKCKVNSKSLPSAMERTISKAK | C1q − |
| | GQPHEPQVYVLPPTQEELSENKVSVTCLIKGF | |
| | HPPDIAVEWEITGQPEPENNYQTTPPQLDSDG | |
| | TYFLYSRLSVDRSHWQRGNTYTCSVSHEALHS | |
| | HHTQKSLTQSPGK | |
| 70 | RKTDHPPGPKPCDCPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Protein A + |
| | LHQDWLKGKEFKCKVNSKSLPSPIERTISKAK | C1q − |
| | GQPHEPQVYVLPPAQEELSENKVSVTCLIKSF | P(198)A |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 71 | RKTDHPPGPKTGEGPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Protein A + |
| | LHQDWLKGKEFKCKVNSKSLPSPIERTISKAK | C1q − |
| | GQPHEPQVYVLPPAQEELSENKVSVTCLIKSF | P(198)A |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 72 | RKTDHPPGPKPCDCPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Protein A + |
| | LHQDWLKGKEFKCKVNSKSLPSPIERTISKDK | C1q − |
| | GQPHEPQVYVLPPAQEELSENKVSVTCLIEGF | P(198)A |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 73 | RKTDHPPGPKTGEGPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Protein A + |
| | LHQDWLKGKEFKCKVNSKSLPSPIERTISKDK | C1q − |
| | GQPHEPQVYVLPPAQEELSENKVSVTCLIEGF | P(198)A |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 74 | PVPEPLGGPSVLIFPPKPKDILRITRTPEVTC | Exemplary variant canine |
| | VVLDLGREDPEVQISWFVDGKEVHTAKTQSRE | IgG-A Fc |
| | QQFNGTYRVVSVLPIEHQDWLTGKEFKCRVNH | Heterodimer chain 1 |
| | IDLPSPIERTISKARGRAHKPSVYVLPPSPKE | T(138)Y |
| | LSSSDTVSIYCLIKDFYPPDIDVEWQSNGQQE | |
| | PERKHRMTPPQLDEDGSYFLYSKLSVDKSRWQ | |
| | QGDPFTCAVMHETLQNHYTDLSLSHSPGK | |
| 75 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Heterodimer chain 1 |
| | KALPSPIERTISKARGQAHQPSVYVLPPSREE | T(137)Y |
| | LSKNTVSLYCLIKDFFPPDIDVEWQSNGQQEP | |
| | ESKYRTTPPQLDEDGSYFLYSKLSVDKSRWQR | |
| | GDTFICAVMHEALHNHYTQESLSHSPGK | |
| 76 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Heterodimer chain 1 |
| | KALPSPIEEIISKTPGQAHQPNVYVLPPSRDE | T(137)Y |
| | MSKNTVTLYCLVKDFFPPEIDVEWQSNGQQEP | |
| | ESKYRMTPPQLDEDGSYFLYSKLSVDKSRWQR | |
| | GDTFICAVMHEALHNHYTQISLSHSPGK | |
| 77 | PVPESLGGPSVFIFPPKPKDILRITRTPEITC | Exemplary variant canine |
| | VVLDLGREDPEVQISWFVDGKEVHTAKTQPRE | IgG-D Fc |
| | QQFNSTYRVVSVLPIEHQDWLTGKEFKCRVNH | Heterodimer chain 1 |
| | IGLPSPIERTISKARGQAHQPSVYVLPPSPKE | T(138)Y |
| | LSSSDTVTLYCLIKDFFPPEIDVEWQSNGQPE | |
| | PESKYHTTAPQLDEDGSYFLYSKLSVDKSRWQ | |
| | QGDTFTCAVMHEALQNHYTDLSLSHSPGK | |
| 78 | PVPEPLGGPSVLIFPPKPKDILRITRTPEVTC | Exemplary variant canine |
| | VVLDLGREDPEVQISWFVDGKEVHTAKTQSRE | IgG-A Fc |
| | QQFNGTYRVVSVLPIEHQDWLTGKEFKCRVNH | Heterodimer chain 1 |
| | IDLPSPIERTISKARGRAHKPSVYVLPPSPKE | T(138)W |
| | LSSSDTVSIWCLIKDFYPPDIDVEWQSNGQQE | |
| | PERKHRMTPPQLDEDGSYFLYSKLSVDKSRWQ | |
| | QGDPFTCAVMHETLQNHYTDLSLSHSPGK | |
| 79 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Heterodimer chain 1 |
| | KALPSPIERTISKARGQAHQPSVYVLPPSREE | T(137)W |
| | LSKNTVSLWCLIKDFFPPDIDVEWQSNGQQEP | |
| | ESKYRTTPPQLDEDGSYFLYSKLSVDKSRWQR | |
| | GDTFICAVMHEALHNHYTQESLSHSPGK | |
| 80 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Heterodimer chain 1 |
| | KALPSPIEEIISKTPGQAHQPNVYVLPPSRDE | T(137)W |
| | MSKNTVTLWCLVKDFFPPEIDVEWQSNGQQEP | |
| | ESKYRMTPPQLDEDGSYFLYSKLSVDKSRWQR | |
| | GDTFICAVMHEALHNHYTQISLSHSPGK | |
| 81 | PVPESLGGPSVFIFPPKPKDILRITRTPEITC | Exemplary variant canine |
| | VVLDLGREDPEVQISWFVDGKEVHTAKTQPRE | IgG-D Fc |
| | QQFNSTYRVVSVLPIEHQDWLTGKEFKCRVNH | Heterodimer chain 1 |
| | IGLPSPIERTISKARGQAHQPSVYVLPPSPKE | T(138)W |
| | LSSSDTVTLWCLIKDFFPPEIDVEWQSNGQPE | |
| | PESKYHTTAPQLDEDGSYFLYSKLSVDKSRWQ | |
| | QGDTFTCAVMHEALQNHYTDLSLSHSPGK | |
| 82 | PVPEPLGGPSVLIFPPKPKDILRITRTPEVTC | Exemplary variant canine |
| | VVLDLGREDPEVQISWFVDGKEVHTAKTQSRE | IgG-A Fc |
| | QQFNGTYRVVSVLPIEHQDWLTGKEFKCRVNH | Heterodimer chain 2 |
| | IDLPSPIERTISKARGRAHKPSVYVLPPSPKE | Y(181)T |
| | LSSSDTVSITCLIKDFYPPDIDVEWQSNGQQE | |
| | PERKHRMTPPQLDEDGSYFLTSKLSVDKSRWQ | |
| | QGDPFTCAVMHETLQNHYTDLSLSHSPGK | |
| 83 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Heterodimer chain 2 |
| | KALPSPIERTISKARGQAHQPSVYVLPPSREE | Y(180)T |
| | LSKNTVSLTCLIKDFFPPDIDVEWQSNGQQEP | |
| | ESKYRTTPPQLDEDGSYFLTSKLSVDKSRWQR | |
| | GDTFICAVMHEALHNHYTQESLSHSPGK | |
| 84 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Heterodimer chain 2 |
| | KALPSPIEEIISKTPGQAHQPNVYVLPPSRDE | Y(180)T |
| | MSKNTVTLTCLVKDFFPPEIDVEWQSNGQQEP | |
| | ESKYRMTPPQLDEDGSYFLTSKLSVDKSRWQR | |
| | GDTFICAVMHEALHNHYTQISLSHSPGK | |
| 85 | PVPESLGGPSVFIFPPKPKDILRITRTPEITC | Exemplary variant canine |
| | VVLDLGREDPEVQISWFVDGKEVHTAKTQPRE | IgG-D Fc |
| | QQFNSTYRVVSVLPIEHQDWLTGKEFKCRVNH | Heterodimer chain 2 |
| | IGLPSPIERTISKARGQAHQPSVYVLPPSPKE | Y(181)T |
| | LSSSDTVTLTCLIKDFFPPEIDVEWQSNGQPE | |
| | PESKYHTTAPQLDEDGSYFLTSKLSVDKSRWQ | |
| | QGDTFTCAVMHEALQNHYTDLSLSHSPGK | |
| 86 | PVPEPLGGPSVLIFPPKPKDILRITRTPEVTC | Exemplary variant canine |
| | VVLDLGREDPEVQISWFVDGKEVHTAKTQSRE | IgG-A Fc |
| | QQFNGTYRVVSVLPIEHQDWLTGKEFKCRVNH | Heterodimer chain 2 |
| | IDLPSPIERTISKARGRAHKPSVYVLPPSPKE | T(138)S |
| | LSSSDTVSISCAIKDFYPPDIDVEWQSNGQQE | L(140)A |
| | PERKHRMTPPQLDEDGSYFLTSKLSVDKSRWQ | Y(181)T |
| | QGDPFTCAVMHETLQNHYTDTSLSHSPGK | |
| 87 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Heterodimer chain 2 |
| | KALPSPIERTISKARGQAHQPSVYVLPPSREE | T(137)S |
| | LSKNTVSLSCAIKDFFPPDIDVEWQSNGQQEP | L(139)A |
| | ESKYRTTPPQLDEDGSYFLTSKLSVDKSRWQR | Y(180)T |
| | GDTFICAVMHEALHNHYTQESLSHSPGK | |
| 88 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Heterodimer chain 2 |
| | KALPSPIEEIISKTPGQAHQPNVYVLPPSRDE | T(137)S |
| | MSKNTVTLSCAVKDFFPPEIDVEWQSNGQQEP | L(139)A |
| | ESKYRMTPPQLDEDGSYFLTSKLSVDKSRWQR | Y(180)T |
| | GDTFICAVMHEALHNHYTQTSLSHSPGK | |
| 89 | PVPESLGGPSVFIFPPKPKDILRITRTPEITC | Exemplary variant canine |
| | VVLDLGREDPEVQISWFVDGKEVHTAKTQPRE | IgG-D Fc |
| | QQFNSTYRVVSVLPIEHQDWLTGKEFKCRVNH | Heterodimer chain 2 |
| | IGLPSPIERTISKARGQAHQPSVYVLPPSPKE | T(138)S |
| | LSSSDTVTLSCAIKDFFPPEIDVEWQSNGQPE | L(140)A |
| | PESKYHTTAPQLDEDGSYFLTSKLSVDKSRWQ | Y(181)T |
| | QGDIFTCAVMHEALQNHYTDTSLSHSPGK | |
| 90 | PVPEPLGGPSVLIFPPKPKDILRITRTPEVTC | Exemplary variant canine |
| | VVLDLGREDPEVQISWFVDGKEVHTAKTQSRE | IgG-A Fc |
| | QQFNGTYRVVSVLPIEHQDWLTGKEFKCRVNH | Heterodimer chain 2 |
| | IDLPSPIERTISKARGRAHKPSVYVLPPSPKE | T(138)S |
| | LSSSDTVSISCAIKDFYPPDIDVEWQSNGQQE | L(140)A |
| | PERKHRMTPPQLDEDGSYFLYSKLSVDKSRWQ | |
| | QGDPFTCAVMHETLQNHYTDLSLSHSPGK | |
| 91 | PAPEMLGGPSVFIFPPKPKDTLLIARTPEVTC | Exemplary variant canine |
| | VVVDLDPEDPEVQISWFVDGKQMQTAKTQPRE | IgG-B Fc |
| | EQFNGTYRVVSVLPIGHQDWLKGKQFTCKVNN | Heterodimer chain 2 |
| | KALPSPIERTISKARGQAHQPSVYVLPPSREE | T(137)S |
| | LSKNTVSLSCAIKDFFPPDIDVEWQSNGQQEP | L(139)A |
| | ESKYRTTPPQLDEDGSYFLYSKLSVDKSRWQR | |
| | GDTFICAVMHEALHNHYTQESLSHSPGK | |
| 92 | PGCGLLGGPSVFIFPPKPKDILVTARTPTVTC | Exemplary variant canine |
| | VVVDLDPENPEVQISWFVDSKQVQTANTQPRE | IgG-C Fc |
| | EQSNGTYRVVSVLPIGHQDWLSGKQFKCKVNN | Heterodimer chain 2 |
| | KALPSPIEEIISKTPGQAHQPNVYVLPPSRDE | T(137)S |
| | MSKNTVTLSCAVKDFFPPEIDVEWQSNGQQEP | L(139)A |
| | ESKYRMTPPQLDEDGSYFLYSKLSVDKSRWQR | |
| | GDTFICAVMHEALHNHYTQISLSHSPGK | |
| 93 | PVPESLGGPSVFIFPPKPKDILRITRTPEITC | Exemplary variant canine |
| | VVLDLGREDPEVQISWFVDGKEVHTAKTQPRE | IgG-D Fc |
| | QQFNSTYRVVSVLPIEHQDWLTGKEFKCRVNH | Heterodimer chain 2 |
| | IGLPSPIERTISKARGQAHQPSVYVLPPSPKE | T(138)S |
| | LSSSDTVTLSCAIKDFFPPEIDVEWQSNGQPE | L(140)A |
| | PESKYHTTAPQLDEDGSYFLYSKLSVDKSRWQ | |
| | QGDTFTCAVMHEALQNHYTDLSLSHSPGK | |
| 94 | RKTDHPPGPKTGEGPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 1 |
| | LHQDWLKGKEFKCKVNSKSLPSPIERTISKAK | T(154)Y |
| | GQPHEPQVYVLPPAQEELSENKVSVYCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 95 | RKTDHPPGPKPCDCPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 1 |
| | LHQDWLKGKEFKCKVNSKSLPSPIERTISKAK | T(154)Y |
| | GQPHEPQVYVLPPAQEELSENKVSVYCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 96 | RKTDHPPGPKTGEGPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 1 |
| | LHQDWLKGKEFKCKVNSKSLPSPIERTISKDK | T(154)Y |
| | GQPHEPQVYVLPPAQEELSENKVSVYCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 97 | RKTDHPPGPKPCDCPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 1 |
| | LHQDWLKGKEFKCKVNSKSLPSPIERTISKDK | T(154)Y |
| | GQPHEPQVYVLPPAQEELSENKVSVYCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 98 | PKTASTIESKTGEGPKCPVPEIPGAPSVFIFP | Exemplary variant feline |
| | PKPKDTLSISRTPEVTCLVVDLGPDDSNVQIT | IgG2 Fc |
| | WFVDNTEMHTAKTRPREEQFNSTYRVVSVLPI | Heterodimer chain 1 |
| | LHQDWLKGKEFKCKVNSKSLPSAMERTISKAK | T(154)W |
| | GQPHEPQVYVLPPTQEELSENKVSVWCLIKGF | |
| | HPPDIAVEWEITGQPEPENNYQTTPPQLDSDG | |
| | TYFLYSRLSVDRSHWQRGNTYTCSVSHEALHS | |
| | HHTQKSLTQSPGK | |
| 99 | RKTDHPPGPKTGEGPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 1 |
| | LHQDWLKGKEFKCKVNSKSLPSPIERTISKAK | T(154)W |
| | GQPHEPQVYVLPPAQEELSENKVSVWCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 100 | RKTDHPPGPKPCDCPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 1 |
| | LHQDWLKGKEFKCKVNSKSLPSPIERTISKAK | T(154)W |
| | GQPHEPQVYVLPPAQEELSENKVSVWCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 101 | RKTDHPPGPKTGEGPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 1 |
| | LHQDWLKGKEFKCKVNSKSLPSPIERTISKDK | T(154)W |
| | GQPHEPQVYVLPPAQEELSENKVSVWCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 102 | RKTDHPPGPKPCDCPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 1 |
| | LHQDWLKGKEFKCKVNSKSLPSPIERTISKDK | T(154)W |
| | GQPHEPQVYVLPPAQEELSENKVSVWCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 103 | PKTASTIESKTGEGPKCPVPEIPGAPSVFIFP | Exemplary variant feline |
| | PKPKDTLSISRTPEVTCLVVDLGPDDSNVQIT | IgG2 Fc |
| | WFVDNTEMHTAKTRPREEQFNSTYRVVSVLPI | Heterodimer chain 1 |
| | LHQDWLKGKEFKCKVNSKSLPSAMERTISKAK | T(154)W |
| | GQPHEPQVYVLPPTQEELSENKVSVWCLIKGF | |
| | HPPDIAVEWEITGQPEPENNYQTTPPQLDSDG | |
| | TYFLYSRLSVDRSHWQRGNTYTCSVSHEALHS | |
| | HHTQKSLTQSPGK | |
| 104 | RKTDHPPGPKTGEGPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 2 |
| | LHQDWLKGKEFKCKVNSKSLPSPIERTISKAK | T(154)S |
| | GQPHEPQVYVLPPAQEELSENKVSVSCAIKSF | L(156)A |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 105 | RKTDHPPGPKTGEGPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 2 |
| | LHQDWLKGKEFKCKVNSKSLPSPIERTISKAK | T(154)S |
| | GQPHEPQVYVLPPAQEELSENKVSVSCAIKSF | L(156)A |
| | HPPDIAVEWEITGQPEPENNYRTTPPQLDSDG | Y(197)T |
| | TYFVTSKLSVDRSHWQRGNTYTCSVSHEALHS | |
| | HHTQKSLTQSPGK | |
| 106 | RKTDHPPGPKPCDCPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 2 |
| | LHQDWLKGKEFKCKVNSKSLPSPIERTISKAK | T(154)S |
| | GQPHEPQVYVLPPAQEELSENKVSVSCAIKSF | L(156)A |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 107 | RKTDHPPGPKPCDCPKCPPPEMLGGPSIFIFP | Exemplary variant feline |
| | PKPKDTLSISRTPEVTCLVVDLGPDDSDVQIT | IgG1a Fc |
| | WFVDNTQVYTAKTSPREEQFNSTYRVVSVLPI | Heterodimer chain 2 |
| | LHQDWLKGKEFKCKVNSKSLPSPIERTISKAK | T(154)S |
| | GQPHEPQVYVLPPAQEELSENKVSVSCAIKSF | L(156)A |
| | HPPDIAVEWEITGQPEPENNYRTTPPQLDSDG | Y(197)T |
| | TYFVTSKLSVDRSHWQRGNTYTCSVSHEALHS | |
| | HHTQKSLTQSPGK | |
| 108 | RKTDHPPGPKTGEGPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 2 |
| | LHQDWLKGKEFKCKVNSKSLPSPIERTISKDK | T(154)S |
| | GQPHEPQVYVLPPAQEELSENKVSVSCAIEGF | L(156)A |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 109 | RKTDHPPGPKTGEGPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 2 |
| | LHQDWLKGKEFKCKVNSKSLPSPIERTISKDK | T(154)S |
| | GQPHEPQVYVLPPAQEELSENKVSVSCAIEGF | L(156)A |
| | YPSDIAVEWEITGQPEPENNYRTTPPQLDSDG | Y(197)T |
| | TYFLTSRLSVDRSRWQRGNTYTCSVSHEALHS | |
| | HHTQKSLTQSPGK | |
| 110 | RKTDHPPGPKPCDCPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 2 |
| | LHQDWLKGKEFKCKVNSKSLPSPIERTISKDK | T(154)S |
| | GQPHEPQVYVLPPAQEELSENKVSVSCAIEGF | L(156)A |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 111 | RKTDHPPGPKPCDCPKCPPPEMLGGPSIFIFP | Exemplary variant feline |
| | PKPKDTLSISRTPEVTCLVVDLGPDDSDVQIT | IgG1b Fc |
| | WFVDNTQVYTAKTSPREEQFNSTYRVVSVLPI | Heterodimer chain 2 |
| | LHQDWLKGKEFKCKVNSKSLPSPIERTISKDK | T(154)S |
| | GQPHEPQVYVLPPAQEELSENKVSVSCAIEGF | L(156)A |
| | YPSDIAVEWEITGQPEPENNYRTTPPQLDSDG | Y(197)T |
| | TYFLTSRLSVDRSRWQRGNTYTCSVSHEALHS | |
| | HHTQKSLTQSPGK | |
| 112 | PKTASTIESKTGEGPKCPVPEIPGAPSVFIFP | Exemplary variant feline |
| | PKPKDTLSISRTPEVTCLVVDLGPDDSNVQIT | IgG2 Fc |
| | WFVDNTEMHTAKTRPREEQFNSTYRVVSVLPI | Heterodimer chain 2 |
| | LHQDWLKGKEFKCKVNSKSLPSAMERTISKAK | T(154)S |
| | GQPHEPQVYVLPPTQEELSENKVSVSCAIKGF | L(156)A |
| | HPPDIAVEWEITGQPEPENNYQTTPPQLDSDG | |
| | TYFLYSRLSVDRSHWQRGNTYTCSVSHEALHS | |
| | HHTQKSLTQSPGK | |
| 113 | PKTASTIESKTGEGPKCPVPEIPGAPSVFIFP | Exemplary variant feline |
| | PKPKDTLSISRTPEVTCLVVDLGPDDSNVQIT | IgG2 Fc |
| | WFVDNTEMHTAKTRPREEQFNSTYRVVSVLPI | Heterodimer chain 2 |
| | LHQDWLKGKEFKCKVNSKSLPSAMERTISKAK | T(154)S |
| | GQPHEPQVYVLPPTQEELSENKVSVSCAIKGF | L(156)A |
| | HPPDIAVEWEITGQPEPENNYQTTPPQLDSDG | Y(197)T |
| | TYFLTSRLSVDRSHWQRGNTYTCSVSHEALHS | |
| | HHTQKSLTQSPGK | |
| 114 | GGPSVFIFPPNPKDTLMITRTPEVTCVVVDVS | Exemplary variant equine |
| | QENPDVKFNWYMDGVEVRTATTRPKEEQFNST | IgG1 Fc |
| | YRVVSVLRIQHQDWLSGKEFKCKVNNQALPQP | Heterodimer chain 1 |
| | IERTITKTKGRSQEPQVYVLAPHPDELSKSKV | T(131)Y |
| | SVYCLVKDFYPPEINIEWQSNGQPELETKYST | |
| | TQAQQDSDGSYFLYSKLSVDRNRWQQGTTFTC | |
| | GVMHEALHNHYTQKNVSKNPGK | |
| 115 | GGPSVFIFPPNPKDALMISRTPVVTCVVVNLS | Exemplary variant equine |
| | DQYPDVQFSWYVDNTEVHSAITKQREAQFNST | IgG2 Fc |
| | YRVVSVLPIQHQDWLSGKEFKCSVTNVGVPQP | Heterodimer chain 1 |
| | ISRAISRGKGPSRVPQVYVLPPHPDELAKSKV | T(131)Y |
| | SVYCLVKDFYPPDISVEWQSNRWPELEGKYST | |
| | TPAQLDGDGSYFLYSKLSLETSRWQQVESFTC | |
| | AVMHEALHNHFTKTDISESLGK | |
| 116 | GGPSVFIFPPKPKDVLMITRMPEVTCLVVDVS | Exemplary variant equine |
| | HDSSDVLFTWYVDGTEVKTAKTMPNEEQNNST | IgG3 Fc |
| | YRVVSVLRIQHQDWLNGKKFKCKVNNQALPAP | Heterodimer chain 1 |
| | VERTISKATGQTRVPQVYVLAPHPDELSKNKV | T(131)Y |
| | SVYCLVKDFYPPDITVEWQSNEHPEPEGKYRT | |
| | TEAQKDSDGSYFLYSKLTVEKDRWQQGTTFTC | |
| | VVMHEALHNHVMQKNISKNPGK | |
| 117 | VGPSVFIFPPKPKDVLMISRTPTVTCVVVDVG | Exemplary variant equine |
| | HDFPDVQFNWYVDGVETHTATTEPKQEQFNST | IgG4 Fc |
| | YRVVSVLPIQHKDWLSGKEFKCKVNNKALPAP | Heterodimer chain 1 |
| | VERTISAPTGQPREPQVYVLAPHRDELSKNKV | T(131)Y |
| | SVYCLVKDFYPPDIDIEWKSNGQPEPETKYST | |
| | TPAQLDSDGSYFLYSKLTVETNRWQQGTTFTC | |
| | AVMHEALHNHYTEKSVSKSPGK | |
| 118 | GGPSVFIFPPKPKDVLMISRKPEVTCVVVDLG | Exemplary variant equine |
| | HDDPDVQFTWFVDGVETHTATTEPKEEQFNST | IgG5 Fc |
| | YRVVSVLPIQHQDWLSGKEFKCSVTSKALPAP | Heterodimer chain 1 |
| | VERTISKAKGQLRVPQVYVLAPHPDELAKNTV | T(131)Y |
| | SVYCLVKDFYPPEIDVEWQSNEHPEPEGKYST | |
| | TPAQLNSDGSYFLYSKLSVETSRWKQGESFTC | |
| | GVMHEAVENHYTQKNVSHSPGK | |
| 119 | GRPSVFIFPPNPKDTLMISRTPEVTCVVVDVS | Exemplary variant equine |
| | QENPDVKFNWYVDGVEAHTATTKAKEKQDNST | IgG6 Fc |
| | YRVVSVLPIQHQDWRRGKEFKCKVNNRALPAP | Heterodimer chain 1 |
| | VERTITKAKGELQDPKVYILAPHREEVTKNTV | T(131)Y |
| | SVYCLVKDFYPPDINVEWQSNEEPEPEVKYST | |
| | TPAQLDGDGSYFLYSKLTVETDRWEQGESFTC | |
| | VVMHEAIRHTYRQKSITNFPGK | |
| 120 | VGPSVFIFPPKPKDVLMISRTPTVTCVVVDVG | Exemplary variant equine |
| | HDFPDVQFNWYVDGVETHTATTEPKQEQNNST | IgG7 Fc |
| | YRVVSILAIQHKDWLSGKEFKCKVNNQALPAP | Heterodimer chain 1 |
| | VQKTISKPTGQPREPQVYVLAPHPDELSKNKV | T(131)Y |
| | SVYCLVKDFYPPDIDIEWKSNGQPEPETKYST | |
| | TPAQLDGDGSYFLYSKLTVETNRWQQGTTFTC | |
| | AVMHEALHNHYTEKSVSKSPGK | |
| 121 | GGPSVFIFPPNPKDTLMITRTPEVTCVVVDVS | Exemplary variant equine |
| | QENPDVKFNWYMDGVEVRTATTRPKEEQFNST | IgG1 Fc |
| | YRVVSVLRIQHQDWLSGKEFKCKVNNQALPQP | Heterodimer chain 1 |
| | IERTITKTKGRSQEPQVYVLAPHPDELSKSKV | T(131)W |
| | SVWCLVKDFYPPEINIEWQSNGQPELETKYST | |
| | TQAQQDSDGSYFLYSKLSVDRNRWQQGTTFTC | |
| | GVMHEALHNHYTQKNVSKNPGK | |
| 122 | GGPSVFIFPPNPKDALMISRTPVVTCVVVNLS | Exemplary variant equine |
| | DQYPDVQFSWYVDNTEVHSAITKQREAQFNST | IgG2 Fc |
| | YRVVSVLPIQHQDWLSGKEFKCSVTNVGVPQP | Heterodimer chain 1 |
| | ISRAISRGKGPSRVPQVYVLPPHPDELAKSKV | T(131)W |
| | SVWCLVKDFYPPDISVEWQSNRWPELEGKYST | |
| | TPAQLDGDGSYFLYSKLSLETSRWQQVESFTC | |
| | AVMHEALHNHFTKTDISESLGK | |
| 123 | GGPSVFIFPPKPKDVLMITRMPEVTCLVVDVS | Exemplary variant equine |
| | HDSSDVLFTWYVDGTEVKTAKTMPNEEQNNST | IgG3 Fc |
| | YRVVSVLRIQHQDWLNGKKFKCKVNNQALPAP | Heterodimer chain 1 |
| | VERTISKATGQTRVPQVYVLAPHPDELSKNKV | T(131)W |
| | SVWCLVKDFYPPDITVEWQSNEHPEPEGKYRT | |
| | TEAQKDSDGSYFLYSKLTVEKDRWQQGTTFTC | |
| | VVMHEALHNHVMQKNISKNPGK | |
| 124 | VGPSVFIFPPKPKDVLMISRTPTVTCVVVDVG | Exemplary variant equine |
| | HDFPDVQFNWYVDGVETHTATTEPKQEQFNST | IgG4 Fc |
| | YRVVSVLPIQHKDWLSGKEFKCKVNNKALPAP | Heterodimer chain 1 |
| | VERTISAPTGQPREPQVYVLAPHRDELSKNKV | T(131)W |
| | SVWCLVKDFYPPDIDIEWKSNGQPEPETKYST | |
| | TPAQLDSDGSYFLYSKLTVETNRWQQGTTFTC | |
| | AVMHEALHNHYTEKSVSKSPGK | |
| 125 | GGPSVFIFPPKPKDVLMISRKPEVTCVVVDLG | Exemplary variant equine |
| | HDDPDVQFTWFVDGVETHTATTEPKEEQFNST | IgG5 Fc |
| | YRVVSVLPIQHQDWLSGKEFKCSVTSKALPAP | Heterodimer chain 1 |
| | VERTISKAKGQLRVPQVYVLAPHPDELAKNTV | T(131)W |
| | SVWCLVKDFYPPEIDVEWQSNEHPEPEGKYST | |
| | TPAQLNSDGSYFLYSKLSVETSRWKQGESFTC | |
| | GVMHEAVENHYTQKNVSHSPGK | |
| 126 | GRPSVFIFPPNPKDTLMISRTPEVTCVVVDVS | Exemplary variant equine |
| | QENPDVKFNWYVDGVEAHTATTKAKEKQDNST | IgG6 Fc |
| | YRVVSVLPIQHQDWRRGKEFKCKVNNRALPAP | Heterodimer chain 1 |
| | VERTITKAKGELQDPKVYILAPHREEVTKNTV | T(131)W |
| | SVWCLVKDFYPPDINVEWQSNEEPEPEVKYST | |
| | TPAQLDGDGSYFLYSKLTVETDRWEQGESFTC | |
| | VVMHEAIRHTYRQKSITNFPGK | |
| 127 | VGPSVFIFPPKPKDVLMISRTPTVTCVVVDVG | Exemplary variant equine |
| | HDFPDVQFNWYVDGVETHTATTEPKQEQNNST | IgG7 Fc |
| | YRVVSILAIQHKDWLSGKEFKCKVNNQALPAP | Heterodimer chain 1 |
| | VQKTISKPTGQPREPQVYVLAPHPDELSKNKV | T(131)W |
| | SVWCLVKDFYPPDIDIEWKSNGQPEPETKYST | |
| | TPAQLDGDGSYFLYSKLTVETNRWQQGTTFTC | |
| | AVMHEALHNHYTEKSVSKSPGK | |
| 128 | GGPSVFIFPPNPKDTLMITRTPEVTCVVVDVS | Exemplary variant equine |
| | QENPDVKFNWYMDGVEVRTATTRPKEEQFNST | IgG1 Fc |
| | YRVVSVLRIQHQDWLSGKEFKCKVNNQALPQP | Heterodimer chain 2 |
| | IERTITKTKGRSQEPQVYVLAPHPDELSKSKV | T(131)S |
| | SVSCAVKDFYPPEINIEWQSNGQPELETKYST | L(133)A |
| | TQAQQDSDGSYFLYSKLSVDRNRWQQGTTFTC | |
| | GVMHEALHNHYTQKNVSKNPGK | |
| 129 | GGPSVFIFPPNPKDALMISRTPVVTCVVVNLS | Exemplary variant equine |
| | DQYPDVQFSWYVDNTEVHSAITKQREAQFNST | IgG2 Fc |
| | YRVVSVLPIQHQDWLSGKEFKCSVTNVGVPQP | Heterodimer chain 2 |
| | ISRAISRGKGPSRVPQVYVLPPHPDELAKSKV | T(131)S |
| | SVSCAVKDFYPPDISVEWQSNRWPELEGKYST | L(133)A |
| | TPAQLDGDGSYFLYSKLSLETSRWQQVESFTC | |
| | AVMHEALHNHFTKTDISESLGK | |
| 130 | GGPSVFIFPPKPKDVLMITRMPEVTCLVVDVS | Exemplary variant equine |
| | HDSSDVLFTWYVDGTEVKTAKTMPNEEQNNST | IgG3 Fc |
| | YRVVSVLRIQHQDWLNGKKFKCKVNNQALPAP | Heterodimer chain 2 |
| | VERTISKATGQTRVPQVYVLAPHPDELSKNKV | T(131)S |
| | SVSCAVKDFYPPDITVEWQSNEHPEPEGKYRT | L(133)A |
| | TEAQKDSDGSYFLYSKLTVEKDRWQQGTTFTC | |
| | VVMHEALHNHVMQKNISKNPGK | |
| 131 | VGPSVFIFPPKPKDVLMISRTPTVTCVVVDVG | Exemplary variant equine |
| | HDFPDVQFNWYVDGVETHTATTEPKQEQFNST | IgG4 Fc |
| | YRVVSVLPIQHKDWLSGKEFKCKVNNKALPAP | Heterodimer chain 2 |
| | VERTISAPTGQPREPQVYVLAPHRDELSKNKV | T(131)S |
| | SVSCAVKDFYPPDIDIEWKSNGQPEPETKYST | L(133)A |
| | TPAQLDSDGSYFLYSKLTVETNRWQQGTTFTC | |
| | AVMHEALHNHYTEKSVSKSPGK | |
| 132 | GGPSVFIFPPKPKDVLMISRKPEVTCVVVDLG | Exemplary variant equine |
| | HDDPDVQFTWFVDGVETHTATTEPKEEQFNST | IgG5 Fc |
| | YRVVSVLPIQHQDWLSGKEFKCSVTSKALPAP | Heterodimer chain 2 |
| | VERTISKAKGQLRVPQVYVLAPHPDELAKNTV | T(131)S |
| | SVSCAVKDFYPPEIDVEWQSNEHPEPEGKYST | L(133)A |
| | TPAQLNSDGSYFLYSKLSVETSRWKQGESFTC | |
| | GVMHEAVENHYTQKNVSHSPGK | |
| 133 | GRPSVFIFPPNPKDTLMISRTPEVTCVVVDVS | Exemplary variant equine |
| | QENPDVKFNWYVDGVEAHTATTKAKEKQDNST | IgG6 Fc |
| | YRVVSVLPIQHQDWRRGKEFKCKVNNRALPAP | Heterodimer chain 2 |
| | VERTITKAKGELQDPKVYILAPHREEVTKNTV | T(131)S |
| | SVSCAVKDFYPPDINVEWQSNEEPEPEVKYST | L(133)A |
| | TPAQLDGDGSYFLYSKLTVETDRWEQGESFTC | |
| | VVMHEAIRHTYRQKSITNFPGK | |
| 134 | VGPSVFIFPPKPKDVLMISRTPTVTCVVVDVG | Exemplary variant equine |
| | HDFPDVQFNWYVDGVETHTATTEPKQEQNNST | IgG7 Fc |
| | YRVVSILAIQHKDWLSGKEFKCKVNNQALPAP | Heterodimer chain 2 |
| | VQKTISKPTGQPREPQVYVLAPHPDELSKNKV | T(131)S |
| | SVSCAVKDFYPPDIDIEWKSNGQPEPETKYST | L(133)A |
| | TPAQLDGDGSYFLYSKLTVETNRWQQGTTFTC | |
| | AVMHEALHNHYTEKSVSKSPGK | |
| 135 | GGPSVFIFPPNPKDTLMITRTPEVTCVVVDVS | Exemplary variant equine |
| | QENPDVKFNWYMDGVEVRTATTRPKEEQFNST | IgG1 Fc |
| | YRVVSVLRIQHQDWLSGKEFKCKVNNQALPQP | Heterodimer chain 2 |
| | IERTITKTKGRSQEPQVYVLAPHPDELSKSKV | T(131)S |
| | SVSCAVKDFYPPEINIEWQSNGQPELETKYST | L(133)A |
| | TQAQQDSDGSYFLTSKLSVDRNRWQQGTTFTC | Y(174)T |
| | GVMHEALHNHYTQKNVSKNPGK | |
| 136 | GGPSVFIFPPNPKDALMISRTPVVTCVVVNLS | Exemplary variant equine |
| | DQYPDVQFSWYVDNTEVHSAITKQREAQFNST | IgG2 Fc |
| | YRVVSVLPIQHQDWLSGKEFKCSVTNVGVPQP | Heterodimer chain 2 |
| | ISRAISRGKGPSRVPQVYVLPPHPDELAKSKV | T(131)S |
| | SVSCAVKDFYPPDISVEWQSNRWPELEGKYST | L(133)A |
| | TPAQLDGDGSYFLTSKLSLETSRWQQVESFTC | Y(174)T |
| | AVMHEALHNHFTKTDISESLGK | |
| 137 | GGPSVFIFPPKPKDVLMITRMPEVTCLVVDVS | Exemplary variant equine |
| | HDSSDVLFTWYVDGTEVKTAKTMPNEEQNNST | IgG3 Fc |
| | YRVVSVLRIQHQDWLNGKKFKCKVNNQALPAP | Heterodimer chain 2 |
| | VERTISKATGQTRVPQVYVLAPHPDELSKNKV | T(131)S |
| | SVSCAVKDFYPPDITVEWQSNEHPEPEGKYRT | L(133)A |
| | TEAQKDSDGSYFLTSKLTVEKDRWQQGTTFTC | Y(174)T |
| | VVMHEALHNHVMQKNISKNPGK | |
| 138 | VGPSVFIFPPKPKDVLMISRTPTVTCVVVDVG | Exemplary variant equine |
| | HDFPDVQFNWYVDGVETHTATTEPKQEQFNST | IgG4 Fc |
| | YRVVSVLPIQHKDWLSGKEFKCKVNNKALPAP | Heterodimer chain 2 |
| | VERTISAPTGQPREPQVYVLAPHRDELSKNKV | T(131)S |
| | SVSCAVKDFYPPDIDIEWKSNGQPEPETKYST | L(133)A |
| | TPAQLDSDGSYFLTSKLTVETNRWQQGTTFTC | Y(174)T |
| | AVMHEALHNHYTEKSVSKSPGK | |
| 139 | GGPSVFIFPPKPKDVLMISRKPEVTCVVVDLG | Exemplary variant equine |
| | HDDPDVQFTWFVDGVETHTATTEPKEEQFNST | IgG5 Fc |
| | YRVVSVLPIQHQDWLSGKEFKCSVTSKALPAP | Heterodimer chain 2 |
| | VERTISKAKGQLRVPQVYVLAPHPDELAKNTV | T(131)S |
| | SVSCAVKDFYPPEIDVEWQSNEHPEPEGKYST | L(133)A |
| | TPAQLNSDGSYFLYSKLSVETSRWKQGESFTC | Y(174)T |
| | GVMHEAVENHYTQKNVSHSPGK | |
| 140 | GRPSVFIFPPNPKDTLMISRTPEVTCVVVDVS | Exemplary variant equine |
| | QENPDVKFNWYVDGVEAHTATTKAKEKQDNST | IgG6 Fc |
| | YRVVSVLPIQHQDWRRGKEFKCKVNNRALPAP | Heterodimer chain 2 |
| | VERTITKAKGELQDPKVYILAPHREEVTKNTV | T(131)S |
| | SVSCAVKDFYPPDINVEWQSNEEPEPEVKYST | L(133)A |
| | TPAQLDGDGSYFLTSKLTVETDRWEQGESFTC | Y(174)T |
| | VVMHEAIRHTYRQKSITNFPGK | |
| 141 | VGPSVFIFPPKPKDVLMISRTPTVTCVVVDVG | Exemplary variant equine |
| | HDFPDVQFNWYVDGVETHTATTEPKQEQNNST | IgG7 Fc |
| | YRVVSILAIQHKDWLSGKEFKCKVNNQALPAP | Heterodimer chain 2 |
| | VQKTISKPTGQPREPQVYVLAPHPDELSKNKV | T(131)S |
| | SVSCAVKDFYPPDIDIEWKSNGQPEPETKYST | L(133)A |
| | TPAQLDGDGSYFLYSKLTVETNRWQQGTTFTC | Y(174)T |
| | AVMHEALHNHYTEKSVSKSPGK | |
| 142 | ASTTAPSVFPLAPSCGSTSGSTVALACLVSGY | Wild-type canine IgG-A CH1 |
| | FPEPVTVSWNSGSLTSGVHTFPSVLQSSGLHS | |
| | LSSMVTVPSSRWPSETFTCNVVHPASNTKVDK | |
| | PV | |
| 143 | ASTTAPSVFPLAPSCGSTSGSTVALACLVSGY | Wild-type canine IgG-B CH1 |
| | FPEPVTVSWNSGSLTSGVHTFPSVLQSSGLYS | |
| | LSSMVTVPSSRWPSETFTCNVAHPASKTKVDK | |
| | PV | |
| 144 | ASTTAPSVFPLAPSCGSQSGSTVALACLVSGY | Wild-type canine IgG-CCH1 |
| | IPEPVTVSWNSVSLTSGVHTFPSVLQSSGLYS | |
| | LSSMVTVPSSRWPSETFTCNVAHPATNTKVDK | |
| | PV | |
| 145 | ASTTAPSVFPLAPSCGSTSGSTVALACLVSGY | Wild-type canine IgG-D CH1 |
| | FPEPVTVSWNSGSLTSGVHTFPSVLQSSGLYS | |
| | LSSTVTVPSSRWPSETFTCNVVHPASNTKVDK | |
| | PV | |
| 146 | ASTTAPSVFPLAPSCGSTSGSTVLLACLVDGY | Variant canine IgG-A CH1 |
| | FPEPVTVSWNSGSLTSGVHTFPSVLQSSGLHS | A(24)L |
| | LSSMVTVPSSRWPSETFTCNVVHPASNTKVDK | S(30)D |
| | PV | |
| 147 | ASTTAPSVFPLAPSCGSTSGSTVLLACLVDGY | Variant canine IgG-B CH1 |
| | FPEPVTVSWNSGSLTSGVHTFPSVLQSSGLYS | A(24)L |
| | LSSMVTVPSSRWPSETFTCNVAHPASKTKVDK | S(30)D |
| | PV | |
| 148 | ASTTAPSVFPLAPSCGSQSGSTVLLACLVDGY | Variant canine IgG-CCH1 |
| | IPEPVTVSWNSVSLTSGVHTFPSVLQSSGLYS | A(24)L |
| | LSSMVTVPSSRWPSETFTCNVAHPATNTKVDK | S(30)D |
| | PV | |
| 149 | ASTTAPSVFPLAPSCGSTSGSTVLLACLVDGY | Variant canine IgG-D CH1 |
| | FPEPVTVSWNSGSLTSGVHTFPSVLQSSGLYS | A(24)L |
| | LSSTVTVPSSRWPSETFTCNVVHPASNTKVDK | S(30)D |
| | PV | |
| 150 | RNDAQPAVYLFQPSPDQLHTGSASVVCLLNSF | Wild-type canine κ constant |
| | YPKDINVKWKVDGVIQDTGIQESVTEQDKDST | region |
| | YSLSSTLTMSSTEYLSHELYSCEITHKSLPST | |
| | LIKSFQRSECQRVD | |
| 151 | RNDAQPAVYLAQPSPDQLHTGRASVVCLLNSF | Variant canine κ constant |
| | YPKDINVKWKVDGVIQDTGIQESVTEQDKDST | region |
| | YSLSSTLTMSSTEYLSHELYSCEITHKSLPST | F(11)A |
| | LIKSFQRSECQRVD | S(22)R |
| 152 | ASTTAPSVFPLAPSCGTTSGATVALACLVSGY | Wild-type feline IgG1 CH1 |
| | FPEPVTVSWNSGALTSGVHTFPAVLQASGLYS | |
| | LSSMVTVPSSRWLSDTFTCNVAHPPSNTKVDK | |
| | TV | |
| 153 | ASTTASSVFPLAPSCGTTSGATVALACLSLGY | Wild-type feline IgG2 CH1 |
| | FPEPVTVSWNSGALTSGVHTFPSVLQASGLYS | |
| | LSSMVTVPSSRWLSDTFTCNVAHRPSSTKVDK | |
| | TV | |
| 154 | ASTTAPSVFPLAPSCGTTSGATVLLACLVDGY | Variant feline IgG1 CH1 |
| | FPEPVTVSWNSGALTSGVHTFPAVLQASGLYS | A(24)L |
| | LSSMVTVPSSRWLSDTFTCNVAHPPSNTKVDK | S(30)D |
| | TV | |
| 155 | ASTTASSVFPLAPSCGTTSGATVLLACLDLGY | Variant feline IgG2 CH1 |
| | FPEPVTVSWNSGALTSGVHTFPSVLQASGLYS | A(24)L |
| | LSSMVTVPSSRWLSDTFTCNVAHRPSSTKVDK | S(29)D |
| | TV | |
| 156 | RSDAQPSVFLFQPSLDELHTGSASIVCILNDF | Wild-type feline κ constant |
| | YPKEVNVKWKVDGVVQNKGIQESTTEQNSKDS | region |
| | TYSLSSTLTMSSTEYQSHEKFSCEVTHKSLAS | |
| | TLVKSFNRSECQRE | |
| 157 | RSDAQPSVFLAQPSLDELHTGRASIVCILNDF | Variant feline κ constant |
| | YPKEVNVKWKVDGVVQNKGIQESTTEQNSKDS | region |
| | TYSLSSTLTMSSTEYQSHEKFSCEVTHKSLAS | F(11)A |
| | TLVKSFNRSECQRE | S(22)R |
| 158 | G | 1G extension |
| 159 | GG | 2G extension |
| 160 | GGG | 3G extension |
| 161 | GGGG | 4G extension |
| 162 | GGGGG | 5G extension |
| 163 | GGGGGG | 6G extension |
| 164 | GGGGGGG | 7G extension |
| 165 | GGGGGGGG | 8G extension |
| 166 | PKTASTIESKTGECPKCPVPEIPGAPSVFIFP | Exemplary variant feline |
| | PKPKDTLSISRTPEVTCLVVDLGPDDSNVQIT | IgG2 Fc |
| | WFVDNTEMHTAKTRPREEQFNSTYRVVSVLPI | Hinge Cys |
| | LHQDWLKGKEFKCKVNSKSLPSAMERTISKAK | G(14)C |
| | GQPHEPQVYVLPPTQEELSENKVSVTCLIKGF | |
| | HPPDIAVEWEITGQPEPENNYQTTPPQLDSDG | |
| | TYFLYSRLSVDRSHWQRGNTYTCSVSHEALHS | |
| | HHTQKSLTQSPGK | |
| 167 | RKTDHPPGPKTGEGPPCPPPEMLGGPSIFIFP | Exemplary variant feline |
| | PKPKDTLSISRTPEVTCLVVDLGPDDSDVQIT | IgG1a Fc with modified hinge |
| | WFVDNTQVYTAKTSPREEQFNSTYRVVSVLPI | K(16)P |
| | LHQDWLKGKEFKCKVNSKSLPSPIERTISKAK | |
| | GQPHEPQVYVLPPAQEELSENKVSVTCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 168 | RKTDHPPGPKPCDCPPCPPPEMLGGPSIFIFP | Exemplary variant feline |
| | PKPKDTLSISRTPEVTCLVVDLGPDDSDVQIT | IgG1a Fc with modified hinge |
| | WFVDNTQVYTAKTSPREEQFNSTYRVVSVLPI | K(16)P |
| | LHQDWLKGKEFKCKVNSKSLPSPIERTISKAK | |
| | GQPHEPQVYVLPPAQEELSENKVSVTCLIKSF | |
| | HPPDIAVEWEITGQPEPENNYRTTPPQLDSDG | |
| | TYFVYSKLSVDRSHWQRGNTYTCSVSHEALHS | |
| | HHTQKSLTQSPGK | |
| 169 | RKTDHPPGPKTGEGPPCPPPEMLGGPSIFIFP | Exemplary variant feline |
| | PKPKDTLSISRTPEVTCLVVDLGPDDSDVQIT | IgG1b Fc with modified hinge |
| | WFVDNTQVYTAKTSPREEQFNSTYRVVSVLPI | K(16)P |
| | LHQDWLKGKEFKCKVNSKSLPSPIERTISKDK | |
| | GQPHEPQVYVLPPAQEELSENKVSVTCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 170 | RKTDHPPGPKPCDCPPCPPPEMLGGPSIFIFP | Exemplary variant feline |
| | PKPKDTLSISRTPEVTCLVVDLGPDDSDVQIT | IgG1b Fc with modified hinge |
| | WFVDNTQVYTAKTSPREEQFNSTYRVVSVLPI | K(16)P |
| | LHQDWLKGKEFKCKVNSKSLPSPIERTISKDK | |
| | GQPHEPQVYVLPPAQEELSENKVSVTCLIEGF | |
| | YPSDIAVEWEITGQPEPENNYRTTPPQLDSDG | |
| | TYFLYSRLSVDRSRWQRGNTYTCSVSHEALHS | |
| | HHTQKSLTQSPGK | |
| 171 | PKTASTIESKTGEGPPCPVPEIPGAPSVFIFP | Exemplary variant feline |
| | PKPKDTLSISRTPEVTCLVVDLGPDDSNVQIT | IgG2 Fc with modified hinge |
| | WFVDNTEMHTAKTRPREEQFNSTYRVVSVLPI | K(16)P |
| | LHQDWLKGKEFKCKVNSKSLPSAMERTISKAK | |
| | GQPHEPQVYVLPPTQEELSENKVSVTCLIKGF | |
| | HPPDIAVEWEITGQPEPENNYQTTPPQLDSDG | |
| | TYFLYSRLSVDRSHWQRGNTYTCSVSHEALHS | |
| | HHTQKSLTQSPGK | |
| 172 | PPCVLSAEGVIPIPSVPKPPCPPYTHSKFLGG | Exemplary variant equine |
| | PSVFIFPPNPKDALMISRTPVVTCVVVNLSDQ | IgG2 Fc with modified hinge |
| | YPDVQFSWYVDNTEVHSAITKQREAQFNSTYR | Protein A − |
| | VVSVLPIQHQDWLSGKEFKCSVTNVGVPQPIS | C1q − |
| | RAISRGKGPSRVPQVYVLPPHPDELAKSKVSV | Q(20)P |
| | TCLVKDFYPPDISVEWQSNRWPELEGKYSTTP | |
| | AQLDGDGSYFLYSKLSLETSRWQQVESFTCAV | |
| | MHEALHNHFTKTDISESLGK | |
| 173 | PPSVLSAEGVIPIPSVPKPQCPPYTHSKFLGG | Exemplary variant equine |
| | PSVFIFPPNPKDALMISRTPVVTCVVVNLSDQ | IgG2 Fc with modified hinge |
| | YPDVQFSWYVDNTEVHSAITKQREAQFNSTYR | Protein A − |
| | VVSVLPIQHQDWLSGKEFKCSVTNVGVPQPIS | C1q − |
| | RAISRGKGPSRVPQVYVLPPHPDELAKSKVSV | C(3)S |
| | TCLVKDFYPPDISVEWQSNRWPELEGKYSTTP | |
| | AQLDGDGSYFLYSKLSLETSRWQQVESFTCAV | |
| | LHNHFTKTDISESLGK | |
| 174 | PPSVLSAEGVIPIPSVPKPPCPPYTHSKFLGG | Exemplary variant equine |
| | PSVFIFPPNPKDALMISRTPVVTCVVVNLSDQ | IgG2 Fc with modified hinge |
| | YPDVQFSWYVDNTEVHSAITKQREAQFNSTYR | Protein A − |
| | VVSVLPIQHQDWLSGKEFKCSVTNVGVPQPIS | C1q − |
| | RAISRGKGPSRVPQVYVLPPHPDELAKSKVSV | C(3)S |
| | TCLVKDFYPPDISVEWQSNRWPELEGKYSTTP | Q(20)P |
| | AQLDGDGSYFLYSKLSLETSRWQQVESFTCAV | |
| | MHEALHNHFTKTDISESLGK | |
| 175 | PPCVLSAEGVIPIPSVPKPQCPPYTHSKFLGG | Exemplary variant equine |
| | PSVFIFPPNPKDTLMISRTPVVTCVVVNLSDQ | IgG2 Fc with hinge |
| | YPDVQFSWYVDNTEVHSAITKQREAQFNSTYR | Protein A + |
| | VVSVLPIQHQDWLSGKEFKCSVTNVGVPQPIS | C1q − |
| | RAISRGKGPSRVPQVYVLPPHPDELAKSKVSV | A(45)T |
| | TCLVKDFYPPDISVEWQSNRWPELEGKYSTTP | F(233)Y |
| | AQLDGDGSYFLYSKLSLETSRWQQVESFTCAV | |
| | MHEALHNHYTKTDISESLGK | |
| 176 | PPCVLSAEGVIPIPSVPKPPCPPYTHSKFLGG | Exemplary variant equine |
| | PSVFIFPPNPKDTLMISRTPVVTCVVVNLSDQ | IgG2 Fc with modified hinge |
| | YPDVQFSWYVDNTEVHSAITKQREAQFNSTYR | Protein A + |
| | VVSVLPIQHQDWLSGKEFKCSVTNVGVPQPIS | C1q − |
| | RAISRGKGPSRVPQVYVLPPHPDELAKSKVSV | Q(20)P |
| | TCLVKDFYPPDISVEWQSNRWPELEGKYSTTP | A(45)T |
| | AQLDGDGSYFLYSKLSLETSRWQQVESFTCAV | F(233)Y |
| | MHEALHNHYTKTDISESLGK | |
| 177 | PPSVLSAEGVIPIPSVPKPPCPPYTHSKFLGG | Exemplary variant equine |
| | PSVFIFPPNPKDTLMISRTPVVTCVVVNLSDQ | IgG2 Fc with modified hinge |
| | YPDVQFSWYVDNTEVHSAITKQREAQFNSTYR | Protein A + |
| | VVSVLPIQHQDWLSGKEFKCSVTNVGVPQPIS | C1q − |
| | RAISRGKGPSRVPQVYVLPPHPDELAKSKVSV | C(3)S |
| | TCLVKDFYPPDISVEWQSNRWPELEGKYSTTP | Q(20)P |
| | AQLDGDGSYFLYSKLSLETSRWQQVESFTCAV | A(45)T |
| | MHEALHNHYTKTDISESLGK | F(233)Y |
| 178 | RKTDHPPGPKPCDCPKCPPPEMLGGPSVFIFP | Exemplary variant feline |
| | PKPKDTLSISRTPEVTCLVVDLGPDDSNVQIT | IgG2 Fc with feline IgG1 |
| | WFVDNTEMHTAKTRPREEQFNSTYRVVSVLPI | hinge |
| | LHQDWLKGKEFKCKVNSKSLPSAMERTISKAK | |
| | GQPHIPQVYVLPPTQEELSENKVSVTCLIKGF | |
| | HPPDIAVEWEITGQPEPENNYQTTPPQLDSDG | |
| | TYFLYSRLSVDRSHWQRGNTYTCSVSHEALHS | |
| | HHTQKSLTQSPGK | |
| 179 | DMSKCPKCPAPELLGGPSVFIFPPNPKDALMI | Exemplary variant equine Fc |
| | SRTPVVTCVVVNLSDQYPDVQFSWYVDNTEVH | IgG2 (with equine IgG1 |
| | SAITKQREAQFNSTYRVVSVLPIQHQDWLSGK | hinge) |
| | EFKCSVTNVGVPQPISRAISRGKGPSRVPQVY | Protein A − |
| | VLPPHPDELAKSKVSVTCLVKDFYPPDISVEW | C1q − |
| | QSNRWPELEGKYSTTPAQLDGDGSYFLYSKLS | |
| | LETSRWQQVESFICAVMHEALHNHFIKTDISE | |
| | SLGK | |
| 180 | DMSKCPKCPAPELLGGPSVFIFPPNPKDTLMI | Exemplary variant equine |
| | SRTPVVTCVVVNLSDQYPDVQFSWYVDNTEVH | IgG2 Fc (with equine IgG1 |
| | SAITKQREAQFNSTYRVVSVLPIQHQDWLSGK | hinge) |
| | EFKCSVTNVGVPQPISRISRGKGPSRVPQVY | C1q − |
| | VLPPHPDELAKSKVSVTCLVKDFYPPDISVEW | Protein A |
| | QSNRWPELEGKYSTTPAQLDGDGSYFLYSKLS | A(29)T |
| | LETSRWQQVESFICAVMHEALHNHYTKTDISE | F(217)Y |
| | SLGK | |
| 181 | HXEGTFTSDVSSYLEGQAAKEFIAWLVKG | Exemplary variant GLP1 (7-35) |
| | | X8 may be G or S |
| 182 | HSQGTFTSDYSKYLDSRRAQDFVQWLMNT | Glucagon (Gluc) |
| 183 | HGEGTFTSDLSKQMEEEAVRLFIEWLKNGGPS | Extendin-4 |
| | SGAPPPS | |
| 184 | MAVLGLLFCLVTFPSCVLSHGEGIFTSDVSSY | ssGLP1-G8_I_VARfeIgG2 |
| | LEGQAAKEFIAWLVKGGGGSGGGGSGGGGSGG | |
| | GGSPKTASTIESKTGECPKCPVPEIPGAPSVF | |
| | IFPPKPKDTLSISRTPEVTCLVVDLGPDDSNV | |
| | QITWFVDNTEMHTAKTRPREEQFNSTYRVVSV | |
| | LPILHQDWLKGKEFKCKVNSKSLPSAMERTIS | |
| | KAKGQPHEPQVYVLPPTQEELSENKVSVTCLI | |
| | KGFHPPDIAVEWEITGQPEPENNYQTTPPQLD | |
| | SDGTYFLYSRLSVDRSHWQRGNTYTCSVSHEA | |
| | LHSHHTQKSLTQSPGK | |
| 185 | MAVLGLLFCLVTFPSCVLSHGEGIFTSDVSSY | ssGLP1-G8/GLP1-2G_III_ |
| | LEGQAAKEFIAWLVKGGGGSGGGGSGGGGSGG | VARfeIgG2 |
| | GGSPKTASTIESKTGECPKCPVPEIPGAPSVF | |
| | IFPPKPKDTLSISRTPEVTCLVVDLGPDDSNV | |
| | QITWFVDNTEMHTAKTRPREEQFNSTYRVVSV | |
| | LPILHQDWLKGKEFKCKVNSKSLPSAMERTIS | |
| | KAKGQPHEPQVYVLPPTQEELSENKVSVTCLI | |
| | KGFHPPDIAVEWEITGQPEPENNYQTTPPQLD | |
| | SDGTYFLYSRLSVDRSHWQRGNTYTCSVSHEA | |
| | LHSHHTQKSLTQSPGKGGGGSGGGGHAEGTFT | |
| | SDVSSYLEGQAAKEFIAWLVKGGG | |
| 186 | HGEGTFTSDVSSYLEGQAAKEFIAWLVKGGGG | GLP1-G8/GLP1-2G_III_ |
| | SGGGGSGGGGSGGGGSPKTASTIESKTGECPK | VARfeIgG2 |
| | CPVPEIPGAPSVFIFPPKPKDTLSISRTPEVT | |
| | CLVVDLGPDDSNVQITWFVDNTEMHTAKTRPR | |
| | EEQFNSTYRVVSVLPILHQDWLKGKEFKCKVN | |
| | SKSLPSAMERTISKAKGQPHEPQVYVLPPTQE | |
| | ELSENKVSVTCLIKGFHPPDIAVEWEITGQPE | |
| | PENNYQTTPPQLDSDGTYFLYSRLSVDRSHWQ | |
| | RGNTYTCSVSHEALHSHHTQKSLTQSPGKGGG | |
| | GSGGGGHAEGTFTSDVSSYLEGQAAKEFIAWL | |
| | VKGGG | |
| 187 | HGEGTFTSDVSSYLEGQAAKEFIAWLVKGGGG | GLP1-G8_I_VARfeIgG2 |
| | SGGGGSGGGGSGGGGSPKTASTIESKTGECPK | |
| | CPVPEIPGAPSVFIFPPKPKDTLSISRTPEVT | |
| | CLVVDLGPDDSNVQITWFVDNTEMHTAKTRPR | |
| | EEQFNSTYRVVSVLPILHQDWLKGKEFKCKVN | |
| | SKSLPSAMERTISKAKGQPHEPQVYVLPPTQE | |
| | ELSENKVSVTCLIKGFHPPDIAVEWEITGQPE | |
| | PENNYQTTPPQLDSDGTYFLYSRLSVDRSHWQ | |
| | RGNTYTCSVSHEALHSHHTQKSLTQSPGK | |
| 188 | HGEGTFTSDVSSYLEGQAAKEFIAWLVKGGGG | GLP1-G8/GLP-2G_III_ |
| | SGGGGSGGGGSGGGGSPKTASTIESKTGEGPK | WTfeIgG2 |
| | CPVPEIPGAPSVFIFPPKPKDTLSISRTPEVT | |
| | CLVVDLGPDDSNVQITWFVDNTEMHTAKTRPR | |
| | EEQFNSTYRVVSVLPILHQDWLKGKEFKCKVN | |
| | SKSLPSAMERTISKAKGQPHEPQVYVLPPTQE | |
| | ELSENKVSVTCLIKGFHPPDIAVEWEITGQPE | |
| | PENNYQTTPPQLDSDGTYFLYSRLSVDRSHWQ | |
| | RGNTYTCSVSHEALHSHHTQKSLTQSPGKGGG | |
| | GSGGGGHAEGTFTSDVSSYLEGQAAKEFIAWL | |
| | VKGGG | |
| 189 | HGEGTFTSDVSSYLEGQAAKEFIAWLVKGGGG | GLP1-G8_I_WTfeIgG2 |
| | SGGGGSGGGGSGGGGSPKTASTIESKTGEGPK | |
| | CPVPEIPGAPSVFIFPPKPKDTLSISRTPEVT | |
| | CLVVDLGPDDSNVQITWFVDNTEMHTAKTRPR | |
| | EEQFNSTYRVVSVLPILHQDWLKGKEFKCKVN | |
| | SKSLPSAMERTISKAKGQPHEPQVYVLPPTQE | |
| | ELSENKVSVTCLIKGFHPPDIAVEWEITGQPE | |
| | PENNYQTTPPQLDSDGTYFLYSRLSVDRSHWQ | |
| | RGNTYTCSVSHEALHSHHTQKSLTQSPGK | |
| 190 | TETQPPVTNLSVSVENLCTVIWTWDPPEGASP | Exemplary canine IL13R ECD |
| | NCTLRYFSHFDNKQDKKIAPETHRSKEVPLNE | |
| | RICLQVGSQCSTNESDNPSILVEKCTPPPEGD | |
| | PESAVTELQCVWHNLSYMKCTWLPGRNTSPDT | |
| | NYTLYYWHSSLGKILQCEDIYREGQHIGCSFA | |
| | LTNLKDSSFEQHSVQIVVKDNAGKIRPSFNIV | |
| | PLTSHVKPDPPHIKRLFFQNGNLYVQWKNPQN | |
| | FYSRCLSYQVEVNNSQTETNDIFYVEEAKCQN | |
| | SEFEGNLEGTICFMVPGVLPDTLNTVRIRVRT | |
| | NKLCYEDDKLWSNWSQAMSIGENTDPT | |
| 191 | QPPVTNLSVSVENLCTVIWTWDPPEGASPNCT | Exemplary canine IL13R ECD |
| | LRYFSHFDNKQDKKIAPETHRSKEVPLNERIC | |
| | LQVGSQCSTNESDNPSILVEKCTPPPEGDPES | |
| | AVTELQCVWHNLSYMKCTWLPGRNTSPDTNYT | |
| | LYYWHSSLGKILQCEDIYREGQHIGCSFALTN | |
| | LKDSSFEQHSVQIVVKDNAGKIRPSFNIVPLT | |
| | SHVKPDPPHIKRLFFQNGNLYVQWKNPQNFYS | |
| | RCLSYQVEVNNSQTETNDIFYVEEAKCQNSEF | |
| | EGNLEGTICFMVPGVLPDTLNTVRIRVRTNKL | |
| | CYEDDKLWSNWSQAMSI | |
| 192 | SQTQPPVTNLSVSVENLCTVIWTWDPPEGASP | Exemplary feline IL13R ECD |
| | NCTLRYFSHFDNKQDKKIAPETHRSKEVPLNE | |
| | RICLQVGSQCSTNESDNPSILVEKCTPPPEGD | |
| | PESAVTELQCVWHNLSYMKCTWLPGRNTSPDT | |
| | NYTLYYWHSSLGKILQCENIYREGQHIGCSFA | |
| | LTNLKDSSFEQHSVQIVVKDNAGKIRPSFNIV | |
| | PLTSHVKPDPPHIKRLFFQNGNLYVQWKNPQN | |
| | FYSRCLSYQVEVNNSQTETHDIFYVEEAKCQN | |
| | SEFEGNLEGTICFMVPGILPDTLNTVRIRVRT | |
| | NKLCYEDDRLWSNWSQAMSIGENTDPT | |
| 193 | QPPVTNLSVSVENLCTVIWTWDPPEGASPNCT | Exemplary feline IL13R ECD |
| | LRYFSHFDNKQDKKIAPETHRSKEVPLNERIC | |
| | LQVGSQCSTNESDNPSILVEKCTPPPEGDPES | |
| | AVTELQCVWHNLSYMKCTWLPGRNTSPDTNYT | |
| | LYYWHSSLGKILQCENIYREGQHIGCSFALTN | |
| | LKDSSFEQHSVQIVVKDNAGKIRPSFNIVPLT | |
| | SHVKPDPPHIKRLFFQNGNLYVQWKNPQNFYS | |
| | RCLSYQVEVNNSQTETHDIFYVEEAKCQNSEF | |
| | EGNLEGTICFMVPGILPDTLNTVRIRVRTNKL | |
| | CYEDDRLWSNWSQAMSI | |
| 194 | TESQPPVTNLSVSVENLCTVIWTWNPPEGVSP | Exemplary equine IL13R ECD |
| | NCSLWYFSHFGNKQDKKIAPETHRSKEVPLNE | |
| | RICLQVGSQCSTNESDNPSILVEKCISPPEGD | |
| | PESAVTELQCVWHNLSYMKCTWLPGKNASPDT | |
| | NYTLYYWHSSLGKILQCEDIYREGQHIGCSFA | |
| | LTEVKDSIFEQHSVQIMVKDNAGKIRPFFNIV | |
| | PLTSHVKPDPPHIKKLFFQNGDLYVQWKNPQN | |
| | FYSRCLSYQVEVNNSQTETRDIFSVEEAKCQN | |
| | PEFEGDLEGTICFMVPGVLPDTVNTVRIRVKT | |
| | NKLCYEDDKLWSNWSQAMSIGKKADPT | |
| 195 | QPPVTNLSVSVENLCTVIWTWNPPEGVSPNCS | Exemplary equine IL13R ECD |
| | LWYFSHFGNKQDKKIAPETHRSKEVPLNERIC | |
| | LQVGSQCSTNESDNPSILVEKCISPPEGDPES | |
| | AVTELQCVWHNLSYMKCTWLPGKNASPDTNYT | |
| | LYYWHSSLGKILQCEDIYREGQHIGCSFALTE | |
| | VKDSIFEQHSVQIMVKDNAGKIRPFFNIVPLT | |
| | SHVKPDPPHIKKLFFQNGDLYVQWKNPQNFYS | |
| | RCLSYQVEVNNSQTETRDIFSVEEAKCQNPEF | |
| | EGDLEGTICFMVPGVLPDTVNTVRIRVKTNKL | |
| | CYEDDKLWSNWSQAMSI | |
| 196 | SGSVKVLHEPSCFSDYISTSVCQWKMDHPTNC | Exemplary canine IL4R ECD |
| | SAELRLSYQLDFMGSENHTCVPENREDSVCVC | |
| | SMPIDDAVEADVYQLDLWAGQQLLWSGSFQPS | |
| | KHVKPRTPGNLTVHPNISHTWLLMWTNPYPTE | |
| | NHLHSELTYMVNVSNDNDPEDFKVYNVTYMGP | |
| | TLRLAASTLKSGASYSARVRAWAQTYNSTWSD | |
| | WSPSTTWLNYYEP | |
| 197 | KVLHEPSCFSDYISTSVCQWKMDHPTNCSAEL | Exemplary canine IL4R ECD |
| | RLSYQLDFMGSENHTCVPENREDSVCVCSMPI | |
| | DDAVEADVYQLDLWAGQQLLWSGSFQPSKHVK | |
| | PRTPGNLTVHPNISHTWLLMWTNPYPTENHLH | |
| | SELTYMVNVSNDNDPEDFKVYNVTYMGPTLRL | |
| | ASTLKSGASYSARVRAWAQTYNS | |
| 198 | SGSVKVLRAPTCFSDYFSTSVCQWNMDAPTNC | Exemplary feline IL4R ECD |
| | SAELRLSYQLNFMGSENRTCVPENGEGAACAC | |
| | SMLMDDFVEADVYQLHLWAGTQLLWSGSFKPS | |
| | SHVKPRAPGNLTVHPNVSHTWLLRWSNPYPPE | |
| | NHLHAELTYMVNISSEDDPTDVSVCASGFLCH | |
| | LLGLRRVETGAPGARLPPWLCAPRPRRVPGSQ | |
| | CAVISCCRWVLIALTSRGGRWRLTPGLRSQTR | |
| | YVSVAEGLFGATPRVLCPGTQAGLASAAREQM | |
| | SPDPSAFHSIDYEP | |
| 199 | KVLRAPTCFSDYFSTSVCQWNMDAPTNCSAEL | Exemplary feline IL4R ECD |
| | RLSYQLNFMGSENRTCVPENGEGAACACSMLM | |
| | DDFVEADVYQLHLWAGTQLLWSGSFKPSSHVK | |
| | PRAPGNLTVHPNVSHTWLLRWSNPYPPENHLH | |
| | AELTYMVNISSEDDPTDVSVCASGFLCHLLGL | |
| | RRVETGAPGARLPPWLCAPRPRRVPGSQCAVI | |
| | SCCRWVLIALTSRGGRWRLTPGLRSQTRYVSV | |
| | AEGLFGATPRVLCPGTQAGLASAAREQMSPDP | |
| | SAFHSIDYEP | |
| 200 | SGSVKVLHLTACFSDYISASTCEWKMDRPTNC | Exemplary equine IL4R ECD |
| | SAQLRLSYQLNDEFSDNLTCIPENREDEVCVC | |
| | RMLMDNIVSEDVYELDLWAGNQLLWNSSFKPS | |
| | RHVKPRAPQNLTVHAISHTWLLTWSNPYPLKN | |
| | HLWSELTYLVNISKEDDPTDFKIYNVTYMDPT | |
| | LRVTASTLKSRATYSARVKARAQNYNSTWSEW | |
| | SPSTTWHNYYEQP | |
| 201 | KVLHLTACFSDYISASTCEWKMDRPTNCSAQL | Exemplary equine IL4R ECD |
| | RLSYQLNDEFSDNLTCIPENREDEVCVCRMLM | |
| | DNIVSEDVYELDLWAGNQLLWNSSFKPSRHVK | |
| | PRAPQNLTVHAISHTWLLTWSNPYPLKNHLWS | |
| | ELTYLVNISKEDDPTDFKIYNVTYMDPTLRVT | |
| | ASTLKSRATYSARVKARAQNYNSTWSEWSPSI | |
| | TWHNYYEQP | |
| 271 | MAVLGLLFCLVTFPSCVLSTETQPPVTNLSVS | IL13R ECD-IL4R ECD- |
| | VENLCTVIWTWDPPEGASPNCTLRYFSHFDNK | wildtype canine IgG-B Fc |
| | QDKKIAPETHRSKEVPLNERICLQVGSQCSTN | |
| | ESDNPSILVEKCTPPPEGDPESAVTELQCVWH | |
| | NLSYMKCTWLPGRNTSPDTNYTLYYWHSSLGK | |
| | ILQCEDIYREGQHIGCSFALTNLKDSSFEQHS | |
| | VQIVVKDNAGKIRPSFNIVPLTSHVKPDPPHI | |
| | KRLFFQNGNLYVQWKNPQNFYSRCLSYQVEVN | |
| | NSQTETNDIFYVEEAKCQNSEFEGNLEGTICF | |
| | MVPGVLPDTLNTVRIRVRTNKLCYEDDKLWSN | |
| | WSQAMSIGENTDPT [symbol] GSVKVLHEPSCF | |
| | SDYISTSVCQWKMDHPTNCSAELRLSYQLDFM | |
| | GSENHTCVPENREDSVCVCSMPIDDAVEADVY | |
| | QLDLWAGQQLLWSGSFQPSKHVKPRTPGNLTV | |
| | HPNISHTWLLMWTNPYPTENHLHSELTYMVNV | |
| | SNDNDPEDFKVYNVTYMGPTLRLAASTLKSGA | |
| | SYSARVRAWAQTYNSTWSDWSPSTTWLNYYEP | |
| | KRENGRVPRPPDCPKCPAPEMLGGPSVFIFPP | |
| | KPKDTLLIARTPEVTCVVVDLDPEDPEVQISW | |
| | FVDGKQMQTAKTQPREEQFNGTYRVVSVLPIG | |
| | HQDWLKGKQFTCKVNNKALPSPIERTISKARG | |
| | QAHQPSVYVLPPSREELSKNTVSLTCLIKDFF | |
| | PPDIDVEWQSNGQQEPESKYRTTPPQLDEDGS | |
| | YFLYSKLSVDKSRWQRGDTFICAVMHEALHNH | |
| | YTQESLSHSPGK | |
| 202 | MAVLGLLFCLVTFPSCVLSTETQPPVTNLSVS | IL13R ECD-IL4R ECD- |
| | VENLCTVIWTWDPPEGASPNCTLRYFSHFDNK | variant canine IgG-B Fc |
| | QDKKIAPETHRSKEVPLNERICLQVGSQCSTN | |
| | ESDNPSILVEKCTPPPEGDPESAVTELQCVWH | |
| | NLSYMKCTWLPGRNTSPDTNYTLYYWHSSLGK | |
| | ILQCEDIYREGQHIGCSFALTNLKDSSFEQHS | |
| | VQIVVKDNAGKIRPSFNIVPLTSHVKPDPPHI | |
| | KRLFFQNGNLYVQWKNPQNFYSRCLSYQVEVN | |
| | NSQTETNDIFYVEEAKCQNSEFEGNLEGTICF | |
| | MVPGVLPDTLNTVRIRVRTNKLCYEDDKLWSN | |
| | WSQAMSIGENTDPT [symbol] SGSVKVLHEPSCF | |
| | SDYISTSVCQWKMDHPTNCSAELRLSYQLDFM | |
| | GSENHTCVPENREDSVCVCSMPIDDAVEADVY | |
| | QLDLWAGQQLLWSGSFQPSKHVKPRTPGNLTV | |
| | HPNISHTWLLMWTNPYPTENHLHSELTYMVNV | |
| | SNDNDPEDFKVYNVTYMGPTLRLAASTLKSGA | |
| | SYSARVRAWAQTYNSTWSDWSPSTTWLNYYEP | |
| | KRENGRVPRPPDCPKCPAPEMLGGPSVFIFPP | |
| | KPKDTLLIARTPEVTCVVVDLDPEDPEVQISW | |
| | FVDGKQMQTAKTQPREEQFNGTYRVVSVLPIG | |
| | HQDWLKGKQFTCRVNNKALPSPIERTISKARG | |
| | QAHQPSVYVLPPSREELSKNTVSLTCLIKDFF | |
| | PPDIDVEWQSNGQQEPESKYRTTPPQLDEDGS | |
| | YFLYSKLSVDKSRWQRGDTFICAVMHEALHNH | |
| | YTQESLSHSPGK | |
| 203 | MGRLGEGLNCTVKNSTCLDDSWIHPRNLTPSS | Exemplary canine IL17Ra ECD |
| | PKDVQVHLDFAQTQHGDLLPIIGIRWTLQTDA | |
| | SILFLEGAELSVLQLNTNERVCVKFEFLSKLK | |
| | HHHKRWHFTFSHFVVEPGQEYEVTVHHLPKPI | |
| | PDGDPNHQSKNFLVPGCEDPRMRMTTPCVSSG | |
| | SLWDPNITAEALEAHQLQVHFTLWNESAQYQI | |
| | LLTSFPHTENRSCFHRVLMVPEPTLKEHHQRA | |
| | NIMLTGSSSNWCCRHQVQIQPFFSSCLNDCLR | |
| | HSVTVPCPEIPDAPVSIADYIPL | |
| 204 | LGEGLNCTVKNSTCLDDSWIHPRNLTPSSPKD | Exemplary canine IL17Ra ECD |
| | VQVHLDFAQTQHGDLLPIIGIRWTLQTDASIL | |
| | FLEGAELSVLQLNTNERVCVKFEFLSKLKHHH | |
| | KRWHFTFSHFVVEPGQEYEVTVHHLPKPIPDG | |
| | DPNHQSKNFLVPGCEDPRMRMTTPCVSSGSLW | |
| | DPNITAEALEAHQLQVHFTLWNESAQYQILLT | |
| | SFPHTENRSCFHRVLMVPEPTLKEHHQRANIM | |
| | LTGSSSNWCCRHQVQIQPFFSSCLNDCLRHSV | |
| | TVPCP | |
| 205 | SLRLLDHRALVCSQPGLNCTVKNSTCLDDSWI | Exemplary human IL17Ra ECD |
| | HPRNLTPSSPKDLQIQLHFAHTQQGDLFPVAH | |
| | IEWTLQTDASILYLEGAELSVLQLNTNERLCV | |
| | RFEFLSKLRHHHRRWRFTFSHFVVDPDQEYEV | |
| | TVHHLPKPIPDGDPNHQSKNFLVPDCEHARMK | |
| | VTTPCMSSGSLWDPDITVETLEAHQLRVSFTL | |
| | WNESTHYQILLTSFPHMENHSCFEHMHHIPAP | |
| | RPEEFHQRSDVTLTLRNLKGCCRHQVQIQPFF | |
| | SSCLNDCLRHSATVSCP | |
| 206 | SPRLLDYPAPVCSQQGLNCVVKNSTCLDDSWI | Exemplary feline IL17Ra ECD |
| | HLRNLTPSSPKDVQVHLDFVQTQHGDLLPVAG | |
| | IRWTLQTDASILYLEGAELSVLQLNTNERLCV | |
| | KFEFLTRLKHHHKRWHFTFSHFVVEPGQEYEV | |
| | TVHHLPKPIPDGDPNHQSRNFPVPGCEDPRMK | |
| | MITPCVGSGSLWDPNITVETLEARQLWVSFTL | |
| | WNESTHYQILLTSFPHTENHSCFQHTLMVPEP | |
| | AYQDSRQRSNVTLTLSDSNWCCRHRVQIQPFF | |
| | SSCLNDCLRHSITVPCPEIPDPPVSIADYI | |
| 207 | SPRLLEHPAPVCSQQGLNCTVKNSTCLDDSWL | Exemplary equine IL17Ra ECD |
| | HPPHLTPSSPKDVQIQLHFAHTQQGDLLPVIH | |
| | IEWTLQTDASILYLEGAELSVLQLSTNERLCV | |
| | TFEFLSRLKHHHKRWRFTFAHFVVEPGQEYEV | |
| | TVHHLPKPFPHGDPNHQSRNFLVPDCMDPRMR | |
| | ITTPCVSSGSLWDPNITVETLEAHRLRVDFTL | |
| | WNESARYQILLSSFPHMENQSCFDDVQNILKH | |
| | TPEASHQRANITLTLSDFNWCCRHHVQIQPFF | |
| | SSCLNDCLRHTVTVPCPEIPDTPDSTADYM | |
| 208 | LERLVGPQDATHCSPVSLEPWGDEERLRVQFL | Exemplary human IL17RC ECD |
| | AQQSLSLAPVTAATARTALSGLSGADGRREER | |
| | GRGKSWVCLSLGGSGNTEPQKKGLSCRLWDSD | |
| | ILCLPGDIVPAPGPVLAPTHLQTELVLRCQKE | |
| | TDCDLCLRVAVHLAVHGHWEEPEDEEKFGGAA | |
| | DSGVEEPRNASLQAQVVLSFQAYPTARCVLLE | |
| | VQVPAALVQFGQSVGSVVYDCFEAALGSEVRI | |
| | WSYTQPRYEKELNHTQQLPDCRGLEVWNSIPS | |
| | CWALPWLNVSADGDNVHLVLNVSEEQHFGLSL | |
| | YWNQVQGPPKPRWHKNLTGPQIITLNHTDLVP | |
| | CLCIQVWPLEPDSVRTNICPFREDPRAHQNLW | |
| | QAARLQLLTLQSWLLDAPCSLPAEAALCWRAP | |
| | GGDPCQPLVPPLSWENVTVDKVLEFPLLKGHP | |
| | NLCVQVNSSEKLQLQECLWADSLGPLKDDVLL | |
| | LETRGPQDNRSL | |
| 209 | VLRCQKETDCDLCLRVAVHLAVHGHWEEPEDE | Exemplary human IL17RC ECD |
| | EKFGGAADSGVEEPRNASLQAQVVLSFQAYPT | |
| | ARCVLLEVQVPAALVQFGQSVGSVVYDCFEAA | |
| | LGSEVRIWSYTQPRYEKELNHTQQLPDCRGLE | |
| | VWNSIPSCWALPWLNVSADGDNVHLVLNVSEE | |
| | QHFGLSLYWNQVQGPPKPRWHKNLTGPQIITL | |
| | NHTDLVPCLCIQVWPLEPDSVRTNICPFREDP | |
| | RAHQNLWQAARLQLLTLQSWLLDAPCSLPAEA | |
| | ALCWRAPGGDPCQPLVPPLSWENVTVDKVLEF | |
| | PLLKGHPNLCVQVNSSEKLQLQECLWADSLGP | |
| | LKDDVLLLETRGPQDNRSL | |
| 210 | LEKLMGPQDTARCSPGLSCHLWDGDVLCLPGS | Exemplary canine IL17RC ECD |
| | IVSAPGPVLVPTRLQTELVLRCYQETDCDLCV | |
| | RVAIHLAVHGHWEEPKDEDKFGRAADPELEEP | |
| | RNAFLQAQVVLSFQAYPTARCVLLEVQVPAAL | |
| | VQPGQSVGSVVFDCFEAALGAEVRIWSYTQPR | |
| | YQKELNFTQQLPDCKGLEVRDSIQSCWALPWL | |
| | NVSADGDDVYLVLDVSEEQRFGLSLYWNQIQG | |
| | PTKPWWHRNLTGPQTITLNHTDLFPCLCIQVW | |
| | PLEPDSVRTSVCPFREDPRAHRNLWRAARLQL | |
| | LPPRGWRLDAPCSLLAEATLCWQAPGGGPCQS | |
| | LVPPLYQANVTVNKTLELPLLNAHPNLCVQVS | |
| | SWEKLQLQECLWADSLRALKDDLLLVETRGLQ | |
| | DNRSL | |
| 211 | RIWSYTQPRYQKELNFTQQLPDCKGLEVRDSI | Exemplary canine IL17RC ECD |
| | QSCWALPWLNVSADGDDVYLVLDVSEEQRFGL | |
| | SLYWNQIQGPTKPWWHRNLTGPQTITLNHTDL | |
| | FPCLCIQVWPLEPDSVRTSVCPFREDPRAHRN | |
| | LWRAARLQLLPPRGWRLDAPCSLLAEATLCWQ | |
| | APGGGPCQSLVPPLYQANVTVNKTLELPLLNA | |
| | HPNLCVQVSSWEKLQLQECLWADSLRALKDDL | |
| | LLVETRGLQDNRSL | |
| 212 | LERLVGPQDTARCSPGLSCHLWDGDVLCLPGS | Exemplary feline IL17RC ECD |
| | IVSAPGPVLVPTRLQTELVLRCYQETDCDLCV | |
| | RVAIHLAVHGHWEEPKGEEKFGGAADPELEES | |
| | RNAFLQAQVVLSFQAYPTARCVLLEVQVPAAL | |
| | VQPGQSVGSVVFDCFEAALGAEVRIWSYTQPR | |
| | YQKELNLTQHLPDCKGLEVRDSIQSCWALPWL | |
| | NVSADGDDVHLVLDVSEDQRFGLSLYWNQVQG | |
| | PTKPWWHRNLTGPQTITLNHTDLFPCLCIQVW | |
| | PLEPDSVRTSICPFREDPRAHRNLWRAARLQL | |
| | LPPRGWRLDAPCSLPAEATLCWQAPGGGPCQS | |
| | LVPPLPPANVTVNKALELPLLNVHPNLCVQVS | |
| | SWEKLQLQECLWVDSLGPLKDDMLLVETRDPH | |
| | NNRSL | |
| 213 | RIWSYTQPRYQKELNLTQHLPDCKGLEVRDSI | Exemplary feline IL17RC ECD |
| | QSCWALPWLNVSADGDDVHLVLDVSEDQRFGL | |
| | SLYWNQVQGPTKPWWHRNLTGPQTITLNHTDL | |
| | FPCLCIQVWPLEPDSVRTSICPFREDPRAHRN | |
| | LWRAARLQLLPPRGWRLDAPCSLPAEATLCWQ | |
| | APGGGPCQSLVPPLPPANVTVNKALELPLLNV | |
| | HPNLCVQVSSWEKLQLQECLWVDSLGPLKDDM | |
| | LLVETRDPHNNRSL | |
| 214 | LERLEGLQDAARCSPGLSCHLWDGDVVCLPGS | Exemplary equine IL17RC ECD |
| | IVSAPGPVLVPTSLQTELVRRCYQETDCDLCV | |
| | RVAVHLAVHGHWEKPEDEEKLGRAADPEPEEP | |
| | RNASLQAQVVLSFQAYPTARCVLLEVQVPAAL | |
| | VQPGQSVGSVVFDCFEAALGTEVQIWSYTQPR | |
| | YQKELNLTRQLPDCRGLEVQDSIQSCRALPWL | |
| | SVTADGDNVHLVLDVSEEQSFGLSLYWNQVQG | |
| | PVKPWWHRNLTGPQTIPLNQTDIVPCLCIQAW | |
| | PLEPDSVRTSICPFTEDPRAHRNLWRAARLQL | |
| | LPPRGWRLDAPCSLHAQATLCWQAPSRGPCQP | |
| | LVPPLPRENVTVNMALEFPLLKGHPNLCVQVS | |
| | SWEKMQLQLQECLWADSLGPLKDDMLLVEAGG | |
| | PQDNRSF | |
| 215 | QIWSYTQPRYQKELNLTRQLPDCRGLEVQDSI | Exemplary equine IL17RC ECD |
| | QSCRALPWLSVTADGDNVHLVLDVSEEQSFGL | |
| | SLYWNQVQGPVKPWWHRNLTGPQTIPLNQTDI | |
| | VPCLCIQAWPLEPDSVRTSICPFTEDPRAHRN | |
| | LWRAARLQLLPPRGWRLDAPCSLHAQATLCWQ | |
| | APSRGPCQPLVPPLPRENVTVNMALEFPLLKG | |
| | HPNLCVQVSSWEKMQLQLQECLWADSLGPLKD | |
| | DMLLVEAGGPQDNRSF | |
| 216 | MGRLGEGLNCTVKNSTCLDDSWIHPRNLTPSS | Exemplary canine IL17Ra |
| | PKDVQVHLDFAQTQHGDLLPIIGIRWTLQTDA | ECD-canine IL17RC |
| | SILFLEGAELSVLQLNTNERVCVKFEFLSKLK | ECD-wildtype IgG-B-Fc |
| | HHHKRWHFTFSHFVVEPGQEYEVTVHHLPKPI | |
| | PDGDPNHQSKNFLVPGCEDPRMRMTTPCVSSG | |
| | SLWDPNITAEALEAHQLQVHFTLWNESAQYQI | |
| | LLTSFPHTENRSCFHRVLMVPEPTLKEHHQRA | |
| | NIMLTGSSSNWCCRHQVQIQPFFSSCLNDCLR | |
| | HSVTVPCPEIPDAPVSIADYIGSLEKLMGPQD | |
| | TARCSPGLSCHLWDGDVLCLPGSIVSAPGPVL | |
| | VPTRLQTELVLRCYQETDCDLCVRVAIHLAVH | |
| | GHWEEPKDEDKFGRAADPELEEPRNAFLQAQV | |
| | VLSFQAYPTARCVLLEVQVPAALVQPGQSVGS | |
| | VVFDCFEAALGAEVRIWSYTQPRYQKELNFTQ | |
| | QLPDCKGLEVRDSIQSCWALPWLNVSADGDDV | |
| | YLVLDVSEEQRFGLSLYWNQIQGPTKPWWHRN | |
| | LTGPQTITLNHTDLFPCLCIQVWPLEPDSVRT | |
| | SVCPFREDPRAHRNLWRAARLQLLPPRGWRLD | |
| | APCSLLAEATLCWQAPGGGPCQSLVPPLYQAN | |
| | VTVNKTLELPLLNAHPNLCVQVSSWEKLQLQE | |
| | CLWADSLRALKDDLLLVETRGLQDNRSL [symbol] PK | |
| | RENGRVPRPPDCPKCPAPEMLGGPSVFIFPPK | |
| | PKDTLLIARTPEVTCVVVDLDPEDPEVQISWF | |
| | VDGKQMQTAKTQPREEQFNGTYRVVSVLPIGH | |
| | QDWLKGKQFTCKVNNKALPSPIERTISKARGQ | |
| | AHQPSVYVLPPSREELSKNTVSLTCLIKDFFP | |
| | PDIDVEWQSNGQQEPESKYRTTPPQLDEDGSY | |
| | FLYSKLSVDKSRWQRGDTFICAVMHEALHNHY | |
| | TQESLSHSPGK | |
| 217 | MGRLGEGLNCTVKNSTCLDDSWIHPRNLTPSS | Exemplary canine IL17Ra |
| | PKDVQVHLDFAQTQHGDLLPIIGIRWTLQTDA | ECD-canine IL17RC |
| | SILFLEGAELSVLQLNTNERVCVKFEFLSKLK | ECD-wildtype IgG-B-Fc |
| | HHHKRWHFTFSHFVVEPGQEYEVTVHHLPKPI | |
| | PDGDPNHQSKNFLVPGCEDPRMRMTTPCVSSG | |
| | SLWDPNITAEALEAHQLQVHFTLWNESAQYQI | |
| | LLTSFPHTENRSCFHRVLMVPEPTLKEHHQRA | |
| | NIMLTGSSSNWCCRHQVQIQPFFSSCLNDCLR | |
| | HSVTVPCPEIPDAPVSIADYIGSRIWSYTQPR | |
| | YQKELNFTQQLPDCKGLEVRDSIQSCWALPWL | |
| | NVSADGDDVYLVLDVSEEQRFGLSLYWNQIQG | |
| | PTKPWWHRNLTGPQTITLNHTDLFPCLCIQVW | |
| | PLEPDSVRTSVCPFREDPRAHRNLWRAARLQL | |
| | LPPRGWRLDAPCSLLAEATLCWQAPGGGPCQS | |
| | LVPPLYQANVTVNKTLELPLLNAHPNLCVQVS | |
| | SWEKLQLQECLWADSLRALKDDLLLVETRGLQ | |
| | DNRSL [symbol] PKRENGRVPRPPDCPKCPAPEMLGG | |
| | PSVFIFPPKPKDTLLIARTPEVTCVVVDLDPE | |
| | DPEVQISWFVDGKQMQTAKTQPREEQFNGTYR | |
| | VVSVLPIGHQDWLKGKQFTCKVNNKALPSPIE | |
| | RTISKARGQAHQPSVYVLPPSREELSKNTVSL | |
| | TCLIKDFFPPDIDVEWQSNGQQEPESKYRTTP | |
| | PQLDEDGSYFLYSKLSVDKSRWQRGDTFICAV | |
| | MHEALHNHYTQESLSHSPGK | |
| 218 | VSFPASVQLHEAVELHHWCIPFSVDGQPAPSL | Exemplary canine TrkA ECD |
| | RWLFNGSVLNETSFIFTEFLEPVANETVRHGC | |
| | LRLNQPTHVNNGNYTLLAANPSGRAAAFVMAA | |
| | FMDNPFEFNPEDPIPVSFSPVDTNSTSGD | |
| 219 | VSFPASVQLHEAVELHHWCIPFSVDGQPAPSL | Exemplary canine TrkA ECD |
| | RWLFNGSVLNETSFIFTEFLEPVANETVRHGC | |
| | LRLNQPTHVNNGNYTLLAANPSGRAAAFVMAA | |
| | FMDNP | |
| 220 | FPASVQLHEAVELHHWCIPFSVDGQPAPSLRW | Exemplary canine TrkA ECD |
| | LFNGSVLNETSFIFTEFLEPVANETVRHGCLR | |
| | LNQPTHVNNGNYTLLAANPSGRAAAFVMAAFM | |
| | DNP | |
| 221 | VSFPASVQLHAAVELHHWCIPFSVDGQPAPSL | Exemplary feline TrkA ECD |
| | RWLFNGSVLNETSFIFTEFLEPAANETVRHGC | |
| | LRLNQPTHVNNGNYTLLAANPSGRAAASVLAA | |
| | FMDNPFEFNPEDPIPVSFSPVDSNSTSGD | |
| 222 | VSFPASVQLHAAVELHHWCIPFSVDGQPAPSL | Exemplary feline TrkA ECD |
| | RWLFNGSVLNETSFIFTEFLEPAANETVRHGC | |
| | LRLNQPTHVNNGNYTLLAANPSGRAAASVLAA | |
| | FMDNP | |
| 223 | FPASVQLHAAVELHHWCIPFSVDGQPAPSLRW | Exemplary feline TrkA ECD |
| | LFNGSVLNETSFIFTEFLEPAANETVRHGCLR | |
| | LNQPTHVNNGNYTLLAANPSGRAAASVLAAFM | |
| | DNP | |
| 224 | VSFPASVHLQTAVEQHHWCIPFSVDGQPAPTL | Exemplary equine TrkA ECD |
| | RWLFNGSVLNETSFIFTEFLESAANETMRHGC | |
| | LRLNQPTHVNNGNYTLLATNPYGQDSASVMVA | |
| | FMDNPFEFNPEDPIPVSFSPVDTNSTSRD | |
| 225 | VSFPASVHLQTAVEQHHWCIPFSVDGQPAPTL | Exemplary equine TrkA ECD |
| | RWLFNGSVLNETSFIFTEFLESAANETMRHGC | |
| | LRLNQPTHVNNGNYTLLATNPYGQDSASVMVA | |
| | FMDNP | |
| 226 | FPASVHLQTAVEQHHWCIPFSVDGQPAPTLRW | Exemplary equine TrkA ECD |
| | LFNGSVLNETSFIFTEFLESAANETMRHGCLR | |
| | LNQPTHVNNGNYTLLATNPYGQDSASVMVAFM | |
| | DNP | |
| 227 | VSFPASVQLHTAVEMHHWCIPFSVDGQPAPSL | Exemplary human TrkA ECD |
| | RWLFNGSVLNETSFIFTEFLEPAANETVRHGC | |
| | LRLNQPTHVNNGNYTLLAANPFGQASASIMAA | |
| | FMDNPFEFNPEDPIPVSFSPVDTNSTSGD | |
| 228 | VSFPASVQLHTAVEMHHWCIPFSVDGQPAPSL | Exemplary human TrkA ECD |
| | RWLFNGSVLNETSFIFTEFLEPAANETVRHGC | |
| | LRLNQPTHVNNGNYTLLAANPFGQASASIMAA | |
| | FMDNP | |
| 229 | FPASVQLHTAVEMHHWCIPFSVDGQPAPSLRW | Exemplary human TrkA ECD |
| | LFNGSVLNETSFIFTEFLEPAANETVRHGCLR | |
| | LNQPTHVNNGNYTLLAANPFGQASASIMAAFM | |
| | DNP | |
| 230 | MDMRVPAQLLGLLLLWLRGARCVSFPASVQLH | Exemplary canine TrkA ECD- |
| | EAVELHHWCIPFSVDGQPAPSLRWLFNGSVLN | wildtype canine Fc-IgG-B |
| | ETSFIFTEFLEPVANETVRHGCLRLNQPTHVN | IgG-B |
| | NGNYTLLAANPSGRAAAFVMAAFMDNP [symbol] | |
| | [symbol] RPPDCPKCPAPEMLGGPSVFIFPPKPKD | |
| | ILLIARTPEVTCVVVDLDPEDPEVQISWFVDG | |
| | KQMQTAKTQPREEQFNGTYRVVSVLPIGHQDW | |
| | LKGKQFTCKVNNKALPSPIERTISKARGQAHQ | |
| | PSVYVLPPSREELSKNTVSLTCLIKDFFPPDI | |
| | DVEWQSNGQQEPESKYRTTPPQLDEDGSYFLY | |
| | SKLSVDKSRWQRGDTFICAVMHEALHNHYTQE | |
| | SLSHSPGK | |
| 231 | MDMRVPAQLLGLLLLWLRGARCVSFPASVQLH | Exemplary canine TrkA ECD- |
| | EAVELHHWCIPFSVDGQPAPSLRWLFNGSVLN | wildtype canine IgG-B Fc |
| | ETSFIFTEFLEPVANETVRHGCLRLNQPTHVN | |
| | NGNYTLLAANPSGRAAAFVMAAFMDNPFEFNP | |
| | EDPIPVSFSPVDTNSTSGD [symbol] RPPD | |
| | CPKCPAPEMLGGPSVFIFPPKPKDTLLIARTP | |
| | EVTCVVVDLDPEDPEVQISWFVDGKQMQTAKT | |
| | QPREEQFNGTYRVVSVLPIGHQDWLKGKQFTC | |
| | KVNNKALPSPIERTISKARGQAHQPSVYVLPP | |
| | SREELSKNTVSLTCLIKDFFPPDIDVEWQSNG | |
| | QQEPESKYRTTPPQLDEDGSYFLYSKLSVDKS | |
| | RWQRGDTFICAVMHEALHNHYTQESLSHSPGK | |
| 232 | MDMRVPAQLLGLLLLWLRGARCVSFPASVQLH | Exemplary canine TrkA ECD- |
| | EAVELHHWCIPFSVDGQPAPSLRWLFNGSVLN | variant canine IgG-B Fc |
| | ETSFIFTEFLEPVANETVRHGCLRLNQPTHVN | Variant canine IgG-B Fc |
| | NGNYTLLAANPSGRAAAFVMAAFMDNP [symbol] | Protein A+ |
| | [symbol] RPPDCPKCPAPEPLGGPSVFIFPPKPKD | C1q − |
| | TLLIARTPEVTCVVVDLDREDPEVQISWFVDG | CD16 − |
| | KQMQTAKTQPREEQFNGTYRVVSVLPIGHQDW | |
| | LKGKQFTCRVNNKALPSPIERTISKARGQAHQ | |
| | PSVYVLPPSREELSKNTVSLTCLIKDFFPPDI | |
| | DVEWQSNGQQEPESKYRTTPPQLDEDGSYFLY | |
| | SKLSVDKSRWQRGDTFICAVMHEALHNHYTQE | |
| | SLSHSPGK | |
| 233 | MDMRVPAQLLGLLLLWLRGARCVSFPASVQLH | Exemplary canine TrkA |
| | EAVELHHWCIPFSVDGQPAPSLRWLFNGSVLN | ECD-variant canine |
| | ETSFIFTEFLEPVANETVRHGCLRLNQPTHVN | IgG-B Fc |
| | NGNYTLLAANPSGRAAAFVMAAFMDNPFEFNP | Variant canine IgG-B Fc |
| | EDPIPVSFSPVDTNSTSGD [symbol] RPPD | Protein A+ |
| | CPKCPAPEPLGGPSVFIFPPKPKDTLLIARTP | C1q − |
| | EVTCVVVDLDREDPEVQISWFVDGKQMQTAKT | CD16 − |
| | QPREEQFNGTYRVVSVLPIGHQDWLKGKQFTC | |
| | RVNNKALPSPIERTISKARGQAHQPSVYVLPP | |
| | SREELSKNTVSLTCLIKDFFPPDIDVEWQSNG | |
| | QQEPESKYRTTPPQLDEDGSYFLYSKLSVDKS | |
| | RWQRGDTFICAVMHEALHNHYTQESLSHSPGK | |
| 234 | MDMRVPAQLLGLLLLWLRGARCVSFPASVQLH | Exemplary canine TrkA ECD- |
| | EAVELHHWCIPFSVDGQPAPSLRWLFNGSVLN | wildtype canine IgG-A Fc |
| | ETSFIFTEFLEPVANETVRHGCLRLNQPTHVN | |
| | NGNYTLLAANPSGRAAAFVMAAFMDNP [symbol] | |
| | [symbol] FNECRCTDTPCPVPEPLGGPSVLIFPPK | |
| | PKDILRITRTPEVTCVVLDLGREDPEVQISWF | |
| | VDGKEVHTAKTQSREQQFNGTYRVVSVLPIEH | |
| | QDWLTGKEFKCRVNHIDLPSPIERTISKARGR | |
| | AHKPSVYVLPPSPKELSSSDTVSITCLIKDFY | |
| | PPDIDVEWQSNGQQEPERKHRMTPPQLDEDGS | |
| | YFLYSKLSVDKSRWQQGDPFTCAVMHETLQNH | |
| | YTDLSLSHSPGK | |
| 235 | MDMRVPAQLLGLLLLWLRGARCVSFPASVQLH | Exemplary canine TrkA ECD- |
| | EAVELHHWCIPFSVDGQPAPSLRWLFNGSVLN | wildtype canine IgG-A Fc |
| | ETSFIFTEFLEPVANETVRHGCLRLNQPTHVN | |
| | NGNYTLLAANPSGRAAAFVMAAFMDNPFEFNP | |
| | EDPIPVSFSPVDTNSTSGD [symbol] FNEC | |
| | RCTDTPCPVPEPLGGPSVLIFPPKPKDILRIT | |
| | RTPEVTCVVLDLGREDPEVQISWFVDGKEVHT | |
| | AKTQSREQQFNGTYRVVSVLPIEHQDWLTGKE | |
| | FKCRVNHIDLPSPIERTISKARGRAHKPSVYV | |
| | LPPSPKELSSSDTVSITCLIKDFYPPDIDVEW | |
| | QSNGQQEPERKHRMTPPQLDEDGSYFLYSKLS | |
| | VDKSRWQQGDPFTCAVMHETLQNHYTDLSLSH | |
| | SPGK | |
| 236 | MDMRVPAQLLGLLLLWLRGARCVSFPASVQLH | Exemplary canine TrkA |
| | EAVELHHWCIPFSVDGQPAPSLRWLFNGSVLN | ECD-variant canine |
| | ETSFIFTEFLEPVANETVRHGCLRLNQPTHVN | IgG-A Fc |
| | NGNYTLLAANPSGRAAAFVMAAFMDNP [symbol] | Variant canine IgG-A Fc |
| | [symbol] FNECRCTDTPCPVPEPLGGPSVLIFPPK | Protein A+ |
| | PKDTLLIARTPEVTCVVLDLGREDPEVQISWF | C1q − |
| | VDGKEVHTAKTQSREQQFNGTYRVVSVLPIGH | CD16 − |
| | QDWLTGKEFKCRVNHIDLPSPIERTISKARGR | |
| | AHKPSVYVLPPSPKELSSSDTVSITCLIKDFY | |
| | PPDIDVEWQSNGQQEPERKHRMTPPQLDEDGS | |
| | YFLYSKLSVDKSRWQQGDPFTCAVMHEALHNH | |
| | YTDLSLSHSPGK | |
| 237 | MDMRVPAQLLGLLLLWLRGARCVSFPASVQLH | Exemplary canine TrkA ECD- |
| | EAVELHHWCIPFSVDGQPAPSLRWLFNGSVLN | variant canine IgG-A Fc |
| | ETSFIFTEFLEPVANETVRHGCLRLNQPTHVN | Variant canine IgG-A Fc |
| | NGNYTLLAANPSGRAAAFVMAAFMDNPFEFNP | Protein A+ |
| | EDPIPVSFSPVDTNSTSGD [symbol] FNEC | C1q − |
| | RCTDTPCPVPEPLGGPSVLIFPPKPKDTLLIA | CD16 − |
| | RTPEVTCVVLDLGREDPEVQISWFVDGKEVHT | |
| | AKTQSREQQFNGTYRVVSVLPIGHQDWLTGKE | |
| | FKCRVNHIDLPSPIERTISKARGRAHKPSVYV | |
| | LPPSPKELSSSDTVSITCLIKDFYPPDIDVEW | |
| | QSNGQQEPERKHRMTPPQLDEDGSYFLYSKLS | |
| | VDKSRWQQGDPFTCAVMHEALHNHYTDLSLSH | |
| | SPGK | |
| 238 | MDMRVPAQLLGLLLLWLRGARCVSFPASVQLH | Exemplary canine TrkA ECD- |
| | EAVELHHWCIPFSVDGQPAPSLRWLFNGSVLN | wildtype canine IgG-D Fc |
| | ETSFIFTEFLEPVANETVRHGCLRLNQPTHVN | |
| | NGNYTLLAANPSGRAAAFVMAAFMDNP [symbol] | |
| | [symbol] PKESTCKCISPCPVPESLGGPSVFIFPP | |
| | KPKDILRITRIPEITCVVLDLGREDPEVQISW | |
| | FVDGKEVHTAKTQPREQQFNSTYRVVSVLPIE | |
| | HQDWLTGKEFKCRVNHIGLPSPIERTISKARG | |
| | QAHQPSVYVLPPSPKELSSSDTVTLTCLIKDF | |
| | FPPEIDVEWQSNGQPEPESKYHTTAPQLDEDG | |
| | SYFLYSKLSVDKSRWQQGDTFTCAVMHEALQN | |
| | HYTDLSLSHSPGK | |
| 239 | MDMRVPAQLLGLLLLWLRGARCVSFPASVQLH | Exemplary canine TrkA ECD- |
| | EAVELHHWCIPFSVDGQPAPSLRWLFNGSVLN | wildtype canine IgG-D Fc |
| | ETSFIFTEFLEPVANETVRHGCLRLNQPTHVN | |
| | NGNYTLLAANPSGRAAAFVMAAFMDNPFEFNP | |
| | EDPIPVSFSPVDTNSISGD [symbol] PKES | |
| | TCKCISPCPVPESLGGPSVFIFPPKPKDILRI | |
| | TRIPEITCVVLDLGREDPEVQISWFVDGKEVH | |
| | TAKTQPREQQFNSTYRVVSVLPIEHQDWLTGK | |
| | EFKCRVNHIGLPSPIERTISKARGQAHQPSVY | |
| | VLPPSPKELSSSDTVTLTCLIKDFFPPEIDVE | |
| | WQSNGQPEPESKYHTTAPQLDEDGSYFLYSKL | |
| | SVDKSRWQQGDTFTCAVMHEALQNHYTDLSLS | |
| | HSPGK | |
| 240 | MDMRVPAQLLGLLLLWLRGARCVSFPASVQLH | Exemplary canine TrkA ECD- |
| | EAVELHHWCIPFSVDGQPAPSLRWLFNGSVLN | variant canine IgG-D Fc |
| | ETSFIFTEFLEPVANETVRHGCLRLNQPTHVN | Variant canine IgG-A Fc |
| | NGNYTLLAANPSGRAAAFVMAAFMDNP [symbol] | Protein A+ |
| | [symbol] PKESTCKCISPCPVPESLGGPSVFIFPP | C1q − |
| | KPKDTLLIARTPEITCVVLDLGREDPEVQISW | CD16 − |
| | FVDGKEVHTAKTQPREQQFNSTYRVVSVLPIG | |
| | HQDWLTGKEFKCRVNHIGLPSPIERTISKARG | |
| | QAHQPSVYVLPPSPKELSSSDTVTLTCLIKDF | |
| | FPPEIDVEWQSNGQPEPESKYHTTAPQLDEDG | |
| | SYFLYSKLSVDKSRWQQGDTFTCAVMHEALHN | |
| | HYTDLSLSHSPGK | |
| 241 | MDMRVPAQLLGLLLLWLRGARCVSFPASVQLH | Exemplary canine TrkA ECD- |
| | EAVELHHWCIPFSVDGQPAPSLRWLFNGSVLN | variant canine IgG-D Fc |
| | ETSFIFTEFLEPVANETVRHGCLRLNQPTHVN | Variant canine IgG-A Fc |
| | NGNYTLLAANPSGRAAAFVMAAFMDNPFEFNP | Protein A+ |
| | EDPIPVSFSPVDTNSTSGD [symbol] PKES | C1q − |
| | TCKCISPCPVPESLGGPSVFIFPPKPKDTLLI | CD16 − |
| | ARTPEITCVVLDLGREDPEVQISWFVDGKEVH | |
| | TAKTQPREQQFNSTYRVVSVLPIGHQDWLTGK | |
| | EFKCRVNHIGLPSPIERTISKARGQAHQPSVY | |
| | VLPPSPKELSSSDTVTLTCLIKDFFPPEIDVE | |
| | WQSNGQPEPESKYHTTAPQLDEDGSYFLYSKL | |
| | SVDKSRWQQGDTFTCAVMHEALHNHYTDLSLS | |
| | HSPGK | |
| 242 | MDMRVPAQLLGLLLLWLRGARCVSFPASVQLH | Exemplary feline TrkA ECD- |
| | AAVELHHWCIPFSVDGQPAPSLRWLFNGSVLN | variant feline IgG-2 Fc |
| | ETSFIFTEFLEPAANETVRHGCLRLNQPTHVN | Variant feline IgG2 Fc |
| | NGNYTLLAANPSGRAAASVLAAFMDNP [symbol] | Hinge Cys |
| | [symbol] PKTASTIESKTGECPKCPVPEIPGAPSV | |
| | FIFPPKPKDTLSISRTPEVTCLVVDLGPDDSN | |
| | VQITWFVDNTEMHTAKTRPREEQFNSTYRVVS | |
| | VLPILHQDWLKGKEFKCKVNSKSLPSAMERTI | |
| | SKAKGQPHEPQVYVLPPTQEELSENKVSVTCL | |
| | IKGFHPPDIAVEWEITGQPEPENNYQTTPPQL | |
| | DSDGTYFLYSRLSVDRSHWQRGNTYTCSVSHE | |
| | ALHSHHTQKSLTQSPGK | |
| 243 | MDMRVPAQLLGLLLLWLRGARCVSFPASVQLH | Exemplary feline TrkA ECD- |
| | AAVELHHWCIPFSVDGQPAPSLRWLFNGSVLN | variant feline IgG-2 Fc |
| | ETSFIFTEFLEPAANETVRHGCLRLNQPTHVN | Variant feline IgG2 Fc |
| | NGNYTLLAANPSGRAAASVLAAFMDNPFEFNP | Hinge Cys |
| | EDPIPVSFSPVDSNSTSGD [symbol] PKTA | |
| | STIESKTGECPKCPVPEIPGAPSVFIFPPKPK | |
| | DTLSISRTPEVTCLVVDLGPDDSNVQITWFVD | |
| | NTEMHTAKTRPREEQFNSTYRVVSVLPILHQD | |
| | WLKGKEFKCKVNSKSLPSAMERTISKAKGQPH | |
| | EPQVYVLPPTQEELSENKVSVTCLIKGFHPPD | |
| | IAVEWEITGQPEPENNYQTTPPQLDSDGTYFL | |
| | YSRLSVDRSHWQRGNTYTCSVSHEALHSHHTQ | |
| | KSLIQSPGK | |
| 244 | METDTLLLWVLLLWVPGSTGVSFPASVHLQTA | Exemplary equine TrkA ECD- |
| | VEQHHWCIPFSVDGQPAPTLRWLFNGSVLNET | variant equine IgG2 Fc |
| | SFIFTEFLESAANETMRHGCLRLNQPTHVNNG | Variant equine IgG2 Fc |
| | NYTLLATNPYGQDSASVMVAFMDNPPPCVLSA | Protein A+ |
| | EGVIPIPSVPKPQCPPYTHSKFLGGPSVFIFP | C1q − |
| | PNPKDTLMISRTPVVTCVVVNLSDQYPDVQFS | |
| | WYVDNTEVHSAITKQREAQFNSTYRVVSVLPI | |
| | QHQDWLSGKEFKCSVTNVGVPQPISRAISRGK | |
| | GPSRVPQVYVLPPHPDELAKSKVSVTCLVKDF | |
| | YPPDISVEQQSNRWPELEGKYSTTPAQLDGDG | |
| | SYFLYSKLSLETSRWQQVESFTCAVMHEALHN | |
| | HYTKTDISESLGK | |
| 245 | METDTLLLWVLLLWVPGSTGVSFPASVHLQTA | Exemplary equine TrkA ECD- |
| | VEQHHWCIPFSVDGQPAPTLRWLFNGSVLNET | variant equine IgG2 Fc |
| | SFIFTEFLESAANETMRHGCLRLNQPTHVNNG | Variant equine IgG2 Fc |
| | NYTLLATNPYGQDSASVMVAFMDNPFEFNPED | Protein A+ |
| | PIPVSFSPVDTNSTSRDPPCVLSAEGVIPIPS | C1q − |
| | VPKPQCPPYTHSKFLGGPSVFIFPPNPKDTLM | |
| | ISRTPVVTCVVVNLSDQYPDVQFSWYVDNTEV | |
| | HSAITKQREAQFNSTYRVVSVLPIQHQDWLSG | |
| | KEFKCSVTNVGVPQPISRISRGKGPSRVPQV | |
| | YVLPPHPDELAKSKVSVTCLVKDFYPPDISVE | |
| | WQSNRWPELEGKYSTTPAQLDGDGSYFLYSKL | |
| | SLETSRWQQVESFICAVMHEALHNHYTKTDIS | |
| | ESLGK | |
| 246 | METDTLLLWVLLLWVPGSTGVSFPASVHLQTA | Exemplary equine TrkA ECD- |
| | VEQHHWCIPFSVDGQPAPTLRWLFNGSVLNET | variant equine IgG2 Fc |
| | SFIFTEFLESAANETMRHGCLRLNQPTHVNNG | Variant equine IgG2 Fc |
| | NYTLLATNPYGQDSASVMVAFMDNPDMSKCPK | with equine IgG1 hinge |
| | CPAPELLGGPSVFIFPPNPKDTLMISRTPVVT | Protein A+ |
| | CVVVNLSDQYPDVQFSWYVDNTEVHSAITKQR | C1q− |
| | EAQFNSTYRVVSVLPIQHQDWLSGKEFKCSVT | |
| | NVGVPQPISRAISRGKGPSRVPQVYVLPPHPD | |
| | ELAKSKVSVTCLVKDFYPPDISVEWQSNRWPE | |
| | LEGKYSTTPAQLDGDGSYFLYSKLSLETSRWQ | |
| | QVESFICAVMHEALHNHYTKTDISESLGK | |
| 247 | METDTLLLWVLLLWVPGSTGVSFPASVHLQTA | Exemplary equine TrkA ECD- |
| | VEQHHWCIPFSVDGQPAPTLRWLFNGSVLNET | variant equine IgG2 Fc |
| | SFIFTEFLESAANETMRHGCLRLNQPTHVNNG | Variant equine IgG2 Fc |
| | NYTLLATNPYGQDSASVMVAFMDNPFEFNPED | with equine IgG1 hinge |
| | PIPVSFSPVDTNSTSRDDMSKCPKCPAPELLG | Protein A+ |
| | GPSVFIFPPNPKDTLMISRTPVVTCVVVNLSD | C1q− |
| | QYPDVQFSWYVDNTEVHSAITKQREAQFNSTY | |
| | RVVSVLPIQHQDWLSGKEFKCSVTNVGVPQPI | |
| | SRAISRGKGPSRVPQVYVLPPHPDELAKSKVS | |
| | VTCLVKDFYPPDISVEWQSNRWPELEGKYSTT | |
| | PAQLDGDGSYFLYSKLSLETSRWQQVESFTCA | |
| | VMHEALHNHYTKTDISESLGK | |
| 248 | METDTLLLWVLLLWVPGSTGVSFPASVQLHTA | Exemplary human TrkA ECD- |
| | VEMHHWCIPFSVDGQPAPSLRWLFNGSVLNET | variant human IgG4 Fc |
| | SFIFTEFLEPAANETVRHGCLRLNQPTHVNNG | Variant human IgG4 |
| | NYTLLAANPFGQASASIMAAFMDNPESKYGPP | S to P |
| | CPPCPAPEFLGGPSVFLFPPKPKDTLMISRTP | |
| | EVTCVVVDVSQEDPEVQFNWYVDGVEVHNAKT | |
| | KPREEQFNSTYRVVSVLTVLHQDWLNGKEYKC | |
| | KVSNKGLPSSIEKTISKAKGQPREPQVYTLPP | |
| | SQEEMTKNQVSLTCLVKGFYPSDIAVEWESNG | |
| | QPENNYKTTPPVLDSDGSFFLYSRLTVDKSRW | |
| | QEGNVFSCSVMHEALHNHYTQKSLSLSLGK | |
| 249 | METDTLLLWVLLLWVPGSTGVSFPASVQLHTA | Exemplary human TrkA ECD- |
| | VEMHHWCIPFSVDGQPAPSLRWLFNGSVLNET | variant human IgG4 Fc |
| | SFIFTEFLEPAANETVRHGCLRLNQPTHVNNG | Variant human IgG4 |
| | NYTLLAANPFGQASASIMAAFMDNPFEFNPED | S to P |
| | PIPVSFSPVDTNSTSGDESKYGPPCPPCPAPE | |
| | FLGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKN | |
| | QVSLTCLVKGFYPSDIAVEWESNGQPENNYKT | |
| | TPPVLDSDGSFFLYSRLTVDKSRWQEGNVFSC | |
| | SVMHEALHNHYTQKSLSLSLGK | |
| 250 | VSFPASVQLHEAVELHHWCIPFSVDGQPAPSL | Exemplary canine TrkA ECD- |
| | RWLFNGSVLNETSFIFTEFLEPVANETVRHGC | wildtype canine Fc-IgG-B |
| | LRLNQPTHVNNGNYTLLAANPSGRAAAFVMAA | |
| | FMDNP [symbol] RPPDCPKCPAPEMLGGPS | |
| | VFIFPPKPKDTLLIARTPEVTCVVVDLDPEDP | |
| | EVQISWFVDGKQMQTAKTQPREEQFNGTYRVV | |
| | SVLPIGHQDWLKGKQFTCKVNNKALPSPIERT | |
| | ISKARGQAHQPSVYVLPPSREELSKNTVSLTC | |
| | LIKDFFPPDIDVEWQSNGQQEPESKYRTTPPQ | |
| | LDEDGSYFLYSKLSVDKSRWQRGDTFICAVMH | |
| | EALHNHYTQESLSHSPGK | |
| 251 | VSFPASVQLHEAVELHHWCIPFSVDGQPAPSL | Exemplary canine TrkA ECD- |
| | RWLFNGSVLNETSFIFTEFLEPVANETVRHGC | wildtype canine IgG-B Fc |
| | LRLNQPTHVNNGNYTLLAANPSGRAAAFVMAA | |
| | FMDNPFEFNPEDPIPVSFSPVDTNSISGD [symbol] | |
| | [symbol] RPPDCPKCPAPEMLGGPSVFIFPPKP | |
| | KDTLLIARTPEVTCVVVDLDPEDPEVQISWFV | |
| | DGKQMQTAKTQPREEQFNGTYRVVSVLPIGHQ | |
| | DWLKGKQFTCKVNNKALPSPIERTISKARGQA | |
| | HQPSVYVLPPSREELSKNTVSLTCLIKDFFPP | |
| | DIDVEWQSNGQQEPESKYRTTPPQLDEDGSYF | |
| | LYSKLSVDKSRWQRGDTFICAVMHEALHNHYT | |
| | QESLSHSPGK | |
| 252 | VSFPASVQLHEAVELHHWCIPFSVDGQPAPSL | Exemplary canine TrkA ECD- |
| | RWLFNGSVLNETSFIFTEFLEPVANETVRHGC | variant canine IgG-B Fc |
| | LRLNQPTHVNNGNYTLLAANPSGRAAAFVMAA | Variant canine IgG-B Fc |
| | FMDNP [symbol] RPPDCPKCPAPEPLGGPS | Protein A+ |
| | VFIFPPKPKDTLLIARTPEVTCVVVDLDREDP | C1q − |
| | EVQISWFVDGKQMQTAKTQPREEQFNGTYRVV | CD16 − |
| | SVLPIGHQDWLKGKQFTCRVNNKALPSPIERT | |
| | ISKARGQAHQPSVYVLPPSREELSKNTVSLTC | |
| | LIKDFFPPDIDVEWQSNGQQEPESKYRTTPPQ | |
| | LDEDGSYFLYSKLSVDKSRWQRGDTFICAVMH | |
| | EALHNHYTQESLSHSPGK | |
| 253 | VSFPASVQLHEAVELHHWCIPFSVDGQPAPSL | Exemplary canine TrkA ECD- |
| | RWLFNGSVLNETSFIFTEFLEPVANETVRHGC | variant canine IgG-B Fc |
| | LRLNQPTHVNNGNYTLLAANPSGRAAAFVMAA | Variant canine IgG-B Fc |
| | FMDNPFEFNPEDPIPVSFSPVDTNSTSGD [symbol] | Protein A+ |
| | [symbol] RPPDCPKCPAPEPLGGPSVFIFPPKP | C1q − |
| | KDTLLIARTPEVTCVVVDLDREDPEVQISWFV | CD16 − |
| | DGKQMQTAKTQPREEQFNGTYRVVSVLPIGHQ | |
| | DWLKGKQFTCRVNNKALPSPIERTISKARGQA | |
| | HQPSVYVLPPSREELSKNTVSLTCLIKDFFPP | |
| | DIDVEWQSNGQQEPESKYRTTPPQLDEDGSYF | |
| | LYSKLSVDKSRWQRGDTFICAVMHEALHNHYT | |
| | QESLSHSPGK | |
| 254 | VSFPASVQLHEAVELHHWCIPFSVDGQPAPSL | Exemplary canine TrkA ECD- |
| | RWLFNGSVLNETSFIFTEFLEPVANETVRHGC | wildtype canine IgG-A Fc |
| | LRLNQPTHVNNGNYTLLAANPSGRAAAFVMAA | |
| | FMDNP [symbol] FNECRCTDTPCPVPEPLG | |
| | GPSVLIFPPKPKDILRITRTPEVTCVVLDLGR | |
| | EDPEVQISWFVDGKEVHTAKTQSREQQFNGTY | |
| | RVVSVLPIEHQDWLTGKEFKCRVNHIDLPSPI | |
| | ERTISKARGRAHKPSVYVLPPSPKELSSSDTV | |
| | SITCLIKDFYPPDIDVEWQSNGQQEPERKHRM | |
| | TPPQLDEDGSYFLYSKLSVDKSRWQQGDPFTC | |
| | AVMHETLQNHYTDLSLSHSPGK | |
| 255 | VSFPASVQLHEAVELHHWCIPFSVDGQPAPSL | Exemplary canine TrkA ECD- |
| | RWLFNGSVLNETSFIFTEFLEPVANETVRHGC | wildtype canine IgG-A Fc |
| | LRLNQPTHVNNGNYTLLAANPSGRAAAFVMAA | |
| | FMDNPFEFNPEDPIPVSFSPVDTNSTSGD [symbol] | |
| | [symbol] FNECRCTDTPCPVPEPLGGPSVLIFP | |
| | PKPKDILRITRTPEVTCVVLDLGREDPEVQIS | |
| | WFVDGKEVHTAKTQSREQQFNGTYRVVSVLPI | |
| | EHQDWLTGKEFKCRVNHIDLPSPIERTISKAR | |
| | GRAHKPSVYVLPPSPKELSSSDTVSITCLIKD | |
| | FYPPDIDVEWQSNGQQEPERKHRMTPPQLDED | |
| | GSYFLYSKLSVDKSRWQQGDPFTCAVMHETLQ | |
| | NHYTDLSLSHSPGK | |
| 256 | VSFPASVQLHEAVELHHWCIPFSVDGQPAPSL | Exemplary canine TrkA ECD- |
| | RWLFNGSVLNETSFIFTEFLEPVANETVRHGC | variant canine IgG-A Fc |
| | LRLNQPTHVNNGNYTLLAANPSGRAAAFVMAA | Variant canine IgG-A Fc |
| | FMDNP [symbol] FNECRCTDTPCPVPEPLG | Protein A+ |
| | GPSVLIFPPKPKDTLLIARTPEVTCVVLDLGR | C1q − |
| | EDPEVQISWFVDGKEVHTAKTQSREQQFNGTY | CD16 − |
| | RVVSVLPIGHQDWLTGKEFKCRVNHIDLPSPI | |
| | ERTISKARGRAHKPSVYVLPPSPKELSSSDTV | |
| | SITCLIKDFYPPDIDVEWQSNGQQEPERKHRM | |
| | TPPQLDEDGSYFLYSKLSVDKSRWQQGDPFTC | |
| | AVMHEALHNHYTDLSLSHSPGK | |
| 257 | VSFPASVQLHEAVELHHWCIPFSVDGQPAPSL | Exemplary canine TrkA ECD- |
| | RWLFNGSVLNETSFIFTEFLEPVANETVRHGC | variant canine IgG-A Fc |
| | LRLNQPTHVNNGNYTLLAANPSGRAAAFVMAA | Variant canine IgG-A Fc |
| | FMDNPFEFNPEDPIPVSFSPVDTNSTSGD [symbol] | Protein A+ |
| | [symbol] FNECRCTDTPCPVPEPLGGPSVLIFP | C1q − |
| | PKPKDTLLIARTPEVTCVVLDLGREDPEVQIS | CD16 − |
| | WFVDGKEVHTAKTQSREQQFNGTYRVVSVLPI | |
| | GHQDWLTGKEFKCRVNHIDLPSPIERTISKAR | |
| | GRAHKPSVYVLPPSPKELSSSDTVSITCLIKD | |
| | FYPPDIDVEWQSNGQQEPERKHRMTPPQLDED | |
| | GSYFLYSKLSVDKSRWQQGDPFTCAVMHEALH | |
| | NHYTDLSLSHSPGK | |
| 258 | VSFPASVQLHEAVELHHWCIPFSVDGQPAPSL | Exemplary canine TrkA ECD- |
| | RWLFNGSVLNETSFIFTEFLEPVANETVRHGC | wildtype canine IgG-D Fc |
| | LRLNQPTHVNNGNYTLLAANPSGRAAAFVMAA | |
| | FMDNP [symbol] PKESTCKCISPCPVPESL | |
| | GGPSVFIFPPKPKDILRITRIPEITCVVLDLG | |
| | REDPEVQISWFVDGKEVHTAKTQPREQQFNST | |
| | YRVVSVLPIEHQDWLTGKEFKCRVNHIGLPSP | |
| | IERTISKARGQAHQPSVYVLPPSPKELSSSDT | |
| | VTLTCLIKDFFPPEIDVEWQSNGQPEPESKYH | |
| | TTAPQLDEDGSYFLYSKLSVDKSRWQQGDTFT | |
| | CAVMHEALQNHYTDLSLSHSPGK | |
| 259 | VSFPASVQLHEAVELHHWCIPFSVDGQPAPSL | Exemplary canine TrkA ECD- |
| | RWLFNGSVLNETSFIFTEFLEPVANETVRHGC | wildtype canine IgG-D Fc |
| | LRLNQPTHVNNGNYTLLAANPSGRAAAFVMAA | |
| | FMDNPFEFNPEDPIPVSFSPVDTNSTSGD [symbol] | |
| | [symbol] PKESTCKCISPCPVPESLGGPSVFIF | |
| | PPKPKDILRITRIPEITCVVLDLGREDPEVQI | |
| | SWFVDGKEVHTAKTQPREQQFNSTYRVVSVLP | |
| | IEHQDWLTGKEFKCRVNHIGLPSPIERTISKA | |
| | RGQAHQPSVYVLPPSPKELSSSDTVTLTCLIK | |
| | DFFPPEIDVEWQSNGQPEPESKYHTTAPQLDE | |
| | DGSYFLYSKLSVDKSRWQQGDTFTCAVMHEAL | |
| | QNHYTDLSLSHSPGK | |
| 260 | VSFPASVQLHEAVELHHWCIPFSVDGQPAPSL | Exemplary canine TrkA ECD- |
| | RWLFNGSVLNETSFIFTEFLEPVANETVRHGC | variant canine IgG-D Fc |
| | LRLNQPTHVNNGNYTLLAANPSGRAAAFVMAA | Variant canine IgG-A Fc |
| | FMDNP [symbol] PKESTCKCISPCPVPESL | Protein A+ |
| | GGPSVFIFPPKPKDTLLIARTPEITCVVLDLG | C1q − |
| | REDPEVQISWFVDGKEVHTAKTQPREQQFNST | CD16 − |
| | YRVVSVLPIGHQDWLTGKEFKCRVNHIGLPSP | |
| | IERTISKARGQAHQPSVYVLPPSPKELSSSDT | |
| | VTLTCLIKDFFPPEIDVEWQSNGQPEPESKYH | |
| | TTAPQLDEDGSYFLYSKLSVDKSRWQQGDTFT | |
| | CAVMHEALHNHYTDLSLSHSPGK | |
| 261 | VSFPASVQLHEAVELHHWCIPFSVDGQPAPSL | Exemplary canine TrkA ECD- |
| | RWLFNGSVLNETSFIFTEFLEPVANETVRHGC | variant canine IgG-D Fc |
| | LRLNQPTHVNNGNYTLLAANPSGRAAAFVMAA | Variant canine IgG-A Fc |
| | FMDNPFEFNPEDPIPVSFSPVDTNSTSGD [symbol] | Protein A+ |
| | [symbol] PKESTCKCISPCPVPESLGGPSVFIF | C1q − |
| | PPKPKDTLLIARTPEITCVVLDLGREDPEVQI | CD16 − |
| | SWFVDGKEVHTAKTQPREQQFNSTYRVVSVLP | |
| | IGHQDWLTGKEFKCRVNHIGLPSPIERTISKA | |
| | RGQAHQPSVYVLPPSPKELSSSDTVTLTCLIK | |
| | DFFPPEIDVEWQSNGQPEPESKYHTTAPQLDE | |
| | DGSYFLYSKLSVDKSRWQQGDTFTCAVMHEAL | |
| | HNHYTDLSLSHSPGK | |
| 262 | VSFPASVQLHAAVELHHWCIPFSVDGQPAPSL | Exemplary feline TrkA ECD- |
| | RWLFNGSVLNETSFIFTEFLEPAANETVRHGC | variant feline IgG-2 Fc |
| | LRLNQPTHVNNGNYTLLAANPSGRAAASVLAA | Variant feline IgG2 Fc |
| | FMDNP [symbol] PKTASTIESKTGECPKCP | Hinge Cys |
| | VPEIPGAPSVFIFPPKPKDTLSISRTPEVTCL | |
| | VVDLGPDDSNVQITWFVDNTEMHTAKTRPREE | |
| | QFNSTYRVVSVLPILHQDWLKGKEFKCKVNSK | |
| | SLPSAMERTISKAKGQPHEPQVYVLPPTQEEL | |
| | SENKVSVTCLIKGFHPPDIAVEWEITGQPEPE | |
| | NNYQTTPPQLDSDGTYFLYSRLSVDRSHWQRG | |
| | NTYTCSVSHEALHSHHTQKSLTQSPGK | |
| 263 | VSFPASVQLHAAVELHHWCIPFSVDGQPAPSL | Exemplary feline TrkA ECD- |
| | RWLFNGSVLNETSFIFTEFLEPAANETVRHGC | variant feline IgG-2 Fc |
| | LRLNQPTHVNNGNYTLLAANPSGRAAASVLAA | Variant feline IgG2 Fc |
| | FMDNPFEFNPEDPIPVSFSPVDSNSTSGD [symbol] | Hinge Cys |
| | [symbol] PKTASTIESKTGECPKCPVPEIPGAP | |
| | SVFIFPPKPKDTLSISRTPEVTCLVVDLGPDD | |
| | SNVQITWFVDNTEMHTAKTRPREEQFNSTYRV | |
| | VSVLPILHQDWLKGKEFKCKVNSKSLPSAMER | |
| | TISKAKGQPHEPQVYVLPPTQEELSENKVSVT | |
| | CLIKGFHPPDIAVEWEITGQPEPENNYQTTPP | |
| | QLDSDGTYFLYSRLSVDRSHWQRGNTYTCSVS | |
| | HEALHSHHTQKSLTQSPGK | |
| 264 | VSFPASVHLQTAVEQHHWCIPFSVDGQPAPTL | Exemplary equine TrkA ECD- |
| | RWLFNGSVLNETSFIFTEFLESAANETMRHGC | variant equine IgG2 Fc |
| | LRLNQPTHVNNGNYTLLATNPYGQDSASVMVA | Variant equine IgG2 Fc |
| | FMDNPPPCVLSAEGVIPIPSVPKPQCPPYTHS | Protein A+ |
| | KFLGGPSVFIFPPNPKDTLMISRTPVVTCVVV | C1q − |
| | NLSDQYPDVQFSWYVDNTEVHSAITKQREAQF | |
| | NSTYRVVSVLPIQHQDWLSGKEFKCSVTNVGV | |
| | PQPISRAISRGKGPSRVPQVYVLPPHPDELAK | |
| | SKVSVTCLVKDFYPPDISVEQQSNRWPELEGK | |
| | YSTTPAQLDGDGSYFLYSKLSLETSRWQQVES | |
| | FICAVMHEALHNHYTKTDISESLGK | |
| 265 | VSFPASVHLQTAVEQHHWCIPFSVDGQPAPTL | Exemplary equine TrkA ECD- |
| | RWLFNGSVLNETSFIFTEFLESAANETMRHGC | variant equine IgG2 Fc |
| | LRLNQPTHVNNGNYTLLATNPYGQDSASVMVA | Variant equine IgG2 Fc |
| | FMDNPFEFNPEDPIPVSFSPVDTNSTSRDPPC | Protein A+ |
| | VLSAEGVIPIPSVPKPQCPPYTHSKFLGGPSV | C1q− |
| | FIFPPNPKDTLMISRTPVVTCVVVNLSDQYPD | |
| | VQFSWYVDNTEVHSAITKQREAQFNSTYRVVS | |
| | VLPIQHQDWLSGKEFKCSVTNVGVPQPISRAI | |
| | SRGKGPSRVPQVYVLPPHPDELAKSKVSVTCL | |
| | VKDFYPPDISVEWQSNRWPELEGKYSTTPAQL | |
| | DGDGSYFLYSKLSLETSRWQQVESFTCAVMHE | |
| | ALHNHYTKTDISESLGK | |
| 266 | VSFPASVHLQTAVEQHHWCIPFSVDGQPAPTL | Exemplary equine TrkA ECD- |
| | RWLFNGSVLNETSFIFTEFLESAANETMRHGC | variant equine IgG2 Fc |
| | LRLNQPTHVNNGNYTLLATNPYGQDSASVMVA | Variant equine IgG2 Fc |
| | FMDNPDMSKCPKCPAPELLGGPSVFIFPPNPK | with equine IgG1 hinge |
| | DTLMISRTPVVTCVVVNLSDQYPDVQFSWYVD | Protein A+ |
| | NTEVHSAITKQREAQFNSTYRVVSVLPIQHQD | C1q− |
| | WLSGKEFKCSVTNVGVPQPISRAISRGKGPSR | |
| | VPQVYVLPPHPDELAKSKVSVTCLVKDFYPPD | |
| | ISVEWQSNRWPELEGKYSTTPAQLDGDGSYFL | |
| | YSKLSLETSRWQQVESFTCAVMHEALHNHYTK | |
| | TDISESLGK | |
| 267 | VSFPASVHLQTAVEQHHWCIPFSVDGQPAPTL | Exemplary equine TrkA ECD- |
| | RWLFNGSVLNETSFIFTEFLESAANETMRHGC | variant equine IgG2 Fc |
| | LRLNQPTHVNNGNYTLLATNPYGQDSASVMVA | Variant equine IgG2 Fc |
| | FMDNPFEFNPEDPIPVSFSPVDTNSTSRDDMS | with equine IgG1 hinge |
| | KCPKCPAPELLGGPSVFIFPPNPKDTLMISRT | Protein A+ |
| | PVVTCVVVNLSDQYPDVQFSWYVDNTEVHSAI | C1q− |
| | TKQREAQFNSTYRVVSVLPIQHQDWLSGKEFK | |
| | CSVTNVGVPQPISRAISRGKGPSRVPQVYVLP | |
| | PHPDELAKSKVSVTCLVKDFYPPDISVEWQSN | |
| | RWPELEGKYSTTPAQLDGDGSYFLYSKLSLET | |
| | SRWQQVESFICAVMHEALHNHYTKTDISESLG | |
| | K | |
| 268 | VSFPASVQLHTAVEMHHWCIPFSVDGQPAPSL | Exemplary human TrkA ECD- |
| | RWLFNGSVLNETSFIFTEFLEPAANETVRHGC | variant human IgG4 Fc |
| | LRLNQPTHVNNGNYTLLAANPFGQASASIMAA | Variant human IgG4 |
| | FMDNPESKYGPPCPPCPAPEFLGGPSVFLFPP | S to P |
| | KPKDTLMISRTPEVTCVVVDVSQEDPEVQFNW | |
| | YVDGVEVHNAKTKPREEQFNSTYRVVSVLTVL | |
| | HQDWLNGKEYKCKVSNKGLPSSIEKTISKAKG | |
| | QPREPQVYTLPPSQEEMTKNQVSLTCLVKGFY | |
| | PSDIAVEWESNGQPENNYKTTPPVLDSDGSFF | |
| | LYSRLTVDKSRWQEGNVFSCSVMHEALHNHYT | |
| | QKSLSLSLGK | |
| 269 | VSFPASVQLHTAVEMHHWCIPFSVDGQPAPSL | Exemplary human TrkA ECD- |
| | RWLFNGSVLNETSFIFTEFLEPAANETVRHGC | variant human IgG4 Fc |
| | LRLNQPTHVNNGNYTLLAANPFGQASASIMAA | Variant human IgG4 |
| | FMDNPFEFNPEDPIPVSFSPVDTNSTSGDESK | S to P |
| | YGPPCPPCPAPEFLGGPSVFLFPPKPKDTLMI | |
| | SRTPEVTCVVVDVSQEDPEVQFNWYVDGVEVH | |
| | NAKTKPREEQFNSTYRVVSVLTVLHQDWLNGK | |
| | EYKCKVSNKGLPSSIEKTISKAKGQPREPQVY | |
| | TLPPSQEEMTKNQVSLTCLVKGFYPSDIAVEW | |
| | ESNGQPENNYKTTPPVLDSDGSFFLYSRLTVD | |
| | KSRWQEGNVFSCSVMHEALHNHYTQKSLSLSL | |
| | GK | |
| 270 | ESKYGPPCPPCPAPEFLGGPSVFLFPPKPKDT | Exemplary variant human IgG4 |
| | LMISRTPEVTCVVVDVSQEDPEVQFNWYVDGV | S(10)P |
| | EVHNAKTKPREEQFNSTYRVVSVLTVLHQDWL | |
| | NGKEYKCKVSNKGLPSSIEKTISKAKGQPREP | |
| | QVYTLPPSQEEMTKNQVSLTCLVKGFYPSDIA | |
| | VEWESNGQPENNYKTTPPVLDSDGSFFLYSRL | |
| | TVDKSRWQEGNVFSCSVMHEALHNHYTQKSLS | |
| | LSLGK | |

### DESCRIPTION OF THE EMBODIMENTS

Variant IgG Fc polypeptides from companion animals, such as canine, equine, and feline, are described. In some embodiments, the variant IgG Fc polypeptides have increased binding to Protein A, decreased binding to C1q, decreased binding to CD16, increased stability, increased recombinant production, increased hinge disulfide formation, and/or form heterodimeric polypeptides. In some embodiments, antibodies, antibody fragments, or fusion proteins comprise a variant IgG Fc polypeptide. Methods of producing or purifying variant IgG Fc polypeptides and methods of administering variant IgG Fc polypeptides to companion animals are also provided assay.

For the convenience of the reader, the following definitions of terms used herein are provided.

As used herein, numerical terms such as K_{D} are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### Exemplary Variant IgG Fc Polypeptides

Novel variant IgG Fc polypeptides are provided, for example, variant IgG Fc polypeptides for increased binding to Protein A, for decreased binding to C1q, for decreased binding to CD16, for increased stability, for increased recombinant production, for increased hinge disulfide formation, and/or for forming heterodimeric proteins assay.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or unnatural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“IgX Fc” or “IgX Fc polypeptide” refers to an Fc polypeptide derived from a particular antibody isotype (e.g., IgG, IgA, IgD, IgE, IgM, etc.), where “X” denotes the antibody isotype. Thus, “IgG Fc” denotes that the Fc polypeptide is derived from a γ chain, “IgA Fc” denotes that the Fc polypeptide is derived from an α chain, “IgD Fc” denotes that the Fc polypeptide is derived from a δ chain, “IgE Fc” denotes that the Fc polypeptide is derived from a chain, “IgM Fc” denotes that the Fc polypeptide is derived from μ chain, etc. In some embodiments, the IgG Fc polypeptide comprises the hinge, CH2, and CH3, but does not comprise CH1 or CL. In some embodiments, the IgG Fe polypeptide comprises CH2 and CH3, but does not comprise CH1, the hinge, or CL. In some embodiments, the IgG Fc polypeptide comprises CH1, hinge, CH2, and CH3, with or without CL1. In some embodiments, an Fc polypeptide, such as an IgG Fc polypeptide, lacks one or more C-terminal amino acids, such as 1 to 20, 1 to 15, 1 to 10, 1 to 5, or 1 to 2 amino acids, while retaining a biological activity. In some embodiments, the biological activity is the ability to bind FcRn, the ability to bind C1q, the ability to bind CD16, and/or the ability to bind Protein A. An “effector function” of the Fc polypeptide is an action or activity performed in whole or in part by any antibody in response to a stimulus and may include complement fixation and/or ADCC (antibody-dependent cellular cytotoxicity) induction. “IgX-N Fc” or “IgGXN Fc” denotes that the Fc polypeptide is derived from a particular subclass of antibody isotype (such as canine IgG subclass IgG-A, IgG-B, IgG-C, or IgG-D; feline IgG subclass IgG1a, IgG1b, or IgG2; or equine IgG subclass IgG1, IgG2, IgG3, IgG4, IgG5, IgG6, or IgG7, etc.), where “N” denotes the subclass.

“Hinge” refers to any portion of an Fc polypeptide or variant Fc polypeptide that is proline-rich and comprises at least one cysteine residue located between CH1 and CH2 of a full-length heavy chain constant region.

In some embodiments, a hinge is capable of forming a disulfide linkage within the same hinge region, within the same Fc polypeptide, with a hinge region of a separate Fc polypeptide, or with a separate Fc polypeptide. In some embodiments, a hinge comprises at least one, at least two, at least three, at least four, at least five, at least six, at least seven, at least eight, at least nine, or at least ten proline residues.

The term “companion animal species” refers to an animal suitable to be a companion to humans. In some embodiments, a companion animal species is a canine (or dog), a feline (or cat), or an equine (or horse). In some embodiments, a companion animal species is a small mammal, such as a canine, feline, dog, cat, rabbit, ferret, guinea pig, rodent, etc. In some embodiments, a companion animal species is a farm animal, such as a horse, cow, pig, etc.

In some embodiments, an IgX Fc polypeptide or an IgX-N Fc polypeptide is derived from a companion animal, such as a dog, a cat, or a horse. In some embodiments, IgG Fc polypeptides are isolated from canine γ heavy chains, such as IgG-A, IgG-B, IgG-C, or IgG-D. In some instances, IgG Fc polypeptides are isolated from feline γ heavy chains, such as IgG1a, IgG1b, or IgG2. In other instances, IgG Fc polypeptides are isolated from equine γ heavy chains, such as IgG1, IgG2, IgG3, IgG4, IgG5, IgG6, or IgG7.

The terms “IgX Fc” and “IgX Fc polypeptide” include wild-type IgX Fc polypeptides and variant IgX Fc polypeptides, unless indicated otherwise.

“Wild-type” refers to a non-mutated version of a polypeptide that occurs in nature, or a fragment thereof. A wild-type polypeptide may be produced recombinantly.

In some embodiments, a wild-type IgG Fc polypeptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 65, SEQ ID NO: 66, SEQ ID NO: 67, SEQ ID NO: 68, SEQ ID NO: 69, SEQ ID NO: 142, SEQ ID NO: 143, SEQ ID NO: 144, SEQ ID NO: 145, SEQ ID NO: 152, or SEQ ID NO: 153.

A “variant” is a polypeptide that differs from a reference polypeptide by single or multiple non-native amino acid substitutions, deletions, and/or additions. In some embodiments, a variant retains at least one biological activity of the reference polypeptide. In some embodiments, a variant (e.g., a variant canine IgG-A Fc, a variant canine IgG-C Fc, a variant canine IgG-D Fc, variant equine IgG2 Fc, variant equine IgG5 Fc, or variant equine IgG6 Fc) has an activity that the reference polypeptide substantially lacks. For example, in some embodiments, a variant canine IgG-A Fc, a variant canine IgG-C Fc, a variant canine IgG-D Fc, variant equine IgG2 Fc, variant equine IgG5 Fc, or variant equine IgG6 Fc binds Protein A.

As used herein, “percent (%) amino acid sequence identity” and “homology” with respect to a nucleic acid molecule or polypeptide sequence are defined as the percentage of nucleotide or amino acid residues in a reference sequence that are identical with the nucleotide or amino acid residues in the specific nucleic acid molecule or polypeptide sequence, after aligning the sequences and introducing gaps, if necessary to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 97% sequence identity, at least about 98% sequence identity, or at least about 99% sequence identity with the sequence of the reference nucleic acid or polypeptide.

As used herein, “position corresponding to position n,” wherein n is any number, refers to an amino acid position of a subject polypeptide that aligns with position n of a reference polypeptide after aligning the amino acid sequences of the subject and reference polypeptides and introducing gaps. Alignment for purposes of whether a position of a subject polypeptide corresponds with position n of a reference polypeptide can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for alignment, including any parameters needed to achieve maximal alignment over the full length of two sequences being compared. In some embodiments, the subject polypeptide and the reference polypeptide are of different lengths.

A “point mutation” is a mutation that involves a single amino acid residue. The mutation may be the loss of an amino acid, substitution of one amino acid residue for another, or the insertion of an additional amino acid residue.

An “amino acid substitution” refers to the replacement of one amino acid in a polypeptide with another amino acid. In some embodiments, an amino acid substitution is a conservative substitution. Nonlimiting exemplary conservative amino acid substitutions are shown in Table 2. Amino acid substitutions may be introduced into a molecule of interest and the products screened for a desired activity, for example, retained/improved antigen binding, decreased immunogenicity, or improved ADCC or CDC or enhanced pharmacokinetics.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; Ala; |
| | Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions entail exchanging a member of one of these classes with another class.

A “variant IgG Fc” as used herein is an IgG Fc polypeptide that differs from a reference IgG Fc polypeptide by single or multiple amino acid substitutions, deletions, and/or additions and substantially retains at least one biological activity of the reference IgG Fc polypeptide.

An “amino acid derivative,” as used herein, refers to any amino acid, modified amino acid, and/or amino acid analogue, that is not one of the 20 common natural amino acids found in humans. Exemplary amino acid derivatives include natural amino acids not found in humans (e.g., seleno cysteine and pyrrolysine, which may be found in some microorganisms) and unnatural amino acids. Exemplary amino acid derivatives, include, but are not limited to, amino acid derivatives commercially available through chemical product manufacturers (e.g., sigmaaldrich.com/chemistry/chemistry-products.html?TablePage=16274965, accessed on May 6, 2017, which is incorporated herein by reference). One or more amino acid derivatives may be incorporated into a polypeptide at a specific location using a translation system that utilizes host cells, orthogonal aminoacyl-tRNA synthetases derived from eubacterial synthetases, orthogonal tRNAs, and an amino acid derivative. For further descriptions, see, e.g., U.S. Pat. No. 9,624,485.

In some embodiments, a variant IgG Fc polypeptide comprises an amino acid substitution with an amino acid derivative. In some embodiments, the amino acid derivative is an alanine derivative, a cysteine derivative, an aspartic acid derivative, a glutamic acid derivative, a phenylalanine derivative, a glycine derivative, a histidine derivative, an isoleucine derivative, a lysine derivative, a leucine derivative, a methionine derivative, an asparagine derivative, a proline derivative, a glutamine derivative, an arginine derivative, a serine derivative, a threonine derivative, a valine derivative, a tryptophan derivative, or a tyrosine derivative.

In some embodiments, a variant IgG Fc polypeptide comprises a variant IgG Fc polypeptide of a companion animal species. In some embodiments, a variant IgG Fc polypeptide comprises a variant canine IgG Fc polypeptide, a variant equine IgG Fc polypeptide, or a feline IgG Fc polypeptide.

Exemplary Variant IgG Fc Polypeptides with Modified Protein A Binding

In some embodiments, a variant IgG Fc polypeptide has modified Protein A binding affinity. In some embodiments, a variant IgG Fc polypeptide has increased binding affinity to Protein A. In some embodiments, a variant IgG Fc polypeptide may be purified using Protein A column chromatography.

In some embodiments, a variant IgG Fc polypeptide comprises an amino acid substitution at a position corresponding to position 21, position 23, position 25, position 80, position 205, and/or position 207 of SEQ ID NO: 2. In some embodiments, a variant IgG Fc polypeptide comprises an amino acid substitution at a position corresponding to position 21, position 23, and/or position 24 of SEQ ID NO: 4. In some embodiments, a variant IgG Fc polypeptide comprises an amino acid substitution at a position corresponding to position 21, position 23, position 25, position 80, and/or position 207 of SEQ ID NO: 6.

In some embodiments, a variant IgG Fc polypeptide comprises an amino acid substitution at a position corresponding to position 15, and/or position 203 of SEQ ID NO: 50. In some embodiments, a variant IgG Fc polypeptide comprises an amino acid substitution at a position corresponding to position 199 and/or position 200 of SEQ ID NO: 54. In some embodiments, a variant IgG Fc polypeptide comprises an amino acid substitution at a position corresponding to position 199, position 200, position 201, and/or 202 of SEQ ID NO: 55.

In some embodiments, a variant IgG Fc polypeptide comprises an amino acid substitution at position 21, position 23, position 25, position 80, position 205, and/or position 207 of SEQ ID NO: 2. In some embodiments, a variant IgG Fc polypeptide comprises an amino acid substitution at position 21, position 23, and/or position 24 of SEQ ID NO: 4. In some embodiments, a variant IgG Fc polypeptide comprises an amino acid substitution at position 21, position 23, position 25, position 80, and/or position 207 of SEQ ID NO: 6.

In some embodiments, a variant IgG Fc polypeptide comprises an amino acid substitution at position 15 and/or position 203 of SEQ ID NO: 50. In some embodiments, a variant IgG Fc polypeptide comprises an amino acid substitution at position 199 and/or position 200 of SEQ ID NO: 54. In some embodiments, a variant IgG Fc polypeptide comprises an amino acid substitution at position 199, position 200, position 201, and/or position 202 of SEQ ID NO: 55.

In some embodiments, a variant IgG Fc polypeptide comprises a threonine at a position corresponding to position 21 of SEQ ID NO: 1, a leucine at a position corresponding to position 23 of SEQ ID NO: 1, an alanine at a position corresponding to position 25 of SEQ ID NO: 1, a glycine at a position corresponding to position 80 of SEQ ID NO: 1, an alanine at a position corresponding to position 205 of SEQ ID NO: 1, and/or a histidine at a position corresponding to position 207 of SEQ ID NO: 1. In some embodiments, a variant IgG Fc polypeptide comprises a threonine at a position corresponding to position 21 of SEQ ID NO: 4, a leucine at a position corresponding to position 23 of SEQ ID NO: 4, and/or an isoleucine at a position corresponding to position 24 of SEQ ID NO: 4. In some embodiments, a variant IgG Fc polypeptide comprises a threonine at a position corresponding to position 21 of SEQ ID NO: 6, a leucine at a position corresponding to position 23 of SEQ ID NO: 6, an alanine at a position corresponding to position 25 of SEQ ID NO: 6, a glycine at a position corresponding to position 80 of SEQ ID NO: 6, and/or a histidine at a position corresponding to position 207 of SEQ ID NO: 6.

In some embodiments, a variant IgG Fc polypeptide comprises a threonine or a valine at a position corresponding to position 15 of SEQ ID NO: 50, and/or a tyrosine or a valine at a position corresponding to position 203 of SEQ ID NO: 50. In some embodiments, a variant IgG Fc polypeptide comprises a leucine at a position corresponding to position 199 of SEQ ID NO: 54, and/or a histidine at a position corresponding to position 200 of SEQ ID NO: 54. In some embodiments, a variant IgG Fc polypeptide comprises an isoleucine at a position corresponding to position 199 of SEQ ID NO: 55, a histidine at a position corresponding to position 200 of SEQ ID NO: 55, an asparagine at a position corresponding to position 201 of SEQ ID NO: 55, and/or a histidine at a position corresponding to position 202 of SEQ ID NO: 55.

In some embodiments, a variant IgG Fc polypeptide comprises a threonine at position 21 of SEQ ID NO: 1, a leucine at position 23 of SEQ ID NO: 1, an alanine at position 25 of SEQ ID NO: 1, a glycine at position 80 of SEQ ID NO: 1, an alanine at position 205 of SEQ ID NO: 1, and/or a histidine at position 207 of SEQ ID NO: 1. In some embodiments, a variant IgG Fc polypeptide comprises a threonine at position 21 of SEQ ID NO: 4, a leucine at position 23 of SEQ ID NO: 4, and/or an isoleucine at position 24 of SEQ ID NO: 4. In some embodiments, a variant IgG Fc polypeptide comprise a threonine at a position 21 of SEQ ID NO:6, a leucine at position 23 of SEQ ID NO: 6, an alanine at position 25 of SEQ ID NO: 6, a glycine at position 80 of SEQ ID NO: 4, and/or a histidine at position 207 of SEQ ID NO: 6.

In some embodiments, a variant IgG Fc polypeptide comprises a threonine or a valine at position 15 of SEQ ID NO: 50, and/or a tyrosine or a valine at position 203 of SEQ ID NO: 50. In some embodiments, a variant IgG Fc polypeptide comprises a leucine at position 199 of SEQ ID NO: 54, and/or a histidine at position 200 of SEQ ID NO: 54. In some embodiments, a variant IgG Fc polypeptide comprises an isoleucine at position 199 of SEQ ID NO: 55, a histidine at position 200 of SEQ ID NO: 55, an asparagine at position 201 of SEQ ID NO: 55, and/or a histidine at position 202 of SEQ ID NO: 55.

Exemplary Variant IgG Fc Polypeptides with Modified CD16 Binding

In some embodiments, a variant IgG Fc polypeptide has modified CD16 binding affinity. In some embodiments, a variant IgG Fc polypeptide has decreased binding affinity to CD16. In some embodiments, a variant IgG Fc may have a reduced ADCC immune response.

In some embodiments, a variant IgG Fc polypeptide comprises an amino acid substitution at a position corresponding to position 5, position 38, position 39, position 97, and/or position 98 of SEQ ID NO: 2. In some embodiments, a variant IgG Fc polypeptide comprises an amino acid substitution at a position corresponding to position 5, position 38, position 39, position 97, and/or position 98 of SEQ ID NO: 3.

In some embodiments, a variant IgG Fc polypeptide comprises an amino acid substitution at position 5, position 38, position 39, position 97, and/or position 98 of SEQ ID NO: 2. In some embodiments, a variant IgG Fc polypeptide comprises an amino acid substitution at position 5, position 38, position 39, position 97, and/or position 98 of SEQ ID NO: 4.

In some embodiments, a variant IgG Fc polypeptide comprises a proline at a position corresponding to position 5, a glycine at a position corresponding to position 38, an arginine at a position corresponding to position 39, a isoleucine at a position corresponding to position 97, and/or a glycine at a position corresponding to position 98 of SEQ ID NO: 2. In some embodiments, a variant IgG Fc polypeptide comprises a proline at a position corresponding to position 5, a glycine at a position corresponding to position 38, an arginine at a position corresponding to position 39, a isoleucine at a position corresponding to position 97, and/or a glycine at a position corresponding to position 98 of SEQ ID NO: 4.

In some embodiments, a variant IgG Fc polypeptide comprises a proline at position 5, a glycine at position 38, an arginine at position 39, a isoleucine at position 97, and/or a glycine at position 98 of SEQ ID NO: 2. In some embodiments, a variant IgG Fc polypeptide comprises a proline at position 5, a glycine at position 38, an arginine at position 39, a isoleucine at position 97, and/or a glycine at position 98 of SEQ ID NO: 4.

Exemplary Variant IgG Fc Polypeptides with Modified C1q Binding

In some embodiments, a variant IgG Fc polypeptide has modified C1q binding affinity. In some embodiments, a variant IgG Fc polypeptide has reduced binding affinity to C1q. In some embodiments, a variant IgG Fc polypeptide may have reduced complement fixation. In some embodiments, a variant IgG Fc may have a reduced complement-mediated immune response.

In some embodiments, a variant IgG Fc polypeptide comprises an amino acid substitution at a position corresponding to position 93 of SEQ ID NO: 2. In some embodiments, a variant IgG Fc polypeptide comprises an amino acid substitution at a position corresponding to position 93 of SEQ ID NO: 4. In some embodiments, a variant IgG Fc polypeptide comprises an amino acid substitution at a position corresponding to position 87 of SEQ ID NO: 49. In some embodiments, a variant IgG Fc polypeptide comprises an amino acid substitution at a position corresponding to position 87 of SEQ ID NO: 52. In some embodiments, a variant IgG Fc polypeptide comprises an amino acid substitution at a position corresponding to position 87 of SEQ ID NO: 53. In some embodiments, a variant IgG Fc polypeptide comprises an amino acid substitution at a position corresponding to position 87 of SEQ ID NO: 56. In some embodiments, a variant IgG Fc polypeptide comprises an amino acid substitution at a position corresponding to position 198 of SEQ ID NO: 65, of SEQ ID NO: 66, of SEQ ID NO: 67, or of SEQ ID NO: 68.

In some embodiments, a variant IgG Fc polypeptide comprises an amino acid substitution at position 93 of SEQ ID NO: 2. In some embodiments, a variant IgG Fc polypeptide comprises an amino acid substitution at position 93 of SEQ ID NO: 4. In some embodiments, a variant IgG Fc polypeptide comprises an amino acid substitution at position 87 of SEQ ID NO: 49. In some embodiments, a variant IgG Fc polypeptide comprises an amino acid substitution at position 87 of SEQ ID NO: 52. In some embodiments, a variant IgG Fc polypeptide comprises or an amino acid substitution at position 87 of SEQ ID NO: 53. In some embodiments, a variant IgG Fc polypeptide comprises or an amino acid substitution at position 87 of SEQ ID NO: 56. In some embodiments, a variant IgG Fc polypeptide comprises an amino acid substitution at position 198 of SEQ ID NO: SEQ ID NO: 65, of SEQ ID NO: 66, of SEQ ID NO: 67, or of SEQ ID NO: 68.

In some embodiments, a variant IgG Fc polypeptide comprises an arginine at a position corresponding to position 93 of SEQ ID NO: 2. In some embodiments, a variant IgG Fc polypeptide comprises an arginine at a position corresponding to position 93 of SEQ ID NO: 4. In some embodiments, a variant IgG Fc polypeptide comprises a serine at a position corresponding to position 87 of SEQ ID NO: 49. In some embodiments, a variant IgG Fc polypeptide comprises a serine substitution at a position corresponding to position 87 of SEQ ID NO: 52. In some embodiments, a variant IgG Fc polypeptide comprises a serine at a position corresponding to position 87 of SEQ ID NO: 53. In some embodiments, a variant IgG Fc polypeptide comprises a serine at a position corresponding to position 87 of SEQ ID NO: 56. In some embodiments, a variant IgG Fc polypeptide comprises an alanine at a position corresponding to position 198 of SEQ ID NO: 65, of SEQ ID NO: 66, of SEQ ID NO: 67, or of SEQ ID NO: 68.

In some embodiments, a variant IgG Fc polypeptide comprises an arginine at position 93 of SEQ ID NO: 2. In some embodiments, a variant IgG Fc polypeptide comprises an amino acid substitution at position 93 of SEQ ID NO: 4. In some embodiments, a variant IgG Fc polypeptide comprises a serine at position 87 of SEQ ID NO: 49. In some embodiments, a variant IgG Fc polypeptide comprises a serine at position 87 of SEQ ID NO: 52. In some embodiments, a variant IgG Fc polypeptide comprises a serine at position 87 of SEQ ID NO: 53. In some embodiments, a variant IgG Fc polypeptide comprises a serine at position 87 of SEQ ID NO: 56. In some embodiments, a variant IgG Fc polypeptide comprises an alanine at position 198 of SEQ ID NO: 65, of SEQ ID NO: 66, of SEQ ID NO: 67, or of SEQ ID NO: 68.

Exemplary Variant IgG Fc Polypeptides with a Modified Inter-Chain Disulfide Linkage

In some embodiments, a variant feline IgG Fc polypeptide has at least one additional inter-chain disulfide linkage relative to the wild-type feline IgG Fc polypeptide. In some embodiments, a variant feline IgG Fc polypeptide has at least one additional inter-chain disulfide linkage in the hinge region. In some embodiments, a variant feline IgG2 Fc polypeptide with at least one additional inter-chain disulfide linkage has increased inter-chain stability relative to the wild-type feline IgG Fc polypeptide. In some embodiments, a variant IgG polypeptide has at least one amino acid modification to a hinge region relative to a wild-type IgG Fc polypeptide. In some embodiments, the wild-type IgG Fc polypeptide is a wild-type feline or equine IgG Fc polypeptide. In some embodiments, the variant IgG Fc polypeptide comprises a hinge region or a portion of a hinge region from an IgG Fc polypeptide of a different isotype. In some embodiments, the variant IgG Fc polypeptide comprises a hinge region from a wild-type feline IgG-1a Fc polypeptide, from a wild-type feline IgG-1b Fc polypeptide, or from a wild-type equine IgG1 Fc polypeptide. In some embodiments, a variant IgG2 Fc polypeptide has increased recombinant production and/or increased hinge disulfide formation relative to the wild-type IgG Fc polypeptide. In some embodiments, the increased recombinant production and/or increased hinge disulfide formation can be determined by SDS-PAGE analysis under reducing and/or non-reducing conditions.

In some embodiments, a variant IgG Fc polypeptide comprises a cysteine at a position corresponding to position 8, position 9, position 10, position 11, position 12, position 13, position 14, position 15, or position 16 of SEQ ID NO: 69. In some embodiments, a variant IgG Fc polypeptide comprises a cysteine at position 8, position 9, position 10, position 11, position 12, position 13, position 14, position 15, or position 16 of SEQ ID NO: 69.

In some embodiments, a variant IgG Fc polypeptide comprises an amino acid substitution at a position corresponding to position 16 of SEQ ID NO: 65, SEQ ID NO: 66, SEQ ID NO: 67, SEQ ID NO: 68, or SEQ ID NO: 69. In some embodiments, a variant IgG Fc polypeptide comprises an amino acid substitution at a position corresponding to position 3 and/or at a position corresponding to position 20 of SEQ ID NO: 51.

In some embodiments, a variant IgG Fc polypeptide comprises an amino acid substitution at position 16 of SEQ ID NO: 65, SEQ ID NO: 66, SEQ ID NO: 67, SEQ ID NO: 68, or SEQ ID NO: 69. In some embodiments, a variant IgG Fc polypeptide comprises an amino acid substitution at position 3 and/or at a position corresponding to position 20 of SEQ ID NO: 51.

In some embodiments, a variant IgG Fc polypeptide comprises a proline at a position corresponding to position 16 of SEQ ID NO: 65, SEQ ID NO: 66, SEQ ID NO: 67, SEQ ID NO: 68, or SEQ ID NO: 69. In some embodiments, a variant IgG Fc polypeptide comprises a serine at a position corresponding to position 3 and/or a proline at a position corresponding to position 20 of SEQ ID NO: 51.

In some embodiments, a variant IgG Fc polypeptide comprises a proline at position 16 of SEQ ID NO: 65, SEQ ID NO: 66, SEQ ID NO: 67, SEQ ID NO: 68, or SEQ ID NO: 69. In some embodiments, a variant IgG Fc polypeptide comprises a serine at position 3 and/or a proline at position 20 of SEQ ID NO: 51.

### Exemplary Variant IgG Fc Polypeptides for Multimeric Polypeptides

In certain embodiments, a multimeric polypeptide provided herein is a bispecific antibody. A bispecific antibody has a binding specificity for two different epitopes or target molecules. In some embodiments, a bispecific antibody binds to two different epitopes of the same target molecule. Bispecific antibodies may be full length antibodies or antibody fragments.

In some embodiments, the multimeric polypeptide comprises a first variant IgG Fc polypeptide comprising a “knob” mutation and a second variant IgG Fc polypeptide comprising a “hole” mutation. Nonlimiting exemplary knob and hole mutations are described, for example, in Merchant, A. M. et al. An efficient route to human bispecific IgG. Nat Biotechnol, 16(7):677-81 (1998).

In some embodiments, a variant canine or variant feline IgG Fc polypeptide comprises a knob mutation. In some embodiments, a variant IgG Fc polypeptide comprises a tyrosine or a tryptophan at a position corresponding to position 138 of SEQ ID NO: 1. In some embodiments, a variant IgG Fc polypeptide comprises a tyrosine or a tryptophan at a position corresponding to position 137 of SEQ ID NO: 2. In some embodiments, a variant IgG Fc polypeptide comprises a tyrosine or a tryptophan at a position corresponding to position 137 of SEQ ID NO: 4. In some embodiments, a variant IgG Fc polypeptide comprises a tyrosine or a tryptophan at a position corresponding to position 138 of SEQ ID NO:6. In some embodiments, a variant IgG Fc polypeptide comprises a tyrosine or a tryptophan at a position corresponding to position 154 of SEQ ID NO: 65, SEQ ID NO: 66, SEQ ID NO: 67, SEQ ID NO: 68, or SEQ ID NO: 69. In some embodiments, a variant IgG Fc polypeptide comprises a tyrosine or a tryptophan at a position corresponding to position 131 of SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56.

In some embodiments, a variant IgG Fc polypeptide comprises a tyrosine or a tryptophan at position 138 of SEQ ID NO: 1. In some embodiments, a variant IgG Fc polypeptide comprises a tyrosine or a tryptophan at position 137 of SEQ ID NO: 2. In some embodiments, a variant IgG Fc polypeptide comprises a tyrosine or a tryptophan at position 137 of SEQ ID NO: 4. In some embodiments, a variant IgG Fc polypeptide comprises a tyrosine or a tryptophan at position 138 of SEQ ID NO: 6. In some embodiments, a variant IgG Fc polypeptide comprises a tyrosine or a tryptophan at position 154 of SEQ ID NO: 65, SEQ ID NO: 66, SEQ ID NO: 67, SEQ ID NO: 68, or SEQ ID NO: 69. In some embodiments, a variant IgG Fc polypeptide comprises a tyrosine or a tryptophan at position 131 of SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56.

In some embodiments, a variant canine or a variant feline IgG Fc polypeptide comprises a hole mutation. In some embodiments, a variant IgG Fc polypeptide comprises a serine at a position corresponding to position 138, an alanine at a position corresponding to position 140, and/or a threonine at a position corresponding to position 181 of SEQ ID NO: 1. In some embodiments, a variant IgG Fc polypeptide comprises a serine at a position corresponding to position 137, an alanine at a position corresponding to position 139, and/or a threonine at a position corresponding to position 180 of SEQ ID NO: 2. In some embodiments, a variant IgG Fc polypeptide comprises a serine at a position corresponding to position 137, an alanine at a position corresponding to position 139, and/or a threonine at a position corresponding to position 180 of SEQ ID NO: 4. In some embodiments, a variant IgG Fc polypeptide comprises a serine at a position corresponding to position 138, an alanine at a position corresponding to position 140, and/or a threonine at a position corresponding to position 181 of SEQ ID NO: 6. In some embodiments, a variant IgG Fc polypeptide comprises a serine at a position corresponding to position 154, an alanine at a position corresponding to position 156, and/or a threonine at a position corresponding to position 197 of SEQ ID NO: 65, SEQ ID NO: 66, SEQ ID NO: 67, SEQ ID NO: 68, or SEQ ID NO: 69. In some embodiments, a variant IgG Fc polypeptide comprises a serine at a position corresponding to position 131, an alanine at a position corresponding to position 133, and/or a threonine at a position corresponding to position 174 of SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56.

In some embodiments, a variant IgG Fc polypeptide comprises a serine at position 138, an alanine at position 140, and/or a threonine at position 181 of SEQ ID NO: 1. In some embodiments, a variant IgG Fc polypeptide comprises a serine at position 137, an alanine at position 139, and/or a threonine at position 181 of SEQ ID NO: 2. In some embodiments, a variant IgG Fc polypeptide comprises a serine at position 137, an alanine at position 139, and/or a threonine at position 181 of SEQ ID NO: 4. In some embodiments, a variant IgG Fc polypeptide comprises a serine at position 138, an alanine at position 140, and/or a threonine at position 181 of SEQ ID NO: 6. In some embodiments, a variant IgG Fc polypeptide comprises a serine at position 154, an alanine at position 156, and/or a threonine at position 197 of SEQ ID NO: 65, SEQ ID NO: 66, SEQ ID NO: 67, SEQ ID NO: 68, or SEQ ID NO: 69. In some embodiments, a variant IgG Fc polypeptide comprises a serine at position 131, an alanine at position 133, and/or a threonine at position 174 of SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56.

In some embodiments, a contiguous polypeptide comprises a first therapeutic polypeptide or a first antibody and a variant canine, feline, or equine IgG Fc polypeptide comprising a knob mutation. In some embodiments, a contiguous polypeptide comprises a second therapeutic polypeptide or a second antibody and a variant canine, feline, or equine IgG Fc polypeptide comprising a hole mutation.

### Exemplary Therapeutic Polypeptides and Antibodies

An “extracellular domain” (“ECD”) is the portion of a polypeptide that extends beyond the transmembrane domain into the extracellular space. The term “extracellular domain,” as used herein, may comprise a complete extracellular domain or may comprise a truncated extracellular domain missing one or more amino acids, that binds to its ligand. The composition of the extracellular domain may depend on the algorithm used to determine which amino acids are in the membrane. Different algorithms may predict, and different systems may express, different extracellular domains for a given protein.

A “therapeutic polypeptide” as used herein, is a polypeptide comprising the entirety or a portion of the identified polypeptide from any vertebrate source, including mammals such as primates (e.g., humans and cynomolgus monkeys), rodents (e.g., mice and rats), and companion animals (e.g., dogs, cats, and equine), unless otherwise indicated.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments (such as Fab, F(ab′)2, ScFv, minibody, diabody, triabody, and tetrabody) so long as they exhibit the desired antigen-binding activity. Canine, feline, and equine species have different varieties (classes) of antibodies that are shared by many mammalians.

The term antibody includes, but is not limited to, fragments that are capable of binding to an antigen, such as Fv, single-chain Fv (scFv), Fab, Fab′, di-scFv, sdAb (single domain antibody) and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen combining sites and is still capable of cross-linking antigen. The term antibody also includes, but is not limited to, chimeric antibodies, humanized antibodies, and antibodies of various species such as mouse, human, cynomolgus monkey, canine, feline, equine, etc. Furthermore, for all antibody constructs provided herein, variants having the sequences from other organisms are also contemplated. Thus, if a murine version of an antibody is disclosed, one of skill in the art will appreciate how to transform the murine sequence-based antibody into a cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, mouse scFv or a canine scFv). This denotes the sequences of at least part of the non-CDR regions, rather than the source of the construct. In some embodiments, the antibodies comprise a label or are conjugated to a second moiety.

In some embodiments, a therapeutic polypeptide is an NGF (or Nerve Growth Factor) polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα (or Tumor Necrosis Factor Alpha) polypeptide, a receptor of a TNFα polypeptide, a TNFR (or Tumor Necrosis Factor Receptor) polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 (or Interleukin 5) polypeptide, a receptor of an IL5 polypeptide, an IL5R (or Interleukin 5 Receptor) polypeptide (e.g., an ECD of an IL5R polypeptide), an IL5Rα polypeptide (e.g., an ECD of an IL5Rα polypeptide), an IL6 (or Interleukin 6) polypeptide, a receptor of an IL6 polypeptide, an IL6R (or Interleukin 6 Receptor) polypeptide (e.g., an ECD of an IL6R polypeptide), an IL17 (or Interleukin 17) polypeptide, a receptor of an IL17 polypeptide, an IL17R (or Interleukin 17 Receptor) polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 (or Interleukin 23) polypeptide, a receptor of an IL23 polypeptide, an IL23R (or Interleukin 23 Receptor) polypeptide (e.g., an ECD of an IL23R polypeptide), an IL12β1 polypeptide (e.g., an ECD of an IL12β1 polypeptide), a PDL (or Programmed Cell Death Ligand) polypeptide, a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 (or Cytotoxic T-Lymphocyte Associated Protein 4) polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 (or Lymphocyte Activating Gene 3) polypeptide (e.g., an ECD of a LAG-3 polypeptide), an IL31 (or Interleukin 31) polypeptide, a receptor of an IL31 polypeptide, an IL31RA (an Interleukin 31 Receptor A) polypeptide (e.g., an ECD of an IL31RA polypeptide), an OSMR (or Oncostatin M Receptor) polypeptide (e.g., an ECD of an OSMR polypeptide), an IL4 (or Interleukin 4) polypeptide, a receptor of an IL4R polypeptide, an IL4R (or Interleukin 4 Receptor) polypeptide (e.g., an ECD of an IL4R polypeptide), an IL13 (or Interleukin 13 Receptor) polypeptide, a receptor of an IL13 polypeptide, an IL13RA1 (or Interleukin 13 Receptor A1) polypeptide (e.g., an ECD of an IL13RA1 polypeptide), an IL4R (or Interleukin 4 Receptor) polypeptide (e.g., an ECD of an IL4R polypeptide), an IL13Rα2 (or Interleukin 13 Receptor α2) polypeptide (e.g., an ECD of an IL13Rα2 polypeptide), an IL22 (or Interleukin 22) polypeptide, a receptor of an IL22 polypeptide (e.g., an ECD of an IL22 polypeptide), an IL22Rα1 (or Interleukin 22 Receptor α1) polypeptide (e.g., an ECD of an IL22Rα1 polypeptide), an IL10Rβ2 (or Interleukin 10 Receptor (32) polypeptide (e.g., an ECD of an IL10Rβ2 polypeptide), an IL33 (or Interleukin 33) polypeptide, a receptor of an IL33 polypeptide, an IL1RL1 polypeptide (e.g., an ED of an IL1RL1 polypeptide), an EGF (or Epidermal Growth Factor) polypeptide, a receptor of an EGF polypeptide, a TGFα (or Transforming Growth Factor α) polypeptide, a receptor of a TGFα polypeptide, an EGFR (or Epidermal Growth Factor Receptor) polypeptide (e.g., an ECD of an EGFR polypeptide), an MMP9 (or Matrix Metallopeptidase 9) polypeptide, an FGF (or Fibroblast Growth Factor) polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR (or Fibroblast Growth Factor Receptor) polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF (or Epidermal Growth Factor) polypeptide, a receptor of an EGF polypeptide, a neuregulin polypeptide (e.g., a neuregulin isoform I, II, III, IV, V, or VI polypeptide), a receptor of a neuregulin polypeptide, a HER (Human Epidermal Growth Factor Receptor) polypeptide (e.g., HER1, HER2, HER3, or HER4 polypeptide), an ECD of a HER polypeptide (e.g., an ECD of a HER1, a HER2, a HER3, or a HER4 polypeptide), an EpCAM (or Epithelial Cell Adhesion Molecule) polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP (or Calcitonin Gene-Related Peptide) polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL (or Calcitonin Receptor-Like) polypeptide (e.g., an ECD of a CALCRL polypeptide), a RAMP (or Receptor Activity-Modifying Protein) polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF (or Insulin-Like Growth Factor) polypeptide (e.g., an IGF-1 or an IGF-2 polypeptide), a receptor of an IGF polypeptide (e.g., a receptor of an IGF-1 or an IGF-2 polypeptide), an IGFR (or Insulin-Like Growth Factor Receptor) polypeptide (e.g., an IGFR1 or an IGFR2 polypeptide), an ECD of an IGFR polypeptide (e.g., an ECD of an IGFR1 or an IGFR2 polypeptide), an IGFBP (or Insulin-Like Growth Factor Binding Protein) polypeptide (e.g., IGFBP1, IGFBP2, IGFBP3, IGFBP4, IGFBP5, or IGFBP6 polypeptide), a VEGF (or Vascular Endothelial Growth Factor) polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF (or Placental Growth Factor) polypeptide), a VEGFR (or Vascular Endothelial Growth Factor Receptor) polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 (or FMS-like Tyrosine Kinase 1) receptor polypeptide (e.g., an ECD of an FLT1 receptor polypeptide), an IL36 (or Interleukin 36) polypeptide (e.g., IL36A, IL36B, or IL36G polypeptide), a receptor of an IL36 polypeptide (e.g., a receptor of an IL36A, an IL36B, or an IL36G polypeptide), an IL36R (or Interleukin 36 Receptor) polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 (or Sialic Acid-Binding Ig-Like Lectin 10) polypeptide, a PCSK9 (or Proprotein Convertase Subtilisin/Kexin Type 9) polypeptide, a receptor of a PCSK9 polypeptide, an LDLR (or Low Density Lipoprotein Receptor) polypeptide (e.g., an ECD of an LDLR polypeptide), a CEA (or Carcinoembryonic Antigen) polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF (or B-cell Activating Factor) polypeptide, a receptor of a BAFF polypeptide, a TRAF (or TNF Receptor Associated Factor) polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA (or B-cell Maturation Antigen) polypeptide, a SOST polypeptide, a receptor of a SOST (or Sclerostin) polypeptide, an LRP (or Low-density Lipoprotein Receptor-Related Protein) polypeptide (e.g., an LRP5 or an LRP6 polypeptide), an ECD of an LRP polypeptide (e.g., an ECD of an LRP5 or an LRP6 polypeptide), a DLL (or Delta-like) polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF (or von Willebrand Factor) polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 (or Interleukin 2) polypeptide, a receptor of an IL2 polypeptide, an IL2R (or Interleukin 2 Receptor) polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ (or Transforming Growth Factor (3) polypeptide, a receptor of a TGFβ polypeptide, a Decorin polypeptide, an EIF3I (or Eukaryotic Translation Initiation Factor 3 Subunit 1) polypeptide, a LTBP1 (or Latent-transforming Growth Factor Beta-Binding Protein 1) polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK (or Kallikrein) polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl (or Receptor Activator of Nuclear Factor Kappa-B ligand) polypeptide, a receptor of a Rankl polypeptide, a RANK (or Receptor Activator of Nuclear Factor Kappa-B) polypeptide (e.g., an ECD of a RANK polypeptide), a TSLP (or Thymic Stromal Lymphopoietin) polypeptide, a receptor of a TSLP polypeptide, a CRLF2 (or Cytokine Receptor-like Factor 2) polypeptide (e.g., an ECD of a CRLF2 polypeptide), an IL7Rα polypeptide (e.g., an ECD of an IL7Rα polypeptide), an S1P (or Specificity Protein 1) polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 (or Cytotoxic T-lymphocyte-Associated Protein 4) polypeptide (e.g., an ECD of a CTLA-4 polypeptide), a GnRH (or Gonadotropin-Releasing Hormone) polypeptide, a receptor of a GNRH polypeptide, a GnRHR (or Gonadotropin-Releasing Hormone Receptor) polypeptide (e.g., an ECD of a GnRHR polypeptide), an ICAM (or Intercellular Adhesion Molecule) polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 (or Complement component 5) polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, a glucagon polypeptide, or etc.

In some embodiments, antibody is one that recognizes one or more of the following polypeptides: a NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12β1 polypeptide (e.g., an ECD of an IL12β1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an EPO polypeptide, a somatostatin polypeptide, a GLP1 polypeptide, a glucagon polypeptide, or etc.

### Exemplary Variant IgG Fc Polypeptides and Fusion Molecules

Polypeptides and other molecules may comprise a variant IgG Fc polypeptide. In some embodiments, a fusion molecule comprises a variant IgG Fc polypeptide, such as the variant IgG Fc polypeptides described herein. In some embodiments, an antibody or an antibody fragment comprises a variant IgG Fc polypeptide, such as the variant IgG Fc polypeptides described herein.

A “fusion molecule,” as used herein, refers to a molecule comprising one or more “fusion partners.” In some embodiments, the fusion partners are covalently linked (“fused”). If two fusion partners are both polypeptides, the fusion partner polypeptides may be part of a contiguous amino acid sequence (i.e., a contiguous polypeptide). A first fusion partner polypeptide may be linked to either the N-terminus or the C-terminus of a second fusion partner. In some embodiments, the fusion partners are translated as a single polypeptide from a coding sequence that encodes both fusion partners. Fusion partners may be covalently linked through other means, such as, for example, a chemical linkage other than a peptide bond. Many known methods of covalently linking polypeptides to other molecules (for example, fusion partners) may be used. In other embodiments, the fusion partners are fused through a “linker,” which is comprised of at least one amino acid or chemical moiety. In some embodiments, fusion partners are noncovalently linked. In some such embodiments, they may be linked, for example, using binding pairs. Exemplary binding pairs include, but are not limited to, biotin and avidin or streptavidin, an antibody and its antigen, etc.

In some embodiments, the fusion partners include an IgG Fc polypeptide and at least one therapeutic polypeptide and/or antibody. In some embodiments, the fusion partners include an IgG Fc polypeptide, a first therapeutic polypeptide or antibody, and a second therapeutic polypeptide or antibody. In some embodiments, a therapeutic polypeptide may be linked to either the N-terminus or the C-terminus of an IgG Fc polypeptide. In some embodiments, an antibody may be linked to either the N-terminus or the C terminus of an IgG Fc polypeptide.

The term “contiguous polypeptide” herein is used to mean an uninterrupted sequence of amino acids. A contiguous polypeptide is typically translated from a single continuous DNA sequence. It can be made by genetic engineering, for example, by removing the stop codon from the DNA sequence of the first protein, then appending the DNA sequence of the second protein in frame, so that the DNA sequence is expressed as a single protein. Typically, this is accomplished by cloning a cDNA into an expression vector in frame with an existing gene.

A “linker” refers to one or more amino acid residues that connects a first polypeptide with a second polypeptide.

In some embodiments, the linker is a flexible, non-structural linker. In some embodiments, the linker is a glycine-rich, serine-rich, or glycine- and serine-rich linker. In some embodiments, a linker comprises 100%, at least 95%, at least 90%, or at least 85% serine and/or glycine amino acid residues.

An “extension,” as used herein, refers to one or more amino acid residues that are connected to a polypeptide at its C-terminus or at its N-terminus.

In some embodiments, an extension is flexible. In some embodiments, the extension adds flexibility to the polypeptide without interfering with the biological activity of the polypeptide. In some embodiments, the extension increases solubility of the polypeptide. In some embodiments, the extension comprises one or more glycine residues. In some embodiments, the extension comprises a glycine residue (SEQ ID NO: 88), two glycine residues (SEQ ID NO: 89), a three glycine residues (SEQ ID NO: 90), four glycine residues (SEQ ID NO: 91), five glycine residues (SEQ ID NO: 92), six glycine residues (SEQ ID NO: 93), seven glycine residues (SEQ ID NO: 94), eight glycine residues (SEQ ID NO: 95), or more glycine residues.

In some embodiments, the contiguous polypeptide comprises an IgG Fc polypeptide comprising an amino acid sequence of any one of SEQ ID NOs: 1, 2, 3, 4, 5, 6, 7, 8, 9, 10, 11, 12, 13, 14, 15, 16, 17, 18, 19, 60, 61, 62, 63, 64, 65, 66, 67, 68, 69, 70, 71, 72, 73, 74, 75, 76, 77, 78, 79, 80, 81, 82, 83, 84, 100, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 139, 140, 141, 142, 143, 144, 145, 146, 147, 148, 149, 150, 151, 152, 153, 154, 155, 156, 167, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 and a GLP1 polypeptide comprising an amino acid sequence of SEQ ID NO: 85. In some embodiments, the contiguous polypeptide comprises an IgG Fc polypeptide comprising an amino acid sequence of any one of SEQ ID NOs: 1, 2, 3, 4, 5, 6, 7, 8, 9, 10, 11, 12, 13, 14, 15, 16, 17, 18, 19, 60, 61, 62, 63, 64, 65, 66, 67, 68, 69, 70, 71, 72, 73, 74, 75, 76, 77, 78, 79, 80, 81, 82, 83, 84, 100, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 139, 140, 141, 142, 143, 144, 145, 146, 147, 148, 149, 150, 151, 152, 153, 154, 155, 156, 167, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 and a GLP1 polypeptide comprising an amino acid sequence of SEQ ID NO: 86. In some embodiments, the contiguous polypeptide comprises an IgG Fc polypeptide comprising an amino acid sequence of any one of SEQ ID NOs: 1, 2, 3, 4, 5, 6, 7, 8, 9, 10, 11, 12, 13, 14, 15, 16, 17, 18, 19, 60, 61, 62, 63, 64, 65, 66, 67, 68, 69, 70, 71, 72, 73, 74, 75, 76, 77, 78, 79, 80, 81, 82, 83, 84, 100, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 139, 140, 141, 142, 143, 144, 145, 146, 147, 148, 149, 150, 151, 152, 153, 154, 155, 156, 167, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 and a GLP1 polypeptide comprising an amino acid sequence of SEQ ID NO: 87. In some embodiments, the contiguous polypeptide comprises an IgG Fc polypeptide comprising an amino acid sequence of any one of SEQ ID NOs: 1, 2, 3, 4, 5, 6, 7, 8, 9, 10, 11, 12, 13, 14, 15, 16, 17, 18, 19, 60, 61, 62, 63, 64, 65, 66, 67, 68, 69, 70, 71, 72, 73, 74, 75, 76, 77, 78, 79, 80, 81, 82, 83, 84, 100, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 139, 140, 141, 142, 143, 144, 145, 146, 147, 148, 149, 150, 151, 152, 153, 154, 155, 156, 167, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 and a GLP1 polypeptide comprising an amino acid sequence of SEQ ID NO: 98. In some embodiments, the contiguous polypeptide comprises an IgG Fc polypeptide comprising an amino acid sequence of any one of SEQ ID NOs: 1, 2, 3, 4, 5, 6, 7, 8, 9, 10, 11, 12, 13, 14, 15, 16, 17, 18, 19, 60, 61, 62, 63, 64, 65, 66, 67, 68, 69, 70, 71, 72, 73, 74, 75, 76, 77, 78, 79, 80, 81, 82, 83, 84, 100, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 139, 140, 141, 142, 143, 144, 145, 146, 147, 148, 149, 150, 151, 152, 153, 154, 155, 156, 167, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 and a GLP1 polypeptide comprising an amino acid sequence of SEQ ID NO: 99.

In some embodiments, the contiguous polypeptide comprises an IgG Fc polypeptide comprising an amino acid sequence of any one of SEQ ID NOs: 1, 2, 3, 4, 5, 6, 7, 8, 9, 10, 11, 12, 13, 14, 15, 16, 17, 18, 19, 60, 61, 62, 63, 64, 65, 66, 67, 68, 69, 70, 71, 72, 73, 74, 75, 76, 77, 78, 79, 80, 81, 82, 83, 84, 100, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 139, 140, 141, 142, 143, 144, 145, 146, 147, 148, 149, 150, 151, 152, 153, 154, 155, 156, 167, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 and a glucagon polypeptide comprising an amino acid sequence of SEQ ID NO: 21.

In some embodiments, a contiguous polypeptide comprises:

TPA1-L1-Fc; Formula (I):

Fc-L1-TPA1; Formula (II):

TPA1-L1-Fc-L2-TPA2; Formula (III):

TPA1-L1-TPA2-L2-Fc; or Formula (IV):

Fc-L1-TPA1-L2-TPA2. Formula (V):

wherein TPA1 is a first therapeutic polypeptide and/or antibody, TPA2 is a second therapeutic polypeptide and/or antibody (e.g., the same therapeutic polypeptide, a different therapeutic polypeptide, the same antibody, or a different antibody), L1 and L2 are optional linkers; and Fc is a variant IgG Fc polypeptide of a companion animal species. Optionally, the contiguous polypeptide comprises a signal sequence. The constructs of Formulas I-V may comprise a TPA3, TPA4, TPA5, etc. following or before any TPA1 or TPA2. TPA3, TPA4, TPA5, etc. are third, fourth, fifth, etc. therapeutic polypeptides and/or antibodies (e.g., the same therapeutic polypeptide, a different therapeutic polypeptide, the same antibody, or a different antibody).

In some embodiments, the Fc polypeptide is a human IgG Fc. In some embodiments, the Fc polypeptide is a human IgG1 Fc, a human IgG2 Fc, a human IgG3 Fc, or a human IgG4 Fc. In some embodiments, the Fc polypeptide is a variant human IgG Fc.

In some embodiments, the Fc polypeptide is an IgG Fc from a companion animal. In some embodiments, the Fc polypeptide is a canine IgG-A Fc, a canine IgG-B Fc, a canine IgG-C Fc, a canine IgG-D Fc. In some embodiments, the Fc is an equine IgG1 Fc, an equine IgG2 Fc, an equine IgG3 Fc, an equine IgG4 Fc, an equine IgG5 Fc, an equine IgG6 Fc, or an equine IgG7 Fc. In some embodiments, the Fc is a feline IgG1a Fc, a feline IgG1b Fc, or a feline IgG2 Fc.

In some embodiments, the Fc polypeptide is a variant IgG Fc. In some embodiments, the FC polypeptide is a variant canine IgG-A Fc, a variant canine IgG-B Fc, a variant canine IgG-C Fc, a variant canine IgG-D Fc. In some embodiments, the Fc is a variant equine IgG1 Fc, a variant equine IgG2 Fc, a variant equine IgG3 Fc, a variant equine IgG4 Fc, a variant equine IgG5 Fc, a variant equine IgG6 Fc, or a variant equine IgG7 Fc. In some embodiments, the Fc is a variant feline IgG1a Fc, a variant feline IgG1b Fc, or a variant feline IgG2 Fc.

In some embodiments, L1 and L2, if present, each independently is a flexible linker. In some embodiments, the amino acid sequence of L1 and L2, if present, each independently comprises 100%, at least 95%, at least 90%, at least 85% serine and/or glycine amino acid residues.

In some embodiments, the contiguous polypeptide comprises an extension at its C-terminus. In some embodiments, the contiguous polypeptide comprises a glycine residue, two glycine residues, three glycine residues, four glycine residues, five glycine residues, six glycine residues, seven glycine residues, eight glycine residues, or greater than eight glycine residues at its C-terminus. In some embodiments, the contiguous polypeptide comprises an amino acid sequence of SEQ ID NO: 158, SEQ ID NO: 159, SEQ ID NO: 160, SEQ ID NO: 161, SEQ ID NO: 162, SEQ ID NO: 163, SEQ ID NO: 164, or SEQ ID NO: 165 at its C-terminus.

In some embodiments, the contiguous polypeptide comprises the amino acid sequence of SEQ ID NO: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 57, 58, 59, 60, 61, 62, 63, 64, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 146, 147, 148, 149, 150, 151, 154, 155, 157, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 271, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, and/or 250.

A nucleotide sequence encoding a polypeptide of interest, such as a variant IgG Fc polypeptide or other polypeptide described herein, can be inserted into an expression vector suitable for expression in a selected host cell. A variant IgG Fc polypeptide or other polypeptide described herein may be expressed by culturing a host cell transfected with an expression vector comprising the nucleotide sequence.

A “vector” is a plasmid that can be used to transfer DNA sequences from one organism to another or to express a gene of interest. A vector typically includes an origin of replication and regulatory sequences which regulate the expression of the gene of interest, and may or may not carry a selective marker gene, such as an antibiotic resistance gene. A vector is suitable for the host cell in which it is to be expressed. A vector may be termed a “recombinant vector” when the gene of interest is present in the vector.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NS0 cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.”

A “signal sequence” refers to a sequence of amino acid residues or polynucleotides encoding such, which facilitates secretion of a polypeptide of interest and is typically cleaved upon export of the polypeptide to the outside of the cell surface membrane.

In some embodiments, a variant IgG Fc polypeptide or a contiguous polypeptide comprising a variant Fc polypeptide is isolated using chromatography, such as size exclusion chromatography, ion exchange chromatography, protein A column chromatography, hydrophobic interaction chromatography, and CHT chromatography.

A label can be attached to a variant IgG Fc polypeptides or a contiguous polypeptide comprising a variant Fc polypeptide. A “label” means a moiety attached to a molecule to render it detectable. In some embodiments, a variant IgG Fc polypeptide or a contiguous polypeptide comprising a variant Fc polypeptide is labeled with a detectable moiety including but not limited to radioisotopes, fluorescent labels, and various enzyme-substrate labels known in the art. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, p-galactosidase, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety. General techniques to be used in performing the various immunoassays noted above are known to those of ordinary skill in the art.

Exemplary Variant IgG Fc Polypeptide Affinity to Protein a and/or C1q and/or CD16

The variant IgG Fc polypeptides described herein may have altered binding affinity to Protein A and/or C1q and/or CD16. In some embodiments, a variant IgG Fc polypeptide has increased binding affinity to Protein A relative to the wild-type IgG Fc polypeptide. Such variant IgG Fc polypeptides may be purified by Protein A column chromatography. In some embodiments, a variant IgG Fc polypeptide has reduced binding affinity to C1q relative to the wild-type IgG Fc polypeptide. Such variant IgG Fc polypeptides may have reduced complement-mediated immune responses. In some embodiments, a variant IgG Fc polypeptide has reduced binding affinity to CD16 relative to the wild-type IgG Fc polypeptide. Such variant IgG Fc polypeptides may have reduced ADCC immune responses. In some embodiments, a variant IgG Fc polypeptide has increased binding affinity to Protein A relative to the wild-type IgG Fc polypeptide and/or has reduced binding affinity to C1q relative to the wild-type IgG Fc polypeptide and/or has reduced binding affinity to CD16 relative to the wild-type IgG Fc polypeptide.

“Protein A,” as used herein, is a polypeptide comprising the entirety or a portion of Protein A that is capable of binding a wild-type canine IgG-B Fc, a wild-type equine IgG1 Fc, a wild-type equine IgG3 Fc, a wild-type equine IgG4 Fc, a wild-type equine IgG7 Fc, a wild-type feline IgG1a Fc, a wild-type feline IgG1b Fc, or a wild-type feline IgG2 Fc.

“C1q” or “C1q complex” is used interchangeably to refer to a protein complex involved in the complement system, or a portion thereof, that can bind a wild-type canine IgG-B Fc, a wild-type canine IgG-C Fc, a wild-type equine IgG1 Fc, a wild-type equine IgG3 Fc, a wild-type equine IgG4 Fc, a wild-type equine IgG7 Fc, a wild-type feline IgG1a Fc, or a wild-type feline IgG1b Fc.

“CD16,” as used herein, is a polypeptide comprising the entirety or a portion of CD16 that is capable of binding a wild-type canine IgG-A Fc or a wild-type canine IgG-D Fc. The term “binds” to a substance is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA, KinEx A, biolayer interferometry (BLI), surface plasmon resonance devices, or etc.

“Protein A+,” as used herein, means that the Fc polypeptide has Protein A binding affinity. In some embodiments, a Protein A+Fc polypeptide comprises at least one an amino acid modification that increases Protein A binding affinity.

“Protein A−,” as used herein, means that the Fc polypeptide has low or no Protein A binding affinity.

“C1q+,” as used herein, means that the Fc polypeptide has C1q binding affinity.

“C1q−,” as used herein, means that the Fc polypeptide has low or no C1q binding affinity. In some embodiments, a C1q− Fc polypeptide has at least one an amino acid modification that reduces C1q binding affinity.

“CD16+,” as used herein, means that the Fc polypeptide has CD16 binding affinity.

“CD16−,” as used herein, means that the Fc polypeptide has low or no CD16 binding affinity. In some embodiments, a CD16− Fc polypeptide has at least one an amino acid modification that reduces CD16 binding affinity.

The term “affinity” means the strength of the sum total of noncovalent interactions between a single binding site of a molecule (for example, a receptor) and its binding partner (for example, a ligand). The affinity of a molecule X for its partner Y can generally be represented by the dissociation constant (K_{D}). Affinity can be measured by common methods known in the art, such as, for example, immunoblot, ELISA, KinEx A, biolayer interferometry (BLI), or surface plasmon resonance devices.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

The terms “K_{D},” “K_{d},” “Kd” or “Kd value” as used interchangeably to refer to the equilibrium dissociation constant of a receptor-ligand interaction or antibody-antigen interaction.

In some embodiments, a variant IgG Fc polypeptide binds to Protein A with a dissociation constant (K_{D}) of less than 5×10⁻⁶ M, less than 1×10⁻⁶ M, less than 5×10⁻⁷ M, less than 1×10⁻⁷ M, less than 5×10⁻⁸M, less than 1×10⁻⁸M, less than 5×10⁻⁹M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, a variant IgG Fc polypeptide binds to C1q and/or CD16 with a dissociation constant (K_{D}) of greater than 5×10⁻⁶ M, greater than 1×10⁻⁵ M, greater than 5×10⁻⁵ M, greater than 1×10⁻⁴ M, greater than 5×10⁻⁴ M, or greater than 1×10⁻³M, as measured by biolayer interferometry.

In some embodiments, a variant canine IgG-A or IgG-D Fc polypeptide binds to C1q and/or CD16 with a dissociation constant (K_{D}) of less than 5×10⁻⁶ M, less than 1×10⁻⁶ M, less than 5×10⁻⁷ M, less than 1×10⁻⁷ M, less than 5×10⁻⁸M, less than 1×10⁻⁸M, less than 5×10⁻⁹M, less than 1×10⁻⁹M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, the K_{D} of an IgG Fc polypeptide, such as a variant IgG Fc polypeptide, to Protein A or to C1q or to CD16 is measured by using biolayer interferometry assays using a biosensor, such as an Octet® System (Pall ForteBio LLC, Fremont, Calif.) according to the supplier's instructions. In brief, biotinylated Protein A or C1q or CD16 is bound to the sensor tip and the association of IgG Fc polypeptide is monitored for a specified time or until steady state is reached. Dissociation may be monitored for a specified time or until steady state is reached. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{D}) is calculated as the ratio of k_{off}/k_{off} The term “kₒₙ” refers to the rate constant for association of a molecule X to its partner Y and the term “k_{off}” refers to the rate constant for dissociation of a molecule X or partner Y from the molecule X/partner Y complex.

To “increase” or “stimulate” means to increase, improve, or augment an activity, function, or amount as compared to a reference. In some embodiments, by “increase” or “stimulate” is meant the ability to cause an overall increase of about 5% or greater, of about 10% or greater, of about 20% or greater, of about 30% or greater, of about 40% or greater, of about 50% or greater, of about 60% or greater, of about 70% or greater, of about 80% or greater, of about 90% or greater, of about 100% or greater, of about 125% or greater, of about 200% or greater relative to a reference value. In some embodiments, by “increase” or “stimulate” is meant the ability to cause an overall increase of about 5% to about 50%, of about 10% to about 20%, of about 50% to about 100%, of about 25% to about 70% relative to a reference value. In some embodiments, by “increase” or “stimulate” is meant the ability to cause an overall increase of 50% or greater. In some embodiments, by “increase” or “stimulate” is meant the ability to cause an overall increase of 75%, 85%, 90%, 95%, or greater. In some embodiments, the amount noted above is stimulated or increased over a period of time, relative to a control dose (such as a placebo) over the same period of time.

In some embodiments, a variant IgG Fc polypeptide is capable of binding to Protein A with an increased affinity of about 5% or greater, of about 10% or greater, of about 20% or greater, of about 30% or greater, of about 40% or greater, of about 50% or greater, of about 60% or greater, of about 70% or greater, of about 80% or greater, of about 90% or greater, of about 100% or greater, of about 125% or greater, of about 150% or greater, of about 200% or greater relative to a reference IgG Fc polypeptide. In some embodiments, a variant IgG Fc polypeptide is capable of binding to Protein A with an increased affinity of about 5% to about 50%, of about 10% to about 20%, of about 50% to about 100%, of about 25% to about 70% relative to a reference IgG Fc polypeptide. In some embodiments, the reference IgG Fc polypeptide is a wild-type IgG Fc polypeptide. In some embodiments, the reference IgG Fc polypeptide is a different variant IgG Fc polypeptide.

To “reduce” or “inhibit” means to decrease, reduce, or arrest an activity, function, or amount as compared to a reference. In some embodiments, by “reduce” or “inhibit” is meant the ability to cause an overall decrease of about 5% or greater, of about 10% or greater, of about 20% or greater, of about 30% or greater, of about 40% or greater, of about 50% or greater, of about 60% or greater, of about 70% or greater, of about 80% or greater, or of about 90% or greater relative to a reference IgG Fc polypeptide. In some embodiments, by “reduce” or “inhibit” is meant the ability to cause an overall decrease of about 5% to about 50%, of about 10% to about 20%, of about 50% to about 100%, of about 25% to about 70% relative to a reference value. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time.

In some embodiments, a variant IgG Fc polypeptide is capable of binding to C1q or CD16 with a decreased affinity of about 5% or greater, of about 10% or greater, of about 20% or greater, of about 30% or greater, of about 40% or greater, of about 50% or greater, of about 60% or greater, of about 70% or greater, of about 80% or greater, of about 90% or greater relative to a reference IgG Fc polypeptide. In some embodiments, a variant IgG Fc polypeptide is capable of binding to C1q or CD16 with a decreased affinity of about 5% to about 50%, of about 10% to about 20%, of about 50% to about 100%, of about 25% to about 70% relative to a reference IgG Fc polypeptide. In some embodiments, the reference IgG Fc polypeptide is a wild-type IgG Fc polypeptide. In some embodiments, the reference IgG Fc polypeptide is a different variant IgG Fc polypeptide.

A “reference” as used herein, refers to any sample, standard, or level that is used for comparison purposes. A reference may be a wild-type reference or a variant reference. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

### Exemplary Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where a variant IgG Fc polypeptide or other polypeptide described herein is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

### Certain Uses of Fc Polypeptides and Pharmaceutical Compositions

A polypeptide comprising a variant Fc polypeptide, such as a variant IgG Fc polypeptide, of the invention or pharmaceutical compositions comprising a variant Fc polypeptide of the invention may be useful for extending product half-life in vivo in a companion animal, including, but not limited to, canine, feline, or equine.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, a variant IgG Fc polypeptide or other polypeptide described herein, or a pharmaceutical composition comprising such is administered parenterally, by subcutaneous administration, intravenous infusion, or intramuscular injection. In some embodiments, a variant IgG Fc polypeptide or other polypeptide described herein, or a pharmaceutical composition comprising such is administered as a bolus injection or by continuous infusion over a period of time. In some embodiments, a variant IgG Fc polypeptide or other polypeptide described herein, or a pharmaceutical composition comprising such is administered by an intramuscular, an intraperitoneal, an intracerebrospinal, a subcutaneous, an intra-arterial, an intrasynovial, an intrathecal, or an inhalation route.

In some embodiments, a variant IgG Fc polypeptide or other polypeptide described herein, or a pharmaceutical composition comprising such is administered in an amount in the range of 0.0001 mg/kg body weight to 100 mg/kg body weight per dose, in the range of 0.005 mg/kg body weight to 20 mg/kg body weight per dose, in the range of 1 mg/kg body weight to 10 mg/kg body weight per dose, in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, or in the range of 5 mg/kg body weight to 50 mg/kg body weight.

In some embodiments, a variant IgG Fc polypeptide or other polypeptide described herein, or a pharmaceutical composition comprising such is administered to a companion animal at one time or over a series of treatments. In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

The following examples illustrate particular aspects of the disclosure and are not intended in any way to limit the disclosure.

### EXAMPLES

### Example 1

### Variant Canine IgG Fc Polypeptides for Increased Protein a Binding and/or Decreased Complement Binding and/or Decreased CD16 Binding

Purification of antibodies using Protein A affinity is a well-developed process. However, among four subtypes of canine IgG, only IgG-B Fc (e.g., SEQ ID NO: 2 or SEQ ID NO: 3) has Protein A binding affinity. Canine IgG-A Fc (e.g., SEQ ID NO: 1), IgG-C Fc (e.g., SEQ ID NO: 4 or SEQ ID NO: 5), and IgG-D Fc (e.g., SEQ ID NO: 6) have weak or no measurable Protein A binding affinity. Variant canine IgG-A Fc, IgG-C Fc, and IgG-D Fc polypeptides were designed for altered Protein A binding.

In addition, canine IgG-B Fc and IgG-C Fc have complement activity and bind to C1q, while canine IgG-A Fc and IgG-D Fc have weak or no measurable binding affinity to C1q. To potentially reduce the C1q binding and/or potentially reduce complement-mediated immune responses, variant canine IgG-B Fc and IgG-C Fc polypeptides were designed.

Furthermore, canine IgG-B Fc and IgG-C Fc have CD16 binding activity. To potentially reduce the binding of CD16 to IgG-B Fc and IgG-C Fc, and/or potentially reduce ADCC, variant canine IgG-B Fc and IgG-C Fc polypeptides were designed.

Table 3, below summarizes the Protein A and C1q binding characteristics of canine IgG Fc subtypes. Notably, none of the wild-type canine IgG Fc subtypes lacks C1q binding and binds Protein A.

TABLE 3

| Wild-type | Protein A | C1q | CD16 |
|---|---|---|---|
| Canine IgG Fc | Binding | Binding | Binding |
| IgG-A Fc | − | − | − |
| IgG-B Fc | + | + | + |
| IgG-C Fc | − | + | + |
| IgG-D Fc | − | − | − |

(−) denotes low or no measurable binding activity.

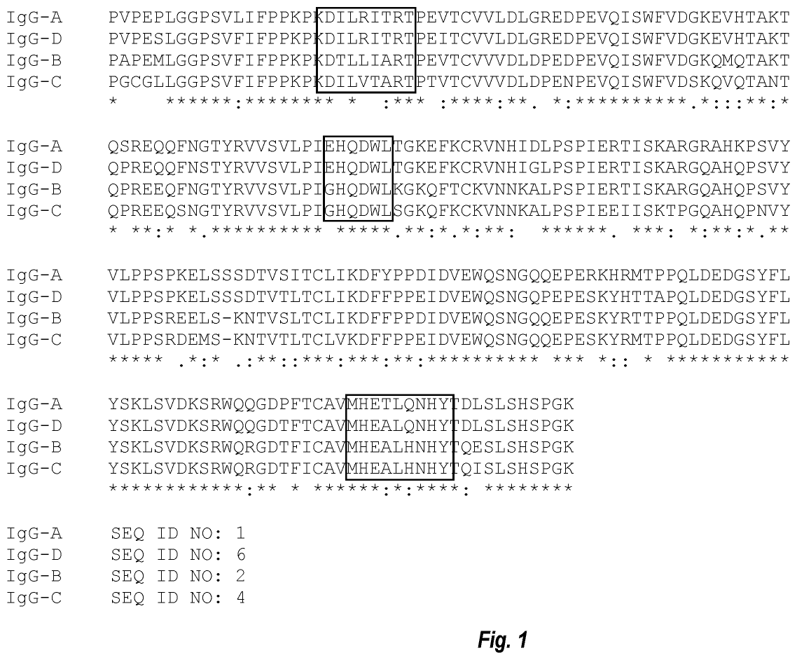

Using three-dimensional protein modeling and protein sequence analysis, the sequences of canine IgG-B Fc that are likely in contact with Protein A were identified. FIG. 1 shows an alignment of canine IgG-A, IgG-B, IgG-C, and IgG-D Fc sequences. The boxes indicate the regions likely in contact with Protein A.

Two approaches were used to design variant canine IgG-A, IgG-C, and IgG-D Fc polypeptides for increased Protein A binding. For the first approach, variant canine IgG-A, IgG-C, and IgG-D Fc polypeptides were designed to have the same Protein A binding motif sequences as canine IgG-B Fc (e.g., SEQ ID NO: 7, SEQ ID NO: 8, and SEQ ID NO: 9, respectively). For the second approach, variant canine IgG-A Fc I(21)T/Q(207)H (SEQ ID NO: 10), variant canine IgG-C Fc I(21)T (SEQ ID NO: 11), and variant canine IgG-D Fc I(21)T/Q(207)H (SEQ ID NO: 12) were designed with one or two amino acid substitutions in the Protein A binding region to correspond with the canine IgG-B Fc sequence.

In addition, variant canine IgG-A Fc, IgG-C Fc, and IgG-D Fc polypeptides with increased Protein A binding may be prepared having one or more of the amino acid substitutions listed in Table 4.

TABLE 4
Variant Canine IgG Fc Amino Acid Substitutions* (Protein A+)

| Canine IgG-A Fc | Canine IgG-C Fc | Canine IgG-D Fc |
|---|---|---|
| (SEQ ID NO: 1) | (SEQ ID NO: 4) | (SEQ ID NO: 6) |
| Ile (21) Thr | Ile (21) Thr | Ile (21) Thr |
| Arg (23) Leu | Val (23) Leu | Arg (23) Leu |
| Thr (25) Ala | Thr (24) Ile | Thr (25) Ala |
| Glu (80) Gly | | Glu (80) Gly |
| Thr (205) Ala | | Gln (207) His |
| Gln (207) His | | |

*The amino acid positions listed are relative to the SEQ ID NO. indicated.

To potentially reduce the binding of C1q to canine IgG-B Fc and IgG-C Fc, and/or potentially reduce complement-mediated immune responses, variant canine IgG-B Fc and IgG-C Fc polypeptides may be prepared having an amino acid substitution of Lys with any amino acid except Lys at an amino acid position corresponding to position 93 of SEQ ID NO: 2 or of SEQ ID NO: 4, respectively. These amino acid substitutions were identified after analysis of the protein sequence and 3-D structure modeling of canine IgG-B Fc and IgG-C Fc compared to canine IgG-A Fc and IgG-D Fc, which are understood to not exhibit complement activity. For example, variant canine IgG-B Fc K(93)R (SEQ ID NO: 13) and variant canine IgG-C Fc K(93)R (SEQ ID NO: 14) may be prepared. Reduced binding between human C1q and a fusion protein comprising variant canine IgG-B Fc K(93)R was observed when compared to a fusion protein comprising wild-type canine IgG-B Fc.

To potentially reduce the binding of CD16 to IgG-B Fc and IgG-C Fc, and/or potentially reduce ADCC, variant canine IgG-B Fc and IgG-C Fc polypeptides may be prepared having one or more of the amino acid substitutions listed in Table 5 (e.g., SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and/or SEQ ID NO: 29). The amino acid substitution(s) were identified after analysis of the protein sequence and 3-D structure modeling of canine IgG-B and IgG-C compared to IgG-A and IgG-D, which are understood to not exhibit ADCC activity.

TABLE 5

Original residue position*

| Canine IgG-B Fc | Canine IgG-C Fc | |
|---|---|---|
| (SEQ ID NO: 2) | (SEQ ID NO: 4) | Substitution(s) |
| Met (5) | Leu (5) | Any amino acid |
| | | except original |
| | | residue, such as Pro |
| Asp (38) | Asp (38) | Any amino acid |
| | | except original |
| | | residue, such as |
| | | Gly |
| Pro (39) | Pro (39) | Any amino acid |
| | | except original |
| | | residue, such as |
| | | Arg |
| Lys (97) | Lys (97) | Any amino acid |
| | | except original |
| | | residue, such as Ile |
| Ala (98) | Ala (98) | Any amino acid |
| | | except original |
| | | residue, such as |
| | | Gly |

*The amino acid positions listed are relative to the SEQ ID NO. indicated.

Since wild-type canine IgG-C Fc lacks Protein A binding and has C1q binding, a double variant canine IgG-C Fc that binds Protein A and has reduced binding to C1q may be prepared by combining one or more of the amino acid substitutions listed in Table 4 with a K(93)R substitution or K(93)X substitution, wherein X is any amino acid except Lys (e.g., SEQ ID NO: 30). A double variant canine IgG-B Fc or double variant canine IgG-C Fc with reduced binding to C1q and reduced binding to CD16 may be prepared by combining one or more of the amino acid substitutions listed in Table 5 with a K(93)R substitution or K(93)X substitution, wherein X is any amino acid except Lys (e.g., SEQ ID NO: 31, SEQ ID NO: 32, SEQ ID NO: 33, and/or SEQ ID NO: 34). A triple variant canine-IgG-C Fc that binds Protein A and has reduced binding to C1q and CD16 may be prepared by combining one or more of the amino acid substitutions listed in Table 4 and one or more of the amino acid substitutions listed in Table 5 with a K(93)R substitution or K(93)X substitution, wherein X is any amino acid except Lys.

The binding of any variant canine IgG Fc to Protein A, CD16, and/or C1q may be determined and compared to the binding of another IgG Fc to Protein A, CD16, and/or C1q (e.g., the corresponding wild-type canine IgG Fc, another wild-type or variant canine IgG Fc, or a wild-type or variant IgG Fc of another companion animal, etc.).

Binding analysis may be performed using an Octet biosensor. Briefly, the target molecule (e.g., Protein A, C1q, CD16, etc.) may be biotinylated and free unreacted biotin removed (e.g., by dialysis). The biotinylated target molecule is captured on streptavidin sensor tips. Association of the target molecule with various concentrations (e.g., 10 μg/mL) of IgG Fc polypeptide is monitored for a specified time or until steady state is reached. Dissociation is monitored for a specified time or until steady state is reached. A buffer only blank curve may be subtracted to correct for any drift. The data are fit to a 1:1 binding model using ForteBio™ data analysis software to determine the kₒₙ, k_{off}, and the K_{d}.

### Example 2

### Variant Canine IgG-A and IgG-D Fc Polypeptides with Increased Protein a Binding and/or Increased Complement Binding and/or Increased CD16 Binding

Based on the amino acids positions identified as being involved in Protein A, C1q, and CD16 binding described in Example 1, to potentially increase binding of Protein A, C1q, and/or CD16 to canine IgG-A Fc and IgG-D Fc, gain of function canine IgG-A Fc and IgG-D Fc polypeptides were designed. For example, variant canine IgG-A and IgG-D Fc polypeptides may be designed with one or multiple amino acid substitutions in the Protein A binding region, the C1q binding region, and/or the CD16 binding region to correspond with the sequences of wild-type canine IgG Fc polypeptides that bind Protein A, C1q, and/or CD16.

Single, double, or triple variant canine IgG-A and/or IgG-D polypeptides may be prepared by combining one or more of the amino acid substitutions listed in Table 6. For example, variant canine IgG-A Fc polypeptides of SEQ ID NO: 35, SEQ ID NO: 36, SEQ ID NO: 37, SEQ ID NO: 38, SEQ ID NO: 39, SEQ ID NO: 40, and/or SEQ ID NO: 41 and variant canine IgG-D Fc polypeptides of SEQ ID NO: 42, SEQ ID NO: 43, SEQ ID NO: 44, SEQ ID NO: 45, SEQ ID NO: 46, SEQ ID NO: 47, and/or SEQ ID NO: 48 may be prepared.

TABLE 6
Substitutions for Increased Protein A, C1q, and/or CD16 Binding

| | Canine IgG-A Fc | Canine IgG-D Fc |
|---|---|---|
| Function | (SEQ ID NO: 1) | (SEQ ID NO: 6) |
| CD16 | Val (2) Ala | Val (2) Ala |
| CD16 | Pro (5) Met or Lys | Ser (5) Met or Lys |
| Protein A | Ile (21) Thr | Ile (21) Thr |
| Protein A | Arg (23) Leu | Arg (23) Leu |
| Protein A | Thr (25) Ala | Thr (25) Ala |
| CD16 | Leu (35) Val | Leu (35) Val |
| CD16 | Gly (38) Asp | Gly (38) Asp |
| CD16 | Arg (39) Pro | Arg (39) Pro |
| CD16 | Gln (65) Glu | Gln (65) Glu |
| Protein A | Glu (80) Gly | Glu (80) Gly |
| C1q | Arg (93) Lys | Arg (93) Lys |
| CD16 | His (96) Asn | His (96) Asn |
| CD16 | Ile (97) Lys | Ile (97) Lys |
| CD16 | Asp (98) Ala | Gly (98) Ala |
| Protein A | Thr (205) Ala | |
| Protein A | Gln (207) His | Gln (207) His |

The binding of any variant canine IgG-A or IgG-D Fc polypeptide to Protein A, C1q, and/or CD16 may be determined and compared to the binding of another IgG Fc to Protein A, C1q, and/or CD16 (e.g., the corresponding wild-type canine IgG Fc, another wild-type or variant canine IgG Fc, or a wild-type or variant IgG Fc of another companion animal, etc.). The binding assay described in Example 1 may be used.

### Example 3

### Variant Equine IgG Fc Polypeptides for Increased Protein a Binding and/or Decreased Complement Binding

Of the seven subtypes of equine IgG, IgG1 Fc (e.g., SEQ ID NO: 49), IgG3 Fc (e.g., SEQ ID NO: 52), IgG4 Fc (e.g., SEQ ID NO: 53), IgG7 Fc (e.g., SEQ ID NO: 56) have Protein A binding affinity. Equine IgG2 Fc (e.g., SEQ ID NO: 50, SEQ ID NO: 51), IgG5 Fc (e.g., SEQ ID NO: 54), and IgG6 Fc (e.g., SEQ ID NO: 55) have weak or no measurable Protein A binding affinity. Variant equine IgG2 Fc, IgG5 Fc, and IgG6 Fc polypeptides were designed for altered Protein A binding.

In addition, equine IgG2 Fc, IgG5 Fc, and IgG6 Fc have weak or no measurable binding affinity to C1q, while equine IgG1 Fc, IgG3 Fc, IgG4 Fc, and IgG7 Fc bind to C1q. To potentially reduce the C1q binding and/or potentially reduce complement-mediated immune responses, variant equine IgG1 Fc, IgG3 Fc, IgG4 Fc, and IgG7 Fc polypeptides were designed.

Table 7, below summarizes the Protein A and C1q binding characteristics of equine IgG Fc subtypes. Notably, none of the wild-type equine IgG Fc subtypes lacks C1q binding and binds Protein A.

TABLE 7

| Wild-type | Protein A | C1q |
|---|---|---|
| Equine IgG Fc | Binding | Binding |
| IgG1 Fc | + | + |
| IgG2 Fc | − | − |
| IgG3 Fc | + | + |
| IgG4 Fc | + | + |
| IgG5 Fc | − | − |
| IgG6 Fc | − | − |
| IgG7 Fc | + | + |

(−) denotes low or no measurable binding activity.

Using three-dimensional protein modeling and protein sequence analysis, the sequences of equine IgG1 Fc, IgG3 Fc, IgG4 Fc, and IgG7 Fc that are likely in contact with Protein A were identified. Variant equine IgG2 Fc, IgG5 Fc, and IgG6 Fc polypeptides with increased Protein A binding may be prepared having one or more of the amino acid substitutions listed in Table 8.

TABLE 8
Variant Equine IgG Fc Amino Acid Substitutions* (Protein A+)

| Equine IgG2 Fc | Equine IgG5 Fc | Equine Ig6 Fc |
|---|---|---|
| (SEQ ID NO: 50) | (SEQ ID NO: 54) | (SEQ ID NO: 55) |
| Ala (15) Thr | Val (199) Leu | Ile (199) Leu |
| Phe (203) Tyr | Glu (200) Tyr | Arg (200) His |
| | | His (201) Asn |
| | | Thr (202) His |

*The amino acid positions listed are relative to the SEQ ID NO. indicated

For example, variant equine IgG2 Fc, IgG5 Fc, and IgG6 Fc polypeptides were designed with one or multiple amino acid substitutions in the Protein A binding region to correspond with the sequence of wild-type equine IgG Fc, which does bind Protein A. Variant equine IgG2 Fc F(203)Y (SEQ ID NO: 57); variant equine IgG2 Fc A(15)T/F(203)Y (SEQ ID NO: 58); variant equine IgG5 Fc V(199)L/E(200)Y (SEQ ID NO: 59); and variant equine IgG6 Fc I(199)L/R(200)H/H(201)N/T(202)H (SEQ ID NO: 60) with increased Protein A binding may be prepared.

To potentially reduce the binding of C1q to equine IgG1 Fc, IgG3 Fc, IgG4 Fc, and IgG7 Fc, and/or potentially reduce complement-mediated immune responses, variant canine IgG1 Fc, IgG3 Fc, IgG4 Fc, and IgG7 Fc polypeptides may be prepared having an amino acid substitution of Lys with any amino acid except Lys at an amino acid position corresponding to position 87 of SEQ ID NO: 49, of SEQ ID NO: 52, of SEQ ID NO: 53, of SEQ ID NO: 56, respectively. These amino acid substitutions were identified after analysis of the protein sequence and 3-D structure modeling of equine IgG1 Fc, IgG3 Fc, IgG4 Fc, and IgG7 Fc compared to equine IgG2 Fc, IgG5 Fc, and IgG6 Fc, which are understood to not exhibit complement activity. For example, variant equine IgG1 Fc K(87)S (SEQ ID NO: 61), variant equine IgG3 Fc K(87)S (SEQ ID NO: 62), variant equine IgG4 Fc K(87)S (SEQ ID NO: 63), and variant equine IgG7 Fc K(87)S (SEQ ID NO: 64) may be prepared.

The binding of any variant equine IgG Fc to Protein A and/or C1q may be determined and compared to the binding of another IgG Fc to Protein A and/or C1q (e.g., the corresponding wild-type equine IgG Fc, another wild-type or variant equine IgG Fc, or a wild-type or variant IgG Fc of another companion animal, etc.). The binding assay described in Example 1 may be used.

### Example 4

### Variant Feline IgG Fc Polypeptides for Decreased Complement Binding

Each of the three subtypes of feline IgG, IgG1a Fc (SEQ ID NO: 65 or SEQ ID NO: 66), IgG1b Fc (SEQ ID NO: 67 or SEQ ID NO: 68), and IgG2 Fc (SEQ ID NO: 69) have Protein A binding affinity. However, only feline IgG2 Fc has weak or no measurable binding affinity to C1q, while feline IgG1a Fc, IgG1b Fc bind to C1q. To potentially reduce the C1q binding and/or potentially reduce complement-mediated immune responses, variant feline IgG1a Fc and IgG1b Fc polypeptides were designed.

Table 9, below summarizes the Protein A and C1q binding characteristics of feline IgG Fc subtypes. Notably, none of the wild-type equine IgG Fc subtypes lacks C1q binding and binds Protein A.

TABLE 9

| Wild-type | Protein A | C1q |
|---|---|---|
| Feline IgG Fc | Binding | Binding |
| IgG1a Fc | + | + |
| IgG1b Fc | + | + |
| IgG2 Fc | + | − |

(−) denotes low or no measurable binding activity.

To potentially reduce the binding of C1q to feline IgG1a Fc and IgG1b Fc, and/or potentially reduce complement-mediated immune responses, variant feline IgG1a Fc and IgG1b Fc polypeptides may be prepared having an amino acid substitution of Pro with any amino acid except Pro at an amino acid position corresponding to position 198 of SEQ ID NO: 65, of SEQ ID NO: 66, of SEQ ID NO: 67, or of SEQ ID NO: 68. These amino acid substitutions were identified after analysis of the protein sequence and 3-D structure modeling of feline IgG1a Fc and IgG1b Fc compared to feline IgG2 Fc, which is understood to not exhibit complement activity. For example, variant feline IgG1a Fc P(198)A (e.g., SEQ ID NO: 70 or SEQ ID NO: 71) and variant feline IgG1b Fc P(198)A (e.g., SEQ ID NO: 72 or SEQ ID NO: 73) may be prepared.

The binding of any variant feline IgG Fc to C1q may be determined and compared to the binding of another IgG Fc to C1q (e.g., the corresponding wild-type feline IgG Fc, another wild-type or variant feline IgG Fc, or a wild-type or variant IgG Fc of another companion animal, etc.). The binding assay described in Example 1 may be used.

### Example 5

### Variant Canine, Feline, and Equine IgG Fc Polypeptides for Heterodimeric Proteins

To enable the preparation of a bispecific canine, feline, or equine antibody or a bifunctional canine, feline, or equine Fc fusion protein using a knob-in-hole heterodimerization approach, pairing of variant canine IgG Fc polypeptides, variant feline IgG Fc polypeptides, and variant equine IgG Fc polypeptides was investigated. Pairing of two Fc polypeptides was designed by introducing CH3 interfacing mutations so that a first Fc polypeptide comprises a bulky amino acid (knob) and a second Fc polypeptide comprises smaller amino acid(s) in the same general location (hole).

An amino acid substitution of threonine to tyrosine or tryptophan at a position corresponding to position 138 of canine IgG-A Fc (SEQ ID NO: 1) or of canine IgG-D Fc (SEQ ID NO: 6) (T138Y or T138W), or at a position corresponding to position 137 of canine IgG-B Fc (SEQ ID NO: 2) or canine IgG-C Fc (SEQ ID NO: 4) (T137Y or T137W) can be introduced to one Fc chain as a knob (heterodimer chain 1). Examples of amino acid sequences of variant canine IgG-A Fc, IgG-B Fc, IgG-C Fc, and IgG-D Fc heterodimer chain 1 are SEQ ID NO: 74, SEQ ID NO: 75, SEQ ID NO: 76, SEQ ID NO: 77, SEQ ID NO: 78, SEQ ID NO: 79, SEQ ID NO: 80, and SEQ ID NO: 81.

An amino acid substitution of threonine to serine at a position corresponding to position 138, and/or of leucine to alanine at a position corresponding to position 140, and/or of tyrosine to threonine at a position corresponding to position 180 of canine IgG-A (SEQ ID NO: 1) or of IgG-D (SEQ ID NO: 6) (T138S, L140A, and/or Y180T), or of threonine to serine at a position corresponding to position 137, and/or of leucine to alanine at a position corresponding to position 139, and/or of tyrosine to threonine at a position corresponding to position 179 of canine IgG-B Fc (SEQ ID NO: 2) or of IgG-C(SEQ ID NO: 4) (T137S, L139A, and/or Y180T) can be introduced to a second Fc chain as a hole (heterodimer chain 2). Examples of amino acid sequences of variant canine IgG-A Fc, IgG-B Fc, IgG-C Fc, and IgG-D Fc heterodimer chain 2 are SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, or SEQ ID NO: 93.

An amino acid substitution of threonine to tyrosine or tryptophan at a position corresponding to position 154 of feline IgG1a Fc (SEQ ID NO: 65 or SEQ ID NO: 66), of feline IgG1b Fc (SEQ ID NO: 67 or SEQ ID NO: 68), or of feline IgG2 (SEQ ID NO: 69) (T154Y or T154W) can be introduced to one Fc chain as a knob (heterodimer chain 1). Examples of amino acid sequences of variant feline IgG1a Fc, IgG1b Fc, and IgG2 heterodimer chain 1 are SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, and SEQ ID NO: 103.

An amino acid substitution of threonine to serine at a position corresponding to position 154, and/or of leucine to alanine at a position corresponding to position 156, and/or of tyrosine to threonine at a position corresponding to position 197 of IgG-1a (SEQ ID NO: 65 or SEQ ID NO: 66), or of IgG-1b Fc (SEQ ID NO: 67 or SEQ ID NO: 68), or of IgG2 (SEQ ID NO: 69) (T154S, L156A, and/or Y197T) can be introduced to a second Fc chain as a hole (heterodimer chain 2). Examples of amino acid sequences of variant feline IgG1a Fc, IgG1b Fc, and IgG2 Fc heterodimer chain 2 are SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, and SEQ ID NO: 113.

An amino acid substitution of threonine to tyrosine or tryptophan at a position corresponding to position 131 of equine IgG1 Fc (SEQ ID NO: 49), of equine IgG2 Fc (SEQ ID NO: 50), of equine IgG3 Fc (SEQ ID NO: 52), of equine IgG4 Fc (SEQ ID NO: 53), of equine IgG5 Fc (SEQ ID NO: 54), of equine IgG6 Fc (SEQ ID NO: 55), or of equine IgG7 Fc (SEQ ID NO: 56) (T131Y or T131W) can be introduced to one Fc chain as a knob (heterodimer chain 1). Examples of amino acid sequences of variant IgG1 Fc, IgG2 Fc, IgG3 Fc, IgG4 Fc, IgG5 Fc, IgG6 Fc, and IgG7 Fc heterodimer chain 1 are SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, and SEQ ID NO: 127.

An amino acid substitution of threonine to serine at a position corresponding to position 131 and/or of leucine to alanine at a position corresponding to position 133 and/or of tyrosine to threonine at a position corresponding to position 174 of equine IgG1 Fc (SEQ ID NO: 49), of equine IgG2 Fc (SEQ ID NO: 50), of equine IgG3 Fc (SEQ ID NO: 52), of equine IgG4 Fc (SEQ ID NO: 53), of equine IgG5 Fc (SEQ ID NO: 54), of equine IgG6 Fc (SEQ ID NO: 55), or of equine IgG7 Fc (SEQ ID NO: 56) (T131W, L133A, and/or Y174T) can be introduced to a second Fc chain as a hole (heterodimer chain 2). Examples of amino acid sequences of variant IgG1 Fc, IgG2 Fc, IgG3 Fc, IgG4 Fc, IgG5 Fc, IgG6 Fc, and IgG7 Fc heterodimer chain 2 are SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, and SEQ ID NO: 141.

The pairing of variant canine IgG Fc heterodimer chains 1 and 2, the pairing of variant feline IgG Fc heterodimer chains 1 and 2, and the pairing of variant equine IgG Fc heterodimer chains 1 and 2 may allow for Fc heterodimerization and prevent or reduce Fc homodimerization. A heterodimer chain 1 of one canine IgG subtype may be combined with a heterodimer chain 2 of the same or a different canine IgG subtype. A heterodimer chain 1 of one feline IgG subtype may be combined with a heterodimer chain 2 of the same or a different feline IgG subtype. A heterodimer chain 1 of one equine IgG subtype may be combined with a heterodimer chain 2 of the same or a different equine IgG subtype. The design can enable dimerization of bispecific canine, feline, or equine antibodies. In addition, two different peptides or proteins or a combination of different proteins (e.g., therapeutic proteins) can be fused to the heterodimeric Fc chains.

For example, a dual GLP1 and glucagon molecule can be created using variant canine IgG Fc heterodimer chains or variant feline IgG Fc heterodimer chains, such as a GLP1 polypeptide (e.g., SEQ ID NO: 181) fused to a variant canine IgG Fc heterodimer chain 1 (e.g., SEQ ID NO: 74, 75, 76, 77, 78, 79, 80, or 81) and a glucagon polypeptide (e.g., SEQ ID NO: 182) fused to a variant canine IgG Fc heterodimer chain 2 (e.g., SEQ ID NO: 82, 83, 84, 85, 86, 87, 88, 89, 90, 91, 92, or 93).

Bispecific antibodies combine specificities of two antibodies. To facilitate a heavy chain to specifically pair with its intended light chain, interface amino acids between CH1 and the light chain may be mutated to be complementary in shape and/or charge-charge interaction. An amino acid substitution of alanine to leucine at a position corresponding to position 24 and/or of serine to asparagine at a position corresponding to position 30 of canine IgG-A CH1 (SEQ ID NO: 142), canine IgG-B CH1 (SEQ ID NO: 143), canine IgG-C CH1 (SEQ ID NO: 144), or canine IgG-D CH1 (SEQ ID NO: 145) (A24L and/or S30D) may be introduced. Examples of amino acid sequences of variant canine IgG-A CH1, IgG-B CH1, IgG-C CH1, and IgG-D CH1 are SEQ ID NO: 146, SEQ ID NO: 147, SEQ ID NO: 148, and SEQ ID NO: 149.

An amino acid substitution of phenylalanine to alanine at a position corresponding to position 11 and/or of serine to arginine at a position corresponding to position 22 of a canine κ constant region (SEQ ID NO: 150) (F11A and/or S22R) may be introduced. An example of an amino acid sequence of a variant canine κ constant region is SEQ ID NO: 151.

An amino acid substitution of alanine to leucine at a position corresponding to position 24 and/or of serine to asparagine at a position corresponding to position 30 of feline IgG1 CH1 (SEQ ID NO: 152), or an amino acid substitution of alanine to leucine at a position corresponding to position 24 and/or of serine to asparagine at a position corresponding to position 29 of feline IgG2 CH1 (SEQ ID NO: 153) may be introduced. Examples of amino acid sequences of a variant feline IgG1 CH1 and IgG2 CH1 are SEQ ID NO: 154 and SEQ ID NO: 155.

An amino acid substitution of a phenylalanine to alanine at a position corresponding to position 11 and/or of serine to arginine at a position corresponding to position 22 of a feline κ constant region (SEQ ID NO: 156) (F11A and/or S22R) may be introduced. An example of an amino acid sequence of a variant feline κ constant region is SEQ ID NO: 157.

### Example 6

### Variant IgG Fc Fusion Proteins

Contiguous polypeptides comprising at least one therapeutic polypeptide and/or at least one antibody, and a variant feline, canine, or equine IgG Fc polypeptide described herein (e.g., an IgG Fc having altered C1q, CD16, and/or Protein A binding affinity) may be prepared.

For example, the following constructs may be designed:

TPA1-L1-Fc; Formula (I):

Fc-L1-TPA1; Formula (II):

TPA1-L1-Fc-L2-TPA2; Formula (III):

TPA1-L1-TPA2-L2-Fc; or Formula (IV):

Fc-L1-TPA1-L2-TPA2. Formula (V):

wherein TPA1 is a first therapeutic polypeptide and/or antibody, TPA2 is a second therapeutic polypeptide and/or antibody (e.g., the same therapeutic polypeptide, a different therapeutic polypeptide, the same antibody, or a different antibody), L1 and L2 are optional linkers; and Fc is a variant IgG Fc polypeptide of a companion animal species. Optionally, the contiguous polypeptide comprises a signal sequence. The constructs of Formulas I-V may comprise a TPA3, TPA4, TPA5, etc. following or before any TPA1 or TPA2. TPA3, TPA4, TPA5, etc. are third, fourth, fifth, etc. therapeutic polypeptides and/or antibodies (e.g., the same therapeutic polypeptide, a different therapeutic polypeptide, the same antibody, or a different antibody).

For example, a contiguous polypeptide may comprise a therapeutic polypeptide and a variant feline IgG1a Fc polypeptide (e.g., SEQ ID NO: 70, 71, 94, 95, 99, 100, 104, 105, 106, 107, 154, 167, or 168), a variant feline IgG1b Fc polypeptide (e.g., SEQ ID NO: 72, 73, 96, 97, 101, 102, 108, 109, 110, 111, 154, 169, or 170), or a variant feline IgG2 Fc polypeptide (e.g., SEQ ID NO: 98, 103, 112, 113, 155, 166, 171, or 178) as described herein.

A contiguous polypeptide may comprise a variant canine IgG-A Fc polypeptide (e.g., SEQ ID NO: 7, 10, 35, 36, 37, 38, 39, 40, 41, 74, 78, 82, 86, 90, or 146), a variant canine IgG-B Fc polypeptide (e.g., SEQ ID NO: 13, 15, 16, 17, 18, 19, 20, 21, 22, 31, 32, 75, 79, 83, 87, 91, or 147), a variant canine IgG-C Fc polypeptide (e.g., SEQ ID NO: 8, 11, 14, 23, 24, 25, 26, 27, 28, 29, 30, 33, 34, 76, 80, 84, 88, 92, or 148), or a variant canine IgG-D Fc polypeptide (e.g., SEQ ID NO: 9, 12, 42, 43, 44, 45, 46, 47, 48, 77, 81, 85, 89, 93, or 149) as described herein.

A contiguous polypeptides may comprise a variant equine IgG1Fc polypeptide (e.g., SEQ ID NO: 61, 114, 121, 128, or 135), a variant equine IgG2 Fc polypeptide (e.g., SEQ ID NO: 57, 58, 115, 122, 129, 136, 172, 173, 174, 175, 176, or 177), a variant equine IgG3 Fc polypeptide (e.g., SEQ ID NO: 62, 116, 123, 130, or 137), a variant equine IgG4 Fc polypeptide (e.g., SEQ ID NO: 63, 117, 124, 131, or 138), a variant equine IgG5 Fc polypeptide (e.g., SEQ ID NO: 59, 118, 125, 132, or 139), a variant equine IgG6 Fc polypeptide (e.g., SEQ ID NO: 60, 119, 126, 133, or 140), or a variant equine IgG7 Fc polypeptide (e.g., SEQ ID NO: 64, 120, 127, 134, or 141).

The linker may be a flexible, non-structural linker, such as a glycine- and serine-rich linker. A flexible extension may be added to the C-terminus of the contiguous polypeptide. The extension may comprise a glycine residue (SEQ ID NO: 158), two glycine residues (SEQ ID NO: 159), a three glycine residues (SEQ ID NO: 160), four glycine residues (SEQ ID NO: 161), five glycine residues (SEQ ID NO: 162), six glycine residues (SEQ ID NO: 163), seven glycine residues (SEQ ID NO: 164), eight glycine residues (SEQ ID NO: 165), or more glycine residues.

A contiguous polypeptide may comprise a TPA1, TPA2, TPA3, TPA4, TPA5, etc. or at least one therapeutic polypeptide selected from an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, a glucagon polypeptide, or etc.

A contiguous polypeptide may comprise a TPA1, TPA2, TPA3, TPA4, TPA5, etc. or at least one antibody selected from an antibody that recognizes one or more of the following polypeptides: a NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12β1 polypeptide (e.g., an ECD of an IL12β1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an EPO polypeptide, a somatostatin polypeptide, a GLP1 polypeptide, a glucagon polypeptide, or etc.

### Example 7

### Isolation of Variant IgG Fc Fusion Proteins

Nucleotide sequences encoding contiguous polypeptides comprising at least one therapeutic polypeptide or antibody and a variant feline, canine, or equine IgG Fc polypeptide described herein (e.g., an IgG Fc having altered C1q, CD16, and/or Protein A binding affinity), such as contiguous polypeptides of Formula I, II, III, IV, and/or V may be synthesized and cloned into separate mammalian expression vectors.

The resulting vectors may be separately transfected into CHO cells. For contiguous polypeptides comprising a signal sequence, the supernatant containing the contiguous polypeptides without the signal peptide may be collected and filtered. Contiguous polypeptides comprising an Fc IgG polypeptide having Protein A binding may be affinity purified using a Protein A column (CaptivA® Protein A Affinity Resin, Repligen). Dimerization, aggregation, and/or the presence of sulfide linkage of resultant proteins may be assessed by HPLC gel filtration and/or SDS-PAGE analysis in the absence and presence of reducing agent (DTT).

### Example 8

### Variant IgG Fc Polypeptides for Increased and/or Enhanced Disulfide Formation

Three-dimensional protein modeling analysis of several ortholog hinge structures was used to determine the approximate locations for modifying the feline IgG2 hinge to increase disulfide formation. To increase disulfide formation at the feline IgG2 hinge, the hinge sequence may be modified by substituting an amino acid with cysteine. For example, a variant feline IgG2 Fc (SEQ ID NO: 166) having a modified hinge was prepared by substituting glycine with cysteine at an amino acid position corresponding to position 14 of SEQ ID NO: 69.

Additional three-dimensional protein modeling analysis of several ortholog hinge structures was used to modify feline and equine IgG hinges to enhance disulfide formation. To enhance disulfide formation at the feline IgG hinge, the hinge sequence may be modified by substituting lysine with proline at a position corresponding to position 16 of a wildtype or variant feline IgG1a (SEQ ID NO: 65 or SEQ ID NO: 66), of feline IgG1b (SEQ ID NO: 67 or SEQ ID NO: 68), or of feline IgG2 (SEQ ID NO: 69) (e.g., K16P). Examples of amino acid sequences of variant feline IgG polypeptides having a modified hinge include SEQ ID NO: 167, SEQ ID NO: 168, and SEQ ID NO: 169, SEQ ID NO: 170, and SEQ ID NO: 171.

To enhance disulfide formation at the equine IgG hinge, the hinge sequence may be modified by substituting cysteine with serine at a position corresponding to position 3 of a wildtype or variant equine IgG with a hinge (e.g., IgG2 Fc (SEQ ID NO: 51)) and/or substituting glutamine with proline at a position corresponding to position 20 of an equine IgG with a hinge (e.g., IgG2 Fc (SEQ ID NO: 51) (e.g., C3S and/or Q20P). Examples of amino acid sequences of variant equine IgG polypeptides having a modified hinge include SEQ ID NO: 172, SEQ ID NO: 173, SEQ ID NO: 174, SEQ ID NO: 175, SEQ ID NO: 176, and SEQ ID NO: 177.

The amino acid substitutions described above may be incorporated into the hinge of a wildtype or variant Fc polypeptide described herein.

Three-dimensional protein modeling was used to design feline and equine variant IgG Fc polypeptides comprising sequences from the hinge region from a different IgG isotype for enhanced recombinant production and improved hinge disulfide formation. Variant feline IgG2 Fc polypeptides may be prepared that comprise sequences from the hinge region of feline IgG1a or IgG1b (e.g., SEQ ID NO: 178). In addition, variant equine IgG2 Fc polypeptides may be prepared that comprise sequences from the hinge region of equine IgG1 (e.g., SEQ ID NO: 179 and SEQ ID NO: 180).

Levels of recombinant production of variant IgG Fc polypeptides and/or levels of hinge disulfide formation may be determined and compared to that of another IgG Fc by SDS-PAGE analysis under reducing and non-reducing conditions (e.g., the corresponding wild-type IgG Fc of the same or different isotype, or a wild-type or variant IgG Fc of another companion animal, etc.).

### Example 9

### Exemplary Contiguous Polypeptides Comprising a GLP1 and a Variant Fc Polypeptide

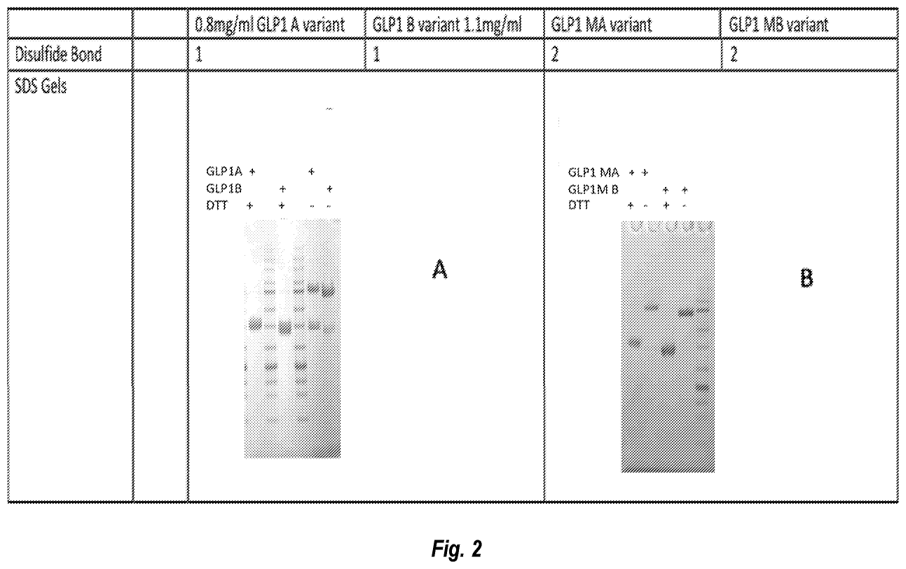

Exemplary contiguous polypeptides comprising a Glucagon-like peptide-1 (GLP1) polypeptide and variant feline IgG Fc with the cysteine hinge modification were designed based on Formula I (ssGLP1-G8_I_VARfeIgG2 (SEQ ID NO: 184)) and Formula III (ssGLP1-G8/GLP1-2G_III_WTfeIgG2 (SEQ ID NO: 185)), expressed in CHO cells, and purified by Protein A chromatography. The amino acid sequences of the secreted proteins after cleavage of the signal sequence are SEQ ID NOs 186 and 187, respectively. The SDS-PAGE analysis of the variant feline IgG2 constructs showed a decrease in the amount of protein in the lower molecular weight band in absence of reducing agent compared to the wild-type feline IgG2 constructs (compare FIG. 2 B to FIG. 2A). These results suggest that the Fc covalent pairing was improved for both variant feline IgG2 constructs.

Furthermore, differential scanning fluorimetry was used to assess the stability of the contiguous polypeptides at various pH, as reflected by mean melting point temperature (n=3) (Table 10, below). The increased stability of the variant feline IgG2 hinge is most evident at pH 6. For example, the constructs having variant feline IgG2 (SEQ ID NOs: 186 and 187) exhibited a higher Tm at pH 6 (56.9 and 59.7° C.) than the corresponding constructs having wild-type feline IgG2 (SEQ ID NOs: 188 and 189), which had a Tm of 55.2 and 56.9° C., respectively.

TABLE 10

| | Mean Melting Point Temperature | | | | | |
|---|---|---|---|---|---|---|
| | (Tm ° C.) (n = 3) | | | | | |
| Construct (10 μg) | pH 3 | pH 4 | pH 5 | pH 6 | pH 7 | pH 8 |
| GLP1-G8/GLP1- | NC | NC | NC | 55.2 | 55.7 | 54.2 |
| 2G_III_WTfeIgG2 | | | | | | |
| (SEQ ID NO: 23) | | | | | | |
| GLP1-G8_I_WTfeIgG2 | NC | NC | 48.5 | 56.9 | 59.9 | 59 |
| (SEQ ID NO: 24) | | | | | | |
| GLP1-G8/GLP1- | NC | NC | NC | 56.9 | 55 | 52.5 |
| 2G_III_VARfeIgG2 | | | | | | |
| (SEQ ID NO: 25) | | | | | | |
| GLP1-G8_I_VARfeIgG2 | NC | NC | 53.1 | 59.7 | 59.9 | 58.2 |
| (SEQ ID NO: 26) | | | | | | |

NC = no curve because no distinct transition point was observed.

### Example 10

### Protein Binding Kinetics

The binding affinity of a contiguous polypeptide described herein to a target molecule may be assessed using biolayer interferometry (Octet). Briefly, a contiguous polypeptide or target molecule that is biotinylated may be captured to streptavidin sensor tips. The association of different concentrations of the second binding partner may be monitored for ninety seconds. Dissociation may be monitored for 600 seconds. A buffer only blank curve may be subtracted to correct for any drift and the data may be fit to a 1:1 binding model using ForteBio™ data analysis software to determine the kₒₙ, k_{off}, and the K_{d}. The buffer for dilutions and all binding steps may be: 20 mM phosphate, 150 mM NaCl, pH 7.2.

### Example 11

### Exemplary Contiguous Polypeptide Comprising an IL13R ECD, an IL4R ECD, and a Variant Canine IgG Fc Polypeptide

Contiguous polypeptide comprising an extracellular domain of IL13 receptor (IL13R ECD; e.g., SEQ ID NO: 190, 191, 192, 193, 194, or 195), an extracellular domain of IL4R (IL4R ECD; e.g., SEQ ID NO: 196, 197, 198, 199, 200, or 201), and a variant IgG Fc polypeptide described herein may be prepared. For example, contiguous polypeptides comprising a canine IL13R ECD of SEQ ID NO: 190, a linker, a canine IL4R ECD of SEQ ID NO: 196, and either a) a wildtype canine IgG-B Fc polypeptide comprising a hinge and the amino acid sequence of SEQ ID NO: 2, or b) a C1q− variant canine IgG-B Fc polypeptide comprising a hinge and the amino acid sequence of SEQ ID NO: 13 were tested (SEQ ID NOs: 271 and 202, respectively).

A biosensor binding analysis was performed to determine the binding affinity of C1q to IL13R(ECD)-IL4R(ECD)-wild type canine IgG-B Fc (SEQ ID NO: 271) compared to IL13R(ECD)-IL4R(ECD)-variant canine IgG-B Fc (SEQ ID NO: 202). Briefly canine IL4 was biotinylated and captured to streptavidin sensor tips. Either IL13R(ECD)-IL4R(ECD)-wild type canine IgG-B Fc (25 μg/mL) or IL13R(ECD)-IL4R(ECD)-variant canine IgG-B Fc (25 μg/mL) was complexed to the IL4-bound biosensors. Subsequently, the complex was used to bind human C1q at 250 μg/mL (Catalog No. 204876-1MG; Sigma Aldrich). The ability of human C1q to bind either complex was measured. Reduced binding between human C1q and IL13R(ECD)-IL4R(ECD)-variant canine IgG-B Fc was observed when compared to IL13R(ECD)-IL4R(ECD)-wild type canine IgG-B Fc.

### Example 12

### Long-Term Stability

Long-term stability of contiguous polypeptides comprising a variant Fc IgG polypeptide described herein may be assessed. For example, samples may be stored in PBS, pH7.2 at different concentrations (e.g., at a concentration of 1 mg/mL, 1.3 mg/mL, 5 mg/mL, and/or 10 mg/mL) at 2-8° C. for a period of time (e.g., one day, six months, and/or one year). To evaluate stability, the stored sample may be analyzed by protein binding assay and/or a cell-based assay.

### Example 13

### Serum Stability

Serum stability of contiguous polypeptides comprising a variant Fc IgG polypeptide described herein may be assessed. For example, samples may be stored in PBS, pH7.2 with serum at a physiological temperature (e.g., 37° C.) for a period of time (e.g., 6 hours, 12 hours, and/or 24 hours) to test in vitro serum stability. To evaluate stability, the stored sample may be analyzed by protein binding assay and/or a cell-based assay.

### Example 14

### In Vivo Pharmacokinetics

In vivo pharmacokinetics of a contiguous polypeptide comprising a variant Fc IgG polypeptide described herein may be assessed after administering a single dose of the contiguous polypeptide to a companion animal by injection (e.g., subcutaneous or intravenous). Serum samples may be taken before dosing (time 0) and at some period(s) of time later (e.g., 4 hours, 8 hours, 12 hours, 24 hours, 48 hours, 72 hours, and/or 168 hours) and the concentration of the contiguous polypeptide measured by quantitative ELISA or other means. The serum concentration of the contiguous polypeptide may be plotted against time and the mean serum half-life (t½), average Tₘₐₓ, the average Cₘₐₓ, and the mean area under the curve (AUC) may be determined.

A quantitative ELISA may use an antibody directed to the therapeutic polypeptide and an HRP-conjugated antibody directed to the IgG-Fc for quantification of the contiguous polypeptide in serum samples from the in vivo pharmacokinetics study. A 96-well plate may be coated with the antibody directed to the therapeutic target (e.g., 5 μg/mL in coating buffer, 100 μl/well). The plate may be sealed and incubated overnight at 4° C. The plate may be washed in triplicate with 1×TBST and blocking buffer added. After removing the blocking buffer, serial dilutions of reference standard and samples in blocking buffer may be added (e.g., 100 μl/well) and the plate incubated for 2 hours at room temperature. The plate may be washed in triplicate with 1×TBST and HRP-conjugated antibody directed to the IgG-Fc added (e.g., 0.1 μg/mL in blocking buffer, 100 μl/well). After incubation for 1 hour at room temperature, the plate may be washed with 1×TBST. TMB substrate (e.g., ScyTek, Catalog No. TM1999) may be added (100 μl/well) and allowed to incubate at room temperature for 1 minute. The reaction may be stopped by the addition of 2M H2504 (e.g., 50 μl/well). Absorbance at 450 nm may be measured and the concentration of the contiguous polypeptide in the serum samples calculated.

Furthermore, the concentration of the contiguous polypeptide in the same serum samples may be assessed using a cell-based activity assay to determine whether the contiguous polypeptide detected by ELISA is biologically active.

1. A polypeptide comprising at least one therapeutic polypeptide and/or at least one antibody, and a variant IgG Fc polypeptide, wherein the variant IgG Fc polypeptide comprises:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide;
c) at least one amino acid modification relative to a wild-type IgG Fc polypeptide of a companion animal species, wherein the variant IgG Fc polypeptide has reduced binding affinity to CD16 relative to the wild-type IgG Fc polypeptide;
d) a hinge region comprising at least one amino acid modification to relative to a wild-type feline or equine IgG Fc polypeptide, wherein the variant IgG Fc polypeptide has increased recombinant production and/or increased hinge disulfide formation relative to the wild-type IgG Fc polypeptide, as determined by SDS-PAGE analysis under reducing and/or nonreducing conditions;
e) at least one amino acid substitution relative to a wild-type feline IgG Fc polypeptide, wherein the at least one amino acid substitution is a cysteine, and wherein the variant IgG Fc polypeptide is capable of forming at least one additional inter-chain disulfide linkage relative to the wild-type feline IgG Fc polypeptide;
f) at least one amino acid substitution relative to a wild-type canine IgG-A or IgG-D Fc polypeptide, wherein the variant IgG Fc polypeptide has increased binding affinity to C1q and/or CD16 relative to the wild-type canine IgG-A or IgG-D Fc polypeptide; and/or
g) a CH1 region comprising at least one amino acid modification relative to a wild-type canine or feline IgG CH1 region, wherein the variant IgG Fc polypeptide comprises:
i) at least one amino acid substitution at a position corresponding to position 24 and/or position 30 of SEQ ID NO: 142, of SEQ ID NO: 143, of SEQ ID NO: 144, or of SEQ ID NO: 145, or
ii) at least one amino acid substitution at a position corresponding to position 24 and/or position 29 of SEQ ID NO: 152 or of SEQ ID NO: 153.

2. A contiguous polypeptide comprising:
i) a first therapeutic polypeptide and/or antibody (TPA1);
ii) a first linker (L1);
iii) a variant Fc polypeptide (Fc) of a companion animal species;
iv) optionally, a second linker (L2); and
v) optionally, a second therapeutic polypeptide and/or antibody (TPA2),
wherein the variant IgG Fc polypeptide comprises:
a) at least one amino acid modification relative to a wild-type IgG Fc polypeptide of a companion animal species, wherein the variant IgG Fc polypeptide has increased binding affinity to Protein A relative to the wild-type IgG Fc polypeptide;
b) at least one amino acid modification relative to a wild-type IgG Fc polypeptide of a companion animal species, wherein the variant IgG Fc polypeptide has reduced binding affinity to C1q relative to the wild-type IgG Fc polypeptide;
c) at least one amino acid modification relative to a wild-type IgG Fc polypeptide of a companion animal species, wherein the variant IgG Fc polypeptide has reduced binding affinity to CD16 relative to the wild-type IgG Fc polypeptide;
d) a hinge region comprising at least one amino acid modification to relative to a wild-type feline or equine IgG Fc polypeptide, wherein the variant IgG Fc polypeptide has increased recombinant production and/or increased hinge disulfide formation relative to the wild-type IgG Fc polypeptide, as determined by SDS-PAGE analysis under reducing and/or nonreducing conditions; and/or
e) at least one amino acid substitution relative to a wild-type feline IgG Fc polypeptide, wherein the at least one amino acid substitution is a cysteine, and wherein the variant IgG Fc polypeptide is capable of forming at least one additional inter-chain disulfide linkage relative to the wild-type feline IgG Fc polypeptide;
f) at least one amino acid substitution relative to a wild-type canine IgG-A or IgG-D Fc polypeptide, wherein the variant IgG Fc polypeptide has increased binding affinity to C1q and/or CD16 relative to the wild-type canine IgG-A or IgG-D Fc polypeptide; and/or
g) a CH1 region comprising at least one amino acid modification relative to a wild-type canine or feline IgG CH1 region, wherein the variant IgG Fc polypeptide comprises:
i) at least one amino acid substitution at a position corresponding to position 24 and/or position 30 of SEQ ID NO: 142, of SEQ ID NO: 143, of SEQ ID NO: 144, or of SEQ ID NO: 145, or
ii) at least one amino acid substitution at a position corresponding to position 24 and/or position 29 of SEQ ID NO: 152 or of SEQ ID NO: 153.

3. The contiguous polypeptide of claim 2 comprising:
TPA1-L1-Fc formula (I):
Fc-L1-TPA1; formula (II):
TPA1-L1-Fc-L2-TPA2; formula (III):
TPA1-L1-TPA2-L2-Fc; or formula (IV):
Fc-L1-TPA1-L2-TPA2. formula (V):

4. A multimeric protein comprising:
i) a first therapeutic polypeptide and/or an antibody (TPA1), and a first variant IgG Fc polypeptide comprising at least one amino acid modification relative to a first wild-type IgG Fc polypeptide, and
ii) a second therapeutic polypeptide and/or an antibody (TPA2), and a second variant IgG Fc polypeptide comprising at least one amino acid modification relative to a second wild-type IgG Fc polypeptide, wherein
a) the first variant IgG Fc polypeptide comprises:
i) an amino acid substitution at a position corresponding to position 138 of SEQ ID NO: 1, position 137 of SEQ ID NO: 2, position 137 of SEQ ID NO: 4, or position 138 of SEQ ID NO: 6;
ii) an amino acid substitution at a position corresponding to position 154 of SEQ ID NO: 65, of SEQ ID NO: 66, of SEQ ID NO: 67, of SEQ ID NO: 68, or of SEQ ID NO: 69; or
iii) an amino acid substitution at a position corresponding to position 131 of SEQ ID NO: 49, of SEQ ID NO: 50, of SEQ ID NO: 52, of SEQ ID NO: 53, of SEQ ID NO: 54, of SEQ ID NO: 55, or of SEQ ID NO: 56; and
b) the second variant IgG Fc polypeptide comprises:
i) an amino acid substitution at a position corresponding to position 138, position 140, and/or position 181 of SEQ ID NO: 1, position 137, position 139, and/or position 180 of SEQ ID NO: 2, position 137, position 139, and/or position 180 of SEQ ID NO: 3, or position 138, position 140, and/or position 181 of SEQ ID NO: 4;
ii) an amino acid substitution at a position corresponding to position 154, position 156, and/or position 197 of SEQ ID NO: 6, of SEQ ID NO: 80, of SEQ ID NO: 81, of SEQ ID NO: 117, or of SEQ ID NO: 118; or
iii) an amino acid substitution at a position corresponding to position 131, position 133, and/or position 174 of SEQ ID NO: 49, of SEQ ID NO: 50, of SEQ ID NO: 52, of SEQ ID NO: 53, of SEQ ID NO: 54, of SEQ ID NO: 55, or of SEQ ID NO: 56.

5. The multimeric protein of claim 4, wherein the first variant IgG Fc polypeptide and/or the second variant IgG Fc polypeptide comprises:
a) at least one amino acid modification relative to a wild-type IgG Fc polypeptide of a companion animal species, wherein the first and/or second variant IgG Fc polypeptide has increased binding affinity to Protein A relative to the wild-type IgG Fc polypeptide;
b) at least one amino acid modification relative to a wild-type IgG Fc polypeptide of a companion animal species, wherein the first and/or second variant IgG Fc polypeptide has reduced binding affinity to C1q relative to the wild-type IgG Fc polypeptide;
c) at least one amino acid modification relative to a wild-type IgG Fc polypeptide of a companion animal species, wherein the first and/or second variant IgG Fc polypeptide has reduced binding affinity to CD16 relative to the wild-type IgG Fc polypeptide;
d) a hinge region comprising at least one amino acid modification to relative to a wild-type feline or equine IgG Fc polypeptide, wherein the first and/or second variant IgG Fc polypeptide has increased recombinant production and/or increased hinge disulfide formation relative to the wild-type IgG Fc polypeptide, as determined by SDS-PAGE analysis under reducing and/or nonreducing conditions;
e) at least one amino acid substitution relative to a wild-type feline IgG Fc polypeptide, wherein the at least one amino acid substitution is a cysteine, and wherein the first and/or second variant IgG Fc polypeptide is capable of forming at least one additional inter-chain disulfide linkage relative to the wild-type feline IgG Fc polypeptide; and/or
f) at least one amino acid substitution relative to a wild-type canine IgG-A or IgG-D Fc polypeptide, wherein the variant IgG Fc polypeptide has increased binding affinity to C1q and/or CD16 relative to the wild-type canine IgG-A or IgG-D Fc polypeptide; and/or
g) a CH1 region comprising at least one amino acid modification relative to a wild-type canine or feline IgG CH1 region, wherein the variant IgG Fc polypeptide comprises:
i) at least one amino acid substitution at a position corresponding to position 24 and/or position 30 of SEQ ID NO: 142, of SEQ ID NO: 143, of SEQ ID NO: 144, or of SEQ ID NO: 145, or
ii) at least one amino acid substitution at a position corresponding to position 24 and/or position 29 of SEQ ID NO: 152 or of SEQ ID NO: 153.

6. The multimeric protein of claim 4 or claim 5, wherein the first wild-type IgG Fc polypeptide and the second wild-type IgG Fc polypeptide are from the same IgG subtype.

7. The multimeric protein of claim 4 or claim 5, wherein the first wild-type IgG Fc polypeptide and the second wild-type IgG Fc polypeptide are from a different IgG subtype.

8. The polypeptide, the contiguous polypeptide, or the multimeric protein of any one of claims 2 to 7, wherein TPA2, if present, comprises a different amino acid sequence compared to TPA1.

9. The polypeptide, the contiguous polypeptide, or the multimeric protein of any one of claims 2 to 8, wherein TPA1 and TPA2 are different therapeutic polypeptides or are antibodies that bind to different targets.

10. The polypeptide, the contiguous polypeptide, or the multimeric protein of any one of the preceding claims, wherein the variant IgG Fc polypeptide binds to C1q and/or CD16 with a dissociation constant (K_{d}) of greater than 5×10⁻⁶ M, greater than 1×10⁻⁵ M, greater than 5×10⁻⁵M, greater than 1×10⁻⁴ M, greater than 5×10⁻⁴ M, or greater than 1×10⁻³M, as measured by biolayer interferometry.

11. The polypeptide, the contiguous polypeptide, or the multimeric protein of any one of the preceding claims, wherein the variant IgG Fc polypeptide binds to Protein A with a dissociation constant (K_{d}) of less than 5×10⁻⁶ M, less than 1×10⁻⁶ M, less than 5×10⁻⁷M, less than 1×10⁻⁷ M, less than 5×10⁻⁸M, less than 1×10⁻⁸ M, less than 5×10⁻⁹M, less than 1×10⁻⁹M, less than 5×10⁻¹⁰ M, less than 1×10⁻¹⁰ M, less than 5×10⁻¹¹M, less than 1×10⁻¹¹M, less than 5×10⁻¹² M, or less than 1×10⁻¹²M, as measured by biolayer interferometry.

12. The polypeptide, the contiguous polypeptide, or the multimeric protein of any one of the preceding claims, wherein the variant canine IgG-A or variant canine IgG-D Fc polypeptide binds to C1q and/or CD16 with a dissociation constant (K_{d}) of less than 5×10⁻⁶ M, less than 1×10⁻⁶ M, less than 5×10⁻⁷M, less than 1×10⁻⁷M, less than 5×10⁻⁸ M, less than 1×10⁻⁸ M, less than 5×10⁻⁹M, less than 1×10⁻⁹ M, less than 5×10⁻¹⁰ M, less than 1×10⁻¹⁰ M, less than 5×10⁻¹¹M, less than 1×10⁻¹¹M, less than 5×10⁻¹²M, or less than 1×10⁻¹²M, as measured by biolayer interferometry.

13. The polypeptide, the contiguous polypeptide, or the multimeric protein of any one of the preceding claims, wherein the companion animal species is canine, feline, or equine.

14. The polypeptide, the contiguous polypeptide, or the multimeric protein of any one of the preceding claims, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1a Fc, IgG1b Fc, or IgG2 Fc.

15. The polypeptide, the contiguous polypeptide, or the multimeric protein of any one of the preceding claims, wherein the variant IgG Fc polypeptide comprises a CH1 region comprising at least one amino acid modification relative to a wild-type canine or feline IgG CH1 region, wherein the variant IgG Fc polypeptide comprises:
a) at least one amino acid substitution at position 24 and/or position 30 of SEQ ID NO: 142, of SEQ ID NO: 143, of SEQ ID NO: 144; or of SEQ ID NO: 145, or
b) at least one amino acid substitution at position 24 and/or position 29 of SEQ ID NO: 152 or of SEQ ID NO: 153.

16. The polypeptide, the contiguous polypeptide, or the multimeric protein of any one of the preceding claims, wherein the variant IgG Fc polypeptide comprises a CH1 region comprising at least one amino acid modification relative to a wild-type canine or feline IgG CH1 region, wherein the variant IgG Fc polypeptide comprises:
a) a leucine at a position corresponding to position 24 and/or an asparagine at a position corresponding to position 30 of SEQ ID NO: 142, of SEQ ID NO: 143, of SEQ ID NO: 144, or of SEQ ID NO: 145; or
b) a leucine at a position corresponding to position 24 and/or an asparagine at a position corresponding to position 29 of SEQ ID NO: 152 or of SEQ ID NO: 153.

17. The polypeptide, the contiguous polypeptide, or the multimeric protein of any one of the preceding claims, wherein the variant IgG Fc polypeptide comprises a CH1 region comprising at least one amino acid modification relative to a wild-type canine or feline IgG CH1 region, wherein the variant IgG Fc polypeptide comprises:
a) a leucine at position 24 and/or an asparagine at position 30 of SEQ ID NO: 142, of SEQ ID NO: 143, of SEQ ID NO: 144, or of SEQ ID NO: 145; or
b) a leucine at position 24 and/or an asparagine at position 29 of SEQ ID NO: 152 or of SEQ ID NO: 153.

18. The polypeptide, the contiguous polypeptide, or the multimeric protein of any one of the preceding claims comprising a wild-type or a variant canine or feline light chain constant region.

19. The polypeptide, the contiguous polypeptide, or the multimeric protein of any one of the preceding claims comprising a wild-type or a variant canine or feline light chain κ constant region.

20. The polypeptide, the contiguous polypeptide, or the multimeric protein of any one of the preceding claims comprising a variant light chain constant region comprising at least one amino acid modification relative to a wild-type canine or feline light chain κ constant region comprising:
a) at least one amino acid substitution at a position corresponding to position 11 and/or position 22 of SEQ ID NO: 150; or
b) at least one amino acid substitution at a position corresponding to position 11 and/or position 22 of SEQ ID NO: 156.

21. The polypeptide, the contiguous polypeptide, or the multimeric protein of any one of the preceding claims comprising a variant light chain constant region comprising at least one amino acid modification relative to a wild-type canine or feline light chain κ constant region comprising:
a) at least one amino acid substitution at a position corresponding to position 11 and/or position 22 of SEQ ID NO: 150; or
b) at least one amino acid substitution at a position corresponding to position 11 and/or position 22 of SEQ ID NO: 156.

22. The polypeptide, the contiguous polypeptide, or the multimeric protein of any one of the preceding claims comprising a variant light chain constant region comprising at least one amino acid modification relative to a wild-type canine or feline light chain κ constant region comprising:
a) an alanine at a position corresponding to position 11 and/or an arginine at a position corresponding to position 22 of SEQ ID NO: 150; or
b) an alanine at a position corresponding to position 11 and/or an arginine at a position corresponding to position 22 of SEQ ID NO: 156.

23. The polypeptide, the contiguous polypeptide, or the multimeric protein of any one of the preceding claims comprising a variant light chain constant region comprising at least one amino acid modification relative to a wild-type canine or feline light chain κ constant region comprising:
a) an alanine at position 11 and/or an arginine at position 22 of SEQ ID NO: 150; or
b) an alanine at position 11 and/or an arginine at position 22 of SEQ ID NO: 156.

24. The polypeptide, the contiguous polypeptide, or the multimeric protein of any one the preceding claims, wherein the variant IgG Fc polypeptide comprises:
a) at least one amino acid substitution relative to a wild-type feline IgG Fc polypeptide, wherein the variant IgG Fc polypeptide comprises an amino acid substitution at a position corresponding to position 16 of SEQ ID NO: 65, SEQ ID NO: 66, SEQ ID NO: 67, SEQ ID NO: 68, or SEQ ID NO: 69;
b) at least one amino acid substitution relative to a wild-type equine IgG Fc polypeptide, wherein the variant IgG Fc polypeptide comprises an amino acid substitution at a position corresponding to position 3 of SEQ ID NO: 51; and/or
c) at least one amino acid substitution relative to a wild-type equine IgG Fc polypeptide, wherein the variant IgG Fc polypeptide comprises an amino acid substitution at a position corresponding to position 20 of SEQ ID NO: 51.

25. A polypeptide comprising a variant IgG Fc polypeptide, wherein the variant IgG Fc polypeptide comprises:
a) at least one amino acid substitution relative to a wild-type feline IgG Fc polypeptide, wherein the variant IgG Fc polypeptide comprises an amino acid substitution at a position corresponding to position 16 of SEQ ID NO: 65, SEQ ID NO: 66, SEQ ID NO: 67, SEQ ID NO: 68, or SEQ ID NO: 69;
b) at least one amino acid substitution relative to a wild-type equine IgG Fc polypeptide, wherein the variant IgG Fc polypeptide comprises an amino acid substitution at a position corresponding to position 3 of SEQ ID NO: 51; and/or
c) at least one amino acid substitution relative to a wild-type equine IgG Fc polypeptide, wherein the variant IgG Fc polypeptide comprises an amino acid substitution at a position corresponding to position 20 of SEQ ID NO: 51.

26. The polypeptide, the contiguous polypeptide, or the multimeric protein of any one the preceding claims, wherein the variant IgG Fc polypeptide comprises:
a) at least one amino acid substitution relative to a wild-type feline IgG Fc polypeptide, wherein the variant IgG Fc polypeptide comprises an amino acid substitution at position 16 of SEQ ID NO: 65, SEQ ID NO: 66, SEQ ID NO: 67, SEQ ID NO: 68, or SEQ ID NO: 69;
b) at least one amino acid substitution relative to a wild-type equine IgG Fc polypeptide, wherein the variant IgG Fc polypeptide comprises an amino acid substitution at position 3 of SEQ ID NO: 51; and/or
c) at least one amino acid substitution relative to a wild-type equine IgG Fc polypeptide, wherein the variant IgG Fc polypeptide comprises an amino acid substitution at position 20 of SEQ ID NO: 51.

27. The polypeptide, the contiguous polypeptide, or the multimeric protein, wherein the variant IgG Fc polypeptide comprises:
a) at least one amino acid substitution relative to a wild-type feline IgG Fc polypeptide, wherein the variant IgG Fc polypeptide comprises a proline at a position corresponding to position 16 or at position 16 of SEQ ID NO: 65, SEQ ID NO: 66, SEQ ID NO: 67, SEQ ID NO: 68, or SEQ ID NO: 69;
b) at least one amino acid substitution relative to a wild-type equine IgG Fc polypeptide, wherein the variant IgG Fc polypeptide comprises a serine at a position corresponding to position 3 or at position 3 of SEQ ID NO: 51; and/or
c) at least one amino acid substitution relative to a wild-type equine IgG Fc polypeptide, wherein the variant IgG Fc polypeptide comprises a proline at a position corresponding to position 20 or at position 20 of SEQ ID NO: 51.

28. The polypeptide, the contiguous polypeptide, or the multimeric protein of any one the preceding claims, wherein the variant IgG Fc polypeptide comprises a hinge region or a portion of a hinge region from an IgG Fc polypeptide of a different isotype.

29. The polypeptide, the contiguous polypeptide, or the multimeric protein of any one of the preceding claims, wherein the variant IgG Fc polypeptide comprises a hinge region or a portion of a hinge region from a wild-type feline IgG-1 Fc polypeptide or from a wild-type equine IgG1 Fc polypeptide.

30. The polypeptide, the contiguous polypeptide, or the multimeric protein of any one of the preceding claims, wherein the variant IgG Fc polypeptide comprises a cysteine at a position corresponding to position 8, position 9, position 10, position 11, position 12, position 13, position 14, position 15, or position 16 of SEQ ID NO: 69.

31. A polypeptide comprising a variant IgG Fc polypeptide, wherein the variant IgG Fc polypeptide comprises a cysteine at a position corresponding to position 8, position 9, position 10, position 11, position 12, position 13, position 14, position 15, or position 16 of SEQ ID NO: 69.

32. The polypeptide, the contiguous polypeptide, or the multimeric protein of any one of the preceding claims, wherein the variant IgG Fc polypeptide comprises a cysteine at a position corresponding to position 14 of SEQ ID NO: 69.

33. The polypeptide, the contiguous polypeptide, or the multimeric protein of any one of the preceding claims, wherein the variant IgG Fc polypeptide comprises a cysteine at position 14 of SEQ ID NO: 69.

34. The polypeptide, the contiguous polypeptide, or the multimeric protein of any one of the preceding claims, wherein the variant IgG Fc polypeptide comprises:
a) an amino acid substitution at a position corresponding to position 21 of SEQ ID NO: 1, an amino acid substitution at a position corresponding to position 23 of SEQ ID NO: 1, an amino acid substitution at a position corresponding to position 25 of SEQ ID NO: 1, an amino acid substitution at a position corresponding to position 80 of SEQ ID NO: 1, an amino acid substitution at a position corresponding to position 205 of SEQ ID NO: 1, and/or an amino acid substitution at a position corresponding to position 207 of SEQ ID NO: 1;
b) an amino acid substitution at a position corresponding to position 21 of SEQ ID NO: 4, an amino acid substitution at a position corresponding to position 23 of SEQ ID NO: 4, and/or an amino acid substitution at a position corresponding to position 24 of SEQ ID NO: 4;
c) an amino acid substitution at a position corresponding to position 21 of SEQ ID NO: 6, an amino acid substitution at a position corresponding to position 23 of SEQ ID NO: 6, an amino acid substitution at a position corresponding to position 25 of SEQ ID NO: 6, an amino acid substitution at a position corresponding to position 80 of SEQ ID NO: 6, and/or an amino acid substitution at a position corresponding to position 207 of SEQ ID NO: 6;
d) an amino acid substitution at a position corresponding to position 15 of SEQ ID NO: 50, and/or an amino acid substitution at a position corresponding to position 203 of SEQ ID NO: 50;
e) an amino acid substitution at a position corresponding to position 199 of SEQ ID NO: 54, and/or an amino acid substitution at a position corresponding to position 200 of SEQ ID NO: 54; and/or
f) an amino acid substitution at a position corresponding to position 199 of SEQ ID NO: 55, an amino acid substitution at a position corresponding to position 200 of SEQ ID NO: 55, an amino acid substitution at a position corresponding to position 201 of SEQ ID NO: 55, and/or an amino acid substitution at a position corresponding to position 202 of SEQ ID NO: 55.

35. A polypeptide comprising a variant IgG Fc polypeptide, wherein the variant IgG Fc polypeptide comprises:
a) an amino acid substitution at a position corresponding to position 21 of SEQ ID NO: 1, an amino acid substitution at a position corresponding to position 23 of SEQ ID NO: 1, an amino acid substitution at a position corresponding to position 25 of SEQ ID NO: 1, an amino acid substitution at a position corresponding to position 80 of SEQ ID NO: 1, an amino acid substitution at a position corresponding to position 205 of SEQ ID NO: 1, and/or an amino acid substitution at a position corresponding to position 207 of SEQ ID NO: 1;
b) an amino acid substitution at a position corresponding to position 21 of SEQ ID NO: 4, an amino acid substitution at a position corresponding to position 23 of SEQ ID NO: 4, and/or an amino acid substitution at a position corresponding to position 24 of SEQ ID NO: 4;
c) an amino acid substitution at a position corresponding to position 21 of SEQ ID NO: 6, an amino acid substitution at a position corresponding to position 23 of SEQ ID NO: 6, an amino acid substitution at a position corresponding to position 25 of SEQ ID NO: 6, an amino acid substitution at a position corresponding to position 80 of SEQ ID NO: 6, and/or an amino acid substitution at a position corresponding to position 207 of SEQ ID NO: 6;
d) an amino acid substitution at a position corresponding to position 15 of SEQ ID NO: 50, and/or an amino acid substitution at a position corresponding to position 203 of SEQ ID NO: 50;
e) an amino acid substitution at a position corresponding to position 199 of SEQ ID NO: 54, and/or an amino acid substitution at a position corresponding to position 200 of SEQ ID NO: 54; and/or
f) an amino acid substitution at a position corresponding to position 199 of SEQ ID NO: 55, an amino acid substitution at a position corresponding to position 200 of SEQ ID NO: 55, an amino acid substitution at a position corresponding to position 201 of SEQ ID NO: 55, and/or an amino acid substitution at a position corresponding to position 202 of SEQ ID NO: 55.

36. The polypeptide, the contiguous polypeptide, or the multimeric protein of any one of the preceding claims, wherein the variant IgG Fc polypeptide comprises:
a) an amino acid substitution at position 21 of SEQ ID NO: 1, an amino acid substitution at position 23 of SEQ ID NO: 1, an amino acid substitution at position 25 of SEQ ID NO: 1, an amino acid substitution at position 80 of SEQ ID NO: 1, an amino acid substitution at position 205 of SEQ ID NO: 1, and/or an amino acid substitution at position 207 of SEQ ID NO: 1;
b) an amino acid substitution at position 21 of SEQ ID NO: 4, an amino acid substitution at position 23 of SEQ ID NO: 4, and/or an amino acid substitution at position 24 of SEQ ID NO: 4;
c) an amino acid substitution at position 21 of SEQ ID NO: 4, an amino acid substitution at position 23 of SEQ ID NO: 6, an amino acid substitution at position 25 of SEQ ID NO: 6, an amino acid substitution at position 80 of SEQ ID NO: 6, and/or an amino acid substitution at position 207 of SEQ ID NO: 6;
d) an amino acid substitution at position 15 of SEQ ID NO: 50, and/or an amino acid substitution at position 203 of SEQ ID NO: 50;
e) an amino acid substitution at position 199 of SEQ ID NO: 54, and/or an amino acid substitution at position 200 of SEQ ID NO: 54; and/or
f) an amino acid substitution at position 199 of SEQ ID NO: 55, an amino acid substitution at position 200 of SEQ ID NO: 55, an amino acid substitution at position 201 of SEQ ID NO: 55, and/or an amino acid substitution at position 202 of SEQ ID NO: 55.

37. The polypeptide, the contiguous polypeptide, or the multimeric protein of any one of the preceding claims, wherein the variant IgG Fc polypeptide comprises:
a) a threonine at a position corresponding to position 21 of SEQ ID NO: 1, a leucine at a position corresponding to position 23 of SEQ ID NO: 1, an alanine at a position corresponding to position 25 of SEQ ID NO: 1, a glycine at a position corresponding to position 80 of SEQ ID NO: 1, an alanine at a position corresponding to position 205 of SEQ ID NO: 1, and/or a histidine at a position corresponding to position 207 of SEQ ID NO: 1;
b) a threonine at a position corresponding to position 21 of SEQ ID NO: 4, a leucine at a position corresponding to position 23 of SEQ ID NO: 4, and/or an isoleucine at a position corresponding to position 24 of SEQ ID NO: 4;
c) a threonine at a position corresponding to position 21 of SEQ ID NO: 6, a leucine at a position corresponding to position 23 of SEQ ID NO: 6, an alanine at a position corresponding to position 25 of SEQ ID NO: 6, a glycine at a position corresponding to position 80 of SEQ ID NO:6, and/or a histidine at a position corresponding to position 207 of SEQ ID NO: 6;
d) a threonine or a valine at a position corresponding to position 15 of SEQ ID NO: 50, and/or a tyrosine or a valine at a position corresponding to position 203 of SEQ ID NO: 50;
e) a leucine at a position corresponding to position 199 of SEQ ID NO: 54, and/or a histidine at a position corresponding to position 200 of SEQ ID NO: 54; and/or
f) a leucine at a position corresponding to position 199 of SEQ ID NO: 55, a histidine at a position corresponding to position 200 of SEQ ID NO: 55, an asparagine at a position corresponding to position 201 of SEQ ID NO: 55, and/or a histidine at a position corresponding to position 202 of SEQ ID NO: 55.

38. The polypeptide, the contiguous polypeptide, or the multimeric protein of any one of the preceding claims, wherein the variant IgG Fc polypeptide comprises:
a) a threonine at position 21 of SEQ ID NO: 1, a leucine at position 23 of SEQ ID NO: 1, an alanine at position 25 of SEQ ID NO: 1, a glycine at position 80 of SEQ ID NO: 1, an alanine at position 205 of SEQ ID NO: 1, and/or a histidine at position 207 of SEQ ID NO: 1;
b) a threonine at position 21 of SEQ ID NO: 3, a leucine at position 23 of SEQ ID NO: 4, and/or an isoleucine at position 24 of SEQ ID NO: 4;
c) a threonine at a position 21 of SEQ ID NO: 6, a leucine at position 23 of SEQ ID NO: 6, an alanine at position 25 of SEQ ID NO: 6, a glycine at position 80 of SEQ ID NO: 6, and/or a histidine at position 207 of SEQ ID NO: 6;
d) a threonine or a valine at position 15 of SEQ ID NO: 50, and/or a tyrosine or a valine at position 203 of SEQ ID NO: 50;
e) a leucine at position 199 of SEQ ID NO: 54, and/or a histidine at position 200 of SEQ ID NO: 54; and/or
f) a leucine at position 199 of SEQ ID NO: 55, a histidine at position 200 of SEQ ID NO: 55, an asparagine at position 201 of SEQ ID NO: 55, and/or a histidine at position 202 of SEQ ID NO: 55.

39. The polypeptide, the contiguous polypeptide, or the multimeric protein of any one of the preceding claims, wherein the variant IgG Fc polypeptide comprises:
a) an amino acid substitution at a position corresponding to position 93 of SEQ ID NO: 2, or an amino acid substitution at a position corresponding to position 93 of SEQ ID NO: 4;
b) an amino acid substitution at a position corresponding to position 87 of SEQ ID NO: 49, an amino acid substitution at a position corresponding to position 87 of SEQ ID NO: 52, an amino acid substitution at a position corresponding to position 87 of SEQ ID NO: 53, or an amino acid substitution at a position corresponding to position 87 of SEQ ID NO: 56; or
c) an amino acid substitution at a position corresponding to position 198 of SEQ ID NO: 65, an amino acid substitution at a position corresponding to position 198 of SEQ ID NO: 66, an amino acid substitution at a position corresponding to position 198 of SEQ ID NO: 67, or an amino acid substitution at a position corresponding to position 198 of SEQ ID NO: 68.

40. A polypeptide comprising a variant IgG Fc polypeptide, wherein the variant IgG Fc polypeptide comprises:
a) an amino acid substitution at a position corresponding to position 93 of SEQ ID NO: 2, or an amino acid substitution at a position corresponding to position 93 of SEQ ID NO: 4;
b) an amino acid substitution at a position corresponding to position 87 of SEQ ID NO: 49, an amino acid substitution at a position corresponding to position 87 of SEQ ID NO: 52, an amino acid substitution at a position corresponding to position 87 of SEQ ID NO: 53, or an amino acid substitution at a position corresponding to position 87 of SEQ ID NO: 56; or
c) an amino acid substitution at a position corresponding to position 198 of SEQ ID NO: 65, an amino acid substitution at a position corresponding to position 198 of SEQ ID NO: 66, an amino acid substitution at a position corresponding to position 198 of SEQ ID NO: 67, or an amino acid substitution at a position corresponding to position 198 of SEQ ID NO: 68.

41. The polypeptide, the contiguous polypeptide, or the multimeric protein of any one of the preceding claims, wherein the variant IgG Fc polypeptide comprises:
a) an amino acid substitution at position 93 of SEQ ID NO: 2, or an amino acid substitution at position 93 of SEQ ID NO: 4;
b) an amino acid substitution at position 87 of SEQ ID NO: 49, an amino acid substitution at position 87 of SEQ ID NO: 52, an amino acid substitution at position 87 of SEQ ID NO: 53, or an amino acid substitution at position 87 of SEQ ID NO: 56; or
c) an amino acid substitution at position 198 of SEQ ID NO: 65, an amino acid substitution at position 198 of SEQ ID NO: 66, an amino acid substitution at position 198 of SEQ ID NO: 67, or an amino acid substitution at position 198 of SEQ ID NO: 68.

42. The polypeptide, the contiguous polypeptide, or the multimeric protein of any one of the preceding claims, wherein the variant IgG Fc polypeptide comprises:
a) an arginine at a position corresponding to position 93 of SEQ ID NO: 2, or an arginine at a position corresponding to position 93 of SEQ ID NO: 4;
b) a serine at a position corresponding to position 87 of SEQ ID NO: 49, a serine substitution at a position corresponding to position 87 of SEQ ID NO: 52, a serine at a position corresponding to position 87 of SEQ ID NO: 53, or a serine at a position corresponding to position 87 of SEQ ID NO: 56; or
c) an alanine at a position corresponding to position 198 of SEQ ID NO: 65, an alanine at a position corresponding to position 198 of SEQ ID NO: 66, an alanine at a position corresponding to position 198 of SEQ ID NO: 67, or an alanine at a position corresponding to position 198 of SEQ ID NO: 68.

43. The polypeptide, the contiguous polypeptide, or the multimeric protein of any one of the preceding claims, wherein the variant IgG Fc polypeptide comprises:
a) an arginine at position 93 of SEQ ID NO: 2, or an arginine at position 93 of SEQ ID NO: 4;
b) a serine at position 87 of SEQ ID NO: 49, a serine at position 87 of SEQ ID NO: 52, a serine at position 87 of SEQ ID NO: 53, or a serine at position 87 of SEQ ID NO: 56; or
c) an alanine at position 198 of SEQ ID NO: 65, an alanine at position 198 of SEQ ID NO: 66, an alanine at position 198 of SEQ ID NO: 67, or alanine at position 198 of SEQ ID NO: 68.

44. The polypeptide, the contiguous polypeptide, or the multimeric protein of any one of the preceding claims, wherein the variant IgG Fc polypeptide comprises:
a) an amino acid substitution at a position corresponding to position 5 of SEQ ID NO: 2, an amino acid substitution at a position corresponding to position 38 of SEQ ID NO: 2, an amino acid substitution at a position corresponding to position 39 of SEQ ID NO: 2, an amino acid substitution at a position corresponding to position 97 of SEQ ID NO: 2, and/or an amino acid substitution at a position corresponding to position 98 of SEQ ID NO: 2; or
b) an amino acid substitution at a position corresponding to position 5 of SEQ ID NO: 4, an amino acid substitution at a position corresponding to position 38 of SEQ ID NO: 4, an amino acid substitution at a position corresponding to position 39 of SEQ ID NO: 4, an amino acid substitution at a position corresponding to position 97 of SEQ ID NO: 4, and/or an amino acid substitution at a position corresponding to position 98 of SEQ ID NO: 4.

45. A polypeptide comprising a variant IgG Fc polypeptide, wherein the variant IgG Fc polypeptide comprises:
a) an amino acid substitution at a position corresponding to position 5 of SEQ ID NO: 2, an amino acid substitution at a position corresponding to position 38 of SEQ ID NO: 2, an amino acid substitution at a position corresponding to position 39 of SEQ ID NO: 2, an amino acid substitution at a position corresponding to position 97 of SEQ ID NO: 2, and/or an amino acid substitution at a position corresponding to position 98 of SEQ ID NO: 2; or
b) an amino acid substitution at a position corresponding to position 5 of SEQ ID NO: 4, an amino acid substitution at a position corresponding to position 38 of SEQ ID NO: 4, an amino acid substitution at a position corresponding to position 39 of SEQ ID NO: 4, an amino acid substitution at a position corresponding to position 97 of SEQ ID NO: 4, and/or an amino acid substitution at a position corresponding to position 98 of SEQ ID NO: 4.

46. The polypeptide, the contiguous polypeptide, or the multimeric protein of any one of the preceding claims, wherein the variant IgG Fc polypeptide comprises:
a) an amino acid substitution at position 5 of SEQ ID NO: 2, an amino acid substitution at position 38 of SEQ ID NO: 2, an amino acid substitution at position 39 of SEQ ID NO: 2, an amino acid substitution at position 97 of SEQ ID NO: 2, and/or an amino acid substitution at position 98 of SEQ ID NO: 2; or
b) an amino acid substitution at position 5 of SEQ ID NO: 4, an amino acid substitution at position 38 of SEQ ID NO: 4, an amino acid substitution at position 39 of SEQ ID NO: 4, an amino acid substitution at position 97 of SEQ ID NO: 4, and/or an amino acid substitution at position 98 of SEQ ID NO: 4.

47. The polypeptide, the contiguous polypeptide, or the multimeric protein of any one of the preceding claims, wherein the variant IgG Fc polypeptide comprises:
a) a proline at a position corresponding to position 5 of SEQ ID NO: 2, a glycine at a position corresponding to position 38 of SEQ ID NO: 2, an arginine at a position corresponding to position 39 of SEQ ID NO: 2, an isoleucine at a position corresponding to position 97 of SEQ ID NO: 2, and/or a glycine at a position corresponding to position 98 of SEQ ID NO: 2; or
b) a proline at a position corresponding to position 5 of SEQ ID NO: 4, a glycine at a position corresponding to position 38 of SEQ ID NO: 4, an arginine at a position corresponding to position 39 of SEQ ID NO: 4, an isoleucine at a position corresponding to position 97 of SEQ ID NO: 4, and/or a glycine at a position corresponding to position 98 of SEQ ID NO: 4.

48. The polypeptide, the contiguous polypeptide, or the multimeric protein of any one of the preceding claims, wherein the variant IgG Fc polypeptide comprises:
a) a proline at position 5 of SEQ ID NO: 2, a glycine at position 38 of SEQ ID NO: 2, an arginine at position 39 of SEQ ID NO: 2, an isoleucine at position 97 of SEQ ID NO: 2, and/or a glycine at position 98 of SEQ ID NO: 2; or
b) a proline at position 5 of SEQ ID NO: 4, a glycine at position 38 of SEQ ID NO: 4, an arginine at position 39 of SEQ ID NO: 4, an isoleucine at position 97 of SEQ ID NO: 4, and/or a glycine at position 98 of SEQ ID NO: 4.

49. The polypeptide, the contiguous polypeptide, or the multimeric protein of any one of the preceding claims comprises:
a) a variant canine IgG-A Fc polypeptide comprising an alanine at a position corresponding to position 2 of SEQ ID NO: 1, a methionine or a lysine at a position corresponding to position 5 of SEQ ID NO: 1, a threonine at a position corresponding to position 21 of SEQ ID NO: 1, a leucine at a position corresponding to position 23 of SEQ ID NO: 1, an alanine at a position corresponding to position 25 of SEQ ID NO: 1, a valine at a position corresponding to position 35 of SEQ ID NO: 1, an asparagine at a position corresponding to position 38 of SEQ ID NO: 1, a proline at a position corresponding to position 39 of SEQ ID NO: 1, a glutamic acid at a position corresponding to position 65 of SEQ ID NO: 1, a glycine at a position corresponding to position 80 of SEQ ID NO: 1, a lysine at a position corresponding to position 93 of SEQ ID NO: 1, a asparagine at a position corresponding to position 96 of SEQ ID NO: 1, a lysine at a position corresponding to position 97 of SEQ ID NO: 1, an alanine at a position corresponding to position 98 of SEQ ID NO: 1, an alanine at a position corresponding to position 205 of SEQ ID NO: 1, and/or a histidine at a position corresponding to position 207 of SEQ ID NO: 1; or
b) a variant canine IgG-D Fc polypeptide comprising an alanine at a position corresponding to position 2 of SEQ ID NO: 6, a methionine or a lysine at a position corresponding to position 5 of SEQ ID NO: 6, a threonine at a position corresponding to position 21 of SEQ ID NO: 6, a leucine at a position corresponding to position 23 of SEQ ID NO: 6, an alanine at a position corresponding to position 25 of SEQ ID NO: 6, a valine at a position corresponding to position 35 of SEQ ID NO: 6, an asparagine at a position corresponding to position 38 of SEQ ID NO: 6, a proline at a position corresponding to position 39 of SEQ ID NO: 6, a glutamic acid at a position corresponding to position 65 of SEQ ID NO: 6, a glycine at a position corresponding to position 80 of SEQ ID NO: 6, a lysine at a position corresponding to position 93 of SEQ ID NO: 6, a asparagine at a position corresponding to position 96 of SEQ ID NO: 6, a lysine at a position corresponding to position 97 of SEQ ID NO: 6, an alanine at a position corresponding to position 98 of SEQ ID NO: 6, and/or a histidine at a position corresponding to position 207 of SEQ ID NO: 6.

50. A polypeptide comprising:
a) a variant canine IgG-A Fc polypeptide comprising an alanine at a position corresponding to position 2 of SEQ ID NO: 1, a methionine or a lysine at a position corresponding to position 5 of SEQ ID NO: 1, a threonine at a position corresponding to position 21 of SEQ ID NO: 1, a leucine at a position corresponding to position 23 of SEQ ID NO: 1, an alanine at a position corresponding to position 25 of SEQ ID NO: 1, a valine at a position corresponding to position 35 of SEQ ID NO: 1, an asparagine at a position corresponding to position 38 of SEQ ID NO: 1, a proline at a position corresponding to position 39 of SEQ ID NO: 1, a glutamic acid at a position corresponding to position 65 of SEQ ID NO: 1, a glycine at a position corresponding to position 80 of SEQ ID NO: 1, a lysine at a position corresponding to position 93 of SEQ ID NO: 1, a asparagine at a position corresponding to position 96 of SEQ ID NO: 1, a lysine at a position corresponding to position 97 of SEQ ID NO: 1, an alanine at a position corresponding to position 98 of SEQ ID NO: 1, an alanine at a position corresponding to position 205 of SEQ ID NO: 1, and/or a histidine at a position corresponding to position 207 of SEQ ID NO: 1; or
b) a variant canine IgG-D Fc polypeptide comprising an alanine at a position corresponding to position 2 of SEQ ID NO: 6, a methionine or a lysine at a position corresponding to position 5 of SEQ ID NO: 6, a threonine at a position corresponding to position 21 of SEQ ID NO: 6, a leucine at a position corresponding to position 23 of SEQ ID NO: 6, an alanine at a position corresponding to position 25 of SEQ ID NO: 6, a valine at a position corresponding to position 35 of SEQ ID NO: 6, an asparagine at a position corresponding to position 38 of SEQ ID NO: 6, a proline at a position corresponding to position 39 of SEQ ID NO: 6, a glutamic acid at a position corresponding to position 65 of SEQ ID NO: 6, a glycine at a position corresponding to position 80 of SEQ ID NO: 6, a lysine at a position corresponding to position 93 of SEQ ID NO: 6, a asparagine at a position corresponding to position 96 of SEQ ID NO: 6, a lysine at a position corresponding to position 97 of SEQ ID NO: 6, an alanine at a position corresponding to position 98 of SEQ ID NO: 6, and/or a histidine at a position corresponding to position 207 of SEQ ID NO: 6.

51. The polypeptide, the contiguous polypeptide, or the multimeric protein of any one of the preceding claims comprises:
a) a variant canine IgG-A Fc polypeptide comprising an alanine at position 2 of SEQ ID NO: 1, a methionine or a lysine at position 5 of SEQ ID NO: 1, a threonine at position 21 of SEQ ID NO: 1, a leucine at position 23 of SEQ ID NO: 1, an alanine at position 25 of SEQ ID NO: 1, a valine at position 35 of SEQ ID NO: 1, an asparagine at position 38 of SEQ ID NO: 1, a proline at position 39 of SEQ ID NO: 1, a glutamic acid at position 65 of SEQ ID NO: 1, a glycine at position 80 of SEQ ID NO: 1, a lysine at position 93 of SEQ ID NO: 1, a asparagine at position 96 of SEQ ID NO: 1, a lysine at position 97 of SEQ ID NO: 1, an alanine at position 98 of SEQ ID NO: 1, an alanine at position 205 of SEQ ID NO: 1, and/or a histidine at position 207 of SEQ ID NO: 1; or
b) a variant canine IgG-D Fc polypeptide comprising an alanine at position 2 of SEQ ID NO: 6, a methionine or a lysine at position 5 of SEQ ID NO: 6, a threonine at position 21 of SEQ ID NO: 6, a leucine at position 23 of SEQ ID NO: 6, an alanine at position 25 of SEQ ID NO: 6, a valine at position 35 of SEQ ID NO: 6, an asparagine at position 38 of SEQ ID NO: 6, a proline at position 39 of SEQ ID NO: 6, a glutamic acid at position 65 of SEQ ID NO: 6, a glycine at position 80 of SEQ ID NO: 6, a lysine at position 93 of SEQ ID NO: 6, a asparagine at position 96 of SEQ ID NO: 6, a lysine at position 97 of SEQ ID NO: 6, an alanine at position 98 of SEQ ID NO: 6, and/or a histidine at position 207 of SEQ ID NO: 6.

52. The polypeptide, the contiguous polypeptide, or the multimeric protein of any one of the preceding claims comprising a variant IgG Fc polypeptide comprising:
a) a tyrosine or a tryptophan at a position corresponding to position 138 of SEQ ID NO: 1, a tyrosine or a tryptophan at a position corresponding to position 137 of SEQ ID NO: 2, a tyrosine or a tryptophan at a position corresponding to position 137 of SEQ ID NO: 4, or a tyrosine or a tryptophan at a position corresponding to position 138 of SEQ ID NO: 6;
b) a tyrosine or a tryptophan at a position corresponding to position 154 of SEQ ID NO: 69, a tyrosine or a tryptophan at a position corresponding to position 154 of SEQ ID NO: 65 or SEQ ID NO: 66, or a tyrosine or a tryptophan at a position corresponding to position 154 of SEQ ID NO: 67 or SEQ ID NO: 68; or
c) a tyrosine or a tryptophan at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56.

53. A polypeptide comprising a variant IgG Fc polypeptide, wherein the variant IgG Fc polypeptide comprises:
a) a tyrosine or a tryptophan at a position corresponding to position 138 of SEQ ID NO: 1, a tyrosine or a tryptophan at a position corresponding to position 137 of SEQ ID NO: 2, a tyrosine or a tryptophan at a position corresponding to position 137 of SEQ ID NO: 4, or a tyrosine or a tryptophan at a position corresponding to position 138 of SEQ ID NO: 6;
b) a tyrosine or a tryptophan at a position corresponding to position 154 of SEQ ID NO: 69, a tyrosine or a tryptophan at a position corresponding to position 154 of SEQ ID NO: 65 or SEQ ID NO: 66, or a tyrosine or a tryptophan at a position corresponding to position 154 of SEQ ID NO: 67 or SEQ ID NO: 68; or
c) a tyrosine or a tryptophan at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56.

54. The polypeptide, the contiguous polypeptide, or the multimeric protein of any one of the preceding claims, wherein the variant IgG Fc polypeptide comprises:
a) a tyrosine or a tryptophan at position 138 of SEQ ID NO: 1, a tyrosine or a tryptophan at position 137 of SEQ ID NO: 2, a tyrosine or a tryptophan at position 137 of SEQ ID NO: 4, or a tyrosine or a tryptophan at position 138 of SEQ ID NO: 6; or
b) a tyrosine or a tryptophan at position 154 of SEQ ID NO: 69, a tyrosine or a tryptophan at position 154 of SEQ ID NO: 65 or SEQ ID NO: 66, or a tyrosine or a tryptophan at position 154 of SEQ ID NO: 67 or SEQ ID NO: 68; and/or
c) a tyrosine or a tryptophan at position 131 of SEQ ID NO: 49, SEQ ID NO: 50, SEQ ID NO: 52, SEQ ID NO: 53, SEQ ID NO: 54, SEQ ID NO: 55, or SEQ ID NO: 56.

55. The polypeptide, the contiguous polypeptide, or the multimeric protein of any one of the preceding claims comprising a variant IgG Fc polypeptide comprising:
a) a serine at a position corresponding to position 138 of SEQ ID NO: 1, a serine at a position corresponding to position 137 of SEQ ID NO: 2, a serine at a position corresponding to position 137 of SEQ ID NO: 4, a serine at a position corresponding to position 138 of SEQ ID NO: 6, a serine at a position corresponding to position 154 of SEQ ID NO: 69, a serine at a position corresponding to position 154 of SEQ ID NO: 65 or SEQ ID NO: 66, a serine at a position corresponding to position 154 of SEQ ID NO: 67 or SEQ ID NO: 68, or a serine at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56;
b) an alanine at a position corresponding to position 140 of SEQ ID NO: 1, an alanine at a position corresponding to position 139 of SEQ ID NO: 2, an alanine at a position corresponding to position 139 of SEQ ID NO: 4, an alanine at a position corresponding to position 140 of SEQ ID NO: 6, an alanine at a position corresponding to position 156 of SEQ ID NO: 69, an alanine at a position corresponding to position 156 of SEQ ID NO: 65 or SEQ ID NO: 66, an alanine at a position corresponding to position 156 of SEQ ID NO: 67 or SEQ ID NO: 68, or an alanine at a position corresponding to position 133 of SEQ ID NO: 49, SEQ ID NO: 50, SEQ ID NO: 52, SEQ ID NO: 53, SEQ ID NO: 54, SEQ ID NO: 55, or SEQ ID NO: 56; and/or
c) a threonine at a position corresponding to position 181 of SEQ ID NO: 1, a threonine at a position corresponding to position 180 of SEQ ID NO: 2, a threonine at a position corresponding to position 180 of SEQ ID NO: 4, a threonine at a position corresponding to position 181 of SEQ ID NO: 6, a threonine at a position corresponding to position 197 of SEQ ID NO: 69, a threonine at a position corresponding to position 197 of SEQ ID NO: 65 or SEQ ID NO: 66, a threonine at a position corresponding to position 197 of SEQ ID NO: 67 or SEQ ID NO: 68, or a threonine at a position corresponding to position 174 of SEQ ID NO: 49, SEQ ID NO: 50, SEQ ID NO: 52, SEQ ID NO: 53, SEQ ID NO: 54, SEQ ID NO: 55, or SEQ ID NO: 56.

56. A polypeptide comprising a variant IgG Fc polypeptide, wherein the variant IgG Fc polypeptide comprises:
a) a serine at a position corresponding to position 138 of SEQ ID NO: 1, a serine at a position corresponding to position 137 of SEQ ID NO: 2, a serine at a position corresponding to position 137 of SEQ ID NO: 4, a serine at a position corresponding to position 138 of SEQ ID NO: 6, a serine at a position corresponding to position 154 of SEQ ID NO: 69, a serine at a position corresponding to position 154 of SEQ ID NO: 65 or SEQ ID NO: 66, a serine at a position corresponding to position 154 of SEQ ID NO: 67 or SEQ ID NO: 68, or a serine at a position corresponding to position 131 of SEQ ID NO: 49, SEQ ID NO: 50, SEQ ID NO: 52, SEQ ID NO: 53, SEQ ID NO: 54, SEQ ID NO: 55, or SEQ ID NO: 56;
b) an alanine at a position corresponding to position 140 of SEQ ID NO: 1, an alanine at a position corresponding to position 139 of SEQ ID NO: 2, an alanine at a position corresponding to position 139 of SEQ ID NO: 4, an alanine at a position corresponding to position 140 of SEQ ID NO: 6, an alanine at a position corresponding to position 156 of SEQ ID NO: 69, an alanine at a position corresponding to position 156 of SEQ ID NO: 65 or SEQ ID NO: 66, an alanine at a position corresponding to position 156 of SEQ ID NO: 67 or SEQ ID NO: 68, or an alanine at a position corresponding to position 133 of SEQ ID NO: 49, SEQ ID NO: 50, SEQ ID NO: 52, SEQ ID NO: 53, SEQ ID NO: 54, SEQ ID NO: 55, or SEQ ID NO: 56; and/or
c) a threonine at a position corresponding to position 181 of SEQ ID NO: 1, a threonine at a position corresponding to position 180 of SEQ ID NO: 2, a threonine at a position corresponding to position 180 of SEQ ID NO: 4, a threonine at a position corresponding to position 181 of SEQ ID NO: 6, a threonine at a position corresponding to position 197 of SEQ ID NO: 69, a threonine at a position corresponding to position 197 of SEQ ID NO: 65 or SEQ ID NO: 66, a threonine at a position corresponding to position 197 of SEQ ID NO: 67 or SEQ ID NO: 68, or a threonine at a position corresponding to position 174 of SEQ ID NO: 49, SEQ ID NO: 50, SEQ ID NO: 52, SEQ ID NO: 53, SEQ ID NO: 54, SEQ ID NO: 55, or SEQ ID NO: 56.

57. The polypeptide, the contiguous polypeptide, or the multimeric protein of any one of the preceding claims, wherein the variant IgG Fc polypeptide comprises:
a) a serine at position 138 of SEQ ID NO: 1, a serine at position 137 of SEQ ID NO: 2, a serine at position 137 of SEQ ID NO: 4, a serine at position 138 of SEQ ID NO: 6, a serine at position 154 of SEQ ID NO: 69, a serine at position 154 of SEQ ID NO: 65 or SEQ ID NO: 66, a serine at position 154 of SEQ ID NO: 67 or SEQ ID NO: 68, or a serine at position 131 of SEQ ID NO: 49, SEQ ID NO: 50, SEQ ID NO: 52, SEQ ID NO: 53, SEQ ID NO: 54, SEQ ID NO: 55, or SEQ ID NO: 56;
b) an alanine at position 140 of SEQ ID NO: 1, an alanine at position 139 of SEQ ID NO: 2, an alanine at position 139 of SEQ ID NO: 4, an alanine at position 140 of SEQ ID NO: 6, an alanine at position 156 of SEQ ID NO: 69, an alanine at position 156 of SEQ ID NO: 65 or SEQ ID NO: 66, an alanine at position 156 of SEQ ID NO: 67 or SEQ ID NO: 68, or an alanine at position 133 of SEQ ID NO: 49, SEQ ID NO: 50, SEQ ID NO: 52, SEQ ID NO: 53, SEQ ID NO: 54, SEQ ID NO: 55, or SEQ ID NO: 56; and/or;
c) a threonine at position 181 of SEQ ID NO: 1, a threonine at position 181 of SEQ ID NO: 2, a threonine at position 181 of SEQ ID NO: 4, a threonine at position 181 of SEQ ID NO: 6, a threonine at position 197 of SEQ ID NO: 69, a threonine at position 197 of SEQ ID NO: 65 or SEQ ID NO: 66, a threonine at position 197 of SEQ ID NO: 67 or SEQ ID NO: 68, or a threonine at position 174 of SEQ ID NO: 49, SEQ ID NO: 50, SEQ ID NO: 52, SEQ ID NO: 53, SEQ ID NO: 54, SEQ ID NO: 55, or SEQ ID NO: 56.

58. The polypeptide, the contiguous polypeptide, or the multimeric protein of any one of the preceding claims, wherein the polypeptide or the variant Fc polypeptide is glycoslylated.

59. The polypeptide, the contiguous polypeptide, or the multimeric protein of any one of the previous claims, wherein the polypeptide or the variant Fc polypeptide is aglycosylated.

60. The polypeptide, the contiguous polypeptide, or the multimeric protein of any one of the preceding claims, wherein L1 and L2, if present, each independently is a flexible linker.

61. The polypeptide, the contiguous polypeptide, or the multimeric protein of any one of the preceding claims, wherein the amino acid sequence of L1 and L2, if present, each independently comprises 100%, at least 95%, at least 90%, at least 85% serine and/or glycine amino acid residues.

62. The polypeptide, the contiguous polypeptide, or the multimeric protein of any one of the preceding claims, wherein the polypeptide, contiguous polypeptide, or multimeric polypeptide comprises an extension at a C-terminus.

63. The polypeptide, the contiguous polypeptide, or the multimeric protein of any one of the preceding claims, wherein the polypeptide, contiguous polypeptide, or multimeric polypeptide comprises a glycine residue, two glycine residues, three glycine residues, four glycine residues, five glycine residues, six glycine residues, seven glycine residues, eight glycine residues, or greater than eight glycine residues at a C-terminus.

64. The polypeptide, the contiguous polypeptide, or the multimeric protein of any one of the previous claims, wherein the contiguous polypeptide comprises an amino acid sequence of SEQ ID NO: 158, SEQ ID NO: 159, SEQ ID NO: 160, SEQ ID NO: 161, SEQ ID NO: 162, SEQ ID NO: 163, SEQ ID NO: 164, or SEQ ID NO: 165 at its C-terminus.

65. The polypeptide, the contiguous polypeptide, or the multimeric protein of any one of the preceding claims, wherein the therapeutic polypeptide, TPA1, and/or TPA2 is selected from an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12β1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ECD of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.

66. The polypeptide, the contiguous polypeptide, or the multimeric protein of any one of the preceding claims, wherein the therapeutic polypeptide, TPA1, and/or TPA2 is a canine polypeptide, a feline polypeptide, or an equine polypeptide.

67. The polypeptide, the contiguous polypeptide, or the multimeric protein of any one of the preceding claims, wherein the antibody, TPA1, and/or TPA2 is an antibody that binds a target polypeptide selected from an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12β1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.

68. The polypeptide, the contiguous polypeptide, or the multimeric protein of any one of the preceding claims, wherein the antibody binds a canine target polypeptide, a feline target polypeptide, or an equine target polypeptide.

69. The polypeptide, the contiguous polypeptide, or the multimeric protein of any one of the preceding claims, wherein the variant IgG Fc polypeptide comprises an amino acid sequence having at least 90% identity, at least 95% identity, at least 97% identity, or at least 99% identity to the amino acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 166, 167, 168, 169, 170, 171, 172, 173, 174, 175, 176, 177, 178, 179, 180, and/or 250.

70. The polypeptide, the contiguous polypeptide, or the multimeric protein of any one of the preceding claims comprising an amino acid sequence of SEQ ID NO: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 57, 58, 59, 60, 61, 62, 63, 64, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 146, 147, 148, 149, 150, 151, 154, 155, 157, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 271, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, and/or 250.

71. A polypeptide comprising an amino acid sequence of SEQ ID NO: 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 57, 58, 59, 60, 61, 62, 63, 64, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 146, 147, 148, 149, 150, 151, 154, 155, 157, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 271, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, and/or 250.

72. The polypeptide, the multimeric protein, or the contiguous polypeptide of any one of the preceding claims, wherein the at least one amino acid modification or substitution comprises an amino acid substitution with an amino acid derivative.

73. An isolated nucleic acid encoding the polypeptide, the multimeric protein, or the contiguous polypeptide of any one of the preceding claims.

74. A host cell comprising the nucleic acid of claim 73.

75. A method of producing a polypeptide comprising culturing the host cell of claim 74 and isolating the polypeptide.

76. A pharmaceutical composition comprising the polypeptide, the multimeric protein, or the contiguous polypeptide of any one of claims 1 to 72, and a pharmaceutically acceptable carrier.

77. A method of exposing a cell to the polypeptide, the multimeric protein, the contiguous polypeptide, or the pharmaceutical composition of any one of claim 1 to 72 or 76.

78. The method of claim 77, wherein the cell is exposed to the polypeptide, heterodimeric protein, contiguous polypeptide, or the pharmaceutical composition ex vivo.

79. The method of claim 77, wherein the cell is exposed to the polypeptide, heterodimeric protein, contiguous polypeptide, or the pharmaceutical composition in vivo.

80. The method of any one of claims 77 to 79, wherein the cell is a human cell, a canine cell, a feline cell, or an equine cell.

81. A method of delivering a polypeptide to a subject comprising administering the polypeptide, the multimeric protein, the contiguous polypeptide, or the pharmaceutical composition of any one of claim 1 to 72 or 76 parenterally.

82. A method of delivering a polypeptide to a subject comprising administering the polypeptide, the multimeric protein, the contiguous polypeptide, or the pharmaceutical composition of any one of claim 1 to 72 or 76 by an intramuscular route, an intraperitoneal route, an intracerebrospinal route, a subcutaneous route, an intra-arterial route, an intrasynovial route, an intrathecal route, or an inhalation route.

83. A method of treating a subject having diabetes or obesity, the method comprising administering to the subject a therapeutically effective amount of the polypeptide, the multimeric protein, the contiguous polypeptide, or the pharmaceutical composition of any one of claim 1 to 72 or 76.

84. The method of any one of claims 81 to 83, wherein the subject is a human subject.

85. The method of any one of claims 81 to 83, wherein the subject is a companion animal species.

86. The method of claim 85, wherein the companion animal species is canine, equine, or feline.
